(19) United States
(12) Patent Application Publication
(10) Pub. No.: US 2019/0375786 A1
(43) Pub. Date: Dec. 12, 2019

(54) STABLE PEPTIDES AND METHODS OF USE THEREOF

(71) Applicants: Fred Hutchinson Cancer Research Center, Seattle, WA (US); Blaze Bioscience, Inc., Seattle, WA (US)

(72) Inventors: Emily June GIRARD, Renton, WA (US); Colin CORRENTI, Seattle, WA (US); James OLSON, Seattle, WA (US); Natalie Winblade NAIRN, Seattle, WA (US); Mesfin Mulugeta GEWE, Bothell, WA (US); Christopher MEHLIN, Seattle, WA (US); Zachary CROOK, Bothell, WA (US); Roland STRONG, Seattle, WA (US); Scott Ronald PRESNELL, Tacoma, WA (US)

(21) Appl. No.: 16/330,069

(22) PCT Filed: Sep. 9, 2017
(86) PCT No.: PCT/US17/50855

§ 371 Date: Mar. 1, 2019

Related U.S. Application Data
(60) Provisional application No. 62/510,710, filed on May 24, 2017.
(60) Provisional application No. 62/447,869, filed on Jan. 18, 2017.
(60) Provisional application No. 62/432,487, filed on Dec. 9, 2016.
(60) Provisional application No. 62/385,908, filed on Sep. 9, 2016.

(51) Int. Cl. C07K 5/10; C12Q 1/37

(52) U.S. Cl. CPC C07K 5/1002; A61K 38/00; C12Q 1/37

(57) ABSTRACT

Peptides that are stable to denaturation and degradation by reducing agents, proteases, temperature, and low pH environments are disclosed. Pharmaceutical compositions and uses for peptides, peptide-active agent conjugates, and peptide-detectable agent conjugates comprising such peptides are also disclosed. Peptide compositions, peptide conjugate compositions, and pharmaceutical compositions can be formulated for various routes of delivery, such as oral delivery, and for deliver to various compartments of the body. Peptides of this disclosure are stable and display enhanced pharmacokinetics after such delivery.

### CROSS REFERENCE

This application is a U.S. National Phase application under 35 U.S.C. § 371 of International Application No. PCT/US2017/050855, filed Sep. 9, 2017, which claims the benefit of U.S. Provisional Patent Application No. 62/385,908, filed Sep. 9, 2016, U.S. Provisional Patent Application No. 62/432,487, filed Dec. 9, 2016, U.S. Provisional Patent Application No. 62/447,869, filed Jan. 18, 2017, and U.S. Provisional Patent Application No. 62/510,710, filed May 24, 2017, the disclosures of which are incorporated herein in their entireties.

### SEQUENCE LISTING

The instant application contains a Sequence Listing which has been submitted electronically in ASCII format and is hereby incorporated by reference in its entirety. Said ASCII copy, created on Sep. 28, 2017, is named 44189-715_601_SL.txt and is 102,056 bytes in size.

### BACKGROUND

Peptides and proteins can be degraded in the body by specific and non-specific mechanisms. Biologic environments that are reducing can lead to unfolding of proteins and peptides by breaking disulfide bridges. Enzymes such as proteases that are prevalent in various organs and cellular compartments can digest peptides and proteins by clipping peptide bonds. Low pH environments in various organs and cellular compartments can promote denaturation of proteins and peptides. As a result, peptide and protein therapeutics face significant stability challenges after administration in vivo. Poor stability of peptide and proteins can lead to diminished pharmacokinetics and reduced efficacy. Moreover, low thermal and solution stability of peptides and proteins can lead to denaturation and/or precipitation, thus resulting in a short shelf-life or requiring special storage methods such as refrigeration.

### SUMMARY

In various aspects the present disclosure provides a method of delivering a peptide to a target tissue, the method comprising: administering the peptide to a subject; and delivering the peptide to the target tissue, wherein the peptide has at least one of the following characteristics: (a) at least 70% the peptide remains intact after exposure to dithiothreitol (DTT) at a concentration of from 10 mM and a temperature of at least 23° C. for at least 30 minutes; (b) at least 70% of the peptide remains intact after exposure to reduced glutathione (GSH) at a concentration of 10 mM and a temperature of at least 23° C. for at least 30 minutes; (c) at least 70% of the peptide remains intact after exposure to trypsin at a concentration of 500 U/ml and a temperature of at least 23° C. for at least 30 minutes; (d) at least 70% of the peptide remains intact after exposure to pepsin at a concentration of 500 U/ml and a temperature of at least 37° C. for at least 30 minutes; (e) at least 70% of the peptide remains intact after exposure to simulated gastric fluid (SGF; pH 1.05; 2% (w/v) sodium chloride in 0.7% (v/v) hydrochloric acid) and a temperature of at least 23° C. for at least 30 minutes; (f) at least 70% of the peptide remains intact after exposure to a pH of 1.05 and a temperature of at least 23° C. for at least 30 minutes; (g) at least 70% of the peptide remains intact after exposure to the combination of simulated gastric fluid (SGF; pH 1.05; 2% (w/v) sodium chloride in 0.7% (v/v) hydrochloric acid) with 500 U/ml pepsin, 100 mM Tris, and 10 mM DTT (SPTD) and a temperature of at least 23° C. for at least 30 minutes; (h) at least 70% of the peptide remains intact after exposure to at least 70° C. for at least 60 minutes; (i) at least 70% of the peptide remains intact after exposure to at least 100° C. for at least 60 minutes; or (j) at least 10% of the peptide remains intact after passage through the mouth, stomach, small intestine, or the large intestine.

In various aspects, the present disclosure provides a method of delivering a peptide to a target tissue, the method comprising: administering the peptide to a subject; and delivering the peptide to the target tissue, wherein the peptide has at least one of the following characteristics: (a) at least 70%, 72%, 75%, 78%, 80%, 82%, 85, 88%, 90%, 92%, 95%, 98%, or 99% of the peptide remains intact after exposure to dithiothreitol (DTT) at a concentration of from 5 mM to 10 mM and a temperature of at least 23° C., 37° C., or 39° C. for at least 5, 10, 15, 20, 30, or 60 minutes; (b) at least 70%, 72%, 75%, 78%, 80%, 82%, 85, 88%, 90%, 92%, 95%, 98%, or 99% of the peptide remains intact after exposure to reduced glutathione (GSH) at a concentration of from 5 mM to 10 mM and a temperature of at least 23° C., 37° C., or 39° C. for at least 5, 10, 15, 20, 30, or 60 minutes; (c) at least 70%, 72%, 75%, 78%, 80%, 82%, 85, 88%, 90%, 92%, 95%, 98%, or 99% of the peptide remains intact after exposure to trypsin at a concentration of 0.5 U/ml to 5000 U/ml and a temperature of at least 23° C., 37° C., or 39° C. for at least 5, 10, 15, 20, 30, or 60 minutes; (d) at least 70%, 72%, 75%, 78%, 80%, 82%, 85, 88%, 90%, 92%, 95%, 98%, or 99% of the peptide remains intact after exposure to pepsin at a concentration of 0.5 U/ml to 5000 U/ml and a temperature of at least 23° C., 37° C., or 39° C. for at least 5, 10, 15, 20, 30, or 60 minutes; (e) at least 70%, 72%, 75%, 78%, 80%, 82%, 85, 88%, 90%, 92%, 95%, 98%, or 99% of the peptide remains intact after exposure to simulated gastric fluid (SGF; pH 1.05; 2% (w/v) sodium chloride in 0.7% (v/v) hydrochloric acid) and a temperature of at least 23° C., 37° C., or 39° C. for at least 5, 10, 15, 20, 30, or 60 minutes; (f) at least 70%, 72%, 75%, 78%, 80%, 82%, 85, 88%, 90%, 92%, 95%, 98%, or 99% of the peptide remains intact after exposure to a pH of from 1-2, 2-3, 3-4, or 4-5 and a temperature of at least 23° C., 37° C., or 39° C. for at least 5, 10, 15, 20, 30, or 60 minutes; (g) at least 70%, 72%, 75%, 78%, 80%, 82%, 85, 88%, 90%, 92%, 95%, 98%, or 99% of the peptide remains intact after exposure to the combination of simulated gastric fluid (SGF; pH 1.05; 2% (w/v) sodium chloride in 0.7% (v/v) hydrochloric acid) with 0.5 U/ml to 5000 U/ml pepsin, 100 mM Tris, and 10 mM DTT and a temperature of at least 23° C., 37° C., or 39° C. for at least 5, 10, 15, 20, 30, or 60 minutes; (h) at least 70%, 72%, 75%, 78%, 80%, 82%, 85, 88%, 90%, 92%, 95%, 98%, or 99% of the peptide remains intact after exposure to at least 70° C. for at least 5, 10, 15, 20, 30, or 60 minutes; (i) at least 1%, 10%, 20%, 30%, 40%, 50%, 60%, 70%, 72%, 75%, 78%, 80%, 82%, 85, 88%, 90%, 92%, 95%, 98%, or 99% of the peptide remains intact after exposure to at least 100° C. for at least 5, 10, 15, 20, 30, or 60 minutes; or (j) at least 70%, 72%, 75%, 78%, 80%, 82%, 85, 88%, 90%, 92%, 95%, 98%, or 99% of the peptide remains intact after passage through the mouth, stomach, small intestine, or the large intestine.

In some aspects, the peptide has two or more of the characteristics (a) through (j). In further aspects, the peptide has three or more of the characteristics (a) through (j). In still further aspects, the peptide has four or more of the characteristics (a) through (j). In still further aspects, the peptide has five or more of the characteristics (a) through (j). In still further aspects, the peptide has six or more of the characteristics (a) through (j). In still further aspects, the peptide has seven or more of the characteristics (a) through (j). In still further aspects, the peptide has eight or more of the characteristics (a) through (j). In still further aspects, the peptide has all of the characteristics (a) through (j).

In some aspects, the peptide remains intact after exposure to at least 75° C. for at least 5, 10, 15, 20, 30, or 60 minutes. In some aspects, the peptide comprises a motif, and wherein the motif comprises Cys-X_{[0-15}]-Cys-X_{[0-15]}-Cys-X_{[0-15]}-Cys-X_{[0-15]}-Cys-X_{[0-15}]-Cys (SEQ ID NO: 179), wherein X is any amino acid. In further aspects, X is any amino acid or absent. In some aspects, the peptide is a knotted peptide. In some aspects, the peptide comprises 6 or more cysteine residues.

In further aspects, the peptide comprises three or more disulfide bridges formed between cysteine residues, wherein one of the disulfide bridges passes through a loop formed by two other disulfide bridges. In some aspects, the peptide comprises a plurality of disulfide bridges. In some aspects, the peptide is a cystine-dense peptide (CDP). In further aspects, the CDP comprises independent folding domains, wherein the independent folding domains comprise a high density of at least six cysteines.

In some aspects, the CDP is exported to the cell surface or secreted. In some aspects, the CDP comprises a disulfide bond between cysteines 1 and 4, 2 and 5, and 3 and 6. In other aspects, the CDP comprises a disulfide bond between cysteines 1 and 3, 2 and 5, and 4 and 6. In still other aspects, the CDP comprises a disulfide bond between cysteines 1 and 4, 2 and 6, and 3 and 5.

In other aspects, the CDP comprises a disulfide bond between cysteines 1 and 5, 2 and 4, and 3 and 6. In still other aspects, the CDP comprises a disulfide bond between cysteines 1 and 6, 2 and 4, and 3 and 5.

In other aspects, the CDP is a non-knotted CDP. In some aspects, the non-knotted CDP comprises a disulfide bond between cysteines 1 and 6, 2 and 5, and 3 and 4. In some aspects, the peptide comprises a topology of a Cysu-Cysv disulfide bond, a Cysw-Cysx disulfide bond, and a Cysy-Cysz disulfide bond, wherein the Cysw-Cysx disulfide bond passes through a macrocycle comprising the Cysu-Cysv disulfide bondand the Cysy-Cysz disulfide bond. In some aspects, the Cysw-Cysx cysteine-cysteine bond is a knotting cysteine.

In some aspects, the peptide is a hitchin, and wherein the hitchin comprises a topology wherein the Cysu-Cysy disulfide bond is between cysteine 1 and cysteine 4, the Cysw-Cysx disulfide bond is between cysteine 2 and cysteine 5, and wherein the Cysy-Cyszdisulfide bond is between cysteine 3 and cysteine 6.

In some aspects, at least one amino acid residue of the peptide is in an L configuration or, wherein at least one amino acid residue of the knotted peptide is in a D configuration. In some aspects, the peptide is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues long.

In some aspects, any one or more K residues are replaced by an R residue or wherein any one or more R residues are replaced by for a K residue. In further aspects, the peptide is arranged in a multimeric structure with at least one other peptide. In some aspects, the peptide comprises any one of SEQ ID NO: 167-SEQ ID NO: 171. In other aspects, the peptide comprises any one of any one of SEQ ID NO: 172-SEQ ID NO: 176.

In some aspects, the peptide comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 1-SEQ ID NO: 83. In further aspects, the peptide comprises any one of SEQ ID NO: 1-SEQ ID NO: 83.

In other aspects, the peptide comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 84-SEQ ID NO: 166. In further aspects, the peptide comprises any one of SEQ ID NO: 84-SEQ ID NO: 166.

In some aspects, the peptide comprises any one of SEQ ID NO: 31, SEQ ID NO: 27, SEQ ID NO: 24, SEQ ID NO: 6, SEQ ID NO: 10, SEQ ID NO: 12, SEQ ID NO: 57, SEQ ID NO: 77, or SEQ ID NO: 78. In some aspects, the peptide comprises any one of SEQ ID NO: 27, SEQ ID NO: 31, or SEQ ID NO: 57. In some aspects, the peptide comprises any one of SEQ ID NO: 29, SEQ ID NO: 4, SEQ ID NO: 79, or SEQ ID NO: 80. In other aspects, the peptide comprises any one of SEQ ID NO: 26, SEQ ID NO: 81, SEQ ID NO: 82, or SEQ ID NO: 83.

In some aspects, the peptide comprises any one of SEQ ID NO: 2. In other aspects, the peptide comprises any one of SEQ ID NO: 31.

In some aspects, the peptide exhibits an average Tmax of 0.5-12 hours at which the Cmax is reached. In some aspects, the peptide achieves an average bioavailability of the peptide in serum of 0.1%-10% after administering the peptide to the subject by an oral route. In other aspects, the peptide achieves an average bioavailability of the peptide in serum of less than 0.1% after administering the peptide to the subject by an oral route

In other aspects, the peptide achieves an average bioavailability of the peptide in serum of 10%-100% after administering the peptide to a subject by a parenteral route. In some aspects, the peptide achieves an average t½ of 0.1 hours-168 hours in a subject after administering the peptide to the subject. In some aspects, the peptide achieves an average clearance (CL) of 0.5-100 L/hour of the peptide after administering the peptide to a subject. In some aspects, the peptide achieves an average volume of distribution (Vd) of 200-20,000 mL in the subject after administering the peptide to the subject.

In some aspects, the peptide remains intact after exposure to oxidative conditions for 30 minutes. In some aspects, the peptide remains intact after exposure to a pH less than 2 for 30 minutes. In some aspects, the peptide remains intact after passage through the gastrointestinal tract. In some aspects, the peptide remains intact after exposure to Tris(2-carboxyethyl)phosphine HCl (TCEP), or 2-Mercaptoethanol.

In some aspects, the peptide remains intact after exposure to chymotrypsin, serum protease, serine protease, cysteinyl protease, aspartyl protease, elastase, matrix metalloproteases, cytochrome P450 enzymes, carboxypeptidases, or cathepsins. In some aspects, 90-100% of the peptide remains intact after exposure to a temperature of at least 25° C., 30° C., or 40° C. with at least 60%, 65% or 75% relative humidity for at least 3, 6, 12, 18, 24, 36, or 48 months.

In some aspects, the peptide exhibits the characteristics after oral administration, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof, to a subject.

In further aspects, the subject is a human. In still further aspects, the subject is a non-human animal. In some aspects, at least one residue of the peptide comprises a chemical modification. In further aspects, the chemical modification is blocking the N-terminus of the peptide. In still further aspects, the chemical modification is methylation, acetylation, or acylation.

In some aspects, the chemical modification comprises methylation of one or more lysine residues or analogue thereof, methylation of the N-terminus, or methylation of one or more lysine residue or analogue thereof and methylation of the N-terminus. In some aspects, the peptide is linked to an acyl adduct.

In some aspects, the peptide is linked to an active agent. In some aspects, the active agent is fused with the peptide at an N-terminus or a C-terminus of the peptide. In further aspects, the active agent is an Fc domain. In still further aspects, the peptide fused with an Fc domain comprises a contiguous sequence.

In further aspects, 1, 2, 3, 4, 5, 6, 7, 8, 9, or 10 active agents are linked to the peptide. In some aspects, the peptide is linked to the active agent via a cleavable linker. In some aspects, the peptide is linked to the active agent at an N-terminus, at the epsilon amine of an internal lysine residue, at the carboxylic acid of an aspartic acid, or glutamic acid residue, or a C-terminus of the peptide by a linker.

In some aspects, the peptide further comprises a non-natural amino acid, wherein the non-natural amino acid is an insertion, appendage, or substitution for another amino acid. In some aspects, the peptide is linked to the active agent at the non-natural amino acid by a linker. In some aspects, the linker comprises an amide bond, an ester bond, a carbamate bond, a carbonate bond, a hydrazone bond, an oxime bond, a disulfide bond, a thioester bond, a thioether bond, or a carbon-nitrogen bond. In some aspects, the cleavable linker comprises a cleavage site for matrix metalloproteinases, thrombin, cathepsins, or beta-glucuronidase.

In other aspects, the peptide is linked to the active agent via a noncleavable linker. In some aspects, the active agent is: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, an antibody fragment, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic agent, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, a glucocorticoid, an anti-cytokine agent, a pain-reducing agent, a dendrimer, a fatty acid, an Fc region, siderocalin, or a combination thereof.

In some aspects, the peptide is linked to a detectable agent. In some aspects, the detectable agent is fused with the peptide at an N-terminus or a C-terminus of the peptide. In further aspects, 1, 2, 3, 4, 5, 6, 7, 8, 9, or 10 detectable agents are linked to the peptide.

In some aspects, the peptide is linked to the detectable agent via a cleavable linker. In some aspects, the peptide is linked to the detectable agent at an N-terminus, at the epsilon amine of an internal lysine residue, at the carboxylic acid of an internal aspartic acid or glutamic acid residue, or a C-terminus of the peptide by a linker. In some aspects, the peptide is linked to the detectable agent at the non-natural amino acid by a linker. In some aspects, the linker comprises an amide bond, an ester bond, a carbamate bond, a hydrazone bond, an oxime bond, a thioether bond, a thioester bond, or a carbon-nitrogen bond.

In further aspects, the cleavable linker comprises a cleavage site for matrix metalloproteinases, thrombin, cathepsins, or beta-glucuronidase.

In some aspects, the peptide is linked to the detectable agent via a noncleavable linker. In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye.

In some aspects, the peptide is administered orally. In some aspects, the subject has a condition.

In further aspects, the condition is a gastrointestinal infection or chronic gastrointestinal disease. In some aspects, the gastrointestinal infection is a bacterial infection, prokaryotic infection, or fungal infection. In some aspects, the chronic gastrointestinal disease is irritable bowel sydrome, inflammatory bowel disease, Crohn's disease, gastroesphageal reflux disease, ulcerative colitis or constipation.

In other aspects, the condition is cancer. In some aspects, the cancer is colorectal cancer, stomach cancer, or esophageal cancer.

In some aspects, the peptide is administered to treat the condition. In further aspects, the condition is an inflammation, a cancer, a degradation, a growth disturbance, genetic, a tear, an infection, an injury, a rheumatic condition, an immune system disorder, a kidney disease, lung disease, a condition of aging, a degenerative brain condition, a degenerative body condition, a childhood condition, a hepatic disease, a pulmonary disease, a pancreatic condition, or a gastrointestinal condition. In some aspects, the kidney disease is acute kidney injury or chronic kidney disease. In some aspects, the peptide is delivered by oral administration to treat a gastrointestinal condition.

In other aspects, the peptide is delivered by oral administration to treat a non-gastrointestinal condition. In some aspects, the peptide is delivered by oral administration and homes to cartilage. In other aspects, the peptide is delivered by oral administration and homes to kidneys or proximal tubules of the kidneys. In some aspects, the peptide is delivered by oral administration and homes to or accumulates in tumors. In some aspects, the peptide is administered to detect a diseased region, tissue, structure, or cell.

In some aspects, the peptide enters the cell. In some aspects, the peptide is active intracellularly. In some aspects, after the administering, one of the following characteristics of the composition is measured in the subject: (a) an intact peptide or fragment thereof, in plasma; (b) the intact peptide or fragment thereof, in the stomach; (c) the intact peptide or fragment thereof, in the gastrointestinal tract; (d) the intact peptide or fragment thereof, in the colon; (e) the intact peptide or fragment thereof, in the feces; (f) the intact peptide or fragment thereof, in the urine; (g) the intact peptide or fragment thereof, in cartilage; (h) an average Cmax of the intact peptide or fragment thereof, in plasma; (i) an average Tmax at which the Cmax is reached; (j) an average area under the curve (AUC) of the intact peptide or fragment thereof in the subject; (k) an average bioavailability of the intact peptide or fragment thereof in the subject; (1) an average t½ of the intact peptide or fragment thereof in the subject; (m) an average clearance (CL) of the intact peptide or fragment thereof in the subject; or (n) an average volume of distribution (Vd) of the intact peptide or fragment thereof in the subject.

In various aspects, the present disclosure provides a peptide having at least one of the following characteristics: (a) at least 70% the peptide remains intact after exposure to dithiothreitol (DTT) at a concentration of from 10 mM and a temperature of at least 23° C. for at least 30 minutes; (b) at least 70% of the peptide remains intact after exposure to reduced glutathione (GSH) at a concentration of from 10 mM and a temperature of at least 23° C. for at least 30 minutes; (c) at least 70% of the peptide remains intact after exposure to trypsin at a concentration of 500 U/ml and a temperature of at least 23° C. for at least 30 minutes; (d) at least 70% of the peptide remains intact after exposure to pepsin at a concentration of 500 U/ml and a temperature of at least 37° C. for at least 30 minutes; (e) at least 70% of the peptide remains intact after exposure to simulated gastric fluid (SGF; pH 1.05; 2% (w/v) sodium chloride in 0.7% (v/v) hydrochloric acid) and a temperature of at least 23° C. for at least 30 minutes; (f) at least 70% of the peptide remains intact after exposure to a pH of 1.05 and a temperature of at least 23° C. for at least 30 minutes; (g) at least 70% of the peptide remains intact after exposure to the combination of simulated gastric fluid (SGF; pH 1.05; 2% (w/v) sodium chloride in 0.7% (v/v) hydrochloric acid) with 500 U/ml pepsin, 100 mM Tris, and 10 mM DTT (SPTD) and a temperature of at least 23° C. for at least 30 minutes; (h) at least 70% of the peptide remains intact after exposure to at least 70° C. for at least 60 minutes; (i) at least 70% of the peptide remains intact after exposure to at least 100° C. for at least 60 minutes; or (j) at least 10% of the peptide remains intact after passage through the mouth, stomach, small intestine, or the large intestine.

In various aspects, the present disclosure provides a peptide having at least one of the following characteristics: (a) at least 70%, 72%, 75%, 78%, 80%, 82%, 85, 88%, 90%, 92%, 95%, 98%, or 99% of the peptide remains intact after exposure to dithiothreitol (DTT) at a concentration of from 5 mM to 10 mM and a temperature of at least 23° C., 37° C., or 39° C. for at least 5, 10, 15, 20, 30, or 60 minutes; (b) at least 70%, 72%, 75%, 78%, 80%, 82%, 85, 88%, 90%, 92%, 95%, 98%, or 99% of the peptide remains intact after exposure to reduced glutathione (GSH) at a concentration of from 5 mM to 10 mM and a temperature of at least 23° C., 37° C., or 39° C. for at least 5, 10, 15, 20, 30, or 60 minutes; (c) at least 70%, 72%, 75%, 78%, 80%, 82%, 85, 88%, 90%, 92%, 95%, 98%, or 99% of the peptide remains intact after exposure to trypsin at a concentration of 0.5 U/ml to 5000 U/ml a temperature of at least 23° C., 37° C., or 39° C. for at least 5, 10, 15, 20, 30, or 60 minutes; (d) at least 70%, 72%, 75%, 78%, 80%, 82%, 85, 88%, 90%, 92%, 95%, 98%, or 99% of the peptide remains intact after exposure to pepsin at a concentration of 0.5 U/ml to 5000 U/ml and a temperature of at least 23° C., 37° C., or 39° C. for at least 5, 10, 15, 20, 30, or 60 minutes; (e) at least 70%, 72%, 75%, 78%, 80%, 82%, 85, 88%, 90%, 92%, 95%, 98%, or 99% of the peptide remains intact after exposure to simulated gastric fluid (SGF; pH 1.05; 2% (w/v) sodium chloride in 0.7% (v/v) hydrochloric acid) and a temperature of at least 23° C., 37° C., or 39° C. for at least 5, 10, 15, 20, 30, or 60 minutes; (f) at least 70%, 72%, 75%, 78%, 80%, 82%, 85, 88%, 90%, 92%, 95%, 98%, or 99% of the peptide remains intact after exposure to a pH of from 1-2, 2-3, 3-4, or 4-5 and a temperature of at least 23° C., 37° C., or 39° C. for at least 5, 10, 15, 20, 30, or 60 minutes; (g) at least 70%, 72%, 75%, 78%, 80%, 82%, 85, 88%, 90%, 92%, 95%, 98%, or 99% of the peptide remains intact after exposure to the combination of simulated gastric fluid (SGF; pH 1.05; 2% (w/v) sodium chloride in 0.7% (v/v) hydrochloric acid) with 0.5 U/ml to 5000 U/ml pepsin, 100 mM Tris, and 10 mM DTT (SPTD) and a temperature of at least 23° C., 37° C., or 39° C. for at least 5, 10, 15, 20, 30, or 60 minutes; (h) at least 70%, 72%, 75%, 78%, 80%, 82%, 85, 88%, 90%, 92%, 95%, 98%, or 99% of the peptide remains intact after exposure to at least 70° C. for at least 5, 10, 15, 20, 30, or 60 minutes; (i) at least 70%, 72%, 75%, 78%, 80%, 82%, 85, 88%, 90%, 92%, 95%, 98%, or 99% of the peptide remains intact after exposure to at least 100° C. for at least 5, 10, 15, 20, 30, or 60 minutes; or (j) at least 1%, 10%, 20%, 30%, 40%, 50%, 60%, 70%, 72%, 75%, 78%, 80%, 82%, 85, 88%, 90%, 92%, 95%, 98%, or 99% of the peptide remains intact after passage through the mouth, stomach, small intestine, or the large intestine.

In some aspects, the peptide is a non-naturally occurring peptide. In some aspects, the peptide has two or more of the characteristics (a) through (j). In further aspects, the peptide has three or more of the characteristics (a) through (j). In still further aspects, the peptide has four or more of the characteristics (a) through (j). In still further aspects, the peptide has five or more of the characteristics (a) through (j). In still further aspects, the peptide has six or more of the characteristics (a) through (j). In still further aspects, the peptide has seven or more of the characteristics (a) through (j). In still further aspects, the peptide has eight or more of the characteristics (a) through (j). In still further aspects, the peptide has all of the characteristics (a) through (j).

In some aspects, the peptide remains intact after exposure to at least 75° C. for at least 5, 10, 15, 20, 30, or 60 minutes. In some aspects, the peptide comprises a motif, and wherein the motif comprises Cys-X_{[0-15]}-Cys-X_{[0-15]}-Cys-X_{[0-15]}-Cys-X_{[0-15]}-Cys-X_{[0-15]}-Cys (SEQ ID NO: 179), wherein X is any amino acid. In further aspects, X is any amino acid or absent. In some aspects, the peptide is a knotted peptide. In some aspects, the peptide comprises 6 or more cysteine residues.

In further aspects, the peptide comprises three or more disulfide bridges formed between cysteine residues, wherein one of the disulfide bridges passes through a loop formed by two other disulfide bridges. In some aspects, the peptide comprises a plurality of disulfide bridges. In some aspects, the peptide is a cystine-dense peptide (CDP). In some aspects, the CDP comprises independent folding domains, wherein the independent folding domains comprise a high density of at least six cysteines.

In some aspects, the CDP is exported to the cell surface or secreted. In some aspects, the CDP comprises a disulfide bond between cysteines 1 and 4, 2 and 5, and 3 and 6. In other aspects, the CDP comprises a disulfide bond between cysteines 1 and 3, 2 and 5, and 4 and 6. In still other aspects, the CDP comprises a disulfide bond between cysteines 1 and 4, 2 and 6, and 3 and 5.

In other aspects, the CDP comprises a disulfide bond between cysteines 1 and 5, 2 and 4, and 3 and 6. In still other aspects, the CDP comprises a disulfide bond between cysteines 1 and 6, 2 and 4, and 3 and 5.

In other aspects, the CDP is a non-knotted CDP. In some aspects, the non-knotted CDP comprises a disulfide bond between cysteines 1 and 6, 2 and 5, and 3 and 4. In some aspects, the peptide comprises a topology of a Cysu-Cysv disulfide bond, a Cysw-Cysx disulfide bond, and a Cysy-Cysz disulfide bond, wherein the Cysw-Cysx disulfide bond passes through a macrocycle comprising the Cysu-Cysv disulfide bond and the Cysy-Cysz disulfide bond. In some aspects, the Cysw-Cysx cysteine-cysteine bond is a knotting cysteine.

In some aspects, the peptide is a hitchin, and wherein the hitchin comprises a topology wherein the Cysu-Cysy disulfide bond is between cysteine 1 and cysteine 4, the Cysw-Cysx disulfide bond is between cysteine 2 and cysteine 5, and wherein the Cysy-Cyszdisulfide bond is between cysteine 3 and cysteine 6.

In some aspects, at least one amino acid residue of the peptide is in an L configuration or, wherein at least one amino acid residue of the knotted peptide is in a D configuration. In some aspects, the peptide is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues long.

In some aspects, any one or more K residues are replaced by an R residue or wherein any one or more R residues are replaced by for a K residue. In some aspects, the peptide is arranged in a multimeric structure with at least one other peptide.

In some aspects, the peptide comprises any one of SEQ ID NO: 167-SEQ ID NO: 171. In other aspects, the peptide comprises any one of any one of SEQ ID NO: 172-SEQ ID NO: 176.

In still other aspects, the peptide comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 1-SEQ ID NO: 83. In further aspects, the peptide comprises any one of SEQ ID NO: 1-SEQ ID NO: 83.

In some aspects, the peptide comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 84-SEQ ID NO: 166. In further aspects, the peptide comprises any one of SEQ ID NO: 84-SEQ ID NO: 166.

In other aspects, the peptide comprises any one of SEQ ID NO: 31, SEQ ID NO: 27, SEQ ID NO: 24, SEQ ID NO: 6, SEQ ID NO: 10, SEQ ID NO: 12, SEQ ID NO: 57, SEQ ID NO: 77, or SEQ ID NO: 78. In other aspects, the peptide comprises any one of SEQ ID NO: 27, SEQ ID NO: 31, or SEQ ID NO: 57. In some aspects, the peptide comprises any one of SEQ ID NO: 29, SEQ ID NO: 4, SEQ ID NO: 79, or SEQ ID NO: 80. In some aspects, the peptide comprises any one of SEQ ID NO: 26, SEQ ID NO: 81, SEQ ID NO: 82, or SEQ ID NO: 83.

In some aspects, the peptide comprises any one of SEQ ID NO: 2. In other aspects, the peptide comprises any one of SEQ ID NO: 31.

In some aspects, the peptide exhibits an average Tmax of 0.5-12 hours at which the Cmax is reached. In some aspects, the peptide achieves an average bioavailability of the peptide in serum of 0.1%-10% after administering the peptide to the subject by an oral route. In other aspects, the peptide achieves an average bioavailability of the peptide in serum of less than 0.1% after administering the peptide to the subject by an oral route. In still other aspects, the peptide achieves an average bioavailability of the peptide in serum of 10%-100% after administering the peptide to a subject by a parenteral route.

In some aspects, the peptide achieves an average t½ of 0.1 hours-168 hours in a subject after administering the peptide to the subject. In some aspects, the peptide achieves an average clearance (CL) of 0.5-100 L/hour of the peptide after administering the peptide to a subject. In some aspects, the peptide achieves an average volume of distribution (Vd) of 200-20,000 mL in the subject after administering the peptide to the subject.

In some aspects, the peptide remains intact after exposure to oxidative conditions for 30 minutes. In some aspects, the peptide remains intact after exposure to a pH less than 2 for 30 minutes. In further aspects, the peptide remains intact after passage through the gastrointestinal tract.

In some aspects, the peptide remains intact after exposure to Tris(2-carboxyethyl)phosphine HCl (TCEP), or 2-Mercaptoethanol. In some aspects, the peptide remains intact after exposure to chymotrypsin, serum protease, serine protease, cysteinyl protease, aspartyl protease, elastase, matrix metalloproteases, cytochrome P450 enzymes, carboxypeptidases, or cathepsins. In some aspects, 90-100% of the peptide remains intact after exposure to a temperature of at least 25° C., 30° C., or 40° C. with at least 60%, 65% or 75% relative humidity for at least 3, 6, 12, 18, 24, 36, or 48 months.

In some aspects, the peptide exhibits the characteristics after oral administration, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof, to a subject.

In further aspects, the subject is a human. In still further aspects, the subject is a non-human animal.

In some aspects, at least one residue of the peptide comprises a chemical modification. In further aspects, the chemical modification is blocking the N-terminus of the peptide. In still further aspects, the chemical modification is methylation, acetylation, or acylation. In some aspects, the chemical modification comprises methylation of one or more lysine residues or analogue thereof, methylation of the N-terminus, or methylation of one or more lysine residue or analogue thereof and methylation of the N-terminus.

In some aspects, the peptide is linked to an acyl adduct. In some aspects, the peptide is linked to an active agent. In some aspects, the active agent is fused with the peptide at an N-terminus or a C-terminus of the peptide. In some aspects, the active agent is an Fc domain. In further aspects, the peptide fused with an Fc domain comprises a contiguous sequence.

In further aspects, 1, 2, 3, 4, 5, 6, 7, 8, 9, or 10 active agents are linked to the peptide. In some aspects, the peptide is linked to the active agent via a cleavable linker. In some aspects, the peptide is linked to the active agent at an N-terminus, at the epsilon amine of an internal lysine residue, at the carboxylic acid of an aspartic acid, or glutamic acid residue, or a C-terminus of the peptide by a linker.

In some aspects, the peptide further comprises a non-natural amino acid, wherein the non-natural amino acid is an insertion, appendage, or substitution for another amino acid. In some aspects, the peptide is linked to the active agent at the non-natural amino acid by a linker. In some aspects, the linker comprises an amide bond, an ester bond, a carbamate bond, a carbonate bond, a hydrazone bond, an oxime bond, a disulfide bond, a thioester bond, a thioether bond, or a carbon-nitrogen bond. In some aspects, the cleavable linker comprises a cleavage site for matrix metalloproteinases, thrombin, cathepsins, or beta-glucuronidase.

In other aspects, the peptide is linked to the active agent via a noncleavable linker. In some aspects, the active agent is: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, an antibody fragment, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic agent, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, a glucocorticoid, an anti-cytokine agent, a pain-reducing agent, a dendrimer, a fatty acid, an Fc region, siderocalin, or a combination thereof.

In some aspects, the peptide is linked to a detectable agent. In some aspects, the detectable agent is fused with the peptide at an N-terminus or a C-terminus of the peptide. In some aspects, 1, 2, 3, 4, 5, 6, 7, 8, 9, or 10 detectable agents are linked to the peptide.

In other aspects, the peptide is linked to the detectable agent via a cleavable linker. In some aspects, the peptide is linked to the detectable agent at an N-terminus, at the epsilon amine of an internal lysine residue, at the carboxylic acid of an internal aspartic acid or glutamic acid residue, or a C-terminus of the peptide by a linker. In some aspects, the peptide is linked to the detectable agent at the non-natural amino acid by a linker. In some aspects, the linker comprises an amide bond, an ester bond, a carbamate bond, a hydrazone bond, an oxime bond, a thioether bond, a thioester bond, or a carbon-nitrogen bond. In some aspects, the cleavable linker comprises a cleavage site for matrix metalloproteinases, thrombin, cathepsins, or beta-glucuronidase.

In other aspects, the peptide is linked to the detectable agent via a noncleavable linker. In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye.

In some aspects, the peptide is administered orally. In some aspects, the subject has a condition. In some aspects, the condition is a gastrointestinal infection or chronic gastrointestinal disease. In other aspects, the gastrointestinal infection is a bacterial infection, prokaryotic infection, or fungal infection. In some aspects, the chronic gastrointestinal disease is irritable bowel syndrome, inflammatory bowel syndrome, Crohn's disease, gastroesophageal reflux disease, ulcerative colitis or constipation.

In other aspects, the condition is cancer. In further aspects, the cancer is colorectal cancer, stomach cancer, or esophageal cancer. In some aspects, the peptide is administered to treat the condition. In some aspects, the condition is an inflammation, a cancer, a degradation, a growth disturbance, genetic, a tear, an infection, an injury, a rheumatic condition, an immune system disorder, a kidney disease, lung disease, a condition of aging, a degenerative brain condition, a degenerative body condition, a childhood condition, a hepatic disease, a pulmonary disease, a pancreatic condition, or a gastrointestinal condition.

In some aspects, the kidney disease is acute kidney injury or chronic kidney disease. In some aspects, the peptide is delivered by oral administration to treat a gastrointestinal condition. In other aspects, the peptide is delivered by oral administration to treat a non-gastrointestinal condition. In still other aspects the peptide is delivered by oral administration and homes to cartilage. In some aspects, the peptide is delivered by oral administration and homes to kidneys or proximal tubules of the kidneys. In some aspects, the peptide is delivered by oral administration and homes to or accumulates in tumors.

In some aspects, the peptide is administered to detect a diseased region, tissue, structure, or cell. In some aspects, a peptide enters the cell. In some aspects, the peptide is active intracellularly. In some aspects, after the peptide is administered, one of the following characteristics of the composition is measured in the subject: (a) an intact peptide or fragment thereof, in plasma; (b) the intact peptide or fragment thereof, in the stomach; (c) the intact peptide or fragment thereof, in the gastrointestinal tract; (d) the intact peptide or fragment thereof, in the colon; (e) the intact peptide or fragment thereof, in the feces; (f) the intact peptide or fragment thereof, in the urine; (g) the intact peptide or fragment thereof, in cartilage; (h) an average Cmax of the intact peptide or fragment thereof, in plasma; (i) an average Tmax at which the Cmax is reached; (j) an average area under the curve (AUC) of the intact peptide or fragment thereof in the subject; (k) an average bioavailability of the intact peptide or fragment thereof in the subject; (1) an average t½ of the intact peptide or fragment thereof in the subject; (m) an average clearance (CL) of the intact peptide or fragment thereof in the subject; or (n) an average volume of distribution (Vd) of the intact peptide or fragment thereof in the subject.

In various aspects, the present disclosure provides a peptide conjugate comprising: a peptide linked to an agent, wherein the peptide has at least one of the following characteristics: (a) at least 70% the peptide remains intact after exposure to dithiothreitol (DTT) at a concentration of from 10 mM and a temperature of at least 23° C. for at least 30 minutes; (b) at least 70% of the peptide remains intact after exposure to reduced glutathione (GSH) at a concentration of from 10 mM and a temperature of at least 23° C. for at least 30 minutes; (c) at least 70% of the peptide remains intact after exposure to trypsin at a concentration of 500 U/ml and a temperature of at least 23° C. for at least 30 minutes; (d) at least 70% of the peptide remains intact after exposure to pepsin at a concentration of 500 U/ml and a temperature of at least 37° C. for at least 30 minutes; (e) at least 70% of the peptide remains intact after exposure to simulated gastric fluid (SGF; pH 1.05; 2% (w/v) sodium chloride in 0.7% (v/v) hydrochloric acid) and a temperature of at least 23° C. for at least 30 minutes; (f) at least 70% of the peptide remains intact after exposure to a pH of 1.05 and a temperature of at least 23° C. for at least 30 minutes; (g) at least 70% of the peptide remains intact after exposure to the combination of simulated gastric fluid (SGF; pH 1.05; 2% (w/v) sodium chloride in 0.7% (v/v) hydrochloric acid) with 500 U/ml pepsin, 100 mM Tris, and 10 mM DTT (SPTD) and a temperature of at least 23° C. for at least 30 minutes; (h) at least 70% of the peptide remains intact after exposure to at least 70° C. for at least 60 minutes; (i) at least 70% of the peptide remains intact after exposure to at least 100° C. for at least 60 minutes; or (j) at least 10% of the peptide remains intact after passage through the mouth, stomach, small intestine, or the large intestine.

In various aspects, the present disclosure provides a peptide conjugate comprising: a peptide linked to an agent, wherein the peptide has at least one of the following characteristics: (a) at least 70%, 72%, 75%, 78%, 80%, 82%, 85, 88%, 90%, 92%, 95%, 98%, or 99% of the peptide remains intact after exposure to dithiothreitol (DTT) at a concentration of from 5 mM to 10 mM and a temperature of at least 23° C., 37° C., or 39° C. for at least 5, 10, 15, 20, 30, or 60 minutes; (b) at least 70%, 72%, 75%, 78%, 80%, 82%, 85, 88%, 90%, 92%, 95%, 98%, or 99% of the peptide remains intact after exposure to reduced glutathione (GSH) at a concentration of from 5 mM to 10 mM and a temperature of at least 23° C., 37° C., or 39° C. for at least 5, 10, 15, 20, 30, or 60 minutes; (c) at least 70%, 72%, 75%, 78%, 80%, 82%, 85, 88%, 90%, 92%, 95%, 98%, or 99% of the peptide remains intact after exposure to trypsin at a concentration of 0.5 U/ml to 5000 U/ml and a temperature of at least 23° C., 37° C., or 39° C. for at least 5, 10, 15, 20, 30, or 60 minutes; (d) at least 70%, 72%, 75%, 78%, 80%, 82%, 85, 88%, 90%, 92%, 95%, 98%, or 99% of the peptide remains intact after exposure to pepsin at a concentration of 0.5 U/ml to 5000 U/ml and a temperature of at least 23° C., 37° C., or 39° C. for at least 5, 10, 15, 20, 30, or 60 minutes; (e) at least 70%, 72%, 75%, 78%, 80%, 82%, 85, 88%, 90%, 92%, 95%, 98%, or 99% of the peptide remains intact after exposure to simulated gastric fluid (SGF; pH 1.05; 2% (w/v) sodium chloride in 0.7% (v/v) hydrochloric acid) and a temperature of at least 23° C., 37° C., or 39° C. for at least 5, 10, 15, 20, 30, or 60 minutes; (f) at least 70%, 72%, 75%, 78%, 80%, 82%, 85, 88%, 90%, 92%, 95%, 98%, or 99% of the peptide remains intact after exposure to a pH of from 1-2, 2-3, 3-4, or 4-5 and a temperature of at least 23° C., 37° C., or 39° C. for at least 5, 10, 15, 20, 30, or 60 minutes; (g) at least 70%, 72%, 75%, 78%, 80%, 82%, 85, 88%, 90%, 92%, 95%, 98%, or 99% of the peptide remains intact after exposure to the combination of simulated gastric fluid (SGF; pH 1.05; 2% (w/v) sodium chloride in 0.7% (v/v) hydrochloric acid) with 0.5 U/ml to 5000 U/ml pepsin, 100 mM Tris, and 10 mM DTT (SPTD) and a temperature of at least 23° C., 37° C., or 39° C. for at least 5, 10, 15, 20, 30, or 60 minutes; (h) at least 70%, 72%, 75%, 78%, 80%, 82%, 85, 88%, 90%, 92%, 95%, 98%, or 99% of the peptide remains intact after exposure to at least 70° C. for at least 5, 10, 15, 20, 30, or 60 minutes; (i) at least 70%, 72%, 75%, 78%, 80%, 82%, 85, 88%, 90%, 92%, 95%, 98%, or 99% of the peptide remains intact after exposure to at least 100° C. for at least 5, 10, 15, 20, 30, or 60 minutes; or (j) at least 1%, 10%, 20%, 30%, 40%, 50%, 60%, 70%, 72%, 75%, 78%, 80%, 82%, 85, 88%, 90%, 92%, 95%, 98%, or 99% of the peptide remains intact after passage through the mouth, stomach, small intestine, or the large intestine.

In some aspects, the peptide of the peptide conjugate is a non-naturally occurring peptide. In some aspects, the peptide of the peptide conjugate has two or more of the characteristics (a) through (j). In further aspects, the peptide of the peptide conjugate has three or more of the characteristics (a) through (j). In still further aspects, the peptide of the peptide conjugate has four or more of the characteristics (a) through (j). In still further aspects, the peptide of the peptide conjugate has five or more of the characteristics (a) through (j). In still further aspects, the peptide of the peptide conjugate has six or more of the characteristics (a) through (j). In still further aspects, the peptide of the peptide conjugate has seven or more of the characteristics (a) through (j). In still further aspects, the peptide of the peptide conjugate has eight or more of the characteristics (a) through (j). In still further aspects, the peptide of the peptide conjugate has all of the characteristics (a) through (j).

In some aspects, the peptide of the peptide conjugate remains intact after exposure to at least 75° C. for at least 5, 10, 15, 20, 30, or 60 minutes. In some aspects, the peptide of the peptide conjugate comprises a motif, and wherein the motif comprises Cys-X_{[0-15]}-Cys-X_{[0-15]}-Cys-X_{[0-15]}-Cys-X_{[0-15]}-Cys-X_{[0-15]}-Cys (SEQ ID NO: 179), wherein X is any amino acid. In further aspects, X is any amino acid or absent. In some aspects, the peptide of the peptide conjugate is a knotted peptide. In some aspects, the peptide of the peptide conjugate comprises 6 or more cysteine residues. In some aspects, the peptide of the peptide conjugate comprises three or more disulfide bridges formed between cysteine residues, wherein one of the disulfide bridges passes through a loop formed by two other disulfide bridges.

In further aspects, the peptide of the peptide conjugate comprises a plurality of disulfide bridges. In some aspects, the peptide of the peptide conjugate is a cystine-dense peptide (CDP). In some aspects, the CDP comprises independent folding domains, wherein the independent folding domains comprise a high density of at least six cysteines. In some aspects, the CDP is exported to the cell surface or secreted.

In some aspects, the CDP comprises a disulfide bond between cysteines 1 and 4, 2 and 5, and 3 and 6. In other aspects, the CDP comprises a disulfide bond between cysteines 1 and 3, 2 and 5, and 4 and 6. In still other aspects, the CDP comprises a disulfide bond between cysteines 1 and 4, 2 and 6, and 3 and 5. In some aspects, the CDP comprises a disulfide bond between cysteines 1 and 5, 2 and 4, and 3 and 6. In other aspects, the CDP comprises a disulfide bond between cysteines 1 and 6, 2 and 4, and 3 and 5.

In other aspects, the CDP is a non-knotted CDP. In some aspects, the non-knotted CDP comprises a disulfide bond between cysteines 1 and 6, 2 and 5, and 3 and 4. In some aspects, the peptide of the peptide conjugate comprises a topology of a Cysu-Cysv disulfide bond, a Cysw-Cysx disulfide bond, and a Cysy-Cysz disulfide bond, wherein the Cysw-Cysx disulfide bond passes through a macrocycle comprising the Cysu-Cysv disulfide bond and the Cysy-Cysz disulfide bond. In some aspects, the Cysw-Cysx cysteine-cysteine bond is a knotting cysteine.

In some aspects, the knotted peptide is a hitchin, and wherein the hitchin comprises a topology wherein the Cysu-Cysy disulfide bond is between cysteine 1 and cysteine 4, the Cysw-Cysx disulfide bond is between cysteine 2 and cysteine 5, and wherein the Cysy-Cysz disulfide bond is between cysteine 3 and cysteine 6.

In some aspects, at least one amino acid residue of the peptide is in an L configuration or, wherein at least one amino acid residue of the knotted peptide is in a D configuration. In some aspects, the peptide of the peptide conjugate is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues long. In some aspects, any one or more K residues are replaced by an R residue or wherein any one or more R residues are replaced by for a K residue.

In some aspects, the peptide of the peptide conjugate is arranged in a multimeric structure with at least one other peptide. In some aspects, the peptide of the peptide conjugate comprises any one of SEQ ID NO: 167-SEQ ID NO: 171. In other aspects, the peptide of the peptide conjugate comprises any one of any one of SEQ ID NO: 172-SEQ ID NO: 176.

In other aspects, the peptide of the peptide conjugate comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 1-SEQ ID NO: 83. In further aspects, the peptide of the peptide conjugate comprises any one of SEQ ID NO: 1-SEQ ID NO: 83. In some aspects, the peptide of the peptide conjugate comprises at least 70% sequence identity, at least 75% sequence identity, at least 80% sequence identity, at least 85% sequence identity, at least 90% sequence identity, at least 92% sequence identity, at least 95% sequence identity, at least 97% sequence identity, or at least 99% sequence identity with any one of SEQ ID NO: 84-SEQ ID NO: 166. In further aspects, the peptide of the peptide conjugate comprises any one of SEQ ID NO: 84-SEQ ID NO: 166.

In some aspects, the peptide of the peptide conjugate comprises any one of SEQ ID NO: 27, SEQ ID NO: 24, SEQ ID NO: 6, SEQ ID NO: 10, SEQ ID NO: 12, SEQ ID NO: 57, SEQ ID NO: 31, SEQ ID NO: 77, or SEQ ID NO: 78. In some aspects, the peptide of the peptide conjugate comprises any one of SEQ ID NO: 27, SEQ ID NO: 31, or SEQ ID NO: 57. In some aspects, the peptide of the peptide conjugate comprises any one of SEQ ID NO: 29, SEQ ID NO: 4, SEQ ID NO: 79, or SEQ ID NO: 80. In some aspects, the peptide of the peptide conjugate comprises any one of SEQ ID NO: 26, SEQ ID NO: 81, SEQ ID NO: 82, or SEQ ID NO: 83.

In some aspects, the peptide of the peptide conjugate comprises any one of SEQ ID NO: 2. In other aspects, the peptide of the peptide conjugate comprises any one of SEQ ID NO: 31.

In some aspects, the peptide of the peptide conjugate exhibits an average Tmax of 0.5-12 hours at which the Cmax is reached. In some aspects, the peptide of the peptide conjugate achieves an average bioavailability of the peptide in serum of 0.1%-10% after administering the peptide to the subject by an oral route. In other aspects, the peptide of the peptide conjugate achieves an average bioavailability of the peptide in serum of less than 0.1% after administering the peptide to the subject by an oral route. In still other aspects, the peptide of the peptide conjugate achieves an average bioavailability of the peptide in serum of 10%-100% after administering the peptide to a subject by a parenteral route.

In some aspects, the peptide of the peptide conjugate achieves an average t½ of 0.1 hours-168 hours in a subject after administering the peptide to the subject. In some aspects, the peptide of the peptide conjugate achieves an average clearance (CL) of 0.5-100 L/hour of the peptide after administering the peptide to a subject. In some aspects, the peptide of the peptide conjugate achieves an average volume of distribution (Vd) of 200-20,000 mL in the subject after administering the peptide to the subject.

In some aspects, the peptide of the peptide conjugate remains intact after exposure to oxidative conditions for 30 minutes. In some aspects, the peptide of the peptide conjugate remains intact after exposure to a pH less than 2 for 30 minutes. In some aspects, the peptide of the peptide conjugate remains intact after passage through the gastrointestinal tract.

In further aspects, the peptide of the peptide conjugate remains intact after exposure to Tris(2-carboxyethyl)phosphine HCl (TCEP), or 2-Mercaptoethanol. In some aspects, the peptide of the peptide conjugate remains intact after exposure to chymotrypsin, serum protease, serine protease, cysteinyl protease, aspartyl protease, elastase, matrix metalloproteases, cytochrome P450 enzymes, carboxypeptidases, or cathepsins.

In some aspects, 90-100% of the peptide of the peptide conjugate remains intact after exposure to a temperature of at least 25° C., 30° C., or 40° C. with at least 60%, 65% or 75% relative humidity for at least 3, 6, 12, 18, 24, 36, or 48 months. In some aspects, the peptide of the peptide conjugate exhibits the characteristics after oral administration, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof, to a subject.

In further aspects, the subject is a human. In still further aspects, the subject is a non-human animal.

In some aspects, at least one residue of the peptide of the peptide conjugate comprises a chemical modification. In further aspects, the chemical modification is blocking the N-terminus of the peptide. In still further aspects, the chemical modification is methylation, acetylation, or acylation. In some aspects, the chemical modification comprises methylation of one or more lysine residues or analogue thereof, methylation of the N-terminus, or methylation of one or more lysine residue or analogue thereof and methylation of the N-terminus.

In some aspects, the peptide of the peptide conjugate is linked to an acyl adduct. In some aspects, the agent is an active agent. In some aspects, the active agent is fused with the peptide of the peptide conjugate at an N-terminus or a C-terminus of the peptide.

In further aspects, the active agent is an Fc domain. In still further aspects, the peptide of the peptide conjugate fused with an Fc domain comprises a contiguous sequence. In some aspects, 1, 2, 3, 4, 5, 6, 7, 8, 9, or 10 active agents are linked to the peptide of the peptide conjugate. In some aspects, the peptide of the peptide conjugate is linked to the active agent via a cleavable linker.

In some aspects, the peptide of the peptide conjugate is linked to the active agent at an N-terminus, at the epsilon amine of an internal lysine residue, at the carboxylic acid of an aspartic acid, or glutamic acid residue, or a C-terminus of the peptide by a linker. In some aspects, the peptide of the peptide conjugate further comprises a non-natural amino acid, wherein the non-natural amino acid is an insertion, appendage, or substitution for another amino acid.

In some aspects, the peptide conjugate is linked to the active agent at the non-natural amino acid by a linker. In some aspects, the linker comprises an amide bond, an ester bond, a carbamate bond, a carbonate bond, a hydrazone bond, an oxime bond, a disulfide bond, a thioester bond, a thioether bond, or a carbon-nitrogen bond. In further aspects, the cleavable linker comprises a cleavage site for matrix metalloproteinases, thrombin, cathepsins, or beta-glucuronidase. In some aspects, the peptide of the peptide conjugate is linked to the active agent via a noncleavable linker.

In some aspects, the active agent is: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, an antibody fragment, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic agent, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, a glucocorticoid, an anti-cytokine agent, a pain-reducing agent, a dendrimer, a fatty acid, an Fc region, siderocalin, or a combination thereof.

In some aspects, the agent is a detectable agent. In further aspects, the detectable agent is fused with the peptide of the peptide conjugate at an N-terminus or a C-terminus of the peptide. In some aspects, 1, 2, 3, 4, 5, 6, 7, 8, 9, or 10 detectable agents are linked to the peptide of the peptide conjugate. In some aspects, the peptide of the peptide conjugate is linked to the detectable agent via a cleavable linker. In some aspects, the peptide of the peptide conjugate is linked to the detectable agent at an N-terminus, at the epsilon amine of an internal lysine residue, at the carboxylic acid of an internal aspartic acid or glutamic acid residue, or a C-terminus of the peptide by a linker.

In some aspects, the peptide of the peptide conjugate is linked to the active agent at the non-natural amino acid by a linker. In some aspects, the linker comprises an amide bond, an ester bond, a carbamate bond, a hydrazone bond, an oxime bond, a thioether bond, a thioester bond, or a carbon-nitrogen bond. In some aspects, the cleavable linker comprises a cleavage site for matrix metalloproteinases, thrombin, cathepsins, or beta-glucuronidase.

In other aspects, the peptide of the peptide conjugate is linked to the detectable agent via a noncleavable linker. In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye. In some aspects, the peptide of the peptide conjugate is administered orally. In some aspects, the subject has a condition.

In further aspects, the condition is a gastrointestinal infection or chronic gastrointestinal disease. In some aspects, the gastrointestinal infection is a bacterial infection, prokaryotic infection, or fungal infection. In some aspects, the chronic gastrointestinal disease is irritable bowel syndrome, inflammatory bowel syndrome, Crohn's disease, gastroesophageal reflux disease, ulcerative colitis or constipation.

In other aspects, the condition is cancer. In some aspects, the cancer is colorectal cancer, stomach cancer, or esophageal cancer. In some aspects, the peptide conjugate is administered to treat the condition.

In some aspects, the condition is an inflammation, a cancer, a degradation, a growth disturbance, genetic, a tear, an infection, an injury, a rheumatic condition, an immune system disorder, a kidney disease, lung disease, a condition of aging, a degenerative brain condition, a degenerative body condition, a childhood condition, a hepatic disease, a pulmonary disease, a pancreatic condition, or a gastrointestinal condition. In some aspects, the kidney disease is acute kidney injury or chronic kidney disease.

In some aspects, the peptide conjugate is delivered by oral administered to treat a gastrointestinal condition. In other aspects, the peptide conjugate is delivered by oral administered to treat a non-gastrointestinal condition. In some aspects, the peptide conjugate is delivered by oral administration and homes to cartilage. In some aspects, the peptide conjugate is delivered by oral administration and homes to kidneys or proximal tubules of the kidneys. In some aspects, the peptide conjugate is delivered by oral administration and homes to or accumulates in a tumor.

In some aspects, the peptide conjugate is administered to detect a diseased region, tissue, structure, or cell. In some aspects, the peptide conjugate enters the cell. In some aspects, the peptide conjugate is administered to detect a diseased region, tissue, structure, or cell.

In some aspects, the peptide conjugate is active intracellularly. In some aspects, after the administering, one of the following characteristics of the composition is measured in the subject: (a) an intact peptide or fragment thereof, in plasma; (b) the intact peptide or fragment thereof, in the stomach; (c) the intact peptide or fragment thereof, in the gastrointestinal tract; (d) the intact peptide or fragment thereof, in the colon; (e) the intact peptide or fragment thereof, in the feces; (f) the intact peptide or fragment thereof, in the urine; (g) the intact peptide or fragment thereof, in cartilage; (h) an average Cmax of the intact peptide or fragment thereof, in plasma; (i) an average Tmax at which the Cmax is reached; (j) an average area under the curve (AUC) of the intact peptide or fragment thereof in the subject; (k) an average bioavailability of the intact peptide or fragment thereof in the subject; (l) an average t½ of the intact peptide or fragment thereof in the subject; (m) an average clearance (CL) of the intact peptide or fragment thereof in the subject; or (n) an average volume of distribution (Vd) of the intact peptide or fragment thereof in the subject.

In various aspects, the present disclosure provides a pharmaceutical composition comprising the composition of any of the above compositions or a salt thereof, and a pharmaceutically acceptable carrier. In some aspects, the pharmaceutical composition is formulated for administration to a subject. In some aspects, the pharmaceutical composition is formulated for inhalation, intranasal administration, oral administration, topical administration, intravenous administration, subcutaneous administration, intra-articular administration, intramuscular administration, intraperitoneal administration, intra-synovial administration, or a combination thereof.

In some aspects, the pharmaceutical composition further includes a permeation enhancer. In some aspects, the permeation enhancer increases oral absorption. In some aspects, the permeation enhancer is SNAC, 5-CNAC, sodium caprylate, an aromatic alcohol, EDTA, a sodium alkyl sulfate, or a citrate. In some aspects, the pharmaceutical composition is formulated in a buffer. In further aspects, the pharmaceutical composition is delivered within an enteric coating for oral delivery.

In various aspects, the present disclosure provides a method of administering to a subject any one of the above compositions or any one of the above pharmaceutical compositions. In some aspects, the composition or pharmaceutical composition is administered by inhalation, intranasally, orally, topically, intravenously, subcutaneously, intra-articularly, intramuscularly administration, intraperitoneally, intra-synovially, by vaginal route, rectal route, pulmonary route, ocular route, buccal, sublingual, intrathecal, or a combination thereof.

In various aspects, the present disclosure provides a method of making the peptide of any one of the above peptides or peptide conjugates by recombinant expression.

In various aspects, the present disclosure provides a method of making the peptide of any one of the above peptides or peptide conjugates by chemical synthesis.

In various aspects, the present disclosure provides a method of manufacturing any one of the above peptides or any one of the above pharmaceutical compositions, wherein the peptide is more stable during the manufacturing. In some aspects, the peptide is less susceptible to degradation by proteases during the manufacturing. In some aspects, the manufacturing is recombinant expression or purification. In some aspects, the manufacturing yields peptides of high purity. In further aspects, the manufacturing yields a higher quantity of peptide. In some aspects, the manufacturing yields a peptide with a longer shelf life. In some aspects, the manufacturing yields a peptide that is stable at an elevated storage temperature. In further aspects, the elevated storage temperature is 25° C., 30° C., or 40° C.

In some aspects, any one of the above peptides or peptide conjugates remains intact after exposure to pepsin at a concentration of 500 U/ml and a temperature of at least 37° C. for at least 30 minutes. In some aspects, any one of the above peptides or peptide conjugates remains intact after exposure to pepsin at a concentration of 50 U/ml and a temperature of at least 37° C. for at least 30 minutes. In some aspects, any one of the above peptides or peptide conjugates remains intact after exposure to pepsin at a concentration of 5000 U/ml and a temperature of at least 37° C. for at least 30 minutes. In some aspects, any one of the above peptides or peptide conjugates remains intact after exposure to trypsin at a concentration of 500 U/ml and a temperature of at least 37° C. for at least 30 minutes. In some aspects, any one of the above peptides or peptide conjugates remains intact after exposure to trypsin at a concentration of 50 U/ml and a temperature of at least 37° C. for at least 30 minutes.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows HPLC chromatograms of 12.5 μg of a peptide of SEQ ID NO: 2 suspended in various solutions including simulated gastric fluid, pepsin, Tris, and DTT (SPTD), simulated gastric fluid (SGF) at pH 1.05 and 500 U/ml pepsin (P), SGF, dithiothreitol (DTT), and non-reducing (NR) conditions. The peaks seen at 1.5 minutes and 2.25 minutes were DTT (and not peptide).

FIG. 2 shows HPLC chromatograms of 12.5 μg of a peptide of SEQ ID NO: 27 suspended in various solutions including simulated gastric fluid, pepsin, Tris, and DTT (SPTD), simulated gastric fluid (SGF) at pH 1.05 and 500 U/ml pepsin (P), SGF, DTT, and non-reducing (NR) conditions.

FIG. 3 shows HPLC chromatograms of 12.5 μg of a peptide of SEQ ID NO: 31 suspended in various solutions including simulated gastric fluid, pepsin, Tris, and DTT (SPTD), simulated gastric fluid (SGF) at pH 1.05 and 500 U/ml pepsin (P), SGF, DTT, and non-reducing (NR) conditions.

FIG. 4 shows an HPLC chromatogram of 500 U/ml trypsin (T) in 25 mM Tris, 5 μg soybean trypsin inhibitor (I) and 10 mM dithiothreitol (DTT) (T, I, DTT) as well as HPLC chromatograms of 12.5 μg of a peptide of SEQ ID NO: 1 suspended in various solutions including (T, I, DTT), (T,I), DTT, and non-reducing (NR) conditions.

FIG. 5 shows an HPLC chromatogram of 500 U/ml trypsin in 25 mM Tris, 5 μg soybean trypsin inhibitor and 10 mM DTT (T, I, DTT) as well as HPLC chromatograms of 12.5 μg of a peptide of SEQ ID NO: 2 suspended in various solutions including (T, I, DTT), (T,I), DTT, and non-reducing (NR) conditions.

FIG. 6 shows the concentration of a radiolabeled peptide of SEQ ID NO: 27 (SEQ ID NO: 27-r) in plasma after administration of the peptide to a mouse.

FIG. 6A shows the concentration of peptide in plasma after intravenous (IV) administration of 20 nmol of a radiolabeled peptide of SEQ ID NO: 27 (SEQ ID NO: 27-r) and oral (PO) administration of 100 nmol SEQ ID NO: 27-r, as quantified by measuring the ¹⁴C signal using liquid scintillation counting. The delivered dose of ¹⁴C was 4.8 μCi for intravenous administration and 24 for oral administration. Time points examined included 0.08, 0.5, 1, 3, 8, 24, 48 hours and three mice were examined per time point.

FIG. 6B shows the percent of administered peptide dose recovered in plasma after intravenous (IV) administration of 20 nmol of a radiolabeled peptide of SEQ ID NO: 27 (SEQ ID NO: 27-r) and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point.

FIG. 6C shows the intensity of peptide and peptide fragment peaks in plasma as measured by tandem HPLC and liquid scintillation counting after oral administration by gavage of 100 nmol of a radiolabeled peptide of SEQ ID NO: 27 (SEQ ID NO: 27-r). The delivered dose of ¹⁴C was 24 μCi for oral administration. Time points examined included 0.5, 1, and 3 hours.

FIG. 7 shows the concentration of a radiolabeled peptide of SEQ ID NO: 27 (SEQ ID NO: 27-r) in urine after administration of the peptide to a mouse.

FIG. 7A shows the concentration of peptide in urine after intravenous (IV) administration of 20 nmol of a radiolabeled peptide of SEQ ID NO: 27 (SEQ ID NO: 27-r) and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point.

FIG. 7B shows the intensity of peptide and peptide fragment peaks in urine as measured by tandem HPLC and liquid scintillation counting after oral administration by gavage of 100 nmol of a radiolabeled peptide of SEQ ID NO: 27 (SEQ ID NO: 27-r). The delivered dose of ¹⁴C was 24 μCi for oral administration. Time points examined included 0.5, 1, 3, 8, 24, and 48 hours.

FIG. 8 shows the concentration of a radiolabeled peptide of SEQ ID NO: 27 (SEQ ID NO: 27-r) in feces after administration of the peptide to a mouse.

FIG. 8A shows the concentration of peptide in feces after intravenous (IV) administration of 20 nmol of a radiolabeled peptide of SEQ ID NO: 27 (SEQ ID NO: 27-r) and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point.

FIG. 8B shows the intensity of peptide and peptide fragment peaks in feces as measured by tandem HPLC and liquid scintillation counting after oral administration by gavage of 100 nmol of a radiolabeled peptide of SEQ ID NO: 27 (SEQ ID NO: 27-r). The delivered dose of ¹⁴C was 24 μCi for oral administration. Time points examined included 3 and 8 hours.

FIG. 9 illustrates HPLC chromatograms of two peptides after exposure to reducing agents and/or proteinases.

FIG. 9A illustrates the HPLC trace of a peptide of SEQ ID NO: 27 in PBS.

FIG. 9B illustrates the HPLC trace of a peptide of SEQ ID NO: 27 in DTT in PBS.

FIG. 9C illustrates the HPLC trace of a peptide of SEQ ID NO: 27 in 50 U trypsin and 1 mg/ml inhibitor in PBS.

FIG. 9D illustrates the HPLC trace of a peptide of SEQ ID NO: 27 in 50 U trypsin, 1 mg/ml inhibitor, and DTT in PBS.

FIG. 9E illustrates the HPLC trace of a peptide of SEQ ID NO: 31 in PBS.

FIG. 9F illustrates the HPLC trace of a peptide of SEQ ID NO: 31 in DTT in PBS.

FIG. 9G illustrates the HPLC trace of a peptide of SEQ ID NO: 31 in 50 U trypsin and 1 mg/ml inhibitor in PBS.

FIG. 911 illustrates the HPLC trace of a peptide of SEQ ID NO: 31 in 50 U trypsin, 1 mg/ml inhibitor, and DTT in PBS.

FIG. 10 illustrates HPLC chromatograms of two peptides after exposure to reducing agents, proteinases, and/or simulated gastric fluid conditions.

FIG. 10A illustrates the HPLC trace of a peptide of SEQ ID NO: 27 incubated in PBS.

FIG. 10B illustrates the HPLC trace of a peptide of SEQ ID NO: 27 incubated in DTT in PBS.

FIG. 10C illustrates the HPLC trace of a peptide of SEQ ID NO: 27 incubated in simulated gastric fluid (SGF).

FIG. 10D illustrates the HPLC trace of a peptide of SEQ ID NO: 27 incubated in 500 U pepsin in SGF.

FIG. 10E illustrates the HPLC trace of a peptide of SEQ ID NO: 27 incubated in 500 U pepsin, 0.5 M Tris, and DTT in SGF.

FIG. 10F illustrates the HPLC trace of a peptide of SEQ ID NO: 31 incubated in PBS.

FIG. 10G illustrates the HPLC trace of a peptide of SEQ ID NO: 31 incubated in DTT in PBS.

FIG. 1011 illustrates the HPLC trace of a peptide of SEQ ID NO: 31 incubated in simulated gastric fluid (SGF).

FIG. 10I illustrates the HPLC trace of a peptide of SEQ ID NO: 31 incubated in 500 U pepsin in SGF.

FIG. 10J illustrates the HPLC trace of a peptide of SEQ ID NO: 31 incubated in 500 U pepsin, 0.5 M Tris, and DTT in SGF.

FIG. 11 illustrates HPLC chromatograms of a peptide of SEQ ID NO: 31 and a negative control peptide of SEQ ID NO: 177 after exposure to a range of conditions including oxidative, reductive, and acidic conditions as well as after exposure to proteinases.

FIG. 11A illustrates the HPLC trace of a peptide of SEQ ID NO: 31 under reducing and acidic conditions.

FIG. 11B illustrates the HPLC trace of a peptide of SEQ ID NO: 31 under various combinations of reducing agents and proteases including 10 mM DTT in 500 U pepsin (5000 U/ml), 500 U pepsin (5000 U/ml), 10 mM DTT in 50 U (500 U/ml) trypsin, and 50 U (500 U/ml) trypsin.

FIG. 11C illustrates the HPLC trace of a peptide of a negative control peptide of SEQ ID NO: 177 under various protease conditions including in 500 U pepsin, in 50 U trypsin, non-reducing (NR) conditions in simulated gastric fluid (SGF) at pH 1.05, and NR (oxidizing conditions).

FIG. 12 illustrates HPLC chromatograms of a peptide of SEQ ID NO: 3, SEQ ID NO: 23 and SEQ ID NO: 25 in non-reducing (NR) conditions after incubation at room temperature, 70° C., or 100° C. for one hour.

FIG. 12A illustrates the HPLC trace of a peptide of SEQ ID NO: 3 in NR conditions after incubation at room temperature, 70° C., or 100° C. for one hour.

FIG. 12B illustrates the HPLC trace of a peptide of SEQ ID NO: 23 in NR conditions after incubation at room temperature, 70° C., or 100° C. for one hour.

FIG. 12C illustrates the HPLC trace of a peptide of SEQ ID NO: 25 in NR conditions after incubation at room temperature, 70° C., or 100° C. for one hour.

FIG. 13 illustrates the SDS-PAGE and HPLC trace of peptides of SEQ ID NO: 43, 44, and 45 in non-reducing (NR) conditions and reducing (R) conditions.

FIG. 13A illustrates an SDS-PAGE of a soluble peptide of SEQ ID NO: 43 in non-reduced (NR) or reduced (R) conditions.

FIG. 13B illustrates an HPLC chromatogram of a peptide of SEQ ID NO: 43 in non-reduced or reduced conditions.

FIG. 13C illustrates an SDS-PAGE of a soluble peptide of SEQ ID NO: 44 in non-reduced (NR) or reduced (R) conditions.

FIG. 13D illustrates an HPLC chromatogram of a peptide of SEQ ID NO: 44 in non-reduced or reduced conditions.

FIG. 13E illustrates an SDS-PAGE of a soluble peptide of SEQ ID NO: 45 in non-reduced (NR) or reduced (R) conditions.

FIG. 13F illustrates an HPLC chromatogram of a peptide of SEQ ID NO: 45 in non-reduced (NR) or reduced (R) conditions.

FIG. 14 illustrates the stability of peptides of SEQ ID NO: 39 and SEQ ID NO: 43 in the presence of reducing agents.

FIG. 14A illustrates the HPLC chromatograms of a peptide of SEQ ID NO: 43 in non-reducing (NR) conditions or in 10 mM DTT reducing (R) conditions. A representative mass spectrometry peak profile is shown in the inset.

FIG. 14B illustrates HPLC chromatograms of peptides of SEQ ID NO: 39 and SEQ ID NO: 43 with or without incubation in 10 mM reduced glutathione (GSH).

FIG. 15 illustrates the stability of peptides of SEQ ID NO: 39 and SEQ ID NO: 43 after exposure to a reducing agent. Each peptide is expressed on a cell surface and tested for binding to a target protein after cells expressing SDGF-SEQ ID NO: 43 or SEQ ID NO: 39 are exposed to a reducing agent. SDGF is surface display GFP FasL vector.

FIG. 15A illustrates a flow cytometry plot showing binding of HEK-293 suspension cells transfected with SDGF-SEQ ID NO: 39 (GFP) incubated for 5 minutes in PBS, 10 mM DTT, or 10 mM reduced glutathione (GSH) before staining with 20 nM biotinylated target protein, followed by a wash and then incubation with 20 nM AF647-streptavidin.

FIG. 15B illustrates a flow cytometry plot showing binding of HEK-293 suspension cells transfected with SDGF-SEQ ID NO: 44 (GFP) incubated for 5 minutes in PBS, 10 mM DTT, or 10 mM reduced glutathione (GSH) before staining with 20 nM biotinylated target protein, followed by a wash and then incubation with 20 nM AF647-streptavidin.

FIG. 15C illustrates quantification of the AF647 mean fluorescence intensity (MFI) of cells falling within the “slice” gate shown in FIG. 15A and FIG. 15B.

FIG. 16 illustrates protease resistance of a peptide of SEQ ID NO: 43.

FIG. 16A illustrates HPLC chromatograms of a peptide of SEQ ID NO: 43 after incubation with 500U trypsin (T), which was then quenched with trypsin inhibitor (I) and placed in non-reducing (NR) conditions or reducing (R) conditions with 10 mM DTT. SDPR is a variant of the surface display GFP FasL (SDGF) vector, but with all basic and aromatic residues within the stalk removed to prevent trypsin/chymotrypsin cleavage, and with a 6×His tag (SEQ ID NO: 180) added to the C-terminus of the peptide.

FIG. 16B illustrates a flow cytometry plot of HEK-293 suspension cells transfected with protease sensitive SDPR-SK peptide and the treated with 0 or 40 μg/ml trypsin for 20 minutes, and stained with an AF647 anti-6×HIS antibody (“6×HIS” disclosed as SEQ ID NO: 180).

FIG. 16C illustrates a flow cytometry plot of HEK-293 suspension cells transfected with SDPR-SEQ ID NO: 43 peptide and then treated with 0 or 40 μg/ml trypsin for 20 minutes, and stained with an AF647 anti-6×HIS antibody (“6×HIS” disclosed as SEQ ID NO: 180).

FIG. 16D illustrates a flow cytometry plot of HEK-293 suspension cells transfected with SDPR-SK peptide and then treated with 0 or 40 μg/ml chymotrypsin for 20 minutes, and stained with an AF647 anti-6×HIS antibody (“6×HIS” disclosed as SEQ ID NO: 180).

FIG. 16E illustrates a flow cytometry plot of HEK-293 suspension cells transfected with protease sensitive SDPR-SEQ ID NO: 43 peptide and then treated with 0 or 40 μg/ml chymotrypsin for 20 minutes, and stained with an AF647 anti-6×HIS antibody (“6×HIS” disclosed as SEQ ID NO: 180).

FIG. 16F illustrates quantification of flow cytometry data comparing SDPR-SK peptide transfected cells and SDPR-SEQ ID NO: 43 peptide transfected cells, both incubated with trypsin at various concentrations.

FIG. 16G illustrates quantification of flow cytometry data comparing SDPR-SK peptide transfected cells and SDPR-SEQ ID NO: 43 peptide transfected cells, both treated with chymotrypsin at various concentrations.

FIG. 17 illustrates SEQ ID NO: 43 and variants of SEQ ID NO: 43 are stable in extreme heat.

FIG. 17A illustrates circular dichroism spectra of SEQ ID NO: 43, which demonstrates the structure is dominated by α-helical elements, and that this secondary structure signature is identical before (Pre) and after (Post) incubation at 95° C. Inset: relative ellipticity at 220 nm during heating from 20° C. to 95° C.

FIG. 17B illustrates circular dichroism spectra of SEQ ID NO: 44, which demonstrates the structure is dominated by α-helical elements, and that this secondary structure signature is similar before (Pre) and after (Post) incubation at 95° C. Inset: relative ellipticity at 220 nm during heating from 20° C. to 95° C.

FIG. 17C illustrates circular dichroism spectra of SEQ ID NO: 45, which demonstrates the structure is dominated by α-helical elements, and that this secondary structure signature is identical before (Pre) and after (Post) incubation at 95° C. Inset: relative ellipticity at 220 nm during heating from 20° C. to 95° C.

FIG. 17D illustrates a SYPRO Orange melting assay of Peptides. Shown is the slope of the change in relative fluorescence units (dRFU/dtemp) during heating from 20° C. to 95° C. Human siderocalin (HuScn) demonstrated an expected melting temperature of 79° C., as interpreted by the peak of its RFU vs temperature slope. Conversely, no melting temperature could be determined for the three peptides tested (SEQ ID NO: 43, SEQ ID NO: 44, and SEQ ID NO: 45).

FIG. 18 illustrates high performance liquid chromatograph (HPLC) traces of peptides in non-reducing (NR) conditions (solid trace) or in 10 mM DTT reducing (R) conditions (dashed trace).

FIG. 19 illustrates high performance liquid chromatograph (HPLC) traces of various peptides of the present disclosure after incubation at 75° C. for 1 hour (solid trace) or 100° C. for 1 hour (dashed trace).

FIG. 20 illustrates high performance liquid chromatograph (HPLC) traces of peptides after pepsin digestion. The solid trace shows a reaction of peptide and pepsin that was quenched at alkaline pH and run under non-reducing conditions. The dashed trace shows a reaction of peptide and pepsin that was quenched at alkaline pH and run under reducing conditions.

FIG. 21 illustrates high performance liquid chromatograph (HPLC) traces of peptides after trypsin digestion. The solid trace shows a reaction of peptide and trypsin that was quenched at neutral pH with excess trypsin inhibitor and run under non-reducing conditions. The dashed trace shows a reaction of peptide and trypsin that was quenched at neutral pH with excess trypsin inhibitor and run under reducing conditions.

FIG. 22 illustrates circular dichroism results of various peptides of the disclosure.

FIG. 23 illustrates circular dichroism analysis of a peptide of SEQ ID NO: 27 after incubation in phosphate buffer saline at pH 7.2, phosphate buffer saline at pH 7.2 with 1% sodium dodecyl sulfate (SDS), or phosphate buffer saline at pH 4.

FIG. 24 illustrates circular dichroism analysis of a peptide of SEQ ID NO: 37 after incubation in phosphate buffer saline at pH 7.2, phosphate buffer saline at pH 7.2 with 1% sodium dodecyl sulfate (SDS), or phosphate buffer saline at pH 4.

FIG. 25 illustrates a structure-based cystine-dense peptide (CDP) classification scheme.

FIG. 25A illustrates cysteines in cystine-dense peptides (CDPs) numbered sequentially from 1 to 6 in the three-cystine core, which yields 15 theoretically possible cystine covalent bonds (disulfide bonds). The 15 theoretically possible disulfide bonds linking six cysteines pairwise are shown, arranged by the five possible pairings in the first disulfide bond (Cys1-Cys2 (1-2), Cys1-Cys3 (1-3), Cys1-Cys4 (1-4), Cys1-Cys5 (1-5), and Cys1-Cys6 (1-6); with numbering from N- to C-terminal). Subsequent pairings in the remaining disulfide bonds are shown in descending rows. As of April 2017, the Protein Data Bank contained 771 CDPs, comprising 422 knotted CDPs, 199 non-knotted CDPs with three cystines, and 150 non-knotted CDPs with more than three cystines. Percentage class distributions of the 621 knotted CDPs, plus non-knotted, three-cystine CDPs, are shown in parentheses, as the disulfide bonds of the 150 non-knotted CDPs with more than three cystines as there is no knotting element to define which three cystines constitute the focus, core subset. The Cys1-Cys4, Cys2-Cys5, Cys3-Cys6 (1-4, 2-5, 3-6) disulfide bond pattern was the most commonly observed in experimentally-determined CDP structures deposited in the Protein Data Bank (309 peptides of which were 295 knotted and 14 were non-knotted). Conversely, the Cys1-Cys6, Cys2-Cys3, Cys4-Cys5 (1-6, 2-3, 4-5) pattern was absent from the experimentally determined CDP structures in the Protein Data Bank.

FIG. 25B illustrates an overall cysteine-dense peptides (CDP) scheme, which shows the relationship of cysteine density (growth factor cystine knots (GFCKs) vs. CDPs), pseudoknotting (the knotted CDP subset of CDPs), and type classifications based on cystine covalent bond (disulfide bond) class plus knotting topology. Common disulfide bond patterns are grouped within boxes, highlighting that only five of the 15 possible disulfide bond patterns were observed among known knotted CDP structures. Example cartoon schematics of disulfide bonds/topology (numbered circles indicate cysteines; number observed in the Protein Data Bank is indicated in parentheses) are shown for the canonical shankins, hitchins, and knottins, and the simplest non-knotted CDP type, (Cys1-Cys6, Cys2-Cys5, Cys3-Cys4 (1-6, 2-5, 3-4); at upper right). Only types observed in the PDB as of April 2017 are indicated.

FIG. 25C illustrates the distribution of 771 CDP structures in each disulfide bond classification type. Knotted types are shown by white bars and non-knotted classes are shown by hatched bars.

FIG. 26 illustrates identification of stable peptides identified using a surface display peptide folding assay.

FIG. 26A illustrates the taxonomic diversity of the full library that was screened to identify stable peptides. Only classes with greater than 300 library members are specifically named in this pie chart.

FIG. 26B illustrates a dot plot, which on the x-axis shows the protein content displayed at the surface of cells transduced with a surface display GFP FasL(SDGF) vector comprising a peptide that were untreated and on the y-axis shows the protein content displayed at the surface of cells transduced with a surface display GFP FasL(SDGF) vector comprising a peptide that are treated with trypsin as a percentage of untreated surface protein content. The dot plot represents a total of about 4,300 peptides that passed read abundance thresholds out of the approximately 10,000 peptides that were initially cloned into surface display GFP FasL (SDGF) vectors. The diagonal line bisecting samples defines a cutoff between “high protein content and/or trypsin resistant” peptides and “low protein content and/or trypsin sensitive” peptides. Peptides that were further expressed as secreted proteins were classified by HPLC as 1-2 peaks (circle), 3+ peaks (square), 0 peaks (diamond), or no HPLC data (plus). Well-folded peptides appear in the top right quadrant and poorly-folded peptides appear in the bottom left quadrant.

FIG. 26C illustrates a bar graph showing the breakdown of the tested secreted proteins (from FIG. 26B) classified by HPLC (0 Peaks; 3+ Peaks; or 1-2 Peaks). Peptides were categorized by peptide category: All Peptides; Peptides with high protein content/trypsin resistant (High Content/Trypsin Resistant); and Peptides with low protein content/trypsin sensitive (Low Content/Trypsin Sensitive). The correlation between surface folding and HPLC classification (combining 3+ and 0 peaks into one group) was highly significant.

FIG. 26D illustrates HPLC traces of various peptides of this disclosure under native (thin line) or reducing (thick line) conditions. The peptide sequence for each “Plot” is shown in FIG. 26E.

FIG. 26E shows a table of each peptide that was tested by HPLC and for which HPLC chromatograms are shown in FIG. 26D. The table shows Plot number, the number of HPLC peaks that were observed, protein content, trypsin resistance, SEQ ID NO, and sequence.

FIG. 27 shows a sequence alignment and analysis of six hitchin peptides that were highly resistant to reduction, pepsin, and elevated temperatures such as 75° C. and 100° C., and nine hitchin peptides that were not highly resistant to these conditions.

FIG. 27A shows the sequence alignment of SEQ ID NO: 3, SEQ ID NO: 8, SEQ ID NO: 30, SEQ ID NO: 5, SEQ ID NO: 35, SEQ ID NO: 36, SEQ ID NO: 37, SEQ ID NO: 18, and SEQ ID NO: 56, which encode peptides that were not highly resistant to reduction, pepsin and elevated temperature.

FIG. 27B shows the sequence alignment of SEQ ID NO: 27, SEQ ID NO: 24, SEQ ID NO: 6, SEQ ID NO: 10, SEQ ID NO: 12, and SEQ ID NO: 57, which encode peptides that were highly resistant to reduction, pepsin, and elevated temperatures, such as 75° C. and 100° C.

FIG. 27C shows a sequence analysis of SEQ ID NO: 27, SEQ ID NO: 24, SEQ ID NO: 6, SEQ ID NO: 10, SEQ ID NO: 12, and SEQ ID NO: 57 from FIG. 27B.

FIG. 27D shows a sequence alignment of SEQ ID NO: 27, SEQ ID NO: 57, and SEQ ID NO: 24 from FIG. 27B. This subset of peptides that were highly resistant to reduction, pepsin, and elevated temperature, such as 75° C. and 100° C., are referred to as “Subtype A”. The conserved structural proline amino acid residue is circled.

FIG. 27E shows a sequence analysis of Subtype A SEQ ID NO: 27, SEQ ID NO: 57, and SEQ ID NO: 24 with the conserved structural proline amino acid residue circled from FIG. 27D.

FIG. 27F shows a sequence alignment of SEQ ID NO: 6, SEQ ID NO: 10, and SEQ ID NO: 12 from FIG. 27B. This subset of peptides that were highly resistant to reduction, pepsin, and elevated temperature, such as 75° C. and 100° C., are referred to as “Subtype B”. The conserved structural proline amino acid residue is circled.

FIG. 27G shows a sequence analysis of Subtype B SEQ ID NO: 6, SEQ ID NO: 10, and SEQ ID NO: 12 with the conserved structural proline amino acid residue circled from FIG. 27F.

### DETAILED DESCRIPTION

The present disclosure relates to compositions and methods for identifying reduction- and protease-resistant peptides with enhanced stability. The present disclosure further relates to compositions of reduction- and protease-resistant peptides to a subject and methods of use thereof including oral administration, parenteral administration, and delivery of peptides to various compartments of the body, including lungs, nasal regions, buccal regions, joints, skin, vaginal tissue, rectal tissue, ocular tissue, and regions of the gastrointestinal (GI) tract. The present disclosure also relates to delivery of peptides to various cellular compartments including endosomes, lysosomes, and the cytosol.

The stability of peptide and protein drugs can play a key role in the pharmacokinetics, and can ultimately impact the use and efficacy of a therapeutic. After administration in vivo, a peptide can face harsh biological conditions including a range of proteases intended to digest peptides, reducing agents intended to reduce disulfide bridges and break tertiary structure, and low pH environments that promote denaturation of folded peptides and proteins. Peptide therapeutics that are stable to degradation by proteases, reducing agents, and low pH environments can have enhanced or diversified use and better efficacy due to superior biodistribution, higher bioavailability, longer biological half-life, activity in specific compartments of the body and of the cell, and optimal overall pharmacokinetics. A peptide of this disclosure can show the characteristics of resistance against degradation by proteases, and stability in the presence of reducing agents and low pH environments. In some embodiments, a peptide of this disclosure can be shown to have several characteristics indicative of improved stability after oral administration. The peptide can also be linked to an active agent for therapy, diagnosis, imaging, and other applications.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

### Peptides

In some embodiments, the peptides of the present disclosure comprise an amino acid sequence that is resistant to a reducing condition, such as reduced glutathione (GSH), which is a physiologically relevant reducing agent. In some embodiments, peptides described herein are resistant to proteolysis by a protease, such as trypsin, pepsin, chymotrypsin, or other proteases relevant to peptide stability in vivo or during manufacturing. In some embodiments, peptides of the present disclosure are partially resistant in a stronger reducing agent, such as DTT, in addition to being resistant in GSH reducing condition. In some embodiments, peptides of the present disclosure are resistant to elevated temperatures, such as 30° C., 40° C., 75° C., or 100° C. In some embodiments, peptides of the present disclosure are resistant to other conditions that can denature proteins, such as sodium dodecylsulfate (SDS) exposure. In some embodiments, peptides with enhanced stability are knotted peptides, cystine dense peptides, or knottins, or are derived from cystine dense peptides, knottins, or knotted peptides. In other embodiments, peptides with enhanced stability are not knotted peptides, nor are they derived from knotted peptides, but nonetheless possess enhanced stability, such as resistance to proteolysis and/or resistance to reducing conditions.

Knottins are a class of knotted peptides, usually ranging from about 11 to about 81 amino acids in length that are often folded into a compact structure. Knottins are typically assembled into a complex tertiary structure that is characterized by a number of intramolecular disulfide crosslinks and may contain beta strands, alpha helices, and other secondary structures. The presence of the disulfide bonds can give some knottins remarkable environmental stability, allowing them to withstand extremes of temperature and pH and to resist the proteolytic enzymes, such as those of the blood stream and the digestive system. The rigidity of knottins also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The knotted peptides can bind targets with antibody-like affinity. A wider examination of the sequence structure and sequence identity or homology of knottins reveals that they have arisen by convergent evolution in all kinds of animals and plants. In animals, they can be found in venoms, for example, the venoms of spiders and scorpions and have been implicated in the modulation of ion channels. The knottin proteins of plants can inhibit the proteolytic enzymes of animals or have antimicrobial activity, suggesting that knottins can function in the native defense of plants.

The present disclosure provides peptides that can comprise or can be derived from these knotted peptides. As used herein, the term “knotted peptide” can be interchangeable with the terms “knottin” and “optide.” Hitchins, amongst other disulfide-containing peptides, can also be considered “knotted peptides” for the purposes of this disclosure.

The peptides of the present disclosure can comprise cysteine amino acid residues. In some cases, the peptide has at least 6 cysteine amino acid residues. In some cases, the peptide has at least 4 cysteine amino acid residues. In some cases, the peptide has at least 8 cysteine amino acid residues. In other cases, the peptide has at least 10 cysteine amino acid residues, at least 12 cysteine amino acid residues, at least 14 cysteine amino acid residues, or at least 16 cysteine amino acid residues.

A knotted peptide can comprise disulfide bridges. A knotted peptide can be a peptide wherein 5% or more of the residues can be cysteine amino acid residues forming intramolecular disulfide bonds or cystines. A knotted peptide can be a peptide that comprises at least 3 intramolecular disulfide bonds. A disulfide-linked peptide can be a drug scaffold. In some embodiments, the disulfide bridges form a knot. A disulfide bridge can be formed between cysteine amino acid residues, for example, between cysteine amino acid residues 1 and 4, 2 and 5, and 3 and 6. In some cases, one disulfide bridge passes through a loop formed by the other two disulfide bridges, for example, to form the knot. In other cases, the disulfide bridges can be formed between any two cysteine residues.

The present disclosure can also comprise peptides that are not canonical knottins. Some of these peptides can be hitchins, as described herein, or can have other disulfide covalent bonding topologies as compared to canonical knottins. Proteins can be differentiated from simpler peptides by size. In some embodiments, peptides can comprise less than about 50 residues long. In some embodiments, peptides do not fold into defined three-dimensional structures, as they lack enough cooperative interactions to form a stable structure, which can be accomplished through a well-packed hydrophobic core. Some exceptions can include peptides that alternately organize around cores of multiple, tightly-packed disulfide covalent bonds, which can confer extreme thermal, chemical, and proteolytic stability as set forth in Werle et al. (J Drug Target, 14(3): 137-46 (2006)), Gelly et al. (Nucleic Acids Res, 32(Database issue): D156-9 (2004)), Reinwarth et al. (Molecules, 17(11): 12533-52 (2012)), Kolmar et al. (Curr Pharm Des, 17(38): 4329-36 (2011)), Kolmar et al. (Curr Opin Pharmacol, 9(5):608-14 (2009)), Klintzing et al. (Curr Opin Chem Biol, 34: 143-150 (2016)), and Gould et al. (Curr Pharm Des, 17(38): 4294-307 (2011)). Importantly, not all peptides with multiple disulfide covalent bonds, or cysteine-dense peptides, may have high levels of these stabilities. The archetypes of such peptides can include “inhibitor cystine knotted peptides,” also called knottins (described in the present disclosure), and the closely-related “cyclic cystine knotted peptides”, known as cyclotides, which both can have cores of at least three cystines. Examples can include venom toxins from cone snails, spiders, and scorpions; protease inhibitors from plants; and antimicrobial defensins. Knottins and cyclotides can be topologically pseudoknotted, with one cystine crossing through the macrocycle formed by the other two cystines and the interconnecting backbone. Proteins can also incorporate cystine-knotted subdomains, for example, growth factor cystine knots (GFCKs) as set forth in Vitt et al. (Mol Endocrinol, 15(5): 681-94 (2001) and Iyer et al. (FEBS J, 278(22): 4304-22 (2011)). However, the GFCK cystine-knotted element does not dominate the fold of the protein, which can include a conventional hydrophobic core, distinct from knottins and cyclotides. In some embodiments, the minimal common elements defining this class of molecules can be short sequences, constituting independent folding domains, with a high density of at least three cystines. This categorization can be referred to as “cystine-dense peptides” (CDPs), drawing a distinction with larger proteins with cystine-knotted elements, like GFCKs.

A CDP can have a knotted topology and can be defined as comprising a CDP-defining motif: sequences that can comprise six or more cysteine amino acid residues (or at least three cystines), may not be recognizable as a cytoplasmic protein or domain, a zinc finger protein, or a GFCK, can comprise a constrained distribution of cysteine amino acid residues, can be Cys-X_{[0-15]}-Cys-X_{[0-15]}-Cys-X_{[0-15]}-Cys-X_{[0-15]}-Cys-X_{[0-15]}-Cys (SEQ ID NO: 179) wherein X can be any amino acid residue, and can be from 13 to 81 residues long between the motif-bonding cysteine amino acid residues. For example, a candidate CDP can be embedded in a sequence with a recognizable leader peptide such as SignalP (Bendtsen, J. D., J Mol Biol, 340: 783-795 (2004)), can be annotated as a secreted or integral membrane protein, or can be experimentally shown to contain specific cystines, which can be used to confirm the formation of cystines in the peptide to classify the peptide as a CDP. CDPs can be embedded in larger proteins, or in tandem arrays, and can comprise an independent folding unit. The additional criterion of a minimal “cysteine density,” in which the minimal “cysteine density” can be a sequence with a cysteine amino acid residue content of at least 12%, can separate CDPs with dominant cystine cores from small proteins with emergent hydrophobic cores. This threshold CDP-defining cysteine density can be approximately 10-fold higher than the average observed for all proteins (Moura, A., PloS One, 8Ie77319 (2013); The UniProtC., Nucleic Acids Res, 45: D158-D169 (2017)). As of April 2017, there were 771 experimentally-determined structures in the PDB that can conform to this sequence-based definition.

The first level of CDP classification can be determined by disulfide bonds as shown in FIG. 25A. Numbering the cysteines in the three-cystine core/knotting element sequentially from 1 to 6 can yield 15 theoretically possible disulfide bond classes, with most GFCKs and archetypical knottin knotted CDPs falling into the Cys1-Cys4, Cys2-Cys5, Cys3-Cys6 disulfide bond class (1-4, 2-5, 3-6), which can be referred to as the “canonical” disulfide bond class. Four other disulfide bond classes can be observed in deposited knotted CDP structures (with variable representation). Additionally, nine other disulfide bond classes can be observed in other non-knotted CDPs with three cystines as shown in FIG. 25B. The Cys1-Cys6, Cys2-Cys3, Cys4-Cys5 disulfide bond class (1-6, 2-3, 4-5) may not observed in any natural CDPs, though can be found in wholly synthetic, designed CDPs (e.g., 5JI4.pdb (Bhardwaj, G., Nature, 538: 329-335 (2016)). Non-knotted CDPs with more than three cystines cannot be assigned to comparable disulfide bond classes, as the focus subset of three cystines cannot be defined and numbered in the same way in the absence of a knotting element, but can be lumped together in a separate CDP type, which can be referred to as type “z”.

The second level of CDP classification can be based on cystine topology and can be defined as which cystine can pseudoknot the fold, focusing on the three core cystines comprising the knotting element, and ignoring additional, accessory cystines as shown in FIG. 25B. In any disulfide bond class, denoted as Cysu-Cysv, Cysw-Cysx, Cysy-Cysz (u-v, w-x, y-z) to indicate the core disulfide bond, there can be three theoretical topologies, each with a different knotting cystine, represented as u-v, w-x, [y-z], in which the knotting cystine can be indicated by square brackets. Knowledge of the CDP disulfide bond class and its corresponding knotting topology can indicate the structure-based knotted CDP type as shown in FIG. 25B. Non-knotted CDPs with three cystines can be denoted solely by Cysu-Cysv, Cysw-Cysx, Cysy-Cysz disulfide bond class (u-v, w-x, y-z), and non-knotted CDPs with more than three cysteines can be denoted as “z”. Using this nomenclature, archetypical knottins can be classified as type Cys1-Cys4, Cys2-Cys5, [Cys3-Cys6] knotted CDPs (1-4, 2-5, [3-6]), which can be distinct from the [Cys1-Cys4], Cys2-Cy5, Cys3-Cys6 topology ([1-4], 2-5, 3-6) that can be observed in GFCKs despite a common disulfide bond. The second most commonly observed topology type in this knotted CDP disulfide bond class can be Cys1-Cys4, [Cys2-Cys5], Cys3-Cys6 (1-4, [2-5], 3-6). Third topology type can be a GFCK-like, topology in this disulfide bond class: [Cys1-Cys4], Cys2-Cys5, Cys3-Cys6 ([1-4], 2-5, 3-6). Following the knottin nomenclature, the type 1-4, [2-5], 3-6 knotted CDPs can be referred to as “hitchins”, type [1-4], 2-5, 3-6 GFCKs can be referred to as “shanks” (a shank can be a type of knot used to shorten a length of rope), and rare type [1-4], 2-5, 3-6 knotted CDPs can be referred to as “shankins”. Though far fewer described knotted CDP structures can have non-canonical disulfide bond classes, examples of nine additional knotted CDP types have been reported as shown in FIG. 25B. The distribution of 771 CDPs among the different disulfide bond classes and types was predominately in the knottins, z-class, and hitchins as shown in FIG. 25C (non-knotted CDPs: black, and knotted CDPs: red). This proposed scheme can provide an unambiguous method for structural classification and comparison of CDPs independent of source organism, sequence homology, or functional annotation. Advantages can include avoiding broadly-applied annotations, like “defensin”, which can denote cysteine-rich, cationic, antimicrobial host defense peptides, but which can also encompass a wide range of structurally-dissimilar knotted and non-knotted CDP types, including many hitchins and knottins.

The peptides of the present disclosure can include, but are not limited to, knottins, hitchins, or other CDPs, as well as peptides that are not knotted. While the density of the cysteines and the optional presence of a knot can provide resistance to denaturation, reduction, proteases, and other structural degradations, peptides with knots or high cystine density can have varying resistance to such degradations, and some peptides can be much more stable and resistant. The peptides of the present disclosure can be more resistant to one or more chemical or physical degradation pathways.

In some embodiments, the tertiary structure and electrostatics of a peptide of the disclosure can impact stability. Structural analysis or analysis of charge distribution can be a strategy to predict residues important in biological. For example, several peptides of this disclosure that are stable can be grouped into a structural class defined above as “hitchins,” and can share the properties of disulfide linkages between Cys1-Cys4, Cys2-Cys5, and Cys3-Cys6. The folding topologies of peptides knotted through three disulfide linkages (Cys1-Cys4, Cys2-Cys5, and Cys3-Cys6), can be broken down into structural families based on the three-dimensional arrangement of the disulfides. Knottins can have the C3-C6 disulfide linkage passing through the macrocycle formed by the Cys1-Cys4 and Cys2-Cys5 disulfide linkages. Hitchins can have the Cys2-Cys5 disulfide linkage passing through the macrocycle formed by the Cys1-Cys4 and Cys3-Cys6 disulfide linkages. Other structural families can have the Cys1-Cys4 disulfide linkage passing through the macrocycle formed by the Cys2-Cys5 and Cys3-Cys6 disulfide linkages. Variants of “hitchin” class peptides with preserved disulfide linkages at these cysteine residues, primary sequence identity, and/or structural homology can be a method of identifying or predicting other potential knottin peptide candidates that can have high biological stability.

The present disclosure further includes peptide scaffolds that, e.g., can be used as a starting point for generating additional peptides. In some embodiments, these scaffolds can be derived from a variety of knotted peptides. In certain embodiments, knotted peptides can be assembled into a complex tertiary structure that is characterized by a number of intramolecular disulfide crosslinks, and optionally can contain beta strands and other secondary structures such as an alpha helix. For example, knotted peptides can include small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures can include peptide having two parallel helices linked by two disulfide bridges without β-sheets (e.g., hefutoxin).

A knotted peptide can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In still other embodiments, a knotted peptide is 11-81 amino acid residues long. In further embodiments, a knotted peptide is at least 20 amino acid residues long.

These kinds of peptides can be derived from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, or Chilobrachys jingzhao, or another suitable genus or species of scorpion or spider. As additional examples, the peptide can be derived from Pandinus imperator, Lychas mucronatus, Hadrurus gertschi, Centruroides elegans, Macrothele gigas, Centruroides limpidus limpidus, Mesobuthus tamulus, Pentadiplandra brazzeana, Heterometrus fulvipes, or Tachypleus tridentatus. In some cases, a peptide can be derived from a Buthus martensii Karsh (scorpion) toxin. In some embodiments, a peptide can be derived from a member of the pfam005453: Toxin_6 class.

In other embodiments, the present disclosure provides peptides that are not derived from knottins. In these embodiments, peptides can be designed or engineered using in silico techniques and/or random mutagenesis techniques. For example, peptides of SEQ ID NO: 39-SEQ ID NO: 45 and peptides of SEQ ID NO: 122-SEQ ID NO: 128 were designed or engineered using in silico and mutagenesis methods. Experiments with physiologically relevant reducing agent, GSH, showed peptides of SEQ ID NO: 39, SEQ ID NO: 43, SEQ ID NO: 122, and SEQ ID NO: 126 are resistant to GSH reducing condition. Experiments with a stronger reducing agent, DTT, showed peptides of SEQ ID NO: 43-SEQ ID NO: 45 and SEQ ID NO: 126-SEQ ID NO: 128 are partially resistant in DTT reducing conditions. Experiments with trypsin or chymotrypsin showed peptides of SEQ ID NO: 43 and SEQ ID NO: 126 are partially resistant to trypsin and chymotrypsin. In some embodiments, a peptide of the disclosure can be non-naturally occurring. Non-naturally occurring can refer to an article not caused by or existing in nature in its natural form.

TABLE 1 lists exemplary peptides for use with the present disclosure.

TABLE 1
Exemplary Peptide Sequences

| SEQ ID NO | Amino Acid Sequence |
|---|---|
| SEQ ID NO: 1 | GSDCLPHLRRCRADNDCCGRRCRRRGTNAERRCR |
| SEQ ID NO: 2 | GSQFTNVSCTTSRECWSVCQRLHNTSRGRCMNRRCRCYS |
| SEQ ID NO: 3 | GSISIGIKCSPSIDLCEGQCRIRKYFTGYCSGDTCHCSG |
| SEQ ID NO: 4 | GSGDCLPHLKRCKENNDCCSKKCKRRGANPEKRCR |
| SEQ ID NO: 5 | GSNFKVEGACSKPCRKYCIDKGARNGKCINGRCHCYY |
| SEQ ID NO: 6 | GSQKILSNRCNNSSECIPHCIRIFGTRAAKCINRKCYCYP |
| SEQ ID NO: 7 | GSDRDSCIDKSRCSKYGYYQECQDCCKKAGHNGGTCMFFKCKCA |
| SEQ ID NO: 8 | GSAVCNLKRCQLSCRSLGLLGKCIGDKCECVKHG |
| SEQ ID NO: 9 | GSQFCGTNGKPCVNGQCCGALRCVVTYHYADGVCLKMNP |
| SEQ ID NO: 10 | GSRPTDIKCSASYQCFPVCKSRFGKTNGRCVNGLCDCF |
| SEQ ID NO: 11 | GSNCAGYMRECKEKLCCSGYVCSSRWKWCVLPAPWRR |
| SEQ ID NO: 12 | GSQFTDVKCTGSKQCWPVCKQMFGKPNGKCMNGKCRCYS |
| SEQ ID NO: 13 | GSAEIIRCSGTRECYAPCQKLTGCLNAKCMNKACKCYGCV |
| SEQ ID NO: 14 | GSSDYCSNDFCFFSCRRDRCARGDCENGKCVCKNCHLN |
| SEQ ID NO: 15 | GSCIGEGVPCDENDPRCCFGLVCLKPTLHGIWYKSYYCYKK |
| SEQ ID NO: 16 | GSSCAKPGEMCMRIKCCDGQCGCNRGTGRCFCK |
| SEQ ID NO: 17 | GSACQFWSCNSSCISRGYRQGYCWGIQYKYCQCQ |
| SEQ ID NO: 18 | GSQVSTNKKCSNTSQCYKTCEKVVGVAAGKCMNGKCICYP |
| SEQ ID NO: 19 | GSQDKCKKVYENYPVSKCQLANQCNYDCKLDKHARSGECFYDEKRNLQCICDYCEY |
| SEQ ID NO: 20 | GSGHACYRNCWREGNDEETCKERC |
| SEQ ID NO: 21 | GSRCQLQGFNCVVRSYGLPTIPCCRGLTCRSYFPGSTYGRCQRY |
| SEQ ID NO: 22 | GSMCMPCFTTRPDMAQQCRACCKGRGKCFGPQCLCGYD |
| SEQ ID NO: 23 | GSEVIRCSGSKQCYGPCKQQTGCTNSKCMNKVCKCYGCG |
| SEQ ID NO: 24 | GSVRIPVSCKHSGQCLKPCKDAGMRFGKCMNGKCDCTPK |
| SEQ ID NO: 25 | GSACLAEYQKCEGSTVPCCPGLSCSAGRFRKTKLCTK |
| SEQ ID NO: 26 | GSVGCEECPMHCKGKNANPTCDDGVCNCNV |
| SEQ ID NO: 27 | GSGVPINVKCRGSRDCLDPCKKAGMRFGKCINSKCHCTP |
| SEQ ID NO: 28 | GSRCPPCFTTNPNMEADCRKCCGGRGYCASYQCICPGG |
| SEQ ID NO: 29 | GSSEKDCIKHLQRCRENKDCCSKKCSRRGTNPEKRCR |
| SEQ ID NO: 30 | GSQVQTNVKCQGGSCASVCRREIGVAAGKCINGKCVCYRN |
| SEQ ID NO: 31 | GSGVPINVRCRGSRDCLDPCRRAGMRFGRCINSRCHCTP |
| SEQ ID NO: 32 | GSSCAKPRENCNRMNILCCRGECVCPTFGDCFCYGD |
| SEQ ID NO: 33 | GSDCVRFWGKCSQTSDCCPHLACKSKWPRNICVWDGSVG |
| SEQ ID NO: 34 | GSGCFGYKCDYYKGCCSGYVCSPTWKWCVRPGPGR |
| SEQ ID NO: 35 | GSQIDTNVKCSGSSKCVKICIDRYNTRGAKCINGRCTCYP |
| SEQ ID NO: 36 | GSVVIGQRCYRSPDCYSACKKLVGKATGKCTNGRCDC |
| SEQ ID NO: 37 | GSVFINVKCRGSPECLPKCKEAIGKSAGKCMNGKCKCYP |
| SEQ ID NO: 38 | GSECLEIFKACNPSNDQCCKSSKLVCSRKTRWCKYQIG |
| SEQ ID NO: 39 | GSPDEYIERAKECCKKGDIQCCLRYFEESGDPNVMLICLFCP |
| SEQ ID NO: 40 | GSPDEYIERAKECCKKGDIQCCLRYFEESGDPNVMLICAFCP |
| SEQ ID NO: 41 | GSPDEYIERAKECCKKGDIQCCLRYFEESGDPNVMLICLACP |
| SEQ ID NO: 42 | GSFGLYDNQCATSDACSAICKYWTGSGQGKCQNNQCRCY |
| SEQ ID NO: 43 | GSPDEYIERAKECCKKQDIQCCLRIFDESKDPNVMLICLFCW |
| SEQ ID NO: 44 | GSPDEYIERAKECCKKQDIQCCLRIFDESGDPNVMLICLFCW |
| SEQ ID NO: 45 | GSPDEYIERAKECCKKQDIQCCLRIFDESKDPNVMLICLFCP |
| SEQ ID NO: 46 | GSDCKYKFENWGACDGGTGTKVRQGTLKKARYNAQCQETIRVTKPC |
| SEQ ID NO: 47 | GSAQEPVKGPVSTKPGSCPIILIRCAMLNPPNRCLKDTDCPGIKKCCEGSCGMACFVPQ |
| SEQ ID NO: 48 | GSGIGDPVTCLKSGAICHPVFCPRRYKQIGTCGLPGTKCCKKP |
| SEQ ID NO: 49 | GSSFGLCRLRRGFCARGRCRFPSIPIGRCSRFVQCCRRVW |
| SEQ ID NO: 50 | GSLFCKGGSCHFGGCPSHLIKVGSCFGFRSCCKWPWNA |
| SEQ ID NO: 51 | GSMCMPCFTTDHQMARKCDDCCGGKGRGKCYGPQCLCR |
| SEQ ID NO: 52 | GSEGDCPISEAIKCVEKCKEKVEVCEPGVCKCSG |
| SEQ ID NO: 53 | GSLCLPCFTTHHRLADQCDICCGGDGRGKCYGPQCLCR |
| SEQ ID NO: 54 | GSVSCEDCPEHCATKDQRAKCDNDRCVCEPK |
| SEQ ID NO: 55 | GSGIVCKVCKIICGMQGKKVNICKAPIKCKCKKG |
| SEQ ID NO: 56 | GSGVPTDVKCRGSPQCIQPCKDAGMRFGKCMNGKCHCTPK |
| SEQ ID NO: 57 | GSGVIINVKCKISRQCLEPCKKAGMRFGKCMNGKCHCTPK |
| SEQ ID NO: 58 | GSAPPCKRDVDCSFECPKGGFCNDRLGTCDCF |
| SEQ ID NO: 59 | GSRQPCSYYDGVCRDKSDVNCKYIAFTYCENPNQRCCYY |
| SEQ ID NO: 60 | GSERECVGENGHCRSWYNDCCDGYYCSCMQPPNCICRNN |
| SEQ ID NO: 61 | GSKEICERPNGSCRDFCLETEIHVGRCLNSQPCCLPL |
| SEQ ID NO: 62 | GSYTDCTESGQNLCLCEGSNVCGKGNKCILGSQGKDNQCVTG |
| SEQ ID NO: 63 | GSPMTCEQAMASCEHTMCGYCQGPLYMTCIGITTDPECGLP |
| SEQ ID NO: 64 | GSKDYCPGICNVAAVPDCDTLCISLGYSGGYCRAGRICCCNPK |
| SEQ ID NO: 65 | GSYASCEAAEADCIHDDCFSEDTYTDVCQESCQYMYDNCMDD |
| SEQ ID NO: 66 | GSGICACRRRFCLNFEQFSGYCRVNGARYVRCCSRR |
| SEQ ID NO: 67 | GSDLVCYCRKRGCKRREHMNGTCRRGHLMYTLCCR |
| SEQ ID NO: 68 | GSERGCGLLMDACDGKSTFCCSGYNCSPTWKWCVLDCPNLFLLPPTKTLC |
| SEQ ID NO: 69 | GSSVPCVSTRGSCKPPAPACCHPCASCQCRFFRSACSCRVLNVNC |
| SEQ ID NO: 70 | GSRGQCWSYSNCRAVCRDEGYVSGHCNYFGGACWCAS |
| SEQ ID NO: 71 | GSLHRCRIYGTACADCCLARDPYCAWDGNSCSRF |
| SEQ ID NO: 72 | GSSDLCSTRQSRFRDYHCRCYSAWEGACCQTLRPSRCQKR |
| SEQ ID NO: 73 | GSVDGCQETCTKMIRCQFFTYSLFPEDCRGEKCKCSLR |
| SEQ ID NO: 74 | GSGVPIDVKCRGSPQCIQPCKDAGMRFGKCMNGKCHCTPK |
| SEQ ID NO: 75 | GSGVPIDVKCRGSPQCLQPCKDAGMRFGKCMNGKCHCTPK |
| SEQ ID NO: 76 | GSVFTNVKCRGSPECLPKCKERFGKSAGKCMNGKCKCYP |
| SEQ ID NO: 77 | GSVRIPVSCRHSGQCLRPCRDAGIVIRFGRCMNGRCDCTPR |
| SEQ ID NO: 78 | GSRPTDIRCSASYQCFPVCRSRFGRTNGRCVNGLCDCF |
| SEQ ID NO: 79 | GSSERDCIRHLQRCRENRDCCSRRCSRRGTNPERRCR |
| SEQ ID NO: 80 | GSGDCLPHLRRCRENNDCCSRRCRRRGANPERRCR |
| SEQ ID NO: 81 | GSMCIPCFTTNPNMAAKCNACCGSRRGSCRGPQCIC |
| SEQ ID NO: 82 | GSGCLEFWWKCNPNDDKCCRPKLKCSKLFKLCNFSFG |
| SEQ ID NO: 83 | GSECRYWLGTCSKTGDCCSHLSCSPKHGWCVWDWTFRK |
| SEQ ID NO: 84 | DCLPHLRRCRADNDCCGRRCRRRGTNAERRCR |
| SEQ ID NO: 85 | QFTNVSCTTSRECWSVCQRLHNTSRGRCMNRRCRCYS |
| SEQ ID NO: 86 | ISIGIKCSPSIDLCEGQCRIRKYFTGYCSGDTCHCSG |
| SEQ ID NO: 87 | GDCLPHLKRCKENNDCCSKKCKRRGANPEKRCR |
| SEQ ID NO: 88 | NFKVEGACSKPCRKYCIDKGARNGKCINGRCHCYY |
| SEQ ID NO: 89 | QKILSNRCNNSSECIPHCIRIFGTRAAKCINRKCYCYP |
| SEQ ID NO: 90 | DRDSCIDKSRCSKYGYYQECQDCCKKAGHNGGTCNIFFKCKCA |
| SEQ ID NO: 91 | AVCNLKRCQLSCRSLGLLGKCIGDKCECVKHG |
| SEQ ID NO: 92 | QFCGTNGKPCVNGQCCGALRCVVTYHYADGVCLKMNP |
| SEQ ID NO: 93 | RPTDIKCSASYQCFPVCKSRFGKTNGRCVNGLCDCF |
| SEQ ID NO: 94 | NCAGYMRECKEKLCCSGYVCSSRWKWCVLPAPWRR |
| SEQ ID NO: 95 | QFTDVKCTGSKQCWPVCKQMFGKPNGKCMNGKCRCYS |
| SEQ ID NO: 96 | AEIIRCSGTRECYAPCQKLTGCLNAKCMNKACKCYGCV |
| SEQ ID NO: 97 | SDYCSNDFCFFSCRRDRCARGDCENGKCVCKNCHLN |
| SEQ ID NO: 98 | CIGEGVPCDENDPRCCFGLVCLKPTLHGIWYKSYYCYKK |
| SEQ ID NO: 99 | SCAKPGEMCMRIKCCDGQCGCNRGTGRCFCK |
| SEQ ID NO: 100 | ACQFWSCNSSCISRGYRQGYCWGIQYKYCQCQ |
| SEQ ID NO: 101 | QVSTNKKCSNTSQCYKTCEKVVGVAAGKCMNGKCICYP |
| SEQ ID NO: 102 | QDKCKKVYENYPVSKCQLANQCNYDCKLDKHARSGECFYDEKRNLQCICDYCEY |
| SEQ ID NO: 103 | GHACYRNCWREGNDEETCKERC |
| SEQ ID NO: 104 | RCQLQGFNCVVRSYGLPTIPCCRGLTCRSYFPGSTYGRCQRY |
| SEQ ID NO: 105 | MCMPCFTTRPDMAQQCRACCKGRGKCFGPQCLCGYD |
| SEQ ID NO: 106 | EVIRCSGSKQCYGPCKQQTGCTNSKCMNKVCKCYGCG |
| SEQ ID NO: 107 | VRIPVSCKHSGQCLKPCKDAGIVIRFGKCMNGKCDCTPK |
| SEQ ID NO: 108 | ACLAEYQKCEGSTVPCCPGLSCSAGRFRKTKLCTK |
| SEQ ID NO: 109 | VGCEECPMHCKGKNANPTCDDGVCNCNV |
| SEQ ID NO: 110 | GVPINVKCRGSRDCLDPCKKAGMRFGKCINSKCHCTP |
| SEQ ID NO: 111 | RCPPCFTTNPNMEADCRKCCGGRGYCASYQCICPGG |
| SEQ ID NO: 112 | SEKDCIKHLQRCRENKDCCSKKCSRRGTNPEKRCR |
| SEQ ID NO: 113 | QVQTNVKCQGGSCASVCRREIGVAAGKCINGKCVCYRN |
| SEQ ID NO: 114 | GVPINVRCRGSRDCLDPCRRAGMRFGRCINSRCHCTP |
| SEQ ID NO: 115 | SCAKPRENCNRMNILCCRGECVCPTFGDCFCYGD |
| SEQ ID NO: 116 | DCVRFWGKCSQTSDCCPHLACKSKWPRNICVWDGSVG |
| SEQ ID NO: 117 | GCFGYKCDYYKGCCSGYVCSPTWKWCVRPGPGR |
| SEQ ID NO: 118 | QIDTNVKCSGSSKCVKICIDRYNTRGAKCINGRCTCYP |
| SEQ ID NO: 119 | VVIGQRCYRSPDCYSACKKLVGKATGKCTNGRCDC |
| SEQ ID NO: 120 | VFINVKCRGSPECLPKCKEAIGKSAGKCMNGKCKCYP |
| SEQ ID NO: 121 | ECLEIFKACNPSNDQCCKSSKLVCSRKTRWCKYQIG |
| SEQ ID NO: 122 | PDEYIERAKECCKKGDIQCCLRYFEESGDPNVMLICLFCP |
| SEQ ID NO: 123 | PDEYIERAKECCKKGDIQCCLRYFEESGDPNVMLICAFCP |
| SEQ ID NO: 124 | PDEYIERAKECCKKGDIQCCLRYFEESGDPNVMLICLACP |
| SEQ ID NO: 125 | FGLYDNQCATSDACSAICKYWTGSGQGKCQNNQCRCY |
| SEQ ID NO: 126 | PDEYIERAKECCKKQDIQCCLRIFDESKDPNVMLICLFCW |
| SEQ ID NO: 127 | PDEYIERAKECCKKQDIQCCLRIFDESGDPNVMLICLFCW |
| SEQ ID NO: 128 | PDEYIERAKECCKKQDIQCCLRIFDESKDPNVMLICLFCP |
| SEQ ID NO: 129 | DCKYKFENWGACDGGTGTKVRQGTLKKARYNAQCQETIRVTKPC |
| SEQ ID NO: 130 | AQEPVKGPVSTKPGSCPIILIRCAMLNPPNRCLKDTDCPGIKKCCEGSCGMACFVPQ |
| SEQ ID NO: 131 | GIGDPVTCLKSGAICHPVFCPRRYKQIGTCGLPGTKCCKKP |
| SEQ ID NO: 132 | SFGLCRLRRGFCARGRCRFPSIPIGRCSRFVQCCRRVW |
| SEQ ID NO: 133 | LFCKGGSCHFGGCPSHLIKVGSCFGFRSCCKWPWNA |
| SEQ ID NO: 134 | MCMPCFTTDHQMARKCDDCCGGKGRGKCYGPQCLCR |
| SEQ ID NO: 135 | EGDCPISEAIKCVEKCKEKVEVCEPGVCKCSG |
| SEQ ID NO: 136 | LCLPCFTTHHRLADQCDICCGGDGRGKCYGPQCLCR |
| SEQ ID NO: 137 | VSCEDCPEHCATKDQRAKCDNDRCVCEPK |
| SEQ ID NO: 138 | GIVCKVCKIICGMQGKKVNICKAPIKCKCKKG |
| SEQ ID NO: 139 | GVPTDVKCRGSPQCIQPCKDAGMRFGKCMNGKCHCTPK |
| SEQ ID NO: 140 | GVIINVKCKISRQCLEPCKKAGMRFGKCMNGKCHCTPK |
| SEQ ID NO: 141 | APPCKRDVDCSFECPKGGFCNDRLGTCDCF |
| SEQ ID NO: 142 | RQPCSYYDGVCRDKSDVNCKYIAFTYCENPNQRCCYY |
| SEQ ID NO: 143 | ERECVGENGHCRSWYNDCCDGYYCSCMQPPNCICRNN |
| SEQ ID NO: 144 | KEICERPNGSCRDFCLETEIHVGRCLNSQPCCLPL |
| SEQ ID NO: 145 | YTDCTESGQNLCLCEGSNVCGKGNKCILGSQGKDNQCVTG |
| SEQ ID NO: 146 | PMTCEQAMASCEHTMCGYCQGPLYMTCIGITTDPECGLP |
| SEQ ID NO: 147 | KDYCPGICNVAAVPDCDTLCISLGYSGGYCRAGRICCCNPK |
| SEQ ID NO: 148 | YASCEAAEADCIHDDCFSEDTYTDVCQESCQYMYDNCMDD |
| SEQ ID NO: 149 | GICACRRRFCLNFEQFSGYCRVNGARYVRCCSRR |
| SEQ ID NO: 150 | DLVCYCRKRGCKRREHMNGTCRRGHLMYTLCCR |
| SEQ ID NO: 151 | ERGCGLLMDACDGKSTFCCSGYNCSPTWKWCVLDCPNLFLLPPTKTLC |
| SEQ ID NO: 152 | SVPCVSTRGSCKPPAPACCHPCASCQCRFFRSACSCRVLNVNC |
| SEQ ID NO: 153 | RGQCWSYSNCRAVCRDEGYVSGHCNYFGGACWCAS |
| SEQ ID NO: 154 | LHRCRIYGTACADCCLARDPYCAWDGNSCSRF |
| SEQ ID NO: 155 | SDLCSTRQSRFRDYHCRCYSAWEGACCQTLRPSRCQKR |
| SEQ ID NO: 156 | VDGCQETCTKMIRCQFFTYSLFPEDCRGEKCKCSLR |
| SEQ ID NO: 157 | GVPIDVKCRGSPQCIQPCKDAGMRFGKCMNGKCHCTPK |
| SEQ ID NO: 158 | GVPIDVKCRGSPQCLQPCKDAGMRFGKCMNGKCHCTPK |
| SEQ ID NO: 159 | VFTNVKCRGSPECLPKCKERFGKSAGKCMNGKCKCYP |
| SEQ ID NO: 160 | VRIPVSCRHSGQCLRPCRDAGMRFGRCMNGRCDCTPR |
| SEQ ID NO: 161 | RPTDIRCSASYQCFPVCRSRFGRTNGRCVNGLCDCF |
| SEQ ID NO: 162 | SERDCIRHLQRCRENRDCCSRRCSRRGTNPERRCR |
| SEQ ID NO: 163 | GDCLPHLRRCRENNDCCSRRCRRRGANPERRCR |
| SEQ ID NO: 164 | MCIPCFTTNPNMAAKCNACCGSRRGSCRGPQCIC |
| SEQ ID NO: 165 | GCLEFWWKCNPNDDKCCRPKLKCSKLFKLCNFSFG |
| SEQ ID NO: 166 | ECRYWLGTCSKTGDCCSHLSCSPKHGWCVWDWTFRK |

In some embodiments, the peptide can have a sequence comprising GSX₁X₂X₃X₄X₅X₆X₇CX₈X₉SX₁₀X₁₁CX₁₂X₁₃X₁₄CX₁₅X₁₆X₁₇X₁₈GX₁₉X₂₀X₂₁X₂₂X₂₃CX₂₄NX₂₅X₂₆CX₂₇CX₂₈X₂₉X₃₀ (SEQ ID NO: 167), wherein X₁ can be G, Q, V, or null; X₂ can be V, R, K, or null; X₃ can be P, I, F, or null; X₄ can be I, T, or L; X₅ can be N, D, P, or S; X₆ can be V, I, or N; X₇ can be K, R, or S; X₈ can be R, K, N, S, or T; X₉ can be G, I, H, N, or A; X₁₀ can be R, K, G, S, or Y; X₁₁ can be D, Q, or E; X₁₂ can be L, I, F, or W; X₁₃ can be P, D, E, R, or K; X₁₄ can be P, V, or H; X₁₅ can be R, K or I; X₁₆ can be K, R, D, S, or Q; X₁₇ can be A, I, R, K, or M; X₁₈ can be null or F; X₁₉ can be M, K, R, or T; X₂₀ can be R, K, T, or P; X₂₁ can be F, N, or A; X₂₂ can be G or A; X₂₃ can be K or R; X₂₄ can be I, M, or V; X₂₅ can be S, G, R, or K; X₂₆ can be K, R, or L; X₂₇ can be H, D, Y, R, or K; X₂₈ can be T, Y, or F; X₂₉ can be P, S, or null; and X₃₀ can be null, K, or R. In some embodiments, the peptide can have a sequence comprising GSX₁X₂X₃IX₄VX₅CX₆X₇SX₈QCLX₉PCX₅X₁₀AGMX₅FGX₅CX₁₁NGX₅CX₁₂CTPX₁₃ (SEQ ID NO: 168), wherein X₁ can be G, Q, V, or null; X₂ can be V, R, K, or null; X₃ can be P, I, F, or null; X₄ can be N, D, P, or S; X₅ can be K or R; X₆ can be R, K, N, S, or T; X₇ can be G, I, H, N, or A; X₈ can be R, K, G, S, or Y; X₉ can be P, D, E, K, or R; X₁₀ can be K, R, D, S, or Q; X₁₁ can be I, M, or V; X₁₂ can be H, D, Y, K, or R; and X₁₃ can be null, K, or R. In some embodiments, the peptide can have a sequence comprising GSGVX₁INVX₂CX₂X₃SX₂X₄CLX₅PCX₂X₂AGMX₂FGX₂CX₆NX₇X₂CHCTPX₈ (SEQ ID NO: 169), wherein X₁ can be P or I; X₂ can be K or R; X₃ can be G or I; X₄ can be D or Q; X₅ can be D or E; X₆ can be I or M; X₇ can be S or G; and X₈ can be null, K, or R. In some embodiments, the peptide can have a sequence comprising GSX₁X₂X₃IX₄VX₅CX₆X₇SX₈X₉CLX₁₀PCX₆X₁₁AGMRFGX₆CX₁₂NXBX₆CX₁₄CTPX₆ (SEQ ID NO: 170), wherein X₁ can be G or V; X₂ can be V, R, or K; X₃ can be P, I, or null; X₄ can be N or P; X₅ can be K, S, or R; X₆ can be K or R; X₇ can be G, I, or H; X₈ can be R, G, or K; X₉ can be Q or D; X₁₀ can be D, E, K, or R; X₁₁ can be K, D, or R; X₁₂ can be M or I; X₁₃ can be G or S; and X₁₄ can be H or D. In some embodiments, the peptide can have a sequence comprising GSX₁X₂X₃X₄X₅X₆X₇CX₈X₉SX₁₀X₁₁CX₁₂PX₁₃CX₁₄X₁₅X₁₆FGX₁₇X₁₈X₁₉X₂₀X₂₁CX₂₂NX₂₃X₂₄CX₂₅CX₂₆X₂₇ (SEQ ID NO: 171), wherein X₁ can be Q or null; X₂ can be K, R, or null; X₃ can be I, P, or F; X₄ can be T or L; X₅ can be D or S; X₆ can be N, I, or V; X₇ can be K or R; X₈ can be N, S, or T; X₉ can be N, A, or G; X₁₀ can be S, Y, K, or R; X₁₁ can be Q or E; X₁₂ can be I, F, or W; X₁₃ can be V or H; X₁₄ can be K, I, or R; X₁₅ can be R, S, Q, or K; X₁₆ can be I, R, M, or K; X₁₇ can be K, T, or R; X₁₈ can be R, T, P, or K; X₁₉ can be N or A; X₂₀ can be G or A; X₂₁ can be K or R; X₂₂ can be I, V, or M; X₂₃ can be G, R, or K; X₂₄ can be K, L, or R; X₂₅ can be Y, D, R, or K; X₂₆ can be Y or F; and X₂₇ can be P, S, or null.

In some embodiments, the peptide can have a sequence comprising X₁X₂X₃X₄X₅X₆X₇CX₈X₉SX₁₀X₁₁CX₁₂X₁₃X₁₄CX₁₅X₁₆X₁₇X₁₈GX₁₉X₂₀X₂₁X₂₂X₂₃CX₂₄NX₂₅X₂₆CX₂₇CX₂₈X₂₉X₃₀ (SEQ ID NO: 172), wherein X₁ can be G, Q, V, or null; X₂ can be V, R, K, or null; X₃ can be P, I, F, or null; X₄ can be I, T, or L; X₅ can be N, D, P, or S; X₆ can be V, I, or N; X₇ can be K, R, or S; X₈ can be R, K, N, S, or T; X₉ can be G, I, H, N, or A; X₁₀ can be R, K, G, S, or Y; X₁₁ can be D, Q, or E; X₁₂ can be L, I, F, or W; X₁₃ can be P, D, E, R, or K; X₁₄ can be P, V, or H; X₁₅ can be R, K or I; X₁₆ can be K, R, D, S, or Q; X₁₇ can be A, I, R, K, or M; X₁₈ can be null or F; X₁₉ can be M, K, R, or T; X₂₀ can be R, K, T, or P; X₂₁ can be F, N, or A; X₂₂ can be G or A; X₂₃ can be K or R; X₂₄ can be I, M, or V; X₂₅ can be S, G, R, or K; X₂₆ can be K, R, or L; X₂₇ can be H, D, Y, R, or K; X₂₈ can be T, Y, or F; X₂₉ can be P, S, or null; and X₃₀ can be null, K, or R.

In some embodiments, the peptide can have a sequence comprising X₁X₂X₃IX₄VX₅CX₆X₇SX₈QCLX₉PCX₅X₁₀AGMX₅FGX₅CX₁₁NGX₅CX₁₂CTPX₁₃ (SEQ ID NO: 173), wherein X₁ can be G, Q, V, or null; X₂ can be V, R, K, or null; X₃ can be P, I, F, or null; X₄ can be N, D, P, or S; X₅ can be K or R; X₆ can be R, K, N, S, or T; X₇ can be G, I, H, N, or A; X₈ can be R, K, G, S, or Y; X₉ can be P, D, E, K, or R; X₁₀ can be K, R, D, S, or Q; X₁₁ can be I, M, or V; X₁₂ can be H, D, Y, K, or R; and X₁₃ can be null, K, or R. In some embodiments, the peptide can have a sequence comprising GVX₁INVX₂CX₂X₃SX₂X₄CLX₅PCX₂X₂AGMX₂FGX₂CX₆NX₇X₂CHCTPX₈ (SEQ ID NO: 174), wherein X₁ can be P or I; X₂ can be K or R; X₃ can be G or I; X₄ can be D or Q; X₅ can be D or E; X₆ can be I or M; X₇ can be S or G; and X₈ can be null, K, or R. In some embodiments, the peptide can have a sequence comprising X₁X₂X₃IX₄VX₅CX₆X₇SX₈X₉CLX₁₀PCX₆X₁₁AGMRFGX₆CX₁₂NX₁₃X₆CX₁₄CTPX₆ (SEQ ID NO: 175), wherein X₁ can be G or V; X₂ can be V, R, or K; X₃ can be P, I, or null; X₄ can be N or P; X₅ can be K, S, or R; X₆ can be K or R; X₇ can be G, I, or H; X₈ can be R, G, or K; X₉ can be Q or D; X₁₀ can be D, E, K, or R; X₁₁ can be K, D, or R; X₁₂ can be M or I; X₁₃ can be G or S; and X₁₄ can be H or D. In some embodiments, the peptide can have a sequence comprising X₁X₂X₃X₄X₅X₆X₇CX₈X₉SX₁₀X₁₁CX₁₂PX₁₃CX₁₄X₁₅X₁₆FGX₁₇X₁₈X₁₉X₂₀X₂₁CX₂₂NX₂₃X₂₄CX₂₅CX₂₆X₂₇ (SEQ ID NO: 176), wherein X₁ can be Q or null; X₂ can be K, R, or null; X₃ can be I, P, or F; X₄ can be T or L; X₅ can be D or S; X₆ can be N, I, or V; X₇ can be K or R; X₈ can be N, S, or T; X₉ can be N, A, or G; X₁₀ can be S, Y, K, or R; X₁₁ can be Q or E; X₁₂ can be I, F, or W; X₁₃ can be V or H; X₁₄ can be K, I, or R; X₁₅ can be R, S, Q, or K; X₁₆ can be I, R, M, or K; X₁₇ can be K, T, or R; X₁₈ can be R, T, P, or K; X₁₉ can be N or A; X₂₀ can be G or A; X₂₁ can be K or R; X₂₂ can be I, V, or M; X₂₃ can be G, R, or K; X₂₄ can be K, L, or R; X₂₅ can be Y, D, R, or K; X₂₆ can be Y or F; and X₂₇ can be P, S, or null.

In some embodiments, any one of SEQ ID NO: 167-SEQ ID NO: 171 or SEQ ID NO: 172-SEQ ID NO: 176 can be reduction resistant. In some embodiments, any one of SEQ ID NO: 167-SEQ ID NO: 171 or SEQ ID NO: 172-SEQ ID NO: 176 can be also resistant to one or more peptidases. In some embodiments, any one of SEQ ID NO: 167-SEQ ID NO: 171 or SEQ ID NO: 172-SEQ ID NO: 176 can also be resistant to elevated temperature. In some embodiments, SEQ ID NO: 169 or SEQ ID NO: 174 can be trypsin resistant.

In some embodiments, the number of disulfide bonds within a peptide can be at least 1, at least 2, at least 3, at least 4, at least 5, or at least 6.

In some instances, the peptide can contain only one lysine residue, or no lysine residues. In some embodiments, the peptide comprises at least two lysine residues. In some embodiments, the peptide comprises at least two consecutive lysine residues. In some instances, some or all of the lysine residues in the peptide are replaced with arginine residues. In some instances, some or all of the methionine residues in the peptide are replaced by leucine or isoleucine. Some or all of the tryptophan residues in the peptide can be replaced by phenylalanine or tyrosine. In some instances, some or all of the asparagine residues in the peptide are replaced by glutamine. In some embodiments, some or all of the aspartic acid residues can be replaced by glutamic acid residues. In some cases, the N-terminus of the peptide is blocked, such as by an acetyl group. Alternatively or in combination, in some instances, the C-terminus of the peptide is blocked, such as by an amide group. In some embodiments, the peptide is modified by methylation on free amines. For example, full methylation can be accomplished through the use of reductive methylation with formaldehyde and sodium cyanoborohydride.

In some embodiments, a peptide comprises a sequence motif of leucine-X₁-X₂-leucine-phenylalanine (“LX₁X₂LF,” SEQ ID NO: 178), in which X₁ and X₂ can be any amino acid residue, as shown in SEQ ID NO: 40, or variant thereof. In other embodiments, a nucleic acid, vector, plasmid, or donor DNA comprises a sequence that encodes a peptide, or variant or fragment thereof, of the present disclosure.

In some cases, the first two N-terminal amino acids can be GS as shown in SEQ ID NO: 1-SEQ ID NO: 83 or SEQ ID NO: 167-SEQ ID NO: 171, or such N-terminal amino acids (GS) can be absent as shown in SEQ ID NO: 84-SEQ ID NO: 166 or SEQ ID NO: 172-SEQ ID NO: 176, or can be substituted by any other one or two amino acids. In some cases, the first two N-terminal amino acids can be GS as shown in SEQ ID NO: 1-SEQ ID NO: 83 or SEQ ID NO: 167-SEQ ID NO: 171, or such N-terminal amino acids (GS) can be absent as shown in SEQ ID NO: 84-SEQ ID NO: 166 or SEQ ID NO: 172-SEQ ID NO: 176, or can be substituted by the amino acids GG.

In some cases, the C-terminal Arg residues of a peptide is modified to another residue such as Ala, Asn, Asp, Gln, Glu, Gly, His, Leu, Lys, Met, Phe, Pro, Ser, Thr, Trp, Tyr, or Val. For example, the C-terminal Arg residue of a peptide can be modified to Ile. Alternatively, the C-terminal Arg residue of a peptide can be modified to any non-natural amino acid. This modification can prevent clipping of the C-terminal residue during expression, synthesis, processing, storage, in vitro, or in vivo including during treatment, while still allowing maintenance of a key hydrogen bond. A key hydrogen bond can be the hydrogen bond formed during the initial folding nucleation and is critical for forming the initial hairpin.

In some cases the peptide comprises the sequence of any one of SEQ ID NO: 1-SEQ ID NO: 166. A peptide can be a fragment comprising a contiguous fragment of any one of SEQ ID NO: 1-SEQ ID NO: 166 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76 residues long, at least 77, at least 78, at least 79, at least 80, or at least 81 residues long, wherein the peptide fragment is selected from any portion of the peptide. In some embodiments, the peptide sequence is flanked by additional amino acids. One or more additional amino acids can, for example, confer a desired in vivo charge, isoelectric point, chemical conjugation site, stability, or physiologic property to a peptide.

The peptides of the present disclosure can further comprise negatively charged amino acid residues. In some cases, the peptide has 2 or fewer negative amino acid residues. In other cases, the peptide has 4 or fewer negative amino acid residues, 3 or fewer negative amino acid residues, or 1 or fewer negative amino acid residues. The negative amino acid residues can be selected from any negatively charged amino acid residues. The negative amino acid residues can selected from either E or D, or a combination of both E and D.

The peptides of the present disclosure can further comprise basic amino acid residues. In some embodiments, basic residues are added to the peptide sequence to increase the charge at physiological pH. The added basic residues can be any basic amino acid. The added basic residues can be selected from K or R, or a combination of K or R.

In some embodiments, the peptide has a charge distribution comprising an acidic region and a basic region. An acidic region can be a nub. A nub is a portion of a peptide extending out of the peptide's three-dimensional structure. A basic region can be a patch. A patch is a portion of a peptide that does not designate any specific topology characteristic of the peptide's three-dimensional structure. In further embodiments, a knotted peptide can be 6 or more basic residues and 2 or fewer acidic residues.

The peptides of the present disclosure can further comprise positively charged amino acid residues. In some cases, the peptide has at least 1 positively charged residue. In some cases, the peptide has at least 2 positively charged residues. In some cases, the peptide has at least 3 positively charged residues. In other cases, the peptide has at least 4 positively charged residues, at least 5 positively charged residues, at least 6 positively charged residues, at least 7 positively charged residues, at least 8 positively charged residues or at least 9 positively charged residues. While the positively charged residues can be selected from any positively charged amino acid residues, in some embodiments, the positively charged residues are either K, or R, or a combination of K and R.

The peptides of the present disclosure can further comprise neutral amino acid residues. In some cases, the peptide has 35 or fewer neutral amino acid residues. In other cases, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some cases the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any neutral charged amino acid residues, in some embodiments, the negative amino acid residues are either E, or D, or a combination of both E and D.

At physiological pH, peptides can have a net charge, for example, of −5, −4, −3, −2, −1, 0, +1, +2, +3, +4, or +5. When the net charge is zero, the peptide can be uncharged or zwitterionic. In some embodiments, the peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations within a peptide yields a peptide with an altered isoelectric point, charge, surface charge, or rheology at physiological pH. Such engineering of a mutation to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation can facilitate the ability of the peptide to pass through the gastrointestinal tract intact, to have a longer half life in serum or other compartments of the body, or to maintain secondary or tertiary structure in intracellular environments. Suitable amino acid modifications for improving the rheology and potency of a peptide can include conservative or non-conservative mutations. A peptide can comprises at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin, component that the peptide is derived from. In some embodiments, mutations can be in a single loop between disulfide bonds or can be in multiple loops. In other embodiments, mutations can improve pharmacokinetic or biodistribtion properties, or can add, enhance, or decrease biological activities. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom, toxin, or native component that the peptide is derived from. In some embodiments, mutations can be engineered within a peptide to provide a peptide that has a desired charge or stability at physiological pH.

Generally, the NMR solution structures, the x-ray crystal structures, as well as the primary structure sequence alignment of related structural homologs can be used to inform mutational strategies that can improve the folding, stability, and/or manufacturability, while maintaining a particular biological function. They can be used to predict the 3D pharmacophore of a group of structurally homologous scaffolds, as well as to predict possible graft regions of related proteins to create chimeras with improved properties. The general strategy for producing homologs can include identification of a charged surface patch of a protein, mutation of critical amino acid positions and loops, and testing of sequences. This strategy can be used to design peptides with improved properties or to correct deleterious mutations that complicate folding and manufacturability. These key amino acid positions and loops can be retained while other residues in the peptide sequences can be mutated to improve, change, remove, or otherwise modify function, homing, and activity of the peptide. The crystal structure of several peptides of this disclosure were solved and can be used to modify peptide function as described herein.

Improved peptides can also be engineered based upon immunogenicity information, such as immunogenicity information predicted by TEPITOPE and TEPITOPEpan. TEPITOPE is a computational approach which uses position specific scoring matrix to provide prediction rules for whether a peptide will bind to 51 different HLA-DR alleles, and TEPITOPEpan is method that uses TEPITOPE to extrapolate from HLA-DR molecules with known binding specificities to HLA-DR molecules with unknown binding specificities based on pocket similarity. For example, TEPITOPE and TEPITOPEpan can be used to determine immunogenicity of peptides that have improved stability. Comparison of peptides with high immunogenicity to peptides with low immunogenicity can guide engineering strategies for designing stable variants with decreased immunogenicity.

Additionally, the comparison of the primary sequences and the tertiary sequences of two or more peptides can be used to reveal sequence and 3D folding patterns that can be leveraged to improve the peptides and parse out the biological activity of these peptides. For example, comparing two different peptide scaffolds that are reduction resistant or protease resistant can lead to the identification of conserved pharmacophores that can guide engineering strategies, such as designing variants with improved resistance and stability properties.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of knotted peptides or knottins. Some suitable peptide for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, μ-PIIIA, ω-MVIIA, ω-CVID, χ-MrIA, ρ-TIA, conantokin G, contulakin G, GsMTx4, margatoxin, shK, toxin K, and EGF epiregulin core.

Two or more peptides can share a degree of sequence identity or homology and share similar properties in vivo. For instance, a peptide can share a degree of sequence identity or homology with any one of the peptides of SEQ ID NO: 1-SEQ ID NO: 166. In some cases, one or more peptides of the disclosure can have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. Various methods and software programs can be used to determine the homology between two or more peptides, such as NCBI BLAST, Clustal W, MAFFT, Clustal Omega, AlignMe, Praline, or another suitable method or algorithm.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

In some instances, the peptide is any one of SEQ ID NO: 1-SEQ ID NO: 166 or a functional fragment thereof. In other embodiments, the peptide of the disclosure further comprises a peptide with 99%, 95%, 90%, 85%, or 80% sequence identity or homology to any one of SEQ ID NO: 1-SEQ ID NO: 166 or fragment thereof.

In other instances, the peptide can be a peptide that is homologous to any one of SEQ ID NO: 1-SEQ ID NO: 166 or a functional fragment thereof. The term “homologous” is used herein to denote peptides having at least 70%, at least 80%, at least 90%, at least 95%, or greater than 95% sequence identity or homology to a sequence of any one of SEQ ID NO: 1-SEQ ID NO: 166 or a functional fragment thereof.

In still other instances, the variant nucleic acid molecules of a peptide of any one of SEQ ID NO: 1-SEQ ID NO: 166 can be identified by either a determination of the sequence identity or homology of the encoded peptide amino acid sequence with the amino acid sequence of any one of SEQ ID NO: 1-SEQ ID NO: 166, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 166 (or its complement) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 166. Alternatively, peptide variants of any one of SEQ ID NO: 1-SEQ ID NO: 166 can be characterized as nucleic acid molecules (1) that remain hybridized with a nucleic acid molecule having the nucleotide sequence of any one of SEQ ID NO: 1-SEQ ID NO: 166 (or its complement) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 166.

Percent sequence identity or homology is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, the determination of structure can typically be accompanied by evaluating activity of modified molecules.

### Chemical Modifications

A peptide can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation may be accomplished through the use of reductive methylation with formaldehyde and sodium cyanoborohydride.

A chemical modification can, for instance, extend the half-life of a peptide or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g., gly-ala-gly-ala (SEQ ID NO: 181)) that may or may not follow a pattern, or any combination of the foregoing.

The peptides of the present disclosure can be modified such that the modification increases the stability and/or the half-life of the peptides. The attachment of a hydrophobic moiety, such as to the N-terminus, the C-terminus, or an internal amino acid, can be used to extend half-life of a peptide of the present disclosure. The peptides can also be modified to increase or decrease the gut permeability or cellular permeability of the peptide. The peptide of the present disclosure can include post-translational modifications (e.g., methylation and/or amidation and/or glycosylation), which can affect, e.g., serum half-life. In some embodiments, simple carbon chains (e.g., by myristoylation and/or palmitylation) can be conjugated to the fusion proteins or peptides. The simple carbon chains can render the fusion proteins or peptides easily separable from the unconjugated material. For example, methods that can be used to separate the fusion proteins or peptides from the unconjugated material include, but are not limited to, solvent extraction and reverse phase chromatography. Lipophilic moieties can extend half-life through reversible binding to serum albumin. Conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof. In other embodiments, the peptides of the present disclosure can be coupled (e.g., conjugated) to a half-life modifying agent. Examples of half-life modifying agents can include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

In some embodiments, the first two N-terminal amino acids (GS) of SEQ ID NO: 1-SEQ ID NO: 83 or SEQ ID NO: 167-SEQ ID NO: 171 serve as a spacer or linker in order to facilitate conjugation or fusion to another molecule, as well as to facilitate cleavage of the peptide from such conjugated or fused molecules. In some embodiments, the fusion proteins or peptides of the present disclosure can be conjugated to other moieties that, e.g., can modify or effect changes to the properties of the peptides.

### Active Agent Peptide Conjugates

Peptides according to the present disclosure can be conjugated or fused to an agent for use in the treatment of tumors, and cancers, brain diseases and disorders, cartilage disorders, skin disorders, lung disorders, gastrointestinal diseases and disorders, vaginal mucosal diseases, ocular diseases, oral diseases, or other mucosal diseases or disorders. For example, in certain embodiments, the peptides described herein are fused to another molecule, such as an active agent that provides a functional capability. A peptide can be fused with an active agent through expression of a vector containing the sequence of the peptide with the sequence of the active agent. In various embodiments, the sequence of the peptide and the sequence of the active agent can be expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately. In certain embodiments, examples of active agents can include other peptides.

As another example, in certain embodiments, the peptides described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic molecule, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof. In some embodiments, the peptide is covalently or non-covalently linked to an active agent, e.g., directly or via a linker. For example, cytotoxic molecules that can be used include auristatins, MMAE, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, and BACE inhibitors. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). Exemplary linkers suitable for use with the embodiments herein are discussed in further detail below.

As compared to antibody-drug conjugates (e.g., Adcetris, Kadcyla, Mylotarg), in some aspects the peptide conjugated to an active agent as described herein can exhibit better penetration of solid tumors due to its smaller size. In certain aspects, the peptide conjugated to an active agent as described herein can carry different or higher doses of active agents as compared to antibody-drug conjugates. In still other aspects, the peptide conjugated to an active agent as described herein can have better site specific delivery of defined drug ratio as compared to antibody-drug conjugates. In other aspects, the peptide can be amenable to solvation in organic solvents (in addition to water), which can allow more synthetic routes for solvation and conjugation of a drug (which often has low aqueous solubility) and higher conjugation yields, higher ratios of drug conjugated to peptide (versus an antibody), and/or reduce aggregate/high molecular weight species formation during conjugation. Additionally, a unique amino acid residue(s) can be introduced into the peptide via a residue that is not otherwise present in the short sequence or via inclusion of a non-natural amino acid, allowing site specific conjugation to the peptide.

The peptides or fusion peptides of the present disclosure can also be conjugated to other moieties that can serve other roles, such as providing an affinity handle (e.g., biotin) for retrieval of the peptides from tissues or fluids. For example, peptides or fusion peptides of the present disclosure can also be conjugated to biotin. In addition to extension of half-life, biotin can also act as an affinity handle for retrieval of peptides or fusion peptides from tissues or other locations. In some embodiments, fluorescent biotin conjugates that can act both as a detectable label and an affinity handle can be used. Non limiting examples of commercially available fluorescent biotin conjugates can include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates can include chemiluminescent compounds, colloidal metals, luminescent compounds, enzymes, radioisotopes, and paramagnetic labels. In some embodiments, the peptide described herein can also be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding agents, fluorophore, radioisotope, radionuclide chelator, acyl adduct, chemical linker, or sugar). In some embodiments, the peptide can be fused with, or covalently or non-covalently linked to an active agent.

Additionally, more than one peptide sequence derived from a toxin or venom knottin protein can be present on or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Detectable Agent Peptide Conjugates

A peptide can be conjugated to an agent used in imaging, research, therapeutics, theranostics, pharmaceuticals, chemotherapy, chelation therapy, targeted drug delivery, and radiotherapy. In some embodiments, a peptide is conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, or 10 detectable agents can be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Peptides can be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers can include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is fused with, or covalently or non-covalently linked to the agent, e.g., directly or via a linker. Exemplary linkers suitable for use with the embodiments herein are discussed in further detail below.

### Linkers

Peptides according to the present disclosure can be attached to another moiety (e.g., an active agent or an detectable agent), such as a small molecule, a second peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. In the absence of a linker, for example, an active agent or an detectable agent can be fused to the N-terminus or the C-terminus of a peptide to create an active agent or detectable agent fusion peptide. In other embodiments, the link can be made by a peptidic fusion via reductive alkylation.

Direct attachment can be through covalent attachment of a peptide to a region of the other molecule. For example, an active agent or a detectable agent can be fused to the N-terminus or the C-terminus of a peptide to create an active agent or detectable agent fusion peptide. As an additional example, a peptidic linker can be inserted between the N-terminus or C-terminus of a peptide and an active agent or detectable agent, wherein the peptidic linker can be from 1 to 30 amino acid residues and can comprise (GGGS)ₓ, wherein X can be any integer from 1 to 7 (SEQ ID NO: 182). As another example, the peptide can be attached at the N-terminus, an internal lysine, glutamic acid, or aspartic acid residue, or the C-terminus to a terminus of the amino acid sequence of the other molecule by a linker. If the attachment is at an internal lysine residue, the other molecule can be linked to the peptide at the epsilon amine of the internal lysine residue. In some further examples, the peptide can be attached to the other molecule by a side chain, such as the side chain of a lysine, serine, threonine, cysteine, tyrosine, aspartic acid, a non-natural amino acid residue, or glutamic acid residue. A linker can be an amide bond, an ester bond, an ether bond, a carbamate bond, a carbonate bond, a carbon-nitrogen bond, a triazole, a macrocycle, an oxime bond, a thioester bond, a thioether bond a hydrazone bond, a carbon-carbon single, double, or triple bond, a disulfide bond, a two carbon bridge between two cysteines, a three carbon bridge between two cysteines, or a thioether bond. In still other embodiments, the peptide can comprise a non-natural amino acid, wherein the non-natural amino acid can be an insertion, appendage, or substitution for another amino acid, and the peptide can be linked to the active agent at the non-natural amino acid by a linker. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) can be used to link other molecules.

Attachment via a linker involves incorporation of a linker moiety between the other molecule and the peptide. The peptide and the other molecule can both be covalently attached to the linker. The linker can be cleavable, non-cleavable, self-immolating, hydrophilic, or hydrophobic. The linker has at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment can include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds include amino groups; carboxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link molecules together include click chemistry, copper-free click chemistry, HIPS ligation, Staudinger ligation, and hydrazine-iso-Pictet-Spengler.

Non-limiting examples of linkers include:

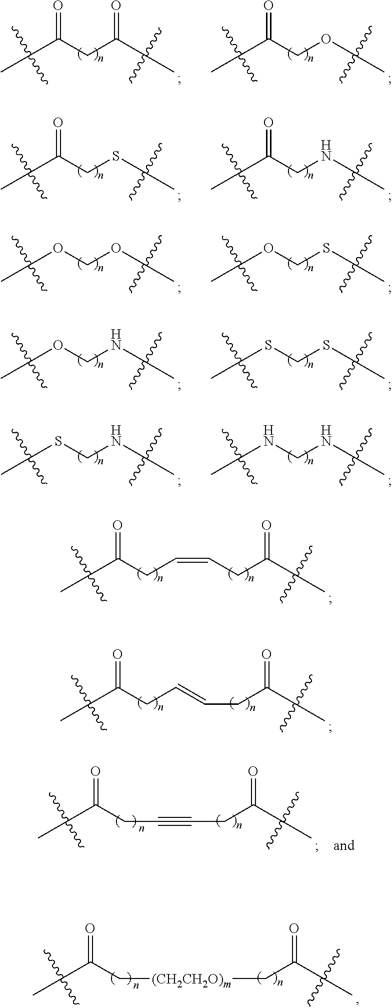

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

The linker may be a noncleavable linker or a cleavable linker. In some embodiments, the noncleavable linker can slowly release the conjugated moiety by an exchange of the conjugated moiety onto the free thiols on serum albumin. In some embodiments, the use of a cleavable linker can permit release of the conjugated moiety (e.g., a therapeutic agent) from the peptide, e.g., after administration to a subject in need thereof. In other embodiments, the use of a cleavable linker can permit the release of the conjugated therapeutic from the peptide. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, peptidases, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters can be faster compared to the hydrolysis of linkers with bulky groups next to an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the target location. For example, when a peptide is cleared from a tumor, or the brain, relatively quickly, the linker can be tuned to rapidly hydrolyze. When a peptide has a longer residence time in the target location, a slower hydrolysis rate would allow for extended delivery of an active agent. “Programmed hydrolysis in designing paclitaxel prodrug for nanocarrier assembly” Sci Rep 2015, 5, 12023 Fu et al., provides an example of modified hydrolysis rates.

### Crystal Structure

In some embodiments, the crystal structure of any peptide of this disclosure can be solved in order to spatially map each atom in a given peptide. Solving the crystal structure of the peptide can yield information on the spatial orientation, positioning, and interaction of amino acids. Thus, in some embodiments, the crystal structure of a peptide can provide information on conserved structural elements that can play a role in stability, in identifying residues that can be mutated, conserved, or internal or external to the surface of a folded peptide to maintain folding, stability, function or biological activity, or in identifying sites for conjugation with active agents or sites of modification to that can affect binding specificity or strength. This information can allow mutants to be designed that preserve a desired function (such as reduction resistance) while changing other aspects of the peptide. For example, the crystal structures of the following peptides were solved and can show the three-dimensional folded crystal structure of each peptide: SEQ ID NO: 3, SEQ ID NO: 27, SEQ ID NO: 22, SEQ ID NO: 34, SEQ ID NO: 10, SEQ ID NO: 12, SEQ ID NO: 13, SEQ ID NO: 36, SEQ ID NO: 37, SEQ ID NO: 20, SEQ ID NO: 51, and SEQ ID NO: 47. Two different crystal structures can be formed for SEQ ID NO: 10, in which one crystal structure can comprise sixteen independent molecules in a symmetric unit cell and the other crystal structure can comprise four molecules, in which the peptides of SEQ ID NO: 10 were interacting with each other through non-covalent bonds in both of these formed crystal structures. One of skill can apply principles to each crystal structure and the underlying coordinate data to identify conserved and internal residues, or determine which residues may be modified without likely affecting the overall structure. This information about a peptide based on its crystal structure and the general sequences of knotted peptides can be used to enhance specific properties of the peptides of this disclosure such as, but not limited to, modifying stability and/or resistance to a variety of agents and conditions in order to make stable variants of the disclosed sequences, enhancing physiologic activity, optimizing manufacturability, and identifying optimal sites for conjugating or linking the peptide to an active agent or detectable agent.

### Peptide as a Delivery Scaffold

In certain embodiments, any peptide of this disclosure can be used as a delivery scaffold for an active agent. A peptide of this disclosure can be used as delivery scaffold for an active agent to various biological environments due to the peptide's enhanced stability in these environments, which can allow for access and treatment of disorders in these biological environments. For example, any peptide of SEQ ID NO: 1-SEQ ID NO: 166 can be stable in a biological environment with a low pH, a protease-rich environment, an acidic environment, a reducing environment, and/or environments with varying temperatures. Such biological environments can be found in the gastrointestinal (GI) tract (including, but not limited to, mouth, nasal cavities, throat, esophagus, stomach, small intestine, large intestine, and rectum), lung, skin, cartilage, vaginal mucosa, or nasal mucosa, or a cellular compartment, such as lysosomes, endosomes, or the cytosol. Therefore, using the peptides of this disclosure as delivery scaffold can be advantageous for delivery of therapeutics to various physiologic environments that can degrade other peptides.

### Peptide Stability

A peptide of the present disclosure can be stable in various biological conditions. For example, any peptide of SEQ ID NO: 1-SEQ ID NO: 166 can exhibit resistance to reducing agents, proteases, oxidative conditions, elevated temperature conditions, or acidic conditions.

In some cases, biologic molecules (such as peptides and proteins) can provide therapeutic functions, but such therapeutic functions are decreased or impeded by instability caused by the in vivo environment (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they may be unable to provide a therapeutic function on intracellular targets. Therefore, peptides that are resistant to reducing agents, proteases, and low pH may be able to provide enhanced therapeutic effects or enhance the therapeutic efficacy of co-formulated or conjugated active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function. Peptides and proteins may not be able to exert therapeutic activity after oral delivery because they may be too rapidly degraded by the GI tract. Therefore, peptides that are resistant to conditions in the GI tract can be used as biologically active peptide therapeutics that can be orally administered.

Furthermore, as described herein, the properties or characteristics of the peptides of this disclosure can be resistant to a variety of physiologic or environmental conditions. This resistance can enable administration via inhalation, intranasally, orally, topically, intravenously, subcutaneously, intra-articularly, intramuscularly administration, intraperitoneally, intra-synovially, by vaginal route, rectal route, pulmonary route, ocular route (including, but limited to, topical, to the cornea, and intravitreal), buccal, sublingual, intrathecal, or any combination thereof.

Peptide Resistance to Reducing Agents.

Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, reduced glutathione (GSH) is a reducing agent that can be present in many areas of the body and in cells, and can reduce disulfide bonds. As another example, a peptide can become reduced during trafficking of a peptide across the gastrointestinal epithelium after oral administration. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to loss of functionality due to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001 M-0.0001 M, 0.0001 M-0.001 M, 0.001 M-0.01 M, 0.01 M-0.05 M, 0.05 M-0.1 M, for 15 minutes or more. In some embodiments, the reducing agent used to determine peptide stability can be dithiothreitol (DTT), Tris(2-carboxyethyl)phosphine HCl (TCEP), 2-Mercaptoethanol, reduced glutathione (GSH), or any combination thereof. In some embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to a reducing agent.

In some embodiments, the peptides of this disclosure can remain at least 70% intact after being exposed to from 0.01 mM to 0.1 mM DTT and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM DTT and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 1 mM to 10 mM DTT and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM DTT and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 0.01 mM to 0.1 mM DTT and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM DTT and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 1 mM to 10 mM DTT and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM DTT and a temperature of at least 30° C. for 1 day, 1 week, 1 month, 6 months, 1 year, 2 years, 3 years, 4 years. In some embodiments, the peptides of this disclosure can remain at least 70% intact after being exposed to from 0.01 mM to 0.1 mM DTT and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM DTT and a temperature of at least 40° C. for 1 day, 1 week, 1 month, 6 months, 1 year, 2 years, 3 years, or 4 years. In some embodiments, the peptides of this disclosure can remain at least 70% intact after being exposed to from 1 mM to 10 mM DTT and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM DTT and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 0.01 mM to 0.1 mM DTT and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 0.1 mM to 1.0 mM DTT and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 1 mM to 10 mM DTT and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 10 mM to 100 mM DTT and a temperature of at least 37° C. for at least 0.5 hours, 1 hour, 8 hours, 16 hours, 24 hours, 36 hours, or 48 hours.

In some embodiments, the peptides of this disclosure can remain at least 70% intact after being exposed to from 0.01 mM to 0.1 mM GSH and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM GSH and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 1 mM to 10 mM GSH and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM GSH and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from 0.01 mM to 0.1 mM GSH and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM GSH and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 1 mM to 10 mM GSH and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM GSH and a temperature of at least 30° C. for 1 day, 1 week, 1 month, 6 months, 1 year, 2 years, 3 years, 4 years. In some embodiments, the peptides of this disclosure can remain at least 70% intact after being exposed to from 0.01 mM to 0.1 mM GSH and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 0.1 mM to 1.0 mM GSH and a temperature of at least 40° C. for 1 day, 1 week, 1 month, 6 months, 1 year, 2 years, 3 years, or 4 years. In some embodiments, the peptides of this disclosure can remain at least 70% intact after being exposed to from 1 mM to 10 mM GSH and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 10 mM to 100 mM GSH and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from 0.01 mM to 0.1 mM GSH and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 0.1 mM to 1.0 mM GSH and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 1 mM to 10 mM GSH and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from 10 mM to 100 mM GSH and a temperature of at least 37° C. for at least 0.5 hours, 1 hour, 8 hours, 16 hours, 24 hours, 36 hours, or 48 hours.

Peptide Resistance to Proteases.

The stability of peptides of this disclosure can be determined by resistance to degradation by proteases. Proteases, also referred to as peptidases or proteinases, are enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of this disclosure can resist degradation by any class of protease. In certain embodiments, peptides of this disclosure resist degradation by pepsin (which can be found in the stomach), trypsin (which can be found in the duodenum), serum proteases, or any combination thereof. In some embodiments, the proteases used to determine peptide stability can be pepsin, trypsin, chymotrypsin, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, and elafin), or any combination thereof. In some embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to a protease.

In some embodiments, the peptides of this disclosure can remain at least 70% intact after being exposed to pepsin at a concentration of from 0.5 U/ml to 5 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 5 U/ml to 50 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 50 U/ml to 500 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 500 U/ml to 5000 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 0.5 U/ml to 5 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 5 U/ml to 50 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 50 U/ml to 500 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 500 U/ml to 5000 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 0.5 U/ml to 5 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 5 U/ml to 50 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 50 U/ml to 500 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 500 U/ml to 5000 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to pepsin at a concentration of from 0.5 U/ml to 5 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to pepsin at a concentration of from 5 U/ml to 50 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to pepsin at a concentration of from 50 U/ml to 500 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to pepsin at a concentration of from 500 U/ml to 5000 U/ml and a temperature of at least 37° C. for at least 0.5 hours, 1 hour, 8 hours, 16 hours, 24 hours, 36 hours, or 48 hours.

In some embodiments, the peptides of this disclosure can remain at least 70% intact after being exposed to trypsin at concentration of from 0.5 U/ml to 5 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 5 U/ml to 50 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 50 U/ml to 500 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 500 U/ml to 5000 U/ml and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 0.5 U/ml to 5 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 5 U/ml to 50 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 50 U/ml to 500 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 500 U/ml to 5000 U/ml and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 0.5 U/ml to 5 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 5 U/ml to 50 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 50 U/ml to 500 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 500 U/ml to 5000 U/ml and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to trypsin at concentration of from 0.5 U/ml to 5 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to trypsin at concentration of from 5 U/ml to 50 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to trypsin at concentration of from 50 U/ml to 500 U/ml and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to trypsin at concentration of from 500 U/ml to 5000 U/ml and a temperature of at least 37° C. for at least 0.5 hours, 1 hour, 8 hours, 16 hours, 24 hours, 36 hours, or 48 hours.

Peptide Stability in Acidic Conditions.

Peptides of this disclosure can be administered in biological environments that are acidic. For example, after oral administration, peptides can experience acidic environmental conditions in the gastric fluids of the stomach and gastrointestinal (GI) tract. The pH of the stomach can range from ˜1-4 and the pH of the GI tract ranges from acidic to normal physiological pH descending from the upper GI tract to the colon. In addition, the vagina, late endosomes, and lysosomes can also have acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide. In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%, at least 10%, at least 10%, at least 20%, at least 20%, at least 30%, at least 30%, at least 40%, at least 40%, at least 50%, at least 60%, at least 70%, at least 80%, at least 90%, at least 99%, or 100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5%, at least 10%, at least 10%, at least 20%, at least 20%, at least 30%, at least 30%, at least 40%, at least 40%, at least 50%, at least 60%, at least 70%, at least 80%, at least 90%, at least 99%, or 100% of the peptide remains intact after exposure to simulated gastric fluid. In some embodiments, low pH solutions such as simulated gastric fluid can be used to determine peptide stability.

In some embodiments, the peptides of this disclosure can remain at least 70% intact after being exposed to from a pH of 0.5 to a pH of 2 and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from a pH of 2 to a pH of 5 and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from a pH of 5 to a pH of 6 trypsin and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from a pH of 6 to a pH of 8 and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to from a pH of 0.5 to a pH of 2 and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from a pH of 2 to a pH of 5 and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to a pH of 5 to a pH of 6 trypsin and a temperature of at least 30° C. for at least 1 day, 1 week, 1 month, 6 months, 1 year, 2 years, 3 years, or 4 years. In some embodiments, the peptides of this disclosure can remain at least 70% intact after being exposed to from a pH of 6 to a pH of 8 and a temperature of at least 30° C. for 1 day, 1 week, 1 month, 6 months, 1 year, 2 years, 3 years, 4 years. In some embodiments, the peptides of this disclosure can remain at least 70% intact after being exposed to from a pH of 0.5 to a pH of 2 and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from a pH of 2 to a pH of 5 and a temperature of at least 40° C. for 1 day, 1 week, 1 month, 6 months, 1 year, 2 years, 3 years, or 4 years. In some embodiments, the peptides of this disclosure can remain at least 70% intact after being exposed to a pH of 5 to a pH of 6 and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from a pH of 6 to a pH of 8 and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to from a pH of 0.5 to a pH of 2 and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from a pH of 2 to a pH of 5 and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from a pH of 5 to a pH of 6 and a temperature of at least 37° C. for at least 0.5 hours, 1 hour, 8 hours, 16 hours, 24 hours, 36 hours, or 48 hours. In some embodiments, the peptides of this disclosure can remain at least 70% intact after being exposed to from a pH of 6 to a pH of 8 trypsin and a temperature of at least 37° C. for at least 0.5 hours, 1 hour, 8 hours, 16 hours, 24 hours, 36 hours, or 48 hours.

In some embodiments, the peptides of this disclosure can remain at least 70% intact after being exposed to SGF and a temperature of at least 23° C. for at least 1 day, 1 week, 1 month, 6 months, 1 year, 2 years, 3 years, or 4 years. In some embodiments, the peptides of this disclosure can remain at least 70% intact after being exposed to SGF and a temperature of at least 30° C. for at least 1 day, 1 week, 1 month, 6 months, 1 year, 2 years, 3 years, or 4 years. In some embodiments, the peptides of this disclosure can remain at least 70% intact after being exposed SGF and a temperature of at least 40° C. for at least 1 day, 1 week, 1 month, 6 months, 1 year, 2 years, 3 years, or 4 years. In some embodiments, the peptides of this disclosure can remain at least 70% intact after being exposed to SGF and a temperature of at least 37° C. for at least 0.5 hours, 1 hour, 8 hours, 16 hours, 24 hours, 36 hours, or 48 hours.

Peptide Stability at High Temperatures.

Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. Stability at a higher temperature can also allow for more efficient room temperature storage. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, at least 99%, or 100% of the peptide remains intact after exposure to 25° C. for at least 6 months to 5 years. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, at least 99%, or 100% of the peptide remains intact after exposure to 70° C. for 15 minutes to 1 hour. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, at least 99%, or 100% of the peptide remains intact after exposure to 100° C. for 15 minutes to 1 hour. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to 25° C. for at least 6 months to 5 years. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to 30° C. for at least 6 months to 5 years. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to 40° C. for at least 6 months to 5 years. In other embodiments, at least 5%, at least 10%, at least 10%, at least 20%, at least 20%, at least 30%, at least 30%, at least 40%, at least 40%, at least 50%, at least 60%, at least 70%, at least 80%, at least 90%, a least 99%, or 100% of the peptide remains intact after exposure to 37° C. for at 0.5 hours to 48 hours. In other embodiments, at least 10% of the peptide remains intact after exposure to 37° C. for at least 0.5 hours, 1 hour, 2 hours, 4 hours, 6 hours, 10 hours, 12 hours, 16 hours, 20 hours, 24 hours, 36 hours, or 48 hours. In other embodiments, at least 1% of the peptide remains intact after exposure to 37° C. for at least 0.5 hours, 1 hour, 2 hours, 4 hours, 6 hours, 10 hours, 12 hours, 16 hours, 20 hours, 24 hours, 36 hours, or 48 hours. In other embodiments, at least 10% of the peptide remains intact after passage through the mouth, stomach, small intestine, or the large intestine. In other embodiments, at least 1% of the peptide remains intact after passage through the mouth, stomach, small intestine, or the large intestine. In other embodiments, at least 50% of the peptide remains intact after passage through the mouth, stomach, small intestine, or the large intestine. In other embodiments, at least 1% of the peptide remains intact after passage through the mouth, stomach, small intestine, or the large intestine. In other embodiments, at least 20% of the peptide remains intact after passage through the mouth, stomach, small intestine, or the large intestine. In other embodiments, at least 70% of the peptide remains intact after passage through the mouth, stomach, small intestine, or the large intestine. In other embodiments, at least 75% of the peptide remains intact after passage through the mouth, stomach, small intestine, or the large intestine. In other embodiments, at least 90% of the peptide remains intact after passage through the mouth, stomach, small intestine, or the large intestine. In other embodiments, at least 95% of the peptide remains intact after passage through the mouth, stomach, small intestine, or the large intestine.

### Pharmacokinetics of Peptides

The pharmacokinetics of any of the peptides of this disclosure can be determined after administration of the peptide via different routes of administration. For example, the pharmacokinetic parameters of a peptide of this disclosure can be quantified after intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, peritoneal, buccal, synovial, or topical administration. Peptides of the present disclosure can be analyzed by using tracking agents such as radiolabels or fluorophores. For example, a radiolabeled peptides of this disclosure can be administered via various routes of administration. Peptide concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein relate to pharmacokinetics of peptide administration via any route to a subject. Pharmacokinetics can be described using methods and models, for example, compartmental models or noncompartmental methods. Compartmental models include but are not limited to monocompartmental model, the two compartmental model, the multicompartmental model or the like. Models are often divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation includes liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed.

Pharmacokinetics includes determining at least one parameter associated with administration of a peptide to a subject. In some aspects, parameters include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, the peptides of any of SEQ ID NO: 1-SEQ ID NO: 166 exhibit optimal pharmacokinetic parameters after oral administration. In other embodiments, the peptides of any of SEQ ID NO: 1-SEQ ID NO: 166 exhibit optimal pharmacokinetic parameters after a any route of administration, such as oral administration, inhalation, intranasal administration, topical administration, intravenous administration, subcutaneous administration, intra-articular administration, intramuscular administration, intraperitoneal administration, intra-synovial, or any combination thereof.

In some embodiments any peptide of SEQ ID NO: 1-SEQ ID NO: 166 exhibits an average Tₘₐₓ of 0.5-12 hours, or 1-48 hours at which the Cₘₐₓ is reached, an average bioavailability in serum of 0.1%-10% in the subject after administering the peptide to the subject by an oral route, an average bioavailability in serum of less than 0.1% after oral administration to a subject for delivery to the GI tract, an average bioavailability in serum of 10-100% after parenteral administration, an average t_{1/2} of 0.1-168 hours, or 0.25-48 hours in a subject after administering the peptide to the subject, an average clearance (CL) of 0.5-100 L/hour or 0.5-50 L/hour of the peptide after administering the peptide to a subject, an average volume of distribution (V_{d}) of 200-20,000 mL in the subject after systemically administering the peptide to the subject, or optionally no systemic uptake, any combination thereof.

### Methods of Manufacture

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, a glutamic acid or aspartic acid residue, a C-terminus, or a non-natural amino acid.

The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

In some embodiments, the peptides of this disclosure can be more stable during manufacturing. For example, peptides of this disclosure can be more stable during recombinant expression and purification, resulting in lower rates of degradation by proteases that are present in the manufacturing process, a higher purity of peptide, a higher yield of peptide, or any combination thereof. In some embodiments, the peptides can also be more stable to degradation at high temperatures and low temperatures during manufacturing, storage, and distribution. For example, in some embodiments peptides of this disclosure can be stable at 25° C., 30° C., 35° C., or 40° C. In other embodiments, peptides of this disclosure can be stable at 70° C. or higher than 70° C. In some embodiments, peptides of this disclosure can be stable at 100° C. or higher than 100° C.

In some embodiments, peptides of this disclosure can remain intact after exposure to a temperature of at least 25° C., 30° C., or 40° C. with at least 60%, 65% or 75% relative humidity for at least 3, 6, 12, 18, 24, 36, or 48 months. In some embodiments, peptides of this disclosure can remain intact after exposure to a temperature of at least 25° C. with at least 60% relative humidity for from 3 months to 48 months. In some embodiments, peptides of this disclosure can remain intact after exposure to a temperature of at least 25° C. with at least 60% relative humidity for from 3 months to 12 months. In some embodiments, peptides of this disclosure can remain intact after exposure to a temperature of at least 25° C. with at least 60% relative humidity for from 12 months to 24 months. In some embodiments, peptides of this disclosure can remain intact after exposure to a temperature of at least 25° C. with at least 65% relative humidity for from 3 months to 48 months. In some embodiments, peptides of this disclosure can remain intact after exposure to a temperature of at least 25° C. with at least 65% relative humidity for from 3 months to 12 months. In some embodiments, peptides of this disclosure can remain intact after exposure to a temperature of at least 25° C. with at least 65% relative humidity for from 12 months to 24 months. In some embodiments, peptides of this disclosure can remain intact after exposure to a temperature of at least 25° C. with at least 70% relative humidity for from 3 months to 48 months. In some embodiments, peptides of this disclosure can remain intact after exposure to a temperature of at least 25° C. with at least 70% relative humidity for from 3 months to 12 months. In some embodiments, peptides of this disclosure can remain intact after exposure to a temperature of at least 25° C. with at least 70% relative humidity for from 12 months to 24 months.

In some embodiments, peptides of this disclosure can remain intact after exposure to a temperature of at least 23° C. with at least 60% relative humidity for from 3 months to 48 months. In some embodiments, peptides of this disclosure can remain intact after exposure to a temperature of at least 23° C. with at least 60% relative humidity for from 3 months to 12 months. In some embodiments, peptides of this disclosure can remain intact after exposure to a temperature of at least 23° C. with at least 60% relative humidity for from 12 months to 24 months. In some embodiments, peptides of this disclosure can remain intact after exposure to a temperature of at least 23° C. with at least 65% relative humidity for from 3 months to 48 months. In some embodiments, peptides of this disclosure can remain intact after exposure to a temperature of at least 23° C. with at least 65% relative humidity for from 3 months to 12 months. In some embodiments, peptides of this disclosure can remain intact after exposure to a temperature of at least 23° C. with at least 65% relative humidity for from 12 months to 24 months. In some embodiments, peptides of this disclosure can remain intact after exposure to a temperature of at least 23° C. with at least 70% relative humidity for from 3 months to 48 months. In some embodiments, peptides of this disclosure can remain intact after exposure to a temperature of at least 23° C. with at least 70% relative humidity for from 3 months to 12 months. In some embodiments, peptides of this disclosure can remain intact after exposure to a temperature of at least 23° C. with at least 70% relative humidity for from 12 months to 24 months.

In some embodiments, peptides of this disclosure can remain intact after exposure to a temperature of at least 30° C. with at least 60% relative humidity for from 3 months to 48 months. In some embodiments, peptides of this disclosure can remain intact after exposure to a temperature of at least 30° C. with at least 60% relative humidity for from 3 months to 12 months. In some embodiments, peptides of this disclosure can remain intact after exposure to a temperature of at least 30° C. with at least 60% relative humidity for from 12 months to 24 months. In some embodiments, peptides of this disclosure can remain intact after exposure to a temperature of at least 30° C. with at least 65% relative humidity for from 3 months to 48 months. In some embodiments, peptides of this disclosure can remain intact after exposure to a temperature of at least 30° C. with at least 65% relative humidity for from 3 months to 12 months. In some embodiments, peptides of this disclosure can remain intact after exposure to a temperature of at least 30° C. with at least 65% relative humidity for from 12 months to 24 months. In some embodiments, peptides of this disclosure can remain intact after exposure to a temperature of at least 30° C. with at least 70% relative humidity for from 3 months to 48 months. In some embodiments, peptides of this disclosure can remain intact after exposure to a temperature of at least 30° C. with at least 70% relative humidity for from 3 months to 12 months. In some embodiments, peptides of this disclosure can remain intact after exposure to a temperature of at least 30° C. with at least 70% relative humidity for from 12 months to 24 months.

In some embodiments, peptides of this disclosure can remain intact after exposure to a temperature of at least 40° C. with at least 60% relative humidity for from 3 months to 48 months. In some embodiments, peptides of this disclosure can remain intact after exposure to a temperature of at least 40° C. with at least 60% relative humidity for from 3 months to 12 months. In some embodiments, peptides of this disclosure can remain intact after exposure to a temperature of at least 40° C. with at least 60% relative humidity for from 12 months to 24 months. In some embodiments, peptides of this disclosure can remain intact after exposure to a temperature of at least 40° C. with at least 65% relative humidity for from 3 months to 48 months. In some embodiments, peptides of this disclosure can remain intact after exposure to a temperature of at least 40° C. with at least 65% relative humidity for from 3 months to 12 months. In some embodiments, peptides of this disclosure can remain intact after exposure to a temperature of at least 40° C. with at least 65% relative humidity for from 12 months to 24 months. In some embodiments, peptides of this disclosure can remain intact after exposure to a temperature of at least 40° C. with at least 70% relative humidity for from 3 months to 48 months. In some embodiments, peptides of this disclosure can remain intact after exposure to a temperature of at least 40° C. with at least 70% relative humidity for from 3 months to 12 months. In some embodiments, peptides of this disclosure can remain intact after exposure to a temperature of at least 40° C. with at least 70% relative humidity for from 12 months to 24 months.

### Pharmaceutical Compositions of Peptides

A pharmaceutical composition of the disclosure can be a combination of any peptide described herein with other chemical components, such as carriers, stabilizers, diluents, dispersing agents, suspending agents, thickening agents, antioxidants, solubilizers, buffers including citric acid, osmolytes, salts, surfactants, amino acids, encapsulating agents, bulking agents, cryoprotectants, mucoadhesive agents, delayed release agents, enteric coatings, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, buccal, intra-articular, intra-synovial, and topical administration. A pharmaceutical composition can be administered in a local or systemic manner, for example, via injection of the peptide described herein directly into an organ, optionally in a depot including biodegradable matrices, thermal gelling agents, and aqueous and non-aqueous solvents.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water soluble form. Suspensions of peptides described herein can be prepared as oily injection suspensions. Suitable lipophilic solvents or vehicles include fatty oils such as sesame oil, N-methyl pyrrolidone, propylene glycol, glycerol, alcohols, fatty acids or omega-3-fatty acids, or synthetic fatty acid esters, such as ethyl oleate or triglycerides, liposomes, micelles, or mixed micelles. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, 5% dextrose in water, isotonic saline solutions, or buffered solutions before use. In some embodiments, a purified peptide is administered intravenously.

A peptide of the disclosure can be applied directly to an organ, or an organ tissue or cells, such as brain or brain tissue or cancer cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions can also include permeation or absorption enhancers (Aungst et al. AAPS J. 14(1):10-8. (2012) and Moroz et al. Adv Drug Deliv Rev 101:108-21. (2016)). Permeation enhancers can facilitate uptake of molecules from the GI tract into systemic circulation. Permeation enhancers can include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates,

Compositions can also include protease inhibitors including soy bean trypsin inhibitor, aprotinin, sodium glycocholate, camostat mesilate, vacitracin, or cyclopentadecalactone.

Pharmaceutical compositions can also include excipients to release an agent in certain parts of the gastrointestinal (GI) tract. For example, but limited to, an excipient can be an enteric coating (e.g., fatty acids, waxes, shellac, plastics, and plant fibers), methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, or zein.

### Use of Peptides in Treatments

In some embodiments, the method includes administering an effective amount of a peptide as described herein to a subject in need thereof.

In one embodiment, the method includes administering an effective amount of a peptide as described herein to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide of the disclosure. The disease may be a cancer or tumor. In treating the disease, the peptide may contact the tumor or cancerous cells. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either intravenously, subcutaneously, intramuscularly, by inhalation, dermally, topically, by intra-articular injection, orally, sublingually, intrathecally, transdermally, intranasally, via a peritoneal route, directly into the brain, e.g., via and intracerebral ventrical route, or directly onto a joint, e.g. via topical, intra-articular injection route. A treatment can comprise administering a peptide-active agent complex to a subject, either intravenously, subcutaneously, intramuscularly, by inhalation, by intra-articular injection, dermally, topically, orally, intrathecally, transdermally, intransally, parenterally, orally, via a peritoneal route, nasally, sublingually, directly onto cancerous tissues, or directly onto or near cartilage.

In some embodiments, a peptide comprising the sequence of any of SEQ ID NO: 1-SEQ ID NO: 166, and any peptide derivative or peptide conjugates, such as a peptide-active agent conjugate, as described herein, can be used to treat a disorder of a region of the body or tissue or an intracellular compartment. In certain embodiments, the peptide can be used as a delivery scaffold for an active agent. For example, a peptide or peptide-active agent conjugate can be used to access and treat disorders of the gastrointestinal (GI) tract, lung, skin, cartilage, vaginal mucosa, or nasal mucosa. Peptides of this disclosure can be used to access and treat these disorders due to their enhanced stability in various biological environments, including low pH, protease-rich environments, acidic environments, reducing environments, or environments with varying temperatures.

A peptide comprising the sequence of any of SEQ ID NO: 1-SEQ ID NO: 166, and any peptide derivative or peptide-active agent conjugate as described herein, can be used to treat upper GI disease and cancers (e.g., throat, oral, esophageal cancer, salivary glands, tonsils, pharynx, adenosarcomas, oral malignant melanoma head, neck cancer, or sarcomas). A peptide comprising the sequence of any of SEQ ID NO: 1-SEQ ID NO: 166, and any peptide derivative or peptide-active agent conjugate as described herein, can be used to target diseases of the esophagus, stomach, the small intestine, the duodenum, the large intestine, and other parts of the GI tract. These diseases can include Crohn's disease, inflammatory bowel disease, irritable bowel syndrome, cancers such as colorectal cancer and stomach cancer, gastroesophageal reflux disease, ulcerative colitis, constipation, opioid-induced constipation, and infections, such as an infection caused by Helicobacter pylori.

A peptide comprising the sequence of any of SEQ ID NO: 1-SEQ ID NO: 166, and any peptide derivative or peptide-detectable agent conjugate as described herein, can be used to diagnose or image upper GI disease and cancers (e.g., throat, oral, esophageal cancer, salivary glands, tonsils, pharynx, adenosarcomas, oral malignant melanoma head, neck cancer, or sarcomas). A peptide comprising the sequence of any of SEQ ID NO: 1-SEQ ID NO: 166, and any peptide derivative or peptide-detectable agent conjugate as described herein, can be used to diagnose or image diseases of the esophagus, stomach, the small intestine, the duodenum, the large intestine, and other parts of the GI tract. These diseases can include Crohn's disease, inflammatory bowel disease, irritable bowel syndrome, cancers such as colorectal cancer and stomach cancer, gastroesophageal reflux disease, ulcerative colitis, constipation, opioid-induced constipation.

A peptide comprising the sequence of any of SEQ ID NO: 1-SEQ ID NO: 166, and any peptide derivative or peptide-active agent conjugate as described herein, can be used to treat upper chronic inflammatory lung diseases such as cystic fibrosis, chronic obstructive pulmonary disease (COPD), and emphysema, which are characterized by higher than normal levels of pulmonary proteases (e.g., neutrophil elastase, alpha-1 antitrypsin, secretory leucoprotease inhibitor, or elafin).

A peptide comprising the sequence of any of SEQ ID NO: 1-SEQ ID NO: 166, and any peptide derivative or peptide-detectable agent conjugate as described herein, can be used to diagnose or image upper chronic inflammatory lung diseases such as cystic fibrosis, chronic obstructive pulmonary disease (COPD), and emphysema, which are characterized by higher than normal levels of pulmonary proteases (e.g., neutrophil elastase, alpha-1 antitrypsin, secretory leucoprotease inhibitor, or elafin).

A peptide comprising the sequence of any of SEQ ID NO: 1-SEQ ID NO: 166, and any peptide derivative or peptide-active agent conjugate as described herein, can be used to treat eye diseases, disorders, or infections such as asacanthamoeba keratitis, blepharitis, CMV retinitis, conjunctivitis, corneal abrasion, dry eye syndrome, ocular herpes, fungal keratitis, trachoma, endophthalmitis, dacryostenosis, uveitis, Sjogren's syndrome, stye, ocular histoplasmosis syndrome, mycosis, toxoplasmosis, chlamydia, gonorrhea, bacterial keratitis, tuberculosis, leprosy, syphilis, hepatitis B, or infections caused by herpes simplex virus, epstein-barr virus, or Candida.

A peptide comprising the sequence of any of SEQ ID NO: 1-SEQ ID NO: 166, and any peptide derivative or peptide-detectable agent conjugate as described herein, can be used to diagnose or image eye diseases such asacanthamoeba keratitis, blepharitis, CMV retinitis, conjunctivitis, corneal abrasion, dry eye syndrome, ocular herpes, fungal keratitis, trachoma, endophthalmitis, dacryostenosis, uveitis, Sjogren's syndrome, stye, ocular histoplasmosis syndrome, mycosis, toxoplasmosis, chlamydia, gonorrhea, bacterial keratitis, tuberculosis, leprosy, syphilis, hepatitis B, or infections caused by herpes simplex virus, epstein-barr virus, or Candida.

A peptide comprising the sequence of any of SEQ ID NO: 1-SEQ ID NO: 166, and any peptide derivative or peptide-active agent conjugate as described herein, can be used to treat vaginal diseases such as mucosa infections, bacterial vaginosis, vaginitis, yeast infection, chlamydia, gonorrhea, pelvic inflammatory disease, genital herpes, aerobic vaginitis, and infections caused by Candida albicans, Candida tropicalis, Candida krusei, Gardnerella vaginalis, Campylobacter, Trichomonas vaginalis, Streptococcus spp, Actinobacteria spp, Anaerococcus spp, Actinomyces naeslundii, Aggregatibacter actinomycetemcomitans, Atopobium vaginae, Bacteroides ureolyticus, Bifidobacterium spp, Clostridiales spp, Collinsella aerofaciens, Eggerthella spp, Eggerthella lenta, Eubacterium spp, Fusobacterium nucleatum, Leptotrichia spp, Leptotrichia amnionii, Megasphaera spp, Mobiluncus spp, Mycoplasma hominis, Mycoplamas parvum, Peptococcus spp, Peptomphilus spp, Peptostreptococcus spp, Peptostreptococcus anerobius, Porphyromonas gingivalis, Prevotella bivia spp, Prevotella disiens, Prevotella intermedia, Slackia spp, Sneathia sanguinegens, Tannerella forsythia, Treponema denticola, Ureaplasma urealyticum, or Veillonella parvula. An active agent can be clindamycin, metronidazole, tinidazole, butoconazole, clotrimazole, fluconazole, miconazole, terconazole, hydrocortisone, or tioconazole.

A peptide comprising the sequence of any of SEQ ID NO: 1-SEQ ID NO: 166, and any peptide derivative or peptide-active agent conjugate as described herein, can be used to treat vaginal diseases such as mucosa infections, bacterial vaginosis, vaginitis, yeast infection, chlamydia, gonorrhea, pelvic inflammatory disease, genital herpes, aerobic vaginitis, and infections caused by Candida albicans, Candida tropicalis, Candida krusei, Gardnerella vaginalis, Campylobacter, Trichomonas vaginalis, Streptococcus spp, Actinobacteria spp, Anaerococcus spp, Actinomyces naeslundii, Aggregatibacter actinomycetemcomitans, Atopobium vaginae, Bacteroides ureolyticus, Bifidobacterium spp, Clostridiales spp, Collinsella aerofaciens, Eggerthella spp, Eggerthella lenta, Eubacterium spp, Fusobacterium nucleatum, Leptotrichia spp, Leptotrichia amnionii, Megasphaera spp, Mobiluncus spp, Mycoplasma hominis, Mycoplamas parvum, Peptococcus spp, Peptomphilus spp, Peptostreptococcus spp, Peptostreptococcus anerobius, Porphyromonas gingivalis, Prevotella bivia spp, Prevotella disiens, Prevotella intermedia, Slackia spp, Sneathia sanguinegens, Tannerella forsythia, Treponema denticola, Ureaplasma urealyticum, or Veillonella parvula.

A peptide comprising the sequence of any of SEQ ID NO: 1-SEQ ID NO: 166, and any peptide derivative or peptide-active agent conjugate as described herein, can be used to treat oral diseases or infections such as thrush, herpangina, syphilis, gonorrhea, acute necrotizing ulcerative gingivitis, tuberculosis, cervicofacial actinomycosis, histoplasmosis, candidiasis, mucous membrane pemphigoid, erthema multiforme, pemphigus vulgaris, lichen planus, aphthous ulcers, or Behcet's syndrome, or infections caused by herpes simplex virus type 1 or type 2, Herpes labiales, Herpes zoster, epstein-barr virus, papillomavirus, coxsakievirus A, coxsakievirus B, or echovirus.

A peptide comprising the sequence of any of SEQ ID NO: 1-SEQ ID NO: 166, and any peptide derivative or peptide-detectable agent conjugate as described herein, can be used to diagnose or image oral diseases such as thrush, herpangina, syphilis, gonorrhea, acute necrotizing ulcerative gingivitis, tuberculosis, cervicofacial actinomycosis, histoplasmosis, candidiasis, mucous membrane pemphigoid, erthema multiforme, pemphigus vulgaris, lichen planus, aphthous ulcers, or Behcet's syndrome, or infections caused by herpes simplex virus type 1 or type 2, Herpes labiales, Herpes zoster, epstein-barr virus, papillomavirus, coxsakievirus A, coxsakievirus B, or echovirus.

In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Types of cartilage diseases or conditions that can be treated with a peptide or peptide-active agent conjugate of the disclosure can include inflammation, pain management, anti-infective, pain relief, anti-cytokine, cancer, injury, degradation, genetic basis, remodeling, hyperplasia, or the like. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis—nephritis, Coloboma of Alar-nasal cartilages with telecanthus, Alar cartilages hypoplasia—coloboma—telecanthus, Pierre Robin syndrome—fetal chondrodysplasia, Dysspondyloenchondromatosis, Achondroplasia regional—dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia—ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

Types of cartilage diseases or conditions that can be diagnosed or imaged with a peptide-detectable agent conjugate of the disclosure can include inflammation, pain management, anti-infective, pain relief, anti-cytokine, cancer, injury, degradation, genetic basis, remodeling, hyperplasia, or the like. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis—nephritis, Coloboma of Alar-nasal cartilages with telecanthus, Alar cartilages hypoplasia—coloboma—telecanthus, Pierre Robin syndrome—fetal chondrodysplasia, Dysspondyloenchondromatosis, Achondroplasia regional—dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia—ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or another cartilage or joint disease or condition.

In some embodiments, a peptide or peptide-active agent conjugate of this disclosure can be administered to a subject in order to target an arthritic joint. In other embodiments, a peptide or peptide-active agent conjugate of this disclosure can be administered to a subject in order to treat an arthritic joint.

In some embodiments, a peptide or peptide-detectable agent conjugate of this disclosure can be administered to a subject in order to diagnose or image an arthritic joint.

In some embodiments, the peptides of the present disclosure can be used to treat chondrosarcoma. Chondrosarcoma is a cancer of cartilage producing cells and is often found in bones and joints. It falls within the family of bone and soft-tissue sarcomas. In certain embodiments, administration of a peptide, peptide-active agent conjugate, or peptide-detectable agent conjugate of the present disclosure can be used to image and diagnose or target and treat a subject with chondrosarcoma. The subject can be a human or an animal.

In some embodiments, the peptides of the present disclosure are conjugated to one or more therapeutic agents. In further embodiments, the therapeutic agent is a chemotherapeutic, anti-cancer drug, or anti-cancer agent selected from, but are not limited to: anti-inflammatories, such as for example a glucocorticoid, a corticosteroid, a protease inhibitor, such as for example collagenase inhibitor or a matrix metalloprotease inhibitor (i.e., MMP-13 inhibitor), an amino sugar, vitamin (e.g., Vitamin D), and antibiotics, antiviral, or antifungal, a statin, and an immune modulator, In other embodiments, the therapeutic agent is any nonsteroidal anti-inflammatory drug (NSAID). The NSAID can be any heterocyclic acetic acid derivatives such as ketorolac, indomethacin, etodolac, or tolemetin, any propionic acid derivatives such as naproxen, any enolic acid derivatives, any anthranilic acid derivatives, any selective COX-2 inhibitors such as celecoxib, any sulfonanilides, any salicylates, aceclofenac, nabumetone, sulindac, diclofenac, or ibuprofen. In other embodiments, the therapeutic agent is any steroid, such as dexamethasone, budesonide, triamcinolone, cortisone, prednisone, rednisolone, triamcinolone hexacetonide, or methylprednisolone. In some embodiments, a treatment consists of administering a combination of any of the above therapeutic agents and a peptide-active agent conjugate, such as a treatment in which both a dexamethasone-peptide conjugate and an NSAID are administered to a patient. Peptides and peptide-active agent conjugates of the current disclosure that target the cartilage can be used to treat the diseases conditions as described herein, for example, any diseases or conditions including tears, injuries (i.e., sports injuries), genetic factors, degradation, thinning, inflammation, cancer or any other disease or condition of the cartilage or to target therapeutically-active substances to treat these diseases amongst others. In other cases, a peptide or a peptide-active agent conjugate of the disclosure can be used to treat traumatic rupture, detachment, chostochondritis, spinal disc herniation, relapsing and non-relapsing polychondritis, injury to the articular cartilage, osteoarthritis, arthritis or achondroplasia. In some cases, the peptide or peptide-active agent conjugate can be used to target cancer in the cartilage, for example benign chondroma or malignant chondrosarcoma, by contacting the cartilage by diffusion into chondrocytes and then having antitumor function, targeted toxicity, inhibiting metastases, etc. Additionally, a peptide-detectable agent conjugate can be used to label, detect, or image such cartilage lesions, including tumors and metastases amongst other lesions, which may be removed through various surgical techniques.

Peptides of the current disclosure that target the cartilage can be used to treat or manage pain associated with a cartilage injury or disorder, or any other cartilage or joint condition as described herein. The peptides can be used either directly or as carriers of active drugs, peptides, or molecules. For example, since ion channels are associated with pain and may be activated in disease states such as arthritis, peptides that interact with ion channels can be used directly to reduce pain. In another embodiment, the peptide is conjugated to an active agent with anti-inflammatory activity, in which the peptide acts as a carrier for the local delivery of the active agent to reduce pain.

In some embodiments, the peptides described herein provide a method of treating a cartilage condition of a subject, the method comprising administering to the subject a therapeutically-effective amount of a peptide comprising the sequence SEQ ID NO: 3-SEQ ID NO: 8, SEQ ID NO: 10, SEQ ID NO: 17, SEQ ID NO: 22, SEQ ID NO: 29-SEQ ID NO: 31, or any fragment thereof.

### Use of Peptides in Gastrointestinal Disease

The peptides of the present disclosure can be used to treat a gastrointestinal (GI) disease, disorder, or infection. Any one of the peptides of SEQ ID NO: 1-SEQ ID NO: 166 can be used to treat a gastrointestinal disease, disorder, or infection.

The peptides of the present disclosure can be used to diagnose or image a gastrointestinal (GI) disease, disorder, or infection. Any one of the peptides of SEQ ID NO: 1-SEQ ID NO: 146 can be used to diagnose or image a gastrointestinal disease, disorder, or infection.

For example, any one of the peptides of SEQ ID NO: 1-SEQ ID NO: 166 can be administered orally alone or as a conjugate with an active agent to treat or prevent a gastrointestinal disease, disorder, or infection. Any one of the peptides of SEQ ID NO: 1-SEQ ID NO: 166 can be administered orally as a conjugate with a detectable agent to diagnose or image a gastrointestinal disease, disorder, or infection.

The peptide can be recombinantly expressed or chemically synthesized. In some embodiments, the peptide can be fused with of chemically conjugated to an active agent or detectable agent to produce a peptide-active agent conjugate or peptide-detectable agent conjugate.

Because the peptides of this disclosure can be resistant to proteases, low pH, and/or reduction conditions found the environment of the GI tract, the peptides of this disclosure can remain intact in the GI tract long enough to have a therapeutic effect, to target a tissue, to accumulate in a tissue or cell, to deliver an active agent, to bind to, antagonize or agonize a receptor or enzyme or ion channel, to activate or block a biological pathway, to allow imaging, or to have another therapeutic or diagnostic effect. For example, linaclotide, which is a knotted peptide that is resistant to low pH and pepsin, can be orally administered and can agonize guanylate cyclase-C (for treatment of irritable bowel syndrome with constipation or chronic idiopathic constipation) in the gastrointestinal tract prior to being degraded in the intestinal lumen. As a result, linoclatide is not significantly systemically absorbed into the plasma, and therefore, sytemic side effects are avoided (see FDA label for Linzess (approved 2012)). Similarly to linaclotide, peptides of the disclosure can be used for treating diseases of the GI tract. The peptides or peptide-active agent conjugates can be orally administered to prevent or treat a GI infection, or a GI cancer. For example, peptides and/or peptide-active agent conjugates can be used to treat any one of the following gastrointestinal diseases, cancer, disorders or infections: infections caused norovirus, rotavirus, intestinal parasites (e.g., Entamoeba hystolytica, Trichomonas, Giardia, Bacteroides, Clostridium peptococcus, pinworm, Strongyloidiasis, Plasmodium falciparum, Cryptosporidium parvum, Cyclospora cayetanensis, Diphyllobothrium latum, Ascaris lumbricoides, Trichuris trichiura, Taenia solium, or Taenia saginata), Campylobacter, Clostridium botulinum, Clostridium perfringens, Escherichia coli, (including Shiga toxin-producing (STEC) strains of E. coli, E. coli O157:H7, E. coli O145, and E. coli O121:H19), Listeria, Salmonella, Shigella, Staphylococcal food poisoning, Typhoid fever, Vibrio, Yersinia, infections of enteric bacteria that can result in secretory or watery diarrhea (e.g., Vibrio cholera, ETECs (Enterotoxigenic E. coli), EPECs (Enteropathogenic E. coli)), invasive/tissue damaging enteric pathogens that can result in bloody diarrhea and dysentery (e.g., EIECs (Enteroinvasive E. coli), Shigella spp, Salmonella spp, EHECs (Enterohemmorhagic E. coli)), and slow bacterial infection pathogens (e.g., Helicobacter pylori), Balantidium, Cryptosporidium, Toxoplasma, Cyclospora, Micropsoridia, Trichomona, Candida, Staphylococcus, Streptococcus pyogenes, Staphylococcus aureus, Bacillus cereus, Yersinia enterocoliticia, Clostridium difficile, Vibrio parahaemolyticus, Aeromona hydrophila, Plesiomonas sp, norwalk virus, astrovirus, adenovirus, caliciviruses, or parvoviruses; chancoroid; granuloma inguinale; anal cancer; attenuated familial adenomatous polyposis; blumer's shelf; carcinoid; digestive system neoplasm; duodenal cancer; esophageal cancer; familial adenomatous polyposis; gardner's syndrome; gastric lymphoma; gastroinestinal stromal tumor; goblet cell carcinoid; hepatoblastoma; inflammatory myeloblastic tumor; intraductal papillary mucinous neoplasm; juvenile polyposis syndrome; krukenberg tumor; linitis plastica; MALT lymphoma; oesophagogastric juntional adenocarinoma; small intestine cancer; tonsil carcinoma; colon cancer; rectal cancer; gastric cancer; stromal tumors; lipomas; hamartomas; carcinoid syndromes; gastrointestinal carcinoid tumors; adenocarcinomas; sarcoma; gastro intestinal stromal tumors; bile duct cancer; colorectal cancer; nasopharyngeal cancer; oropharyngeal cancer; oral cancer; hypopharyngeal cancer; inflammatory bowel disease; irritable bowel syndrome; constipation; diarrhea; infection; ulcers; pain; metabolic disorders; obesity; immune disorders; autoimmune diseases; nausea; vomiting; bloating; motility disorders; achalasia; gastroparesis; dyspepsia; bleeding; gastroesophogeal reflux disease; Barret's esophagus; gastroenteritis; pyloric stenosis; anemia; pernicious anemia; Crohn's disease; ulcerative colitis; enterocolitis; ischemic colitis; radiation colitis; polyps; enteritis; celiac disease; malabsorption; appendicitis; colitis; diverticulitis; hemorrhoids; anal fissure; perianal absecesses; anal fistula; diverticulosis; acid reflex; hirschsprung disease; fecal incontinence, cyclic vomiting syndrome; dumping syndrome; gallstones; gas; gastritis; gastrointestinal bleeding; inguinal hernia; menetrier's disease; peptic ulcers; liver disease; pancreatitis; short bowel syndrome; viral gastroenteritis; whipple disease; zollinger-ellison syndrome; and proctitis.

In some embodiments, probiotic or commensal bacteria can be genetically engineered to produce a peptide of the present disclosure for use in GI disease treatment. In some embodiments, the peptide or peptide-active agent conjugate can be added to food. In other embodiments, the peptide, peptide-active agent, or genetically engineered probiotic or commensal bacteria that expresses the peptide can be taken as a pill similarly to conventional probiotics. Thus, the stable peptides or peptide-active agent conjugates of this disclosure can provide ongoing prophylaxis or treatment of a GI disease in extreme conditions (e.g., temperature) and can be used in settings without requiring additional storage equipment. This can be advantageous for use in developing countries that lack readily available refrigeration or for use by the armed services.

Peptide-active agent conjugates that can be used to treat gastrointestinal diseases, disorders or infections can comprise any one of the following active agents fused or chemically conjugated to any peptide of SEQ ID NO: 1-SEQ ID NO: 166: antibiotics (e.g., clindamycin, fusidic acid, muprirocin, oritavancin, tedizolid, tigecycline, animoglycosides (e.g., amikacin, gentamicin, kanamycin, neomycin, netilmicin, tobramycin, paromomycin, streptomycin, spectinomycin, loncosamides, clincamycin, linkomycin), ansamycin (e.g., geldanamycin, herbimycin, rifaximin), cabapenems (e.g., ertapenem, doripenem, imipenem/cilastatin, meropenem), quinolines/fluorquinolones (e.g., ciprofloxacin, enoxacin, gatifloxacin, gemifloxacin, levofloxacin, moxifloxacin, nalidixic acid, norfloxacin, ofloxacin, trovaloxacin, grepafloxacin, sparfloxacin, temafloxacin, lomefloxacin), piperacillin/tzaobatam, ticarcillin/clavulanic acid, amoxicillin/clavulanate, ampicillin/sulbactam, streptogramins, cephalosporins (e.g., cefadroxil, cefazolin, cefalotin, ceflexin, cefactor, cefamandole, cefoxitin, cefprozil, cefuroxime, cefixime, cefdinir, cefoperazone, cefotaxime, cefpodoxime, ceftazidime, ceftibuten, ceftizoxime, ceftriazone, cefepime, ceftaroline fosamil, ceftobiprole), glycopeptides (e.g., teicoplanin, vancomycin, telavancin, dalbavancin), lipeptide (e.g., daptomycin), macrolides (e.g., azithromycin, clarithromycin, dirithromycin, erthyromycin, roxithromycin, troleandomycin, telithromycin, spiramycin), monobactams (e.g., aztreonam), nitrofurans (e.g., furazolidone, nitrofurantoin), oxazolidinones (e.g., linezolid, posizolid, radezolid, torezolid), pencillin (e.g., amoxicillin, ampicillin, azlocillin, carbencillin, cloxacillin, dicloxacillin, flucloxacillin, mezlocillin, methicillin, nafcillin, oxaillin, penicillin g, temocillin, ticarcillin), polypetides (e.g., bacitracin, colistin, polymyxin B), sulfonamides (e.g., mafenide, sulfacetamide, sulfadiazine, silver sulfadiazine, sulfamethizole, sulfamethoxazole, sulfanilimide, sulfasalazine, sulfisoxazole, trimethoprim-sulfamethoxazole, sulfonamidochrysoidine), tetracyclines (e.g., demclocyline, doxycycline, minocycline, oxytetracycline, tetracycline), clofazimine, dapsone, capreomycin, clycoserine, ethambutol, ethionamede, isoniazid, pyriazinamide, rifampicin, sterptomycin, arsphenamine, chloramphenicol, fosomycin, metronidazole, platensimycin, quinupristin/dalfopristin, thiamphenicol, trimethoprim, extended spectrum penicillins (e.g., ticaracillin, piperacillin), nitrofurantoin, antiparasitics (e.g., nitazoxanide, melarsoprol, eflorinithine, metronidazole, mebendazole, praziquantel, thiobendazole, ivermectin, tinidazole, miltefosine, pyrantel pamoate, thiabendazole, diethylcarbamizine, niclosamide, albendazole, rifampin, amphoteicin B), antifungals (e.g., fumagillin, amphotericin B, candicidin, filipin, hamycin, nataycin, nystatin, rimocidin, bifonazole, butoconazole, clotrimazole, econazole, fenticonazole, isoconazole, ketoconazole, luiconzole, miconazole, omoconazole, sertaconazole, sulconazole, tioconazole, albaconazole, efinaconazole, epoxiconazole, fluconazole, isavuconazole, itraconazole, posaconazole, propiconazole, ravuconazole, teronazole, voriconazole, abafungin, amorolfin, butenafine, naftifine, terbinafine, anidulafungin, caspofungin, micafungin, aurones, benzoic acid, ciclopirox, flucytosine, griseofulvin, haloprogin, tolnafrtate, undecylenic acid, crystal violet, balsam of Peru), antiviral agents (e.g., abacavir, acyclovir, adefovir, amantadine, amprenavir, ampligen, arbidol, atazanavir, atripla, balavir, cidofovir, combivir, dolutegravir, darunavir, delavirdine, delavirdine, didanosine, docosanol, edoxudine, efavirenz, emtricitabine, enfuvirtide, entecavir, ecoliever, famciclovir, fomivirsen, fosamprenavir, foscarnet, fosonet, fusion inhibitor, ganciclovir, ibacitabine, imunovir, idoxuridine, imiquimod, indinavir, inosine, integrase inhibitor, interferon, lamivudine, lopinaivir, loviride, maraviroc, moroxydine, methisazone, nelfinavir, nevirapine, nexavir, nitazoxanide, nucleoside analogs, novir, oseltamivir, perginterone alfa-2a, penciclovir, peramivir, pleconaril, pdodphyllotoxin, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine), anti-inflammatory agents (e.g., naproxen, diclofenac, ibuprofen, indomethacin, piroxicam, nabumetone, etodolac, celecoxib, sulindac, oxaprozin, meloxicam, aspirin, fenoprofen, diflunisal, tolmetin, ketorolac, flurbiprofen, mefenamic acid, ketoprofen, salsalate, valdecoxib, loxoprofen, phenylbutazone) immune modulators (e.g., azathiorpine, mercaptopurine, methotrxate, alefacept, anakinra, certolizumab pegol, etanercept, golimumab, infliximab, natalizumab, rituximab, tocilizumab, ustekinumab), cancer therapeutics (e.g., actinomycin, azacitidine, azathioprine, bleomycin, bortezomib, carboplatin, capecitabine, cisplatin, chlorambucil, cyclophosphamide, cytarabine, daunorubicin, doxifluridine, doxifluridine, doxorubicin, epirubicin, epothilone, etoposide, fluorouracil, gemicitabine, hyroxyurea, idarucibin, imatinib, irinotecan, mechlorethamine, mercaptopurine, methotrexate, mitoxantrone, oxaliplatin, paclitaxel, pemetrexed, teniposide, tioguanine, topetecan, valrubicin, vemurafenib, vinblastine, vincritine, vindesine, vinorelbine), gastric motility modulators (e.g., benzamide, cisapride, domperidone, erythomycin, itopride, mosapride, metoclopramide, prucalopride, renzapride, tegaserod, mitemcinal, levosulpiride, cinitapride), anti-diarrheals (e.g., attapulgite, bismuth subsalicylate, crofelemer, diaraid, diasorb, difenoxin hcl/atropine, diphenoxylate hcl/atropine, imodium, k-pek, kaopectate, lomotil, lonox, loperamide, loperamide/simethicone, mallox, motofen, mytesi, neodiaral, octretoide, opium paregoric, opium tincture, paregoric, rifximin, sandostatin, xifaxan), constipation inhibitors (inaclotide, lactulose, lubiprostone, plecanatide, polyethylene glycol), or ion channel modulators.

Peptide-detectable agent conjugates that can be used to treat gastrointestinal diseases, disorders or infections (as disclosed above) can comprise any detectable agent as disclosed herein or any of the following detectable agents fused or chemically conjugated to any peptide of SEQ ID NO: 1-SEQ ID NO: 166: imaging agents, fluorescent dyes, or radioisotopes.

Other materials can also be conjugated to or formulated (such as in a tablet or capsule) with the peptide, peptide-active agent conjugate, or peptide-detectable agent conjugate to increase residence time in the gut mucosa after oral administration. For example, mucoadhesive polymers can be conjugated to peptides, peptide-active agent conjugates, or peptide-detectable agent conjugates. Mucoadhesive polymers can include hydroxypropyl methylcellulose, hydroxypropyl cellulose (HPC), methylcellulose (MC), and carboxymethyl cellulose (CMC), and insoluble cellulose derivatives such as ethylcellulose and microcrystalline cellulose (MCC), polyacrylates, starch, chitosan, or any polymer described in Chaturvedi et al. (J Adv Pharm Technol Res., 2(4): 215-222 (2011)). Mucoadhesive polymers can also include Carbopol®, Polycarbophil®, sodium alginate, sodium carboxymethylcellulose, hydroxypropylmethylcellulose (HPMC), polyethylene glycol, polyvinylpyrrolidone, hydroxyethycellulose, poloxamer, or any polymer described by Yu et al. in Chapter 2 of das Neves, José, and Bruno Sarmento, eds. Mucosal Delivery of Biopharmaceuticals: Biology, Challenges and Strategies. Springer Science & Business Media, 2014. In some embodiments, peptides and/or peptide-active agent conjugates and/or peptide-detectable agent conjugates further coupled to mucoadhesive polymers can enhance the mucoadhesivity of the peptide-polymer and/or peptide-active agent-polymer conjugates and/or peptide-detectable agent-polymer conjugates. By increasing the residence time in the gastrointestinal tract, these mucoadhesive polymers can facilitate sustained therapeutic efficacy of peptides, peptide-active agent conjugates, and peptide-detectable agent conjugates to treat, diagnose, or image gastrointestinal diseases, disorders, and infections. Additionally, a peptide, peptide-active agent conjugate, or peptide-detectable agent conjugate of this disclosure can be formulated to target delivery of the peptide, peptide-active agent conjugate, or peptide-detectable agent conjugate to a specific part of the GI tract or release of the active agent or detectable agent in a specific part of the GI tract, such as with polymer coatings or with other known formulations in the art.

Any peptide or peptide-conjugate of the present disclosure can also be modified to reduce breakdown and/or degradation of the conjugated active agent, which can thereby prevent degradation of active agents that are sensitive to low pH or intestinal enzymes. For example, in some embodiments, peptide-active agent conjugates can be administered to treat disease in the colon. For the treatment of a disease in the colon, a peptide or peptide-active agent conjugate can be formulated, such as, but limited to, in a suppository, tablet, or capsule, with polymer coatings, or any formulation as described herein or known in the art, to prevent premature release of the active agent in the small intestine or stomach, which can allow the peptide or peptide-active agent conjugate to remain intact until it reaches the colon. Alternatively, a peptide-active agent conjugate can be linked to the active agent via a linker that can be cleaved by enzymes or conditions that are specific to the colon, which can also prevent premature release of the active agent in the small intestine or stomach.

### Peptide Kit

In one aspect, peptides described herein can be provided as a kit. In another embodiment, peptide conjugates described herein can be provided as a kit. In another embodiment, a kit comprises amino acids encoding a peptide described herein, a vector, a host organism, and an instruction manual. In some embodiments, a kit includes written instructions on the use or administration of the peptides.

### EXAMPLES

The following examples are included to further describe some aspects of the present disclosure, and should not be used to limit the scope of the invention.

### Example 1

### Manufacture of Peptides

This example describes the manufacture of the peptides described herein. Peptides derived from knottin proteins were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transduced into HEK-293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen.

### Example 2

### Peptide Expression Using a Mammalian Expression System

This example describes expression of the peptides using a mammalian expression system. Peptides were expressed according to the methods described in in Bandaranayake et al., Nucleic Acids Res. 2011 November; 39(21): e143. Peptides were cleaved from siderocalin using tobacco etch virus protease and purified by FPLC on a hydrophobic columns using a gradient of acetonitrile and 0.1% TFA. Peptides were then lyophilized and stored frozen.

### Example 3

### Peptide Radiolabeling

This example describes the radiolabeling of peptides. Several peptides were radiolabeled by reductive methylation with ¹⁴C formaldehyde and sodium cyanoborohydride with standard techniques (such as those described in Jentoft et al. J Biol Chem. 254(11):4359-65.1979). The sequences were engineered to have the amino acids, “G” and “S” at the N terminus. See Methods in Enzymology V91:1983 p. 570 and JBC 254(11):1979 p. 4359. An excess of formaldehyde was used to drive complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas.

### Example 4

### Peptide Resistance to Pepsin, Low pH, and/or Reduction

This example shows peptide resistance to enzymatic degradation by pepsin, reduction by DTT, or degradation at low pH. Peptides were first suspended in 500 ul of ddH₂O to a stock concentration of 2 mg/ml. Reactions were prepared by adding 12.5 ug of peptide from the stock solution to a 10 mM solution of DTT in PBS and allowed to incubate at room temperature for 30 minutes. Other reactions were prepared with 12.5 μg peptide with or without 500 U/ml pepsin in simulated gastric fluid (SGF; pH 1.05; 2% (w/v) sodium chloride in 0.7% (v/v) hydrochloric acid) and were incubated for 30 minutes at 37.5° C. Some reactions were quenched with a final concentration of 100 mM Tris base and 10 mM dithiothreitol (DTT). Some reactions were performed in all of the above components, including simulated gastric fluid, pepsin, Tris, and DTT (referred to as SPTD). Reversed phase HPLC (RP-HPLC) was run on samples using an Agilent 1260 HPLC equipped with a C-18 Poroshell 120B column. Sample were analyzed by a gradient method with a mobile phase of Solvent A (water with 0.1% TFA) and Solvent B (acetonitrile with 0.1% TFA). Solvent B was ramped up from 5%-45% of the mobile phase over a period of 10 minutes. Peptides were detected at an absorbance of 214 nm and 280 nm.

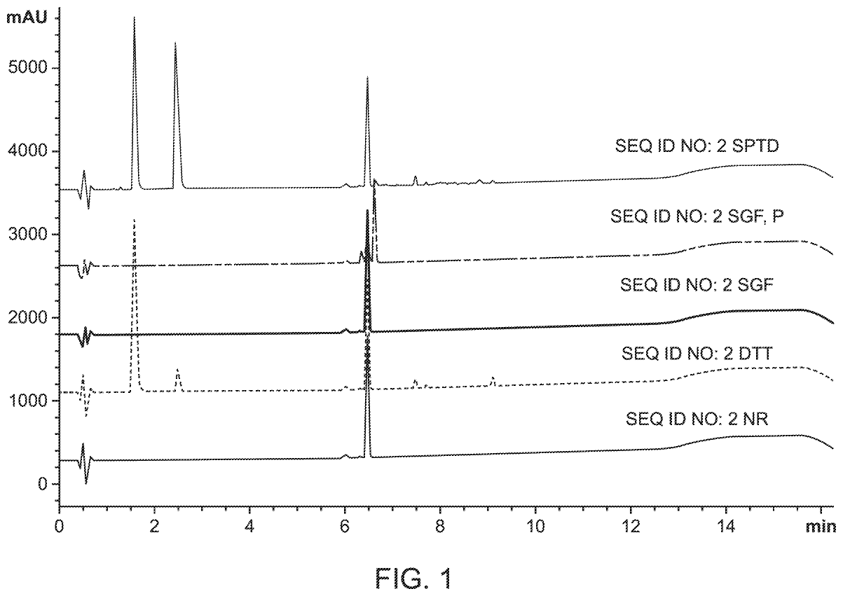

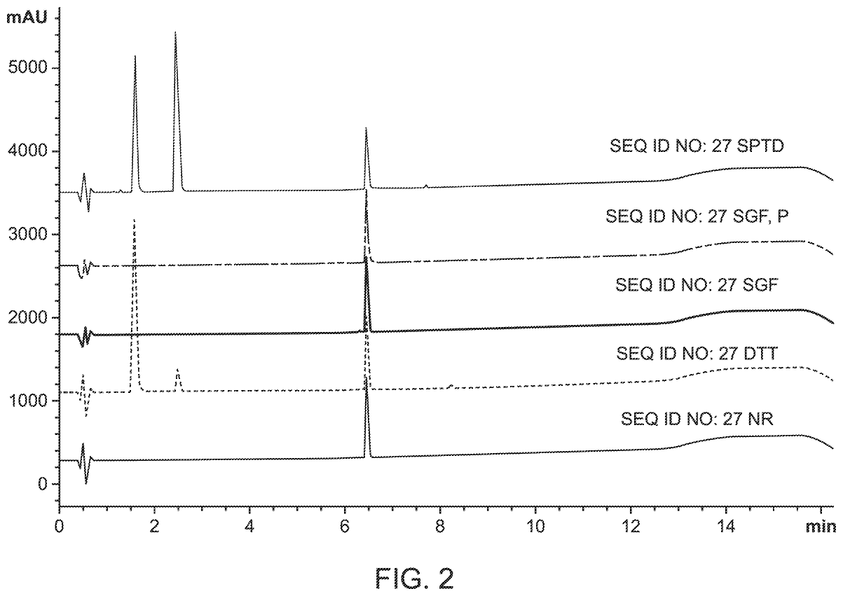

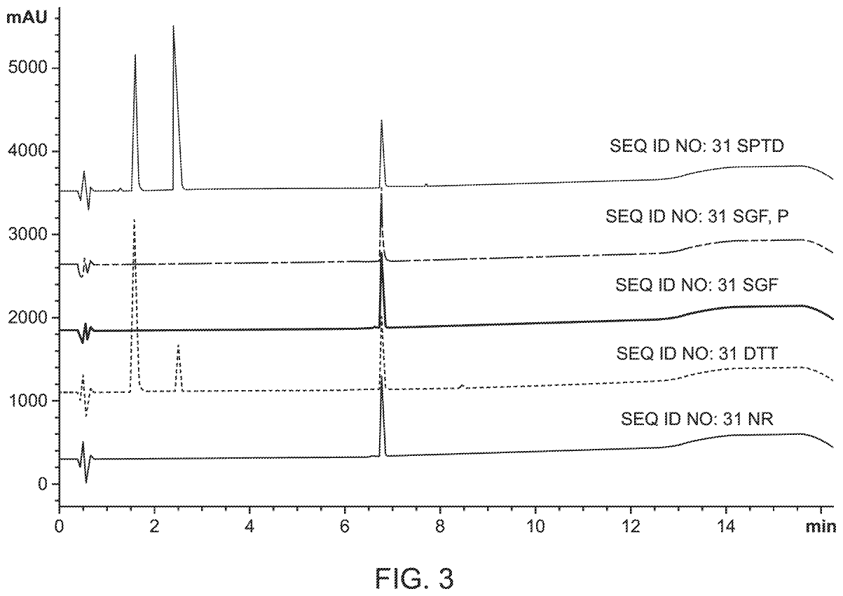

FIG. 1 shows an HPLC chromatogram of a peptide of SEQ ID NO: 2 in various solutions including SPTD, simulated gastric fluid (SGF) and pepsin (P), SGF alone, DTT alone, and non-reduced peptide (starting peptide, no treatment with DTT, SGF, or P). FIG. 2 shows an HPLC chromatogram of a peptide of SEQ ID NO: 27 in various solutions including SPTD, simulated gastric fluid (SGF) and pepsin (P), SGF alone, DTT alone, and non-reduced peptide. FIG. 3 shows an HPLC chromatogram of a peptide of SEQ ID NO: 31 in various solutions including SPTD, simulated gastric fluid (SGF) and pepsin (P), SGF alone, DTT alone, and non-reduced peptide.

FIG. 1 shows HPLC chromatograms of 12.5 μg of a peptide of SEQ ID NO: 2 suspended in various solutions including SPTD, simulated gastric fluid (SGF) at (pH 1.05; 2% (w/v) sodium chloride in 0.7% (v/v) hydrochloric acid) and 500 U/ml pepsin (P), SGF, dithiothreitol (DTT), and non-reducing (NR) conditions. The peak near 6.5 minutes was the intact peptide of SEQ ID NO: 2, the peak near 1.5 minutes was DTT, and the peak near 2.5 minutes was oxidized DTT. The peptide peak of SEQ ID NO: 2 was found to be present near 6.5 minutes in the DTT and SGF chromatograms as in the NR chromatogram, indicating that the peptide remained intact even after incubation with DTT and at low pH. The intact peptide peak was also present in the SPTD chromatogram, indicating that the peptide was resistant to pepsin degradation in SGF with DTT. FIG. 2 shows HPLC chromatograms of 12.5 μg of a peptide of SEQ ID NO: 27 suspended in various solutions including SPTD, simulated gastric fluid (SGF) (pH 1.05; 2% (w/v) sodium chloride in 0.7% (v/v) hydrochloric acid) and 500 U/ml pepsin (P), SGF, DTT, and non-reducing (NR) conditions. FIG. 3 shows HPLC chromatograms of 12.5 μg of a peptide of SEQ ID NO: 31 suspended in various solutions including SPTD, simulated gastric fluid (SGF) (pH 1.05; 2% (w/v) sodium chloride in 0.7% (v/v) hydrochloric acid) and 500 U/ml pepsin (P), SGF, DTT, and non-reducing (NR) conditions. Intact peptides were observed near 6.5 minutes and 6.75 minutes, as shown in the NR chromatograms. The intact peptide peak remained unchanged in the DTT, SGF, SGF and P, and SPTD chromatograms, indicating that the peptides of SEQ ID NO: 27 and SEQ ID NO: 31 were highly resistant to the above reducing, low pH, or enzyme-rich conditions.

### Example 5

### Peptide Resistance to Trypsin Digestion, Pepsin Digestion, Reduction and/or Elevated Temperature

This example shows peptide resistance to trypsin digestion, pepsin digestion, reduction by DTT, and to degradation at high temperatures. Peptides were first suspended in 500 μl of ddH₂O to a stock concentration of 2 mg/ml. Reactions were prepared by adding 12.5 μg of peptide from the stock solution to a 10 mM solution of DTT in PBS and allowed to incubate at room temperature for 30 minutes. Other reactions were prepared with 12.5 μg peptide and 500 U/ml trypsin in 25 mM Tris/75 mM NaCl buffer (pH 7.0) and incubated for 30 minutes at 37.5° C. These reactions were then quenched with 5 μg of soybean trypsin inhibitor (I) and, in some cases, 10 mM dithiothreitol (DTT). Reversed phase HPLC (RP-HPLC) was run on samples using an Agilent 1260 HPLC equipped with a C-18 Poroshell 120B column. Sample were analyzed by a gradient method with a mobile phase of Solvent A (water with 0.1% TFA) and Solvent B (acetonitrile with 0.1% TFA). Solvent B was ramped up from 5%-45% of the mobile phase over a period of 10 minutes. For testing pepsin digestion resistance, reactions were prepared with 12.5 μg peptide with or without 500 U/ml pepsin in simulated gastric fluid (SGF; pH 1.05; 2% (w/v) sodium chloride in 0.7% (v/v) hydrochloric acid) and were incubated for 30 minutes at 37.5° C. For testing temperature resistance, peptides were incubated at 70° C., 75° C., or 100° C. for one hour in 0.5 mM PBS, pelleted, and then the supernatant was analyzed by HPLC.

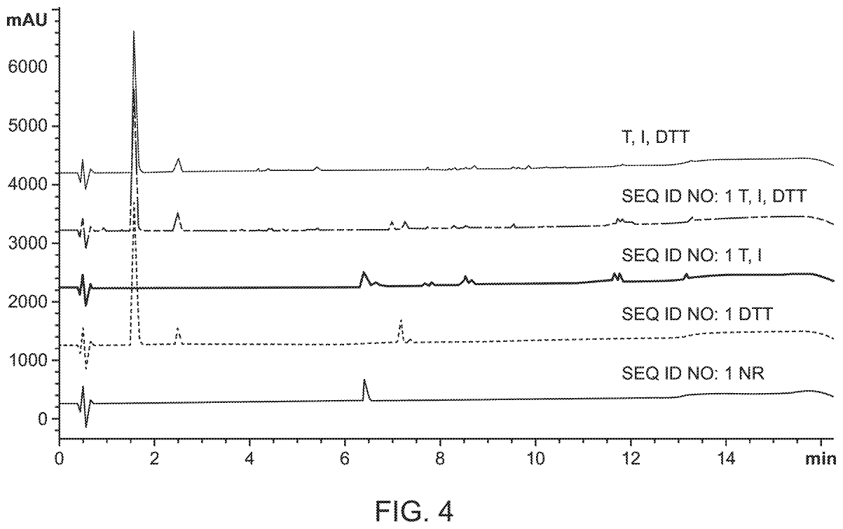

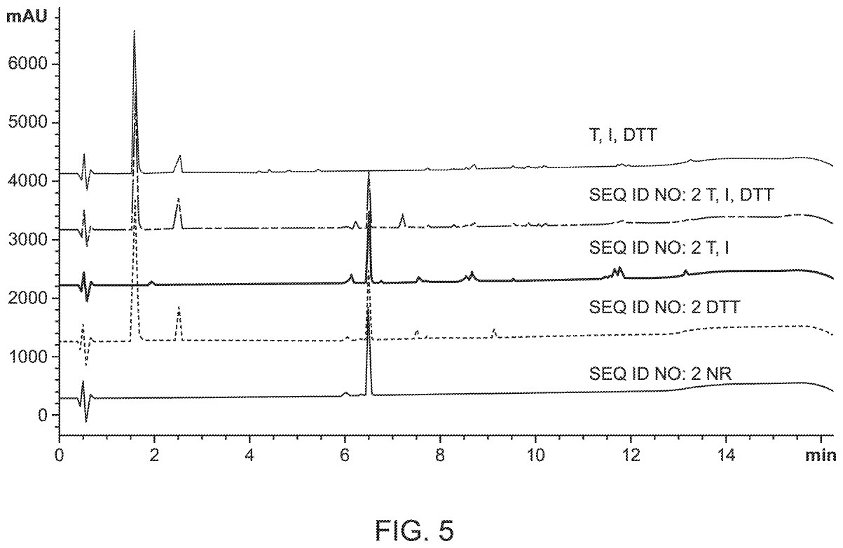

FIG. 4 shows an HPLC chromatogram of 500 U/ml trypsin (T) in 25 mM Tris, 5 μg soybean trypsin inhibitor (I) and 10 mM dithiothreitol (DTT) (T, I, DTT) as well as HPLC chromatograms of 12.5 μg of a peptide of SEQ ID NO: 1 suspended in various solutions including (T, I, DTT), (T, I), DTT, and non-reducing (NR) conditions (starting peptide, not treatment with DTT, T, or I). DTT itself eluted near 1.5 minutes and 2.5 minutes (reduced and oxidized). The NR trace shows that the intact peptide eluted near 6.5 minutes. The DTT treated peptide chromatogram shows minimal intact peptide near 6.5 minutes and new peaks of reduced peptide near 7.25 minutes. The trypsin treated peptide chromatogram also shows some intact peptide at 6.5 minutes as well as various new peaks indicating degradation products. This indicates that the peptide of SEQ ID NO: 1 is partially resistant to degradation by trypsin and not resistant to DTT treatment under these conditions. FIG. 5 shows an HPLC chromatogram of 500 U/ml trypsin in 25 mM Tris, 5 μg soybean trypsin inhibitor and 10 mM DTT (T, I, DTT) as well as HPLC chromatograms of 12.5 μg of a peptide of SEQ ID NO: 2 suspended in various solutions including (T, I, DTT), (T,I), DTT, and non-reducing (NR) conditions. The intact peptide eluted at 6.5 minutes after incubation with DTT and T, indicating that the peptide of SEQ ID NO: 2 was highly resistant to reduction by DTT and digestion by trypsin.

TABLE 2 shows a summary of peptides of this disclosure and their stability under various conditions.

TABLE 2

| | | | Resistance to | Resistance to | Resistance to | Resistance to |
|---|---|---|---|---|---|---|
| | Resistance to | Resistance to | Temperature | Temperature | Temperature | DTT Reduction |
| SEQ ID NO: | Trypsin (500 U/ml) | Pepsin (500 U/ml) | (70° C.) | (75° C.) | (100° C.) | (10 mM) |
| 1 | Moderate | | | | | Low |
| 2 | High | High | | | | High |
| 3 | Low | Moderate | High | High | High | Low |
| 26 | Moderate | | | | | Low |
| 27 | High | High | | High | High | High |
| 22 | Low | High | | High | High | Low |
| 30 | Low | Moderate | | High | Low | Low |
| 4 | Low | High | | High | Low | Low |
| 5 | Moderate | Moderate | | High | High | Low |
| 6 | High | High | | High | High | High |
| 7 | Low | High | | High | High | Low |
| 8 | Low | High | | High | High | Low |
| 9 | Moderate | High | | High | Low | Low |
| 10 | Low | High | | High | High | High |
| 11 | High | High | | High | Low | Low |
| 12 | Moderate | High | | High | High | Moderate |
| 13 | Low | High | | High | High | Low |
| 14 | Low | Moderate | | High | High | Low |
| 15 | Low | High | | High | Low | Low |
| 16 | Low | High | | High | Low | Low |
| 17 | Low | High | | High | High | Moderate |
| 2 | Low | High | | High | High | Low |
| 18 | Low | High | | High | Moderate | Low |
| 19 | High | High | | High | Moderate | Low |
| 20 | High | High | | High | Moderate | Low |
| 31 | High | High | | | | High |
| 21 | Moderate | High | | High | Low | Low |
| 32 | Low | High | High | High | High | Low |
| 33 | Low | Low | High | High | Low | Low |
| 35 | Low | Low | High | High | High | Low |
| 25 | Low | High | High | High | Low | Low |
| 36 | Low | High | High | High | Low | Low |
| 37 | Low | High | High | High | High | Low |
| 38 | Low | High | High | High | Low | Low |
| 39 | | | | | | Low |
| 40 | | | | | | Low |
| 41 | | | | | | Low |
| 42 | | | | | | Low |
| 43 | Moderate | | | | | Moderate |
| 44 | | | | | | Moderate |
| 45 | | | | | | Moderate |
| 46 | Low | Low | | High | Low | Low |
| 47 | Low | Low | | High | Low | Low |
| 48 | Low | High | | High | High | Low |
| 49 | Low | High | | High | Low | Low |
| 50 | Low | Low | | High | Low | Low |
| 24 | Low | High | | High | High | High |
| 29 | Low | High | | High | Low | Low |
| 51 | Low | High | | High | Low | Low |
| 52 | Low | High | | High | High | Low |
| 53 | Low | High | | High | Low | Low |
| 54 | Low | High | | High | High | Low |
| 55 | Low | High | | High | Low | Low |
| 56 | Low | High | | High | Low | Low |
| 23 | Low | High | | High | Low | Low |
| 57 | High | High | | High | High | High |
| 34 | Low | Low | | High | Low | Low |

As shown by the data in TABLE 2, the tested peptides of the present disclosure show a range of resistance to proteases, reduction, and high temperature. The peptides were classified as high, medium, or low for each condition. A peptide classified as low indicates that the majority (over 75%) of the HPLC peak of the treated peptide disappeared as compared to the HPLC peak of the untreated peptide. For example, after a peptide is treated at 100° C., if the HPLC peak of the peptide is mostly absent as compared to HPLC peak of the peptide not treated at 100° C., then the peptide is classified as low or having low resistance to 100° C. After a peptide is treated with reducing conditions, if there is no or very minimal HPLC peak that overlaps with the HPLC peak of the peptide not treated with reducing conditions, then the peptide is classified as low or having low resistance to reducing conditions. A peptide classified as medium indicates that about half (between 25% and 75%) of the HPLC peak of the treated peptide disappeared as compared to the HPLC peak of the untreated peptide. A peptide classified as high indicates that the majority (at least 75%) of the HPLC peak of the treated peptide is still present as compared to the HPLC peak of the untreated peptide.

The conditions tested were quite aggressive, meaning they were often at higher concentrations or temperature than may be encountered in vivo or in manufacturing, handling, transportation, and storage. For instance, 10 mM DTT is a much higher reducing environment than a reducing environment present in many in vivo locations produced by natural reducing agents such as glutathione, and 100° C. (or even 40° C.) is a much higher temperature than the typical storage temperature (room temperature), in which room temperature is a desired storage temperature for drug distribution and storage in warm climates and in geographical areas where refrigeration is not widely available. Thus, even a peptide that showed “low” resistance may be able to resist degradation by these conditions to a much higher degree than many agents or peptides currently being used in medical applications (and are not the subject of this disclosure). Similarly, even a peptide that showed “low” resistance may be able to adequately to resist degradation as needed for its intended use. Moreover, resistance to such conditions as shown by the peptides of this disclosure can be indicative of the utility of these peptides for use as a delivery scaffold under physiologic conditions, thus their utility for medical use.

While all the peptides that were tested at 75° C. were highly resistant to incubation at 75° C. (which can degrade many other peptides and proteins), not all tested peptides were highly resistant to incubation at 100° C. Similarly, not all tested peptides were highly resistant to the proteases or reduction conditions. More specifically, 21 tested peptides were highly resistant to 100° C., 38 tested peptides were highly resistant to pepsin, 7 tested peptides were highly resistant to DTT reduction, and 8 tested peptides were highly resistant to trypsin. Additionally, there was variability between peptides in their resistance to multiple different degrading conditions. More specifically, 5 peptides that were highly resistant to DTT reduction were also highly resistant to pepsin, 75° C., and 100° C., but only 3 of these peptides were also highly resistant to trypsin. Furthermore, the range of resistance of peptides was not due to their CDP classifications or their source organism. For example, both the 100° C. highly resistant peptide subset and the not highly resistant to 100° C. peptide subset comprised peptides that can be classified as hitchins, knottins, or other protein scaffolds and were from a variety of disparate species, including scorpions, spiders, and humans. These data illustrate that not all knotted peptides or other cystine dense peptides are equally resistant to these various conditions.

The peptides with the highest resistance data were further analyzed. These included the five peptides that were highly resistant to reduction, pepsin, 75° C., and 100° C. In addition, the peptide of SEQ ID NO: 12 that was highly resistant to pepsin, 75° C., and 100° C., and was moderately resistant to both DTT and tryspin was also further analyzed. SEQ ID NO: 12 was chosen for further analysis because it represented the highest subset of resistance and has sequence and topology (hitchin) similarity with the other five highly resistant peptides in contrast to SEQ ID NO: 17 which shared the resistance of the other five highly resistant peptides, but has a different topology (knottin) and disulfide bond pattern. Additional other peptides that showed high resistance properties were SEQ ID NO: 31 and SEQ ID NO: 3. The six peptides that were further tested were peptides comprising the sequences of SEQ ID NO: 27, SEQ ID NO: 24, SEQ ID NO: 6, SEQ ID NO: 10, SEQ ID NO: 12, and SEQ ID NO: 57, which were all hitchins. FIG. 27 shows a sequence alignment of these six hitchin peptides that were highly resistant to reduction, pepsin, 75° C., and 100° C. and nine other hitchin peptides that were not highly resistant to reduction, pepsin, trypsin, 75° C., and 100° C. FIG. 27A shows the sequence alignment of SEQ ID NO: 3, SEQ ID NO: 8, SEQ ID NO: 30, SEQ ID NO: 5, SEQ ID NO: 35, SEQ ID NO: 36, SEQ ID NO: 37, SEQ ID NO: 18, and SEQ ID NO: 56, which encode peptides that were not resistant to reduction pepsin, trypsin, 75° C., and 100° C. FIG. 27B shows the sequence alignment of SEQ ID NO: 27, SEQ ID NO: 24, SEQ ID NO: 6, SEQ ID NO: 10, SEQ ID NO: 12, and SEQ ID NO: 57, which encode peptides that were highly resistant to reduction, pepsin, 75° C., and 100° C. The * denotes that the peptide of SEQ ID NO: 12 was only moderately resistant to reduction. FIG. 27C is a sequence analysis of SEQ ID NO: 27, SEQ ID NO: 24, SEQ ID NO: 6, SEQ ID NO: 10, SEQ ID NO: 12, and SEQ ID NO: 57 from FIG. 27B. FIG. 27C identifies which amino acids are present at each position in SEQ ID NO: 27, SEQ ID NO: 24, SEQ ID NO: 6, SEQ ID NO: 10, SEQ ID NO: 12, and SEQ ID NO: 57. For example, at position one, all peptide sequences comprise a G amino acid residue; at position two, all peptide sequences comprise an S amino acid residue; at position three, all peptide sequences comprise either G, Q, or V amino acid, or null; and at position four, all peptide sequences comprise V, R, or K amino acid, or null. The amino acid residues at each position throughout the sequences from FIG. 27B are shown in FIG. 27C. When examining FIG. 27A, it is seen that each of the non-highly resistant peptide sequences deviate from those amino acids in FIG. 27C at one or more positions. One sequence that can be derived from the amino acid residues summarized in FIG. 27C is SEQ ID NO: 165, which also allows for the interchange of K and R amino acid residues in the sequence (SEQ ID NO: 27 and SEQ ID NO: 31 show that K and R amino acid residues can be interchanged): GSX₁X₂X₃X₄X₅X₆X₇CX₈X₉SX₁₀X₁₁CX₁₂X₁₃X₁₄CX₁₅X₁₆X₁₇X₁₈GX₁₉X₂₀X₂₁X₂₂X₂₃CX₂₄NX₂₅X₂₆CX₂₇CX₂₈X₂₉X₃₀ (SEQ ID NO: 167), wherein X₁ can be G, Q, V, or null; X₂ can be V, R, K, or null; X₃ can be P, I, F, or null; X₄ can be I, T, or L; X₅ can be N, D, P, or S; X₆ can be V, I, or N; X₇ can be K, R, or S; X₈ can be R, K, N, S, or T; X₉ can be G, I, H, N, or A; X₁₀ can be R, K, G, S, or Y; X₁₁ can be D, Q, or E; X₁₂ can be L, I, F, or W; X₁₃ can be P, D, E, R, or K; X₁₄ can be P, V, or H; X₁₅ can be R, K or I; X₁₆ can be K, R, D, S, or Q; X₁₇ can be A, I, R, K, or M; X₁₈ can be null or F; X₁₉ can be M, K, R, or T; X₂₀ can be R, K, T, or P; X₂₁ can be F, N, or A; X₂₂ can be G or A; X₂₃ can be K or R; X₂₄ can be I, M, or V; X₂₅ can be S, G, R, or K; X₂₆ can be K, R, or L; X₂₇ can be H, D, Y, R, or K; X₂₈ can be T, Y, or F; X₂₉ can be P, S, or null; and X₃₀ can be null, K, or R. Additionally, SEQ ID NO: 168 can be generated from the more frequent amino acid residues summarized in FIG. 27C. Alignment and analysis of the sequences of peptides that were also highly resistant to trypsin, SEQ ID NO: 27 and SEQ ID NO: 57, was used to generate SEQ ID NO: 169. SEQ ID NO: 167 and SEQ ID NO: 168 describe sequences of peptides that are particularly resistant to reduction, pepsin, 75° C., and 100° C., which would typically degrade peptides. Similar resistance trypsin, pepsin, 75° C. and 100° C. is exhibited by a peptide of SEQ ID NO: 167-SEQ ID NO: 169 when the N-terminal GS is removed. Additionally, SEQ ID NO: 169 describes sequences of peptides that are also particularly resistant to trypsin.

FIGS. 27D-27G show additional conserved features in the highly resistant sequences of FIG. 27B. These sequences all contain a conserved proline amino acid between the second cysteine and third cysteine in the knotted peptide (i.e., Cys2 and Cys3), either as the second amino acid between these cysteines or as the third amino acid between these cysteines. These conserved prolines are circled in FIGS. 27D-27G. A proline amino acid is a unique amino acid in that its side chain forms a 5-membered cyclic ring containing an alpha-amino group. This cyclic nature can cause proline residues to impart structural rigidity to a peptide. Thus, a proline residue can constrain the conformations available to the peptide in that location, e.g., the proline residue and its location can force a peptide to have a specific and constrained conformation, which can stabilize the peptide against degradation by chemical (reduction), enzymatic cleavage, and thermal stresses. A peptide can be so conformationally constrained that even if DTT can reduce the peptide's cystines to cysteines, the cystine bonds can spontaneously reform due to their proximity resulting from the peptide's constrained conformation. It is also possible that the peptide conformation can sterically hinder DTT from having sufficient access to the cysteines. Such conformational constraints might similarly provide resistance to degradation by enzymes or denaturation by thermal stresses.

FIG. 27D shows a sequence alignment of SEQ ID NO: 27, SEQ ID NO: 57, and SEQ ID NO: 24 from FIG. 27B. This subset of peptides that were highly resistant to reduction, pepsin, and elevated temperature, such as 75° C. and 100° C., are referred to as “Subtype A”. The conserved structural proline amino acid residue is circled, showing Subtype A peptides contain a proline amino acid at the third position between Cys2 and Cys3.

FIG. 27E shows a sequence analysis of Subtype A SEQ ID NO: 27, SEQ ID NO: 57, and SEQ ID NO: 24 with the conserved structural proline amino acid residue circled from FIG. 27D. This analysis renders peptide sequences that can be resistant to multiple conditions (e.g., highly resistant to reduction, pepsin, 75° C., and 100° C.) comprising GSX₁X₂X₃IX₄VX₅CX₆X₇SX₈X₉CLX₁₀PCX₆X₁₁AGMRFGX₆CX₁₂NX₁₃X₆CX₁₄CTPX₆ (SEQ ID NO: 170), wherein X₁ can be G or V; X₂ can be V, R, or K; X₃ can be P, I, or null; X₄ can be N or P; X₅ can be K, S, or R; X₆ can be K or R; X₇ can be G, I, or H; X₈ can be R, G, or K; X₉ can be Q or D; X₁₀ can be D, E, K, or R; X₁₁ can be K, D, or R; X₁₂ can be M or I; X₁₃ can be G or S; and X₁₄ can be H or D.

FIG. 27F shows a sequence alignment of SEQ ID NO: 6, SEQ ID NO: 10, and SEQ ID NO: 12 from FIG. 27B. This subset of peptides that were highly resistant to reduction, pepsin, and elevated temperature, such as 75° C. and 100° C., are referred to as “Subtype B”. The conserved structural proline amino acid residue is circled, showing Subtype B peptides contain a proline amino acid at the second position between Cys2 and Cys3.

FIG. 27G shows a sequence analysis of Subtype B SEQ ID NO: 6, SEQ ID NO: 10, and SEQ ID NO: 12 with the conserved structural proline amino acid residue circled from FIG. 27F. This analysis renders peptide sequences that can be resistant to multiple conditions (e.g., highly resistant to reduction, pepsin, 75° C., and 100° C.) comprising GSX₁X₂X₃X₄X₅X₆X₇CX₈X₉SX₁₀X₁₁CX₁₂PX₁₃CX₁₄X₁₅X₁₆FGX₁₇X₁₈X₁₉X₂₀X₂₁CX₂₂NX₂₃X₂₄CX₂₅CX₂₆X₂₇ (SEQ ID NO: 171), wherein X₁ can be Q or null; X₂ can be K, R, or null; X₃ can be I, P, or F; X₄ can be T or L; X₅ can be D or S; X₆ can be N, I, or V; X₇ can be K or R; X₈ can be N, S, or T; X₉ can be N, A, or G; X₁₀ can be S, Y, K, or R; X₁₁ can be Q or E; X₁₂ can be I, F, or W; X₁₃ can be V or H; X₁₄ can be K, I, or R; X₁₅ can be R, S, Q, or K; X₁₆ can be I, R, M, or K; X₁₇ can be K, T, or R; X₁₈ can be R, T, P, or K; X₁₉ can be N or A; X₂₀ can be G or A; X₂₁ can be K or R; X₂₂ can be I, V, or M; X₂₃ can be G, R, or K; X₂₄ can be K, L, or R; X₂₅ can be Y, D, R, or K; X₂₆ can be Y or F; and X₂₇ can be P, S, or null. Similar resistance to trypsin, pepsin, 75° C. and 100° C. is exhibited by a peptide of SEQ ID NO: 170-SEQ ID NO: 171 when the N-terminal GS is removed.

In addition, generation of stable scaffolds of peptides are made by modifying and mutating the peptides based on the conserve amino acids in the above sequences.

### Example 6

### Oral Administration of Peptides

This example describes oral and intravenous administration of peptides of this disclosure. The data shows the results of oral peptide administration, including transit of the intact peptide through the GI tract and presence in the feces. A peptide of SEQ ID NO: 27 was radiolabeled by the methods described in EXAMPLE 4, and was then administered intravenously or orally to female Harlan athymic nude mice, 6-8 weeks of age. Radiolabeled peptides of SEQ ID NO: 27 (SEQ ID NO: 27-r) was administered intravenously (IV) at a dose of 4.8 μCi/20 nmol. SEQ ID NO: 27-r was administered orally (PO) by oral gavage at a dose of 24 μCi/100 nmol. Mice were euthanized at various time points by CO₂ asphyxiation and biological fluids were collected, including blood, urine, and feces. Samples were analyzed by RP-HPLC and liquid scintillation counting to quantify the concentration or dose of radioactivity and/or intact peptide recovered in plasma, urine, and feces. Urine was collected by abdominal palpitation immediately before CO₂ asphyxiation. Blood was collected by cardiac puncture immediately after CO₂ asphyxiation and centrifuged to separate plasma. Feces were collected either before or after CO₂ asphyxiation by palpitation of the colon. Samples were analyzed by HPLC to quantify the concentration or dose of intact peptide recovered in plasma, urine, and feces. For HPLC analysis, urine samples were first diluted at a 1:20 ratio in water and plasma samples were diluted at a 1:5 ratio in water. Feces samples were dissolved in Tris buffer, centrifuged to remove the insoluble fraction, and supernatants were diluted at a 1:1 ratio in water.

TABLE 3 shows a summary of the study design.

TABLE 3

| | SEQ | | Peptide | 14C | | Mice/ |
|---|---|---|---|---|---|---|
| Group | ID NO | Route | Dose | Dose | Time Points (hr) | time |
| 1 | 27-r | IV | 20 nmol | 4.8 μCi | 0.08, 0.5, 1, 3, 8, | 3 |
| | | | | | 24, 48 | |
| 2 | 27-r | Oral | 100 nmol | 24 μCi | 0.08, 0.5, 1, 3, 8, | 3 |
| | | | | | 24, 48 | |

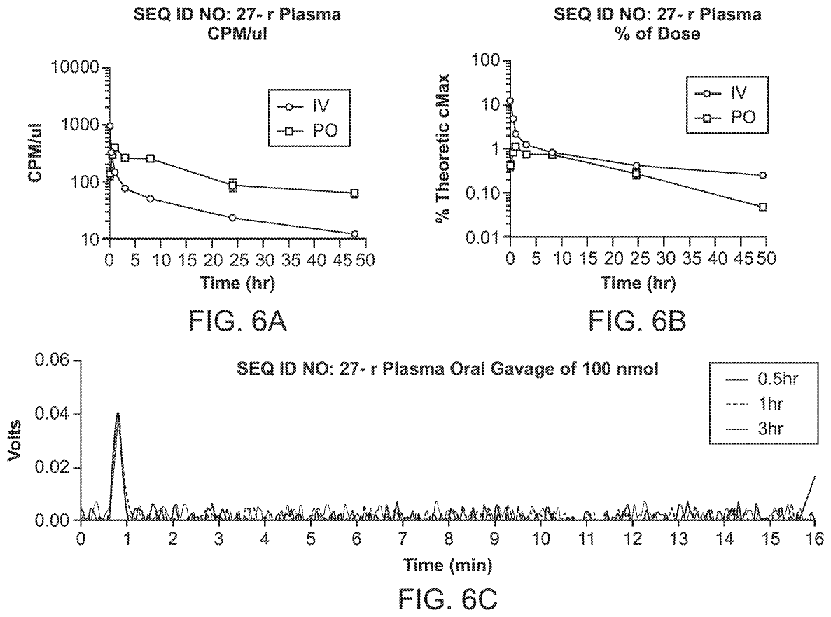

FIG. 6 shows the concentration of SEQ ID NO: 27-r in plasma after administration to a mouse. FIG. 6A shows the concentration of peptide in plasma after intravenous (IV) administration of 20 nmol of SEQ ID NO: 27-r and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point. FIG. 6B shows the percent of administered peptide dose recovered in plasma at various time points after intravenous (IV) administration of 20 nmol SEQ ID NO: 27-r and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point. FIG. 6C shows the intensity of peptide and peptide fragment peaks in plasma as measured by tandem HPLC and liquid scintillation counting after oral administration by gavage of 100 nmol of SEQ ID NO: 27-r. The delivered dose of ¹⁴C was 24 μCi for oral administration. Time points examined included 0.5, 1, and 3 hours. These data indicated that detection of radioactive signal from the dosed peptide was present up to at least 50 hours in plasma and that the plasma radioactivity was near 10% of the IV administered dose and near 1% of the PO administered dose. The intact peptide was expected to elute near 6 minutes in HPLC, whereas cleaved fragments such as the radiolabeled N-terminal Gly residue may elute near 1 minute. Thus, nearly all of the radioactively detected peptide in plasma was due to fragments of the administered peptide.

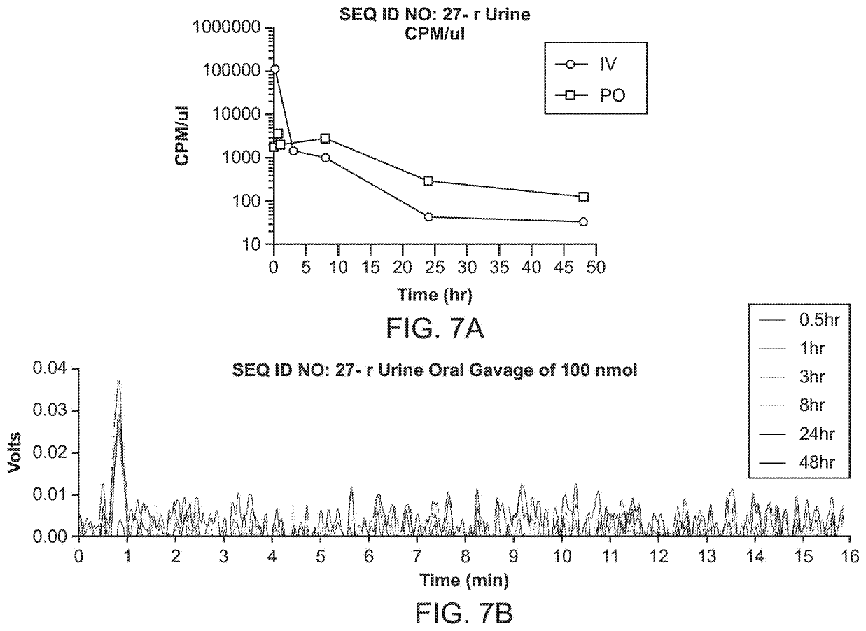

FIG. 7 shows the concentration of SEQ ID NO: 27-r in urine after administration of the peptide to a mouse. FIG. 7A shows the concentration of peptide in urine after intravenous (IV) administration of 20 nmol of SEQ ID NO: 27-r and oral (PO) administration of 100 nmol SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point. FIG. 7B shows the intensity of peptide and peptide fragment peaks in urine as measured by tandem HPLC and liquid scintillation counting after oral administration by gavage of 100 nmol of SEQ ID NO: 27-r. The delivered dose of ¹⁴C was 24 μCi for oral administration. Time points examined included 0.5, 1, 3, 8, 24, and 48 hours. These data showed that some radioactivity from the administered peptide was detected in urine, but this could be from fragments of the administered peptide.

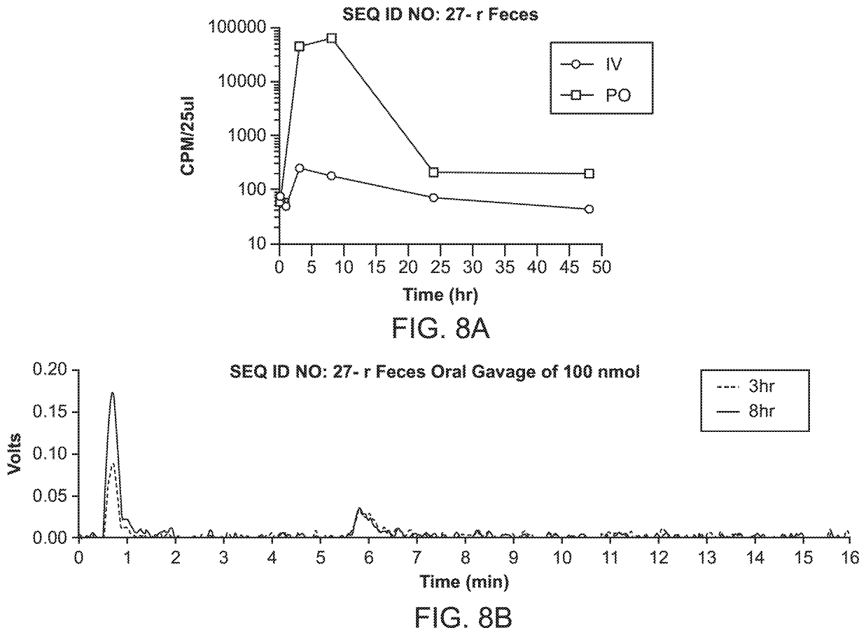

FIG. 8 shows the concentration of SEQ ID NO: 27-r in feces after administration of the peptide to a mouse. FIG. 8A shows the concentration of peptide in feces after intravenous (IV) administration of 20 nmol of SEQ ID NO: 27-r and oral (PO) administration of 100 nmol of SEQ ID NO: 27-r, as quantified by measuring the ¹⁴C signal using liquid scintillation counting. The delivered dose of ¹⁴C was 4.8 μCi for intravenous administration and 24 μCi for oral administration. Time points examined included 0.08, 0.5, 1, 3, 8, 24, 48 hours and three mice were examined per time point. FIG. 8B shows the intensity of peptide and peptide fragment peaks in feces as measured by tandem HPLC and liquid scintillation counting after oral administration by gavage of 100 nmol of SEQ ID NO: 27-r. The delivered dose of ¹⁴C was 24 μCi for oral administration. Time points examined included 3 and 8 hours. This data showed that intact SEQ ID NO: 27-r was detected in feces after oral dosing, indicating that some intact peptide transited through the GI tract.

### Example 7

### Peptide Stability in Biological Conditions

This example illustrates peptide stability in biological conditions including in the presence of reducing agents, proteases, oxidative conditions, acidic conditions, and simulated gastric fluids. This example also shows data comparing peptide stability in the cysteine knotted tertiary structure versus the linearized version of the peptide. Various peptides were suspended in 500 μl of ddH₂O at a stock concentration of 2 mg/ml. This was then diluted in accord with the reaction conditions to prevent adverse buffering effects. Reactions were prepared with 12.5 μg peptide dissolved in solution and, in some samples, additionally suspended in a 10 mM solution of DTT, simulated gastric fluid (pH 1.05; 2% (w/v) sodium chloride in 0.7% (v/v) hydrochloric acid), 500 U pepsin (5000 U/ml pepsin), 50 U trypsin (500 U/ml trypsin) with 1 mg/ml inhibitor in PBS, or a combination of any of these conditions. All protease reactions were incubated for 30 min at room temperature and then quenched. Pepsin reactions were quenched by adding Tris base to a final concentration of 0.075 M. Trypsin reactions were quenched by adding excess soybean trypsin inhibitor. RP-HPLC was then run on samples using an Agilent 1260 HPLC equipped with a C-18 Poroshell 120B column. Sample were analyzed by a gradient method with a mobile phase of Solvent A (water with 0.1% TFA) and Solvent B (acetonitrile with 0.1% TFA). Solvent B was ramped up from 5%-45% of the mobile phase over a period of 10 minutes.

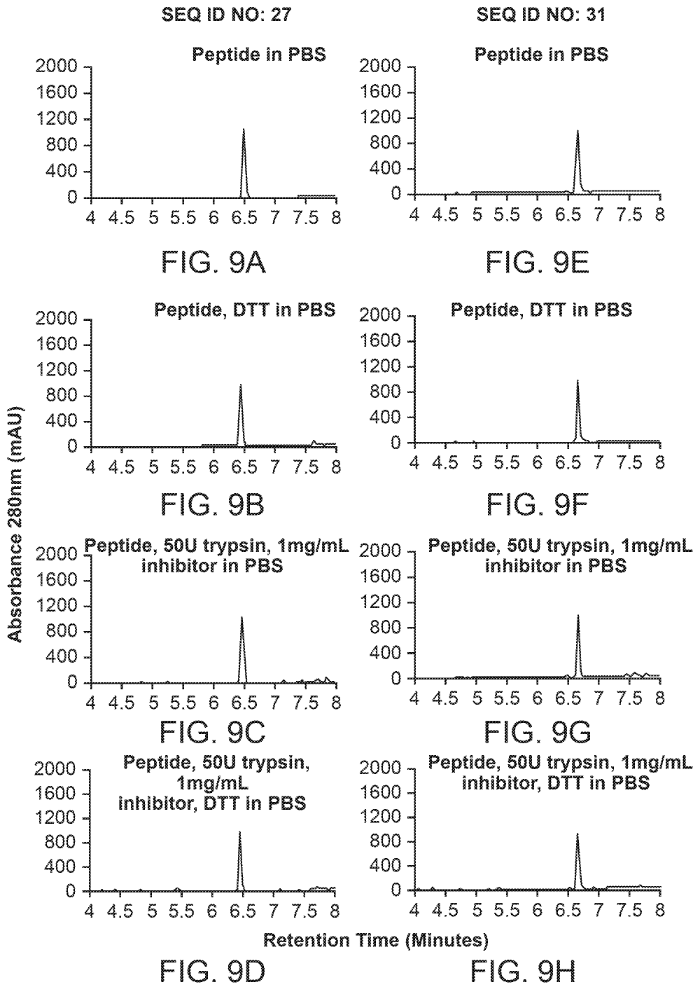

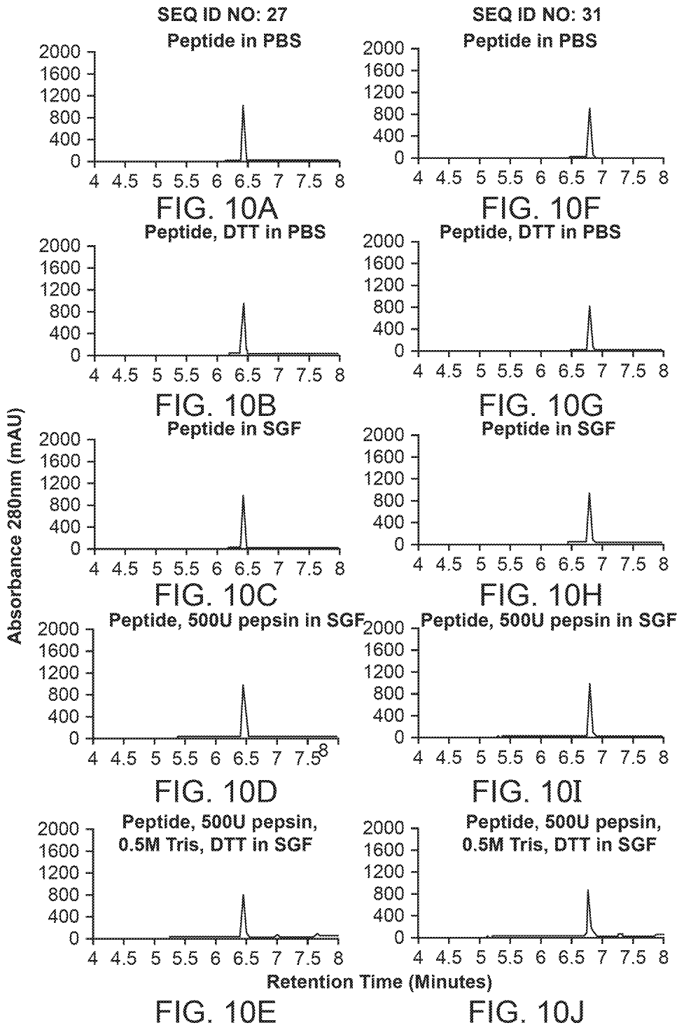

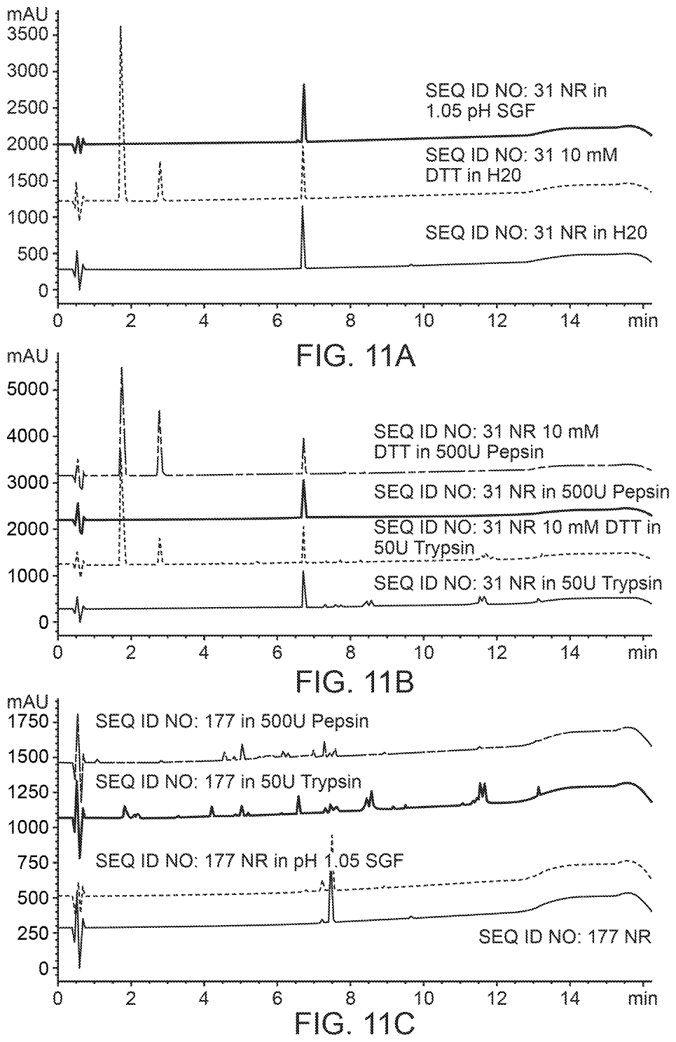

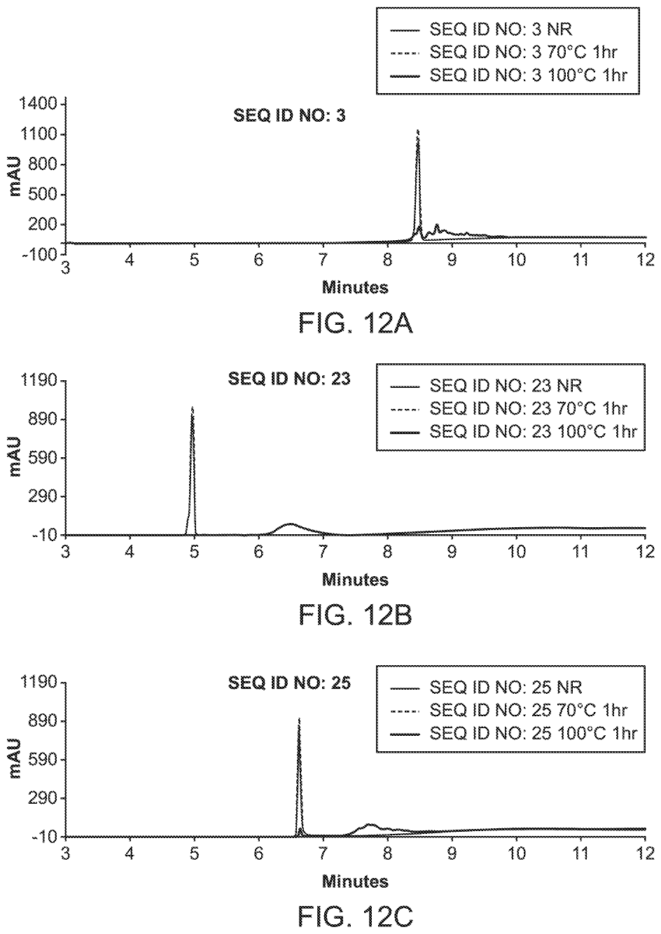

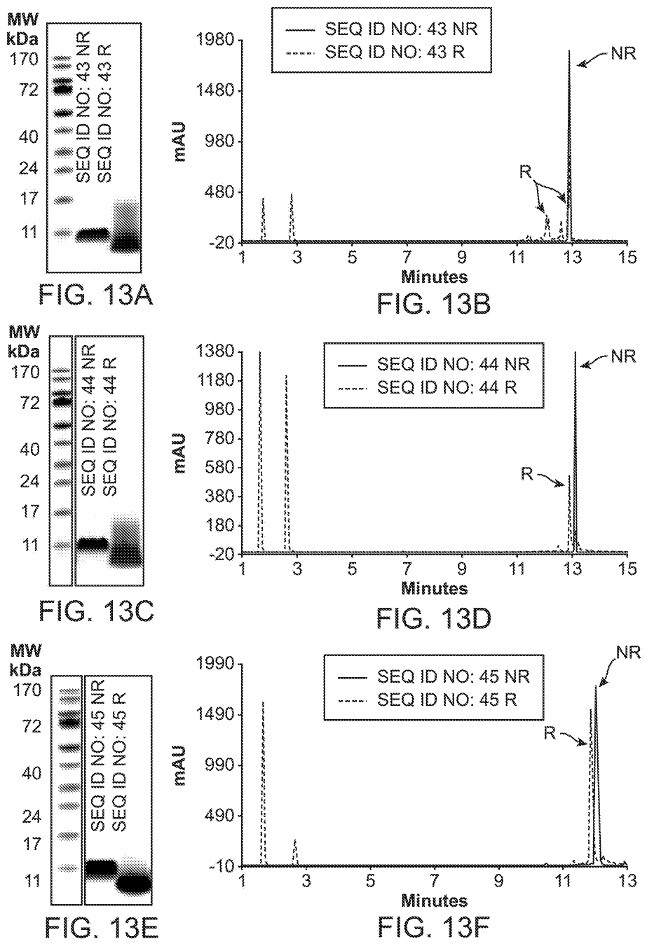

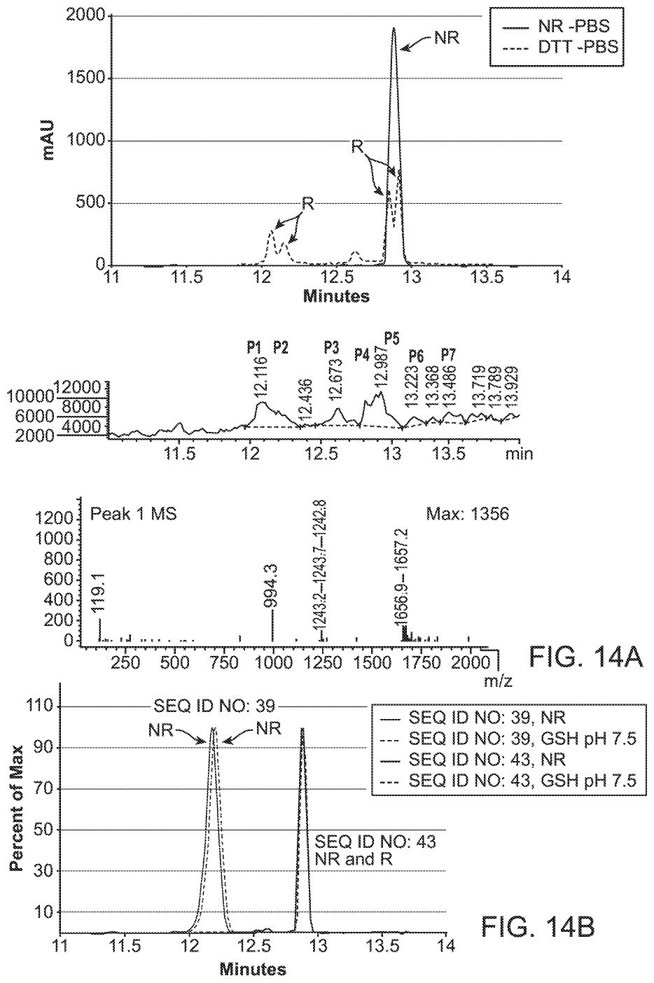

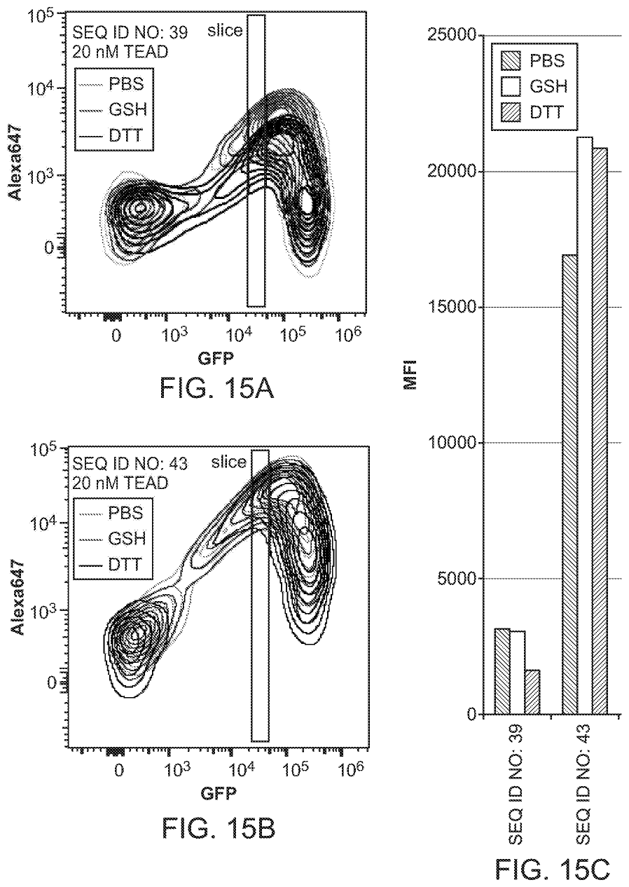

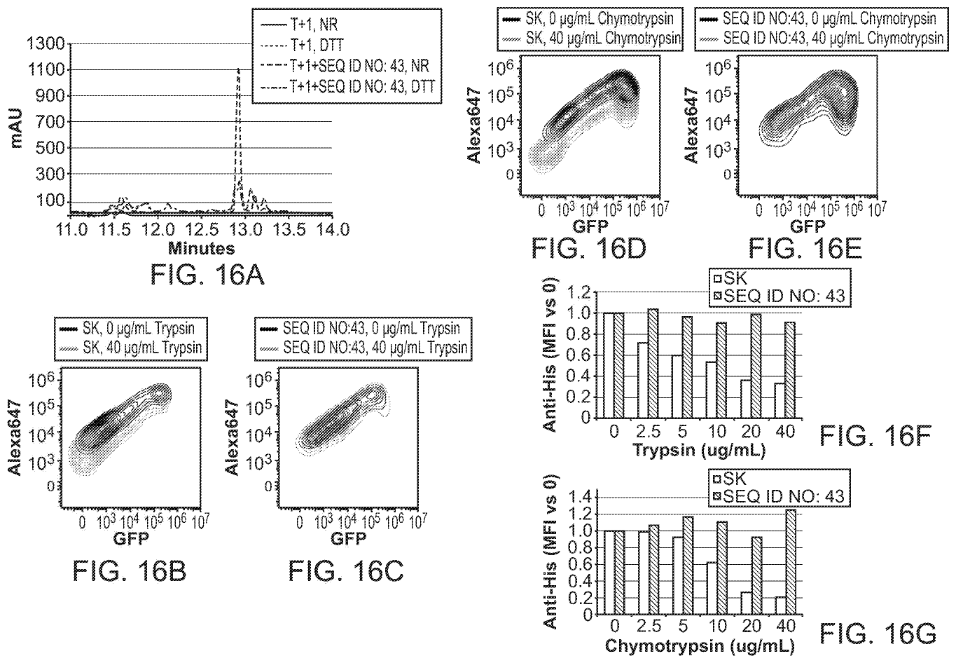

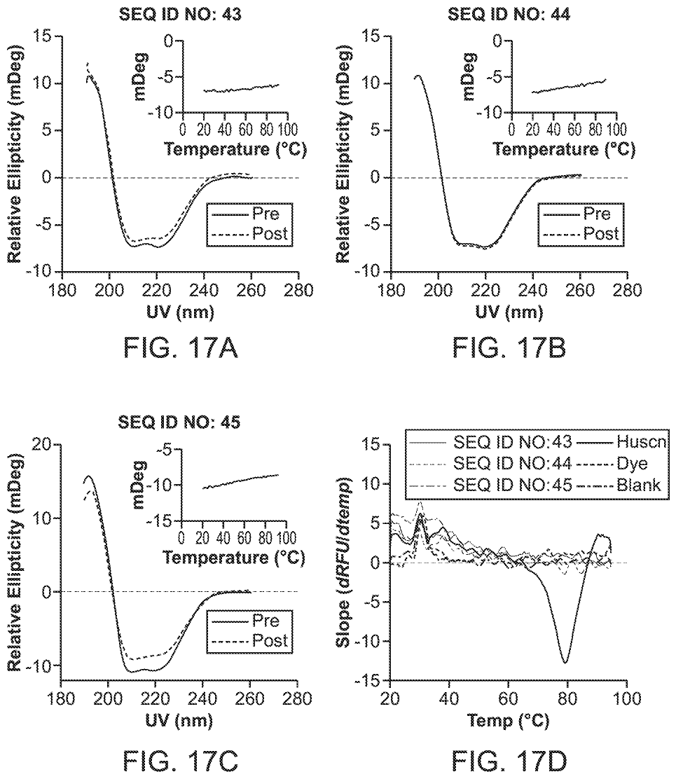

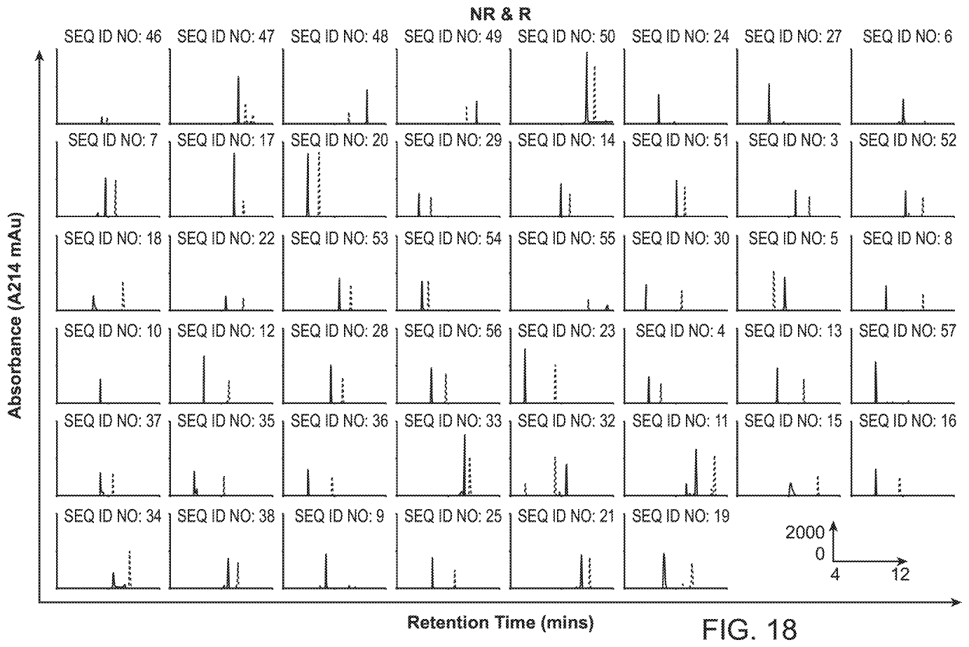

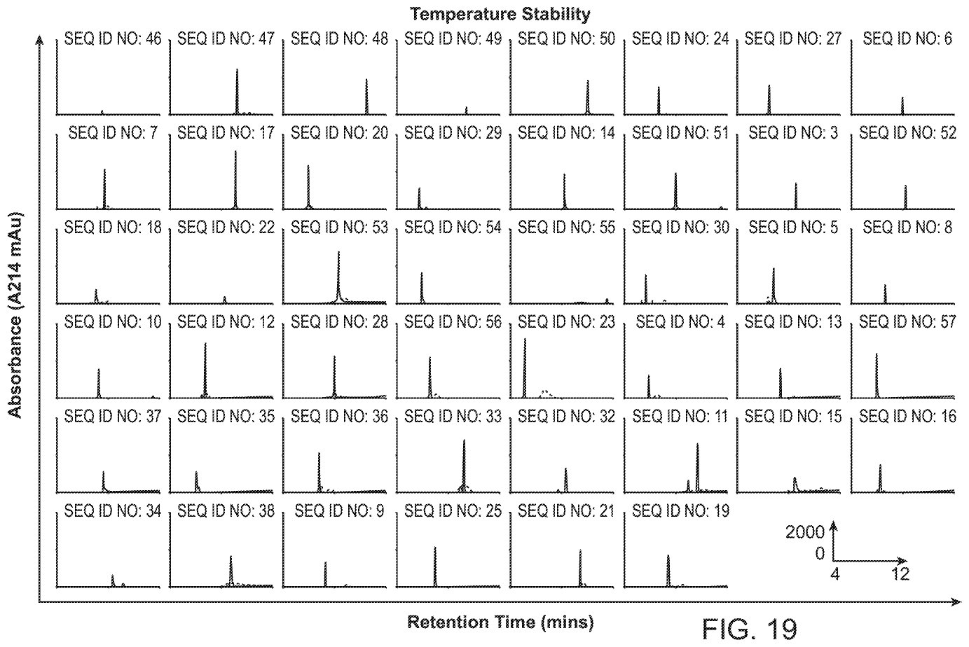

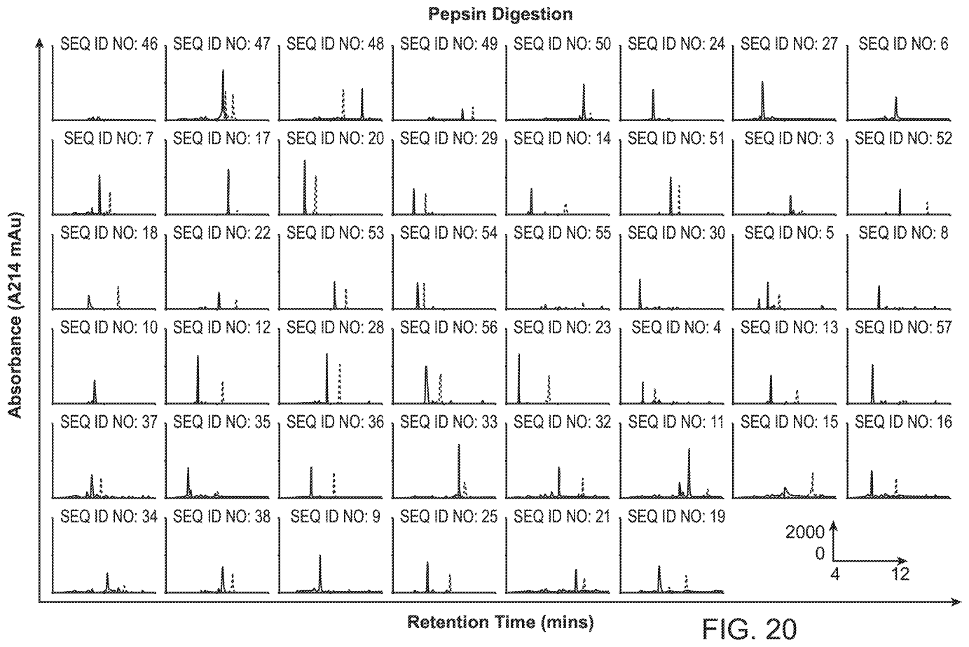

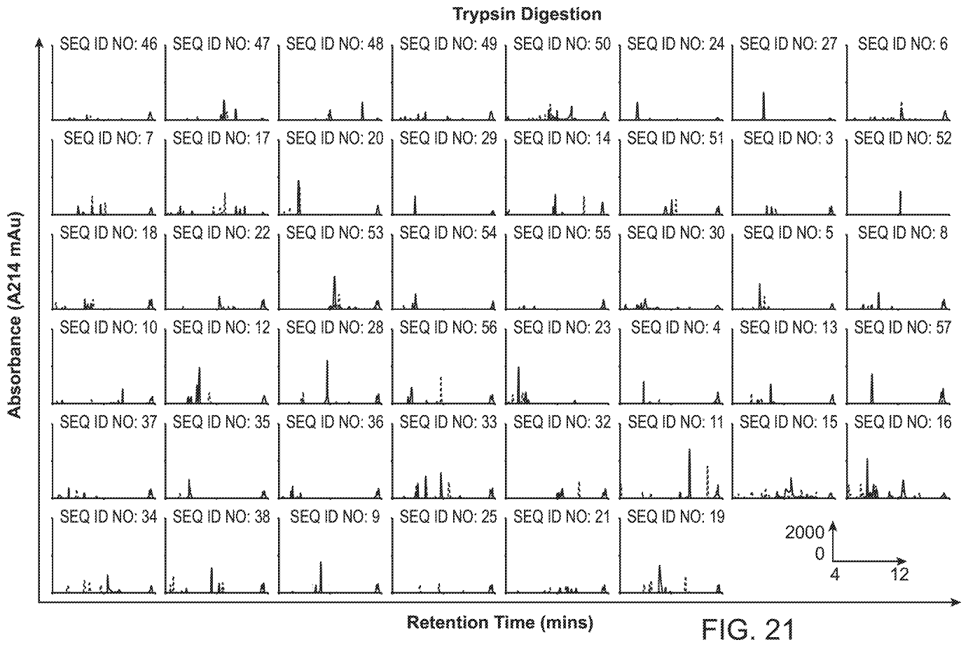

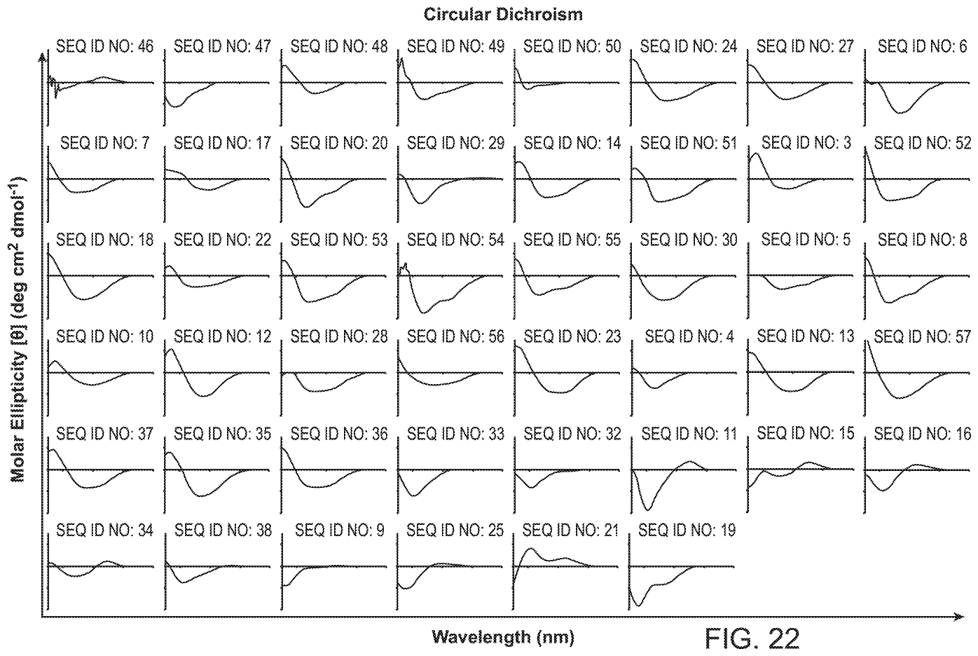

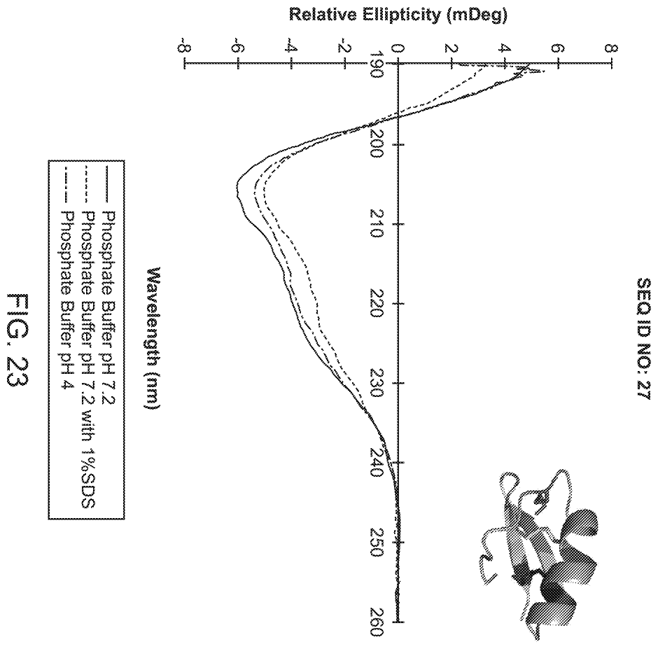

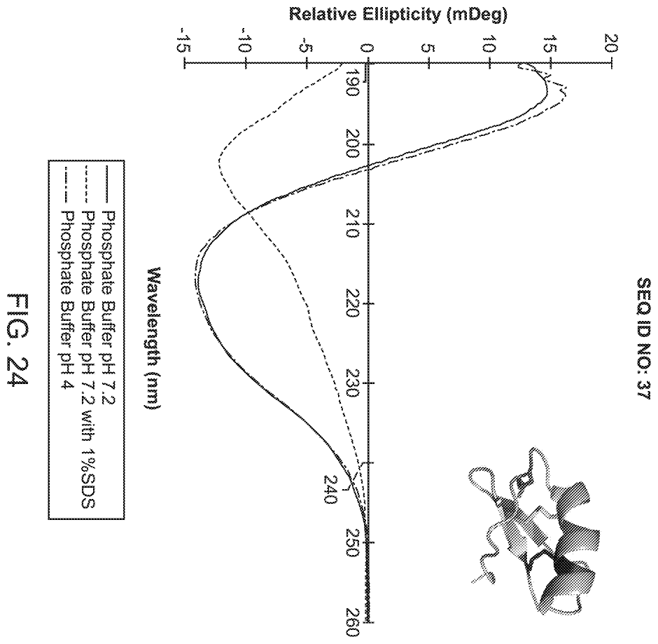

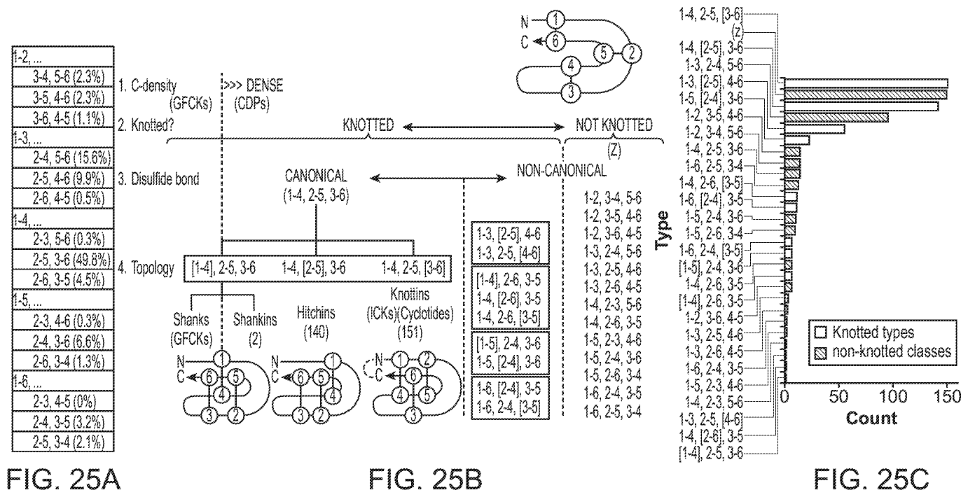

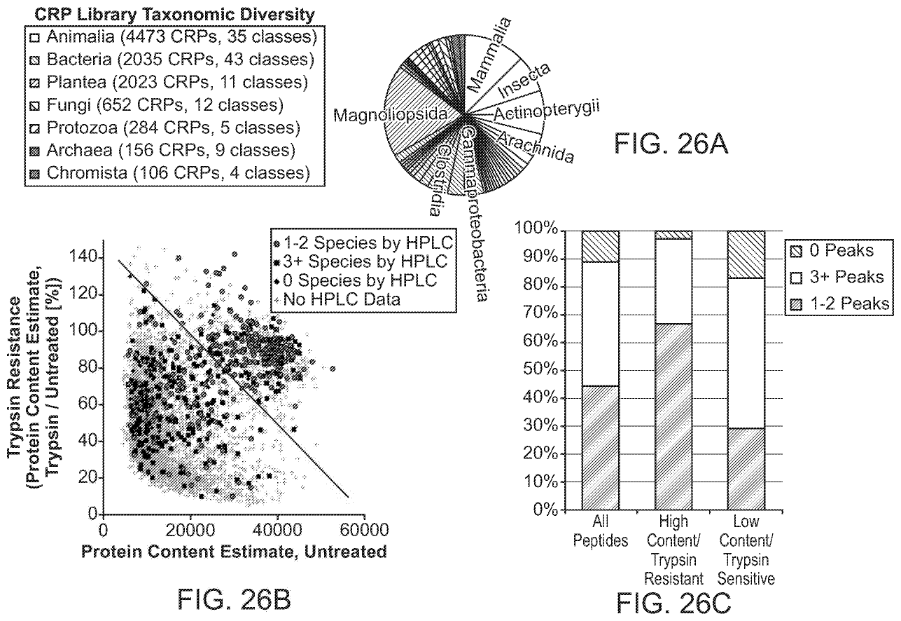

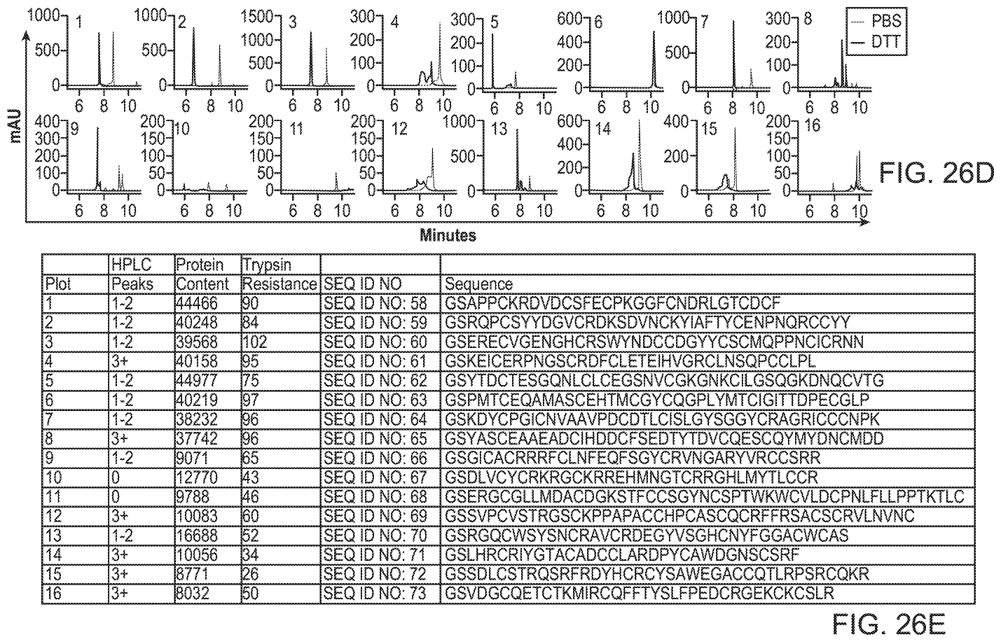

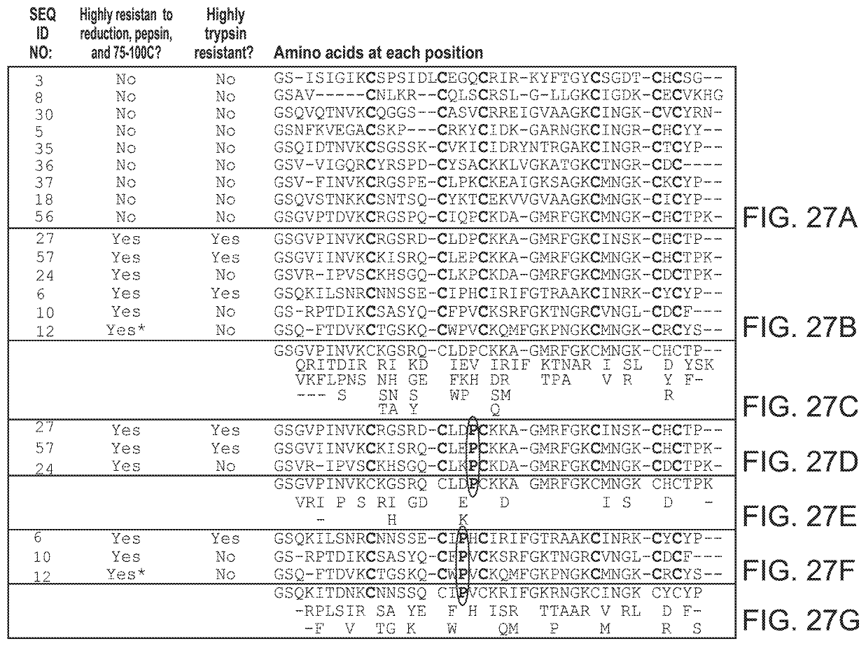

FIG. 9 illustrates HPLC chromatograms of two peptides after exposure to reducing agents and/or proteinases. FIG. 9A illustrates the HPLC trace of a peptide of SEQ ID NO: 27 in PBS. FIG. 9B illustrates the HPLC trace of a peptide of SEQ ID NO: 27 in DTT in PBS. FIG. 9C illustrates the HPLC trace of a peptide of SEQ ID NO: 27 in 50 U trypsin (500 U/ml trypsin) and 1 mg/ml inhibitor in PBS. FIG. 9D illustrates the HPLC trace of a peptide of SEQ ID NO: 27 in 50 U trypsin (500 U/ml trypsin), 1 mg/ml inhibitor, and DTT in PBS. FIG. 9E illustrates the HPLC trace of a peptide of SEQ ID NO: 31 in PBS. FIG. 9F illustrates the HPLC trace of a peptide of SEQ ID NO: 31 in DTT in PBS. FIG. 9G illustrates the HPLC trace of a peptide of SEQ ID NO: 31 in 50 U trypsin (500 U/ml trypsin) and 1 mg/ml inhibitor in PBS. FIG. 911 illustrates the HPLC trace of a peptide of SEQ ID NO: 31 in 50 U trypsin (500 U/ml trypsin), 1 mg/ml inhibitor, and DTT in PBS. The elution time of the peptide and the size of the main peak did not significantly change in FIG. 9B-D versus FIG. 9A or in FIG. 9F-H versus FIG. 9E, indicating that the peptide of SEQ ID NO: 27 and SEQ ID NO: 31 remained intact and were not degraded or reduced by treatment with trypsin or DTT.

FIG. 10 illustrates HPLC chromatograms of two peptides after exposure to reducing agents, proteinases, and/or simulated gastric fluid conditions. FIG. 10A illustrates the HPLC trace of a peptide of SEQ ID NO: 27 incubated in PBS. FIG. 10B illustrates the HPLC trace of a peptide of SEQ ID NO: 27 incubated in DTT in PBS. FIG. 10C illustrates the HPLC trace of a peptide of SEQ ID NO: 27 incubated in simulated gastric fluid (SGF). FIG. 10D illustrates the HPLC trace of a peptide of SEQ ID NO: 27 incubated in 500 U pepsin (5000 U/ml pepsin) in SGF. FIG. 10E illustrates the HPLC trace of a peptide of SEQ ID NO: 27 incubated in 500 U pepsin (5000 U/ml pepsin), 0.5 M Tris, and DTT in SGF. FIG. 10F illustrates the HPLC trace of a peptide of SEQ ID NO: 31 incubated in PBS. FIG. 10G illustrates the HPLC trace of a peptide of SEQ ID NO: 31 incubated in DTT in PBS. FIG. 1011 illustrates the HPLC trace of a peptide of SEQ ID NO: 31 incubated in simulated gastric fluid (SGF). FIG. 10I illustrates the HPLC trace of a peptide of SEQ ID NO: 31 incubated in 500 U pepsin (5000 U/ml pepsin) in SGF. FIG. 10J illustrates the HPLC trace of a peptide of SEQ ID NO: 31 incubated in 500 U pepsin (5000 U/ml pepsin), 0.5 M Tris, and DTT in SGF. The elution time of the peptide and the size of the main peak did not significantly change in FIG. 10B-E versus FIG. 10A or in FIG. 10G-J versus FIG. 10F, indicating that the peptide of SEQ ID NO: 27 and SEQ ID NO: 31 remained intact and were not degraded or reduced by incubation with DTT, SGF at pH 1, or pepsin.

FIG. 11 illustrates HPLC chromatograms of a peptide of SEQ ID NO: 31 and a negative control peptide GSGVPINVRSRGSRDSLDPSRRAGMRFGRSINSRSHSTP (SEQ ID NO: 177) after exposure to a range of conditions including oxidative, reductive, and acidic conditions as well as after exposure to proteinases. The negative control peptide of SEQ ID NO: 177 is the same sequence as a peptide of SEQ ID NO: 31, but with all cysteine residues mutated to serine residues, which eliminated the cysteine-knotted tertiary structure of a peptide of SEQ ID NO: 31 and instead to create a linearized peptide structure. FIG. 11A illustrates the HPLC trace of a peptide of SEQ ID NO: 31 under acidic (SGF pH 1), reducing (DTT), and non-reducing (NR) conditions (oxidative). The intact peptide eluted near 6.75 minutes as seen in the NR chromatogram, and DTT eluted near 1.75 minutes and 2.75 minutes. As shown in FIG. 11A, the main peak near 6.75 minutes is present after exposure to DTT or SGF and no other significant peaks were observed. This indicated that the peptide of SEQ ID NO: 31 was intact and resistant to degradation or reduction by exposure to DTT or SGF at pH 1. FIG. 11B illustrates the HPLC trace of a peptide of SEQ ID NO: 31 under various combinations of reducing agents and proteases including 10 mM DTT in 500 U pepsin (5000 U/ml pepsin), 500 U pepsin (5000 U/ml pepsin), 10 mM DTT in 50 U trypsin (500 U/ml trypsin), and 50 U trypsin (500 U/ml trypsin). The intact peptide peak near 6.75 minutes was present in all chromatograms with similar elution time and intensity, indicating that the peptide of SEQ ID NO: 31 was resistant to degradation by pepsin and trypsin, with or without DTT. FIG. 11C illustrates the HPLC trace of a peptide of SEQ ID NO: 177 under various protease conditions including in 500 U pepsin (5000 U/ml pepsin), in 50 U trypsin (500 U/ml trypsin), non-reducing (NR) in simulated gastric fluid (SGF) at pH 1.05, and NR. The peptide of SEQ ID NO: 177 had a similar main peptide peak near 7.5 minutes in the NR and SGF samples, indicating that it was stable when incubated with SGF. However, after incubation with trypsin or pepsin, the peptide was degraded by the enzymes, as shown by the low level of the main peak near 7.5 minutes and the appearance of many new peaks in the chromatogram. The negative control peptide of SEQ ID NO: 177 was not resistant to degradation by trypsin and pepsin, whereas the peptide of SEQ ID NO: 31 was highly stable against degradation by these enzymes. The presence of the cysteine residues and preservation of the knotted tertiary structure in the peptide of SEQ ID NO: 31 was important in imparting resistance to enzyme degradation for this particular sequence.

### Example 8

### Screening Peptides for Serine Protease Resistance

This example describes screening of any one of the peptides of this disclosure (SEQ ID NO: 1-SEQ ID NO: 166) for degradation resistance. A peptide of this disclosure is recombinantly expressed or chemically synthesized. A peptide of this disclosure is suspended in water. Reactions are prepared with serine protease. Reactions are incubated for 30 minutes at 37.5° C. Reactions are quenched with a serine protease inhibitor. Samples are analyzed by reversed phase HPLC or by other methods, such as circular dichroism, which detects changes in the secondary structure of the peptide.

Peptides of the disclosure are analyzed for degradation to serine protease. Analysis of HPLC chromatograms and peptide peaks is performed to identify serine protease resistant peptides. Serine protease resistant peptides are shown to have less change in their HPLC chromatogram after exposure to a serine protease.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 9

### Oral Delivery of Peptides

This example describes oral delivery of any one of the peptides of this disclosure (SEQ ID NO: 1-SEQ ID NO: 166). A peptide of this disclosure is recombinantly expressed or chemically synthesized. A peptide of this disclosure is formulated and orally administered to a subject. The peptide can be formulated in a pharmaceutical composition. The subject can be an animal or a human. The peptide is delivered by oral gavage, in solid dispersions, genetically encoded in a probiotic, as a sublingual formulation, as a lollipop or lozenge, in liquids, in suspension, in emulsions, in capsule, tablets, or powders, or in semi-solid dispersions. The peptide can be formulated with other agents, which improve stability and/or permeation, such as buffers, protease inhibitors, and permeation enhancers. The peptide can be formulated to improve uptake across the gut wall into the bloodstream, to avoid uptake across the gut wall into the bloodstream, or to allow for longer residency within the gut. Enhanced stability and resistance to denaturation, reduction, or cleavage by enzymes is exhibited by the peptide after oral administration. Consequently, the peptide is stable long enough to act on the target to exhibit a therapeutic effect, rather than being degraded quickly and thus having no effect. A therapeutic effect is exhibited by the peptide.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 10

### Determining Biodistribution and Bioavailability of Orally Administered Peptides

This example describes determining biodistribution and bioavailability after oral administration of any one of the peptides of this disclosure (SEQ ID NO: 1-SEQ ID NO: 166). A peptide of this disclosure is recombinantly expressed or chemically synthesized. The peptide can be labeled with a fluorescent label or a radiolabel and administered orally to a subject. The subject can be an animal or a human.

Biological samples are obtained at various time points from blood, urine, feces, brain, cartilage, joints, cancerous tissues, infected tissue or abscesses, bone marrow, liver, muscle, kidney, placenta or fetal tissues. Samples are analyzed for detection of intact peptide or peptide fragments and signal associated with intact peptide or peptide fragments over time in various biological fluids or samples is quantified.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 11

### Treatment of a Gastrointestinal (GI) Disorder

This example describes treatment of a gastrointestinal (GI) disorder with a peptide or peptide conjugate of this disclosure (SEQ ID NO: 1-SEQ ID NO: 166). A peptide of this disclosure is recombinantly expressed or chemically synthesized. The peptide itself can be administered as the therapeutic or it can be conjugated to an active agent, such as an antibiotic (e.g., carbapenems, penicillins, quinolines, fluorquinolines, etc.), a chemotherapeutic, an anti-apoptotic agent (e.g., a BCL2 inhibitor), a senolytic, or an anti-inflammatory agent (e.g., a steroid). The peptide or peptide conjugate of this disclosure is formulated and orally administered to a subject. The peptide can be formulated in a pharmaceutical composition. The subject can be an animal or a human.

An efficacious amount of the intact peptide or peptide conjugate is administered, which reaches the gut, to treat colorectal cancer, inflammatory bowel disease, constipation, Crohn's disease, lupus, or irritable bowel syndrome.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the gastrointestinal disease is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 12

### Peptides as Delivery Scaffolds

This example describes the use of peptides of this disclosure (SEQ ID NO: 1-SEQ ID NO: 166) as a delivery scaffold. Peptides of this disclosure are recombinantly expressed or chemically synthesized and are fused to an active agent by genetic fusion or by chemical conjugation. Peptide conjugates are administered to a subject in need thereof. The subject is a human or a non-human animal. The peptide conjugates can be formulated in a pharmaceutical composition

Peptides delivery scaffolds are used to deliver the active agent to a tissue or region of the body, such as the gastrointestinal tract, skin, cartilage, vaginal mucosa, or nasal mucosa, or a cellular compartment, such as lysosomes, endosomes, or the cytosol. Enhanced stability and resistance to denaturation, reduction, or cleavage by enzymes is exhibited by the peptide delivery scaffold-active agent conjugate after oral administration. Consequently, the peptide delivery scaffold-active agent conjugate is stable long enough to deliver the active agent or accumulate in one of the tissues, regions of the body, or cellular compartments described above to act on the target in order to exhibit a therapeutic effect, rather than being degraded quickly and thus having no effect. A therapeutic effect is exhibited by the peptide delivery scaffold-active agent conjugate and the targeted disease is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 13

### Temperature Stable Peptides

This example illustrates peptide stability at high temperatures. Peptides were first suspended in 500 μl of ddH₂O to a stock concentration of 2 mg/ml. Reactions were prepared by adding 6.25 μl of peptide from the stock solution with 95 μl ddH₂O and incubated at room temperature, 70° C., or 100° C. for one hour in a Thermocycler. RP-HPLC was then run on samples using an Agilent 1260 HPLC equipped with a C-18 Poroshell 120B column. Sample were analyzed by a gradient method with a mobile phase of Solvent A (water with 0.1% TFA) and Solvent B (acetonitrile with 0.1% TFA). Solvent B was ramped up from 5%-45% of the mobile phase over a period of 10 minutes.

FIG. 12A illustrates the HPLC trace of a peptide of SEQ ID NO: 3 in NR conditions after incubation at room temperature, 70° C., or 100° C. for one hour. FIG. 12B illustrates the HPLC trace of a peptide of SEQ ID NO: 23 in NR conditions after incubation at room temperature, 70° C., or 100° C. for one hour. FIG. 12C illustrates the HPLC trace of a peptide of SEQ ID NO: 25 in NR conditions after incubation at room temperature, 70° C., or 100° C. for one hour.

After incubation at 70° C. for 1 hour, peptides of SEQ ID NO: 3, SEQ ID NO: 23, and SEQ ID NO: 25 showed approximately the same HPLC elution time and peak height as the untreated (NR, nonreduced) samples, indicating the peptides were resistant to heat-induced degradation. After incubation at 100° C. for 1 hour, peptides of SEQ ID NO: 3, SEQ ID NO: 23, and SEQ ID NO: 25 underwent various degrees of degradation as evidenced by the reduced amount of peptide eluting at the original elution time.

FIG. 19 illustrates high performance liquid chromatograph (HPLC) traces of various peptides of the present disclosure after incubation at 75° C. for 1 hour (solid trace) or 100° C. for 1 hour (dashed trace). As seen in the figure, of the 46 peptides tested, 17 were resistant to degradation at 100° C., such as peptides of SEQ ID NO: 17 and SEQ ID NO: 45.

### Example 14

### Peptide Stability in Reducing Conditions

This example describes stability of designed or engineered peptides under reducing conditions. Peptides that are designed or engineered to interact with one or more target proteins in the cell, such as proteins in the nucleus, are exposed to reducing conditions in the cytosolic compartment of cells. Thus, it is advantageous for peptides of this disclosure to display stability in reducing conditions. Stability of peptides of this disclosure was tested in 10 mM DTT and 10 mM GSH. GSH is a more physiologically relevant reducing agent for testing peptide stability in intracellular conditions. An example of a peptide-target protein interaction is a peptide binding to a TEAD protein.

As shown in FIG. 14B, when peptides of SEQ ID NO: 39 and SEQ ID NO: 43 were exposed to reducing conditions under GSH, a reducing agent that is more representative of the reducing environment in the cellular cytosolic compartment, both SEQ ID NO: 39 and SEQ ID NO: 43 were resistant to GSH reducing conditions, as neither peptide displayed peaks that were significantly shifted as observed by HPLC in comparison to non-reduced peptides. In a stronger reducing condition, such as in 10 mM DTT, as shown in FIG. 14A, a peptide of SEQ ID NO: 43 was partially resistant to DTT reduction. As shown in the inset of FIG. 14A, in line mass spectrometry of a peptide of SEQ ID NO: 43 in DTT reducing conditions revealed fragments within ˜6 Da of the non-reduced peptide, thus demonstrating that a peptide of SEQ ID NO: 44 is partially resistant to reduction in DTT. As shown in FIG. 13B, FIG. 13D, and FIG. 13F, peptides of SEQ ID NO: 43, SEQ ID NO: 44, and SEQ ID NO: 45 were also partially resistant to DTT reducing conditions, respectively.

Designed or engineered peptides of this disclosure were also tested to evaluate if the peptides' binding activity to their target protein (e.g., TEAD protein) was affected by exposure to reducing conditions. HEK-293T suspension cells were transfected with a surface display GFP FasL(SDGF) vector comprising SEQ ID NO: 39 (SDGF-SEQ ID NO: 39) or SDGF vector comprising SEQ ID NO: 43 (SDGF-SEQ ID NO: 43) construct. Cultures were grown for two days, followed by incubation in 10 mM GSH or 10 mM DTT. Finally, cells were stained with biotinylated-target protein to evaluate the binding activity of a peptide to the target protein. FIG. 15 illustrates the stability of peptides of SEQ ID NO: 39 and SEQ ID NO: 43 after exposure to a reducing agent. Each peptide was expressed on a cell surface and tested for binding to a target protein after cells expressing SDGF-SEQ ID NO: 43 or SEQ ID NO: 39 were exposed to a reducing agent. FIG. 15A illustrates a flow cytometry plot showing binding of HEK-293 suspension cells transfected with SDGF-SEQ ID NO: 39 (GFP) incubated for 5 minutes in PBS, 10 mM DTT, or 10 mM reduced glutathione (GSH) before staining with 20 nM biotinylated target protein and 20 nM AF647-streptavidin. FIG. 15B illustrates a flow cytometry plot showing binding of HEK-293 suspension cells transfected with SDGF-SEQ ID NO: 43 (GFP) incubated for 5 minutes in PBS, 10 mM DTT, or 10 mM reduced glutathione (GSH) before staining with 20 nM biotinylated target protein and 20 nM AF647-streptavidin. FIG. 15C illustrates quantification of the AF647 mean fluorescence intensity (MFI) of cells falling within the “slice” gate shown in FIG. 15A and FIG. 15B. The results of the binding assays indicated that cells displaying a peptide of SEQ ID NO: 39 showed partial loss in binding to target protein after DTT treatment and no loss in binding to target protein after GSH treatment. The results of the binding assays also indicated that cells displaying a peptide of SEQ ID NO: 43 showed no loss in binding to target protein regardless of whether DTT or GSH was used as the reducing agent. In other words, peptides of SEQ ID NO: 39 and SEQ ID NO: 43 were resistant to reduction by GSH.

FIG. 18 illustrates high performance liquid chromatograph (HPLC) traces of peptides in non-reducing (NR) conditions (solid trace) or in 10 mM DTT reducing (R) conditions (dashed trace). Lyophilized peptides of various sequences of the present disclosure were suspended at a stock concentration of 0.5 mM in phosphate buffered saline (PBS) and diluted to final concentration of 0.015 mM in PBS, with or without 10 mM DTT. Samples were allowed to incubate at room temperature for 30 min prior to analysis by HPLC-reverse phase chromatograph (RPC). As seen in the figure, of the 46 peptides tested, 5 were resistant to reduction, such as peptides of SEQ ID NO: 5 and SEQ ID NO: 45.

### Example 15

### Peptide Stability to Protease

This example illustrates stability of peptides of this disclosure to proteases. Tumor environments generally contain a high amount of proteases. Furthermore, resistance to proteolysis (degradation or cleaving by proteases) reduces the likelihood that a peptide will be degraded and then displayed to the immune system by MHC. In addition, resistance to proteolysis can increase peptide half-life in serum after administration prior to trafficking to a tumor. Thus, it is advantageous for peptides of the present disclosure to be resistant to degradation by proteases.

Soluble peptides were exposed to 500 U/mL porcine trypsin and analyzed by HPLC. Protease resistance was also assessed by using SDPR-displaying peptides. The SDPR vector is similar to the a surface display GFP FasL(SDGF) vector, but contains a C-terminal 6×His tag (SEQ ID NO: 180) and all basic or aromatic amino acid residues on the stalk removed. Protease resistance is then tested by incubation of cells displaying peptides with trypsin or chymotrypsin, followed by incubation in 10 mM DTT, and staining with an anti-6×His fluorophore-labeled antibody (“6×His” disclosed as SEQ ID NO: 180). If peptides are uncleaved, they retain the His tag and are stained with by the antibody. A control protease-sensitive knottin peptide (SK) was used as a positive control. Cells displaying peptides were treated with up to 40 μg/ml trypsin or chymotrypsin.

FIG. 16 illustrates protease resistance of a peptide of SEQ ID NO: 43. FIG. 16A illustrates HPLC chromatograms of a peptide of SEQ ID NO: 43 after incubation with 500U trypsin (T), which was then quenched with trypsin inhibitor (I) and placed in non-reducing (NR) conditions or reducing (R) conditions with 10 mM DTT. The products were then run on HPLC. The chromatograms appear similar (though not identical) to those seen in experiments lacking trypsin.

To determine the impact of protease treatment or reducing condition on the function of the peptides, i.e., binding activity, various binding or functional assays can be performed. An example of a functional binding assay is a cell surface display method, wherein cells were engineered to express a SDPR vector comprising a sequence that encoded a peptide attached to a transmembrane domain using a stalk or linker sequence at the N-terminus of the peptide. Cells that expressed the peptide construct displayed the peptide on the cell surface and were positive for GFP fluorescence. The peptide presented on the cell surface was tested for interaction with a target protein. An example of a target protein tested was a TEAD protein. All the basic or aromatic residues within the linker or stalk sequence between the peptide and the transmembrane domain were removed to prevent trypsin/chymotrypsin cleavage in sequences outside the peptide itself. A 6×His tag (SEQ ID NO: 180) was also added to the C-terminus of the peptide construct for assessing protease and/or reduction resistance of the peptide, which can be accomplished by staining for anti-6×His (SEQ ID NO: 180). In various embodiments, in experiments using this construct, cells expressing the constructs can be incubated with a protease, such as trypsin or chymotrypsin, followed by treatment with a reducing agent. After treatment with the protease and reducing agent, any linearized peptide product or degradation product resulting from reduction and/or proteolysis by treatment with a protease can be detected or analyzed by staining against the 6×His tag (SEQ ID NO: 180). For example, a peptide can become linearized in the presence of a reducing agent, which can make it more susceptible to proteolysis, or a reduction-resistant peptide can be cleaved by a protease. The presence of a single cleavage event in the peptide backbone can result in a linearized, displayed peptide, which can lose the 6×His tag (SEQ ID NO: 180) on the C-terminus, causing cells with such peptides to lose staining against 6×His (SEQ ID NO: 180).

FIG. 16B, FIG. 16C, FIG. 16D, and FIG. 16E illustrate the flow cytometry analyses of HEK-293 suspension cells transfected with either a protease sensitive knottin peptide SK cloned into the SDPR construct (SDPR-SK) or SEQ ID NO: 43 cloned into the SDPR construct, and treated with 0 or 40 μg/ml trypsin or chymotrypsin for 20 minutes, and then stained with an AF647 anti-6×HIS antibody (“6×HIS” disclosed as SEQ ID NO: 180). Loss of anti-6×HIS signal (“6×HIS” disclosed as SEQ ID NO: 180) is indicative of peptide destabilization or degradation. FIG. 16B and FIG. 16D illustrate a flow cytometry plot of HEK-293 suspension cells transfected with protease sensitive SDPR-SK peptide and then treated with 0 or 40 μg/ml trypsin or chymotrypsin for 20 minutes, and stained with an AF647 anti-6×HIS antibody (“6×HIS” disclosed as SEQ ID NO: 180). Loss of staining after treatment with trypsin or chymotrypsin was visualized as a reduction in APC signal when cells were stained with Alexa647-conjugated anti-6×His antibody (“6×His” disclosed as SEQ ID NO: 180). FIG. 16C illustrates a flow cytometry plot of HEK-293 suspension cells transfected with SDPR-SEQ ID NO: 43 peptide and then treated with 0 or 40 μg/ml trypsin for 20 minutes, and stained with an AF647 anti-6×HIS antibody (“6×HIS” disclosed as SEQ ID NO: 180). FIG. 16E illustrates a flow cytometry plot of HEK-293 suspension cells transfected with SDPR-SEQ ID NO: 43 peptide and then treated with 0 or 40 μg/ml chymotrypsin for 20 minutes, and stained with an AF647 anti-6×HIS antibody (“6×HIS” disclosed as SEQ ID NO: 180). FIG. 16F illustrates quantification of flow cytometry data comparing SDPR-SK peptide transfected cells and SDPR-SEQ ID NO: 43 peptide transfected cells, both treated with trypsin at various concentrations. FIG. 16G illustrates quantification of flow cytometry data comparing SDPR-SK peptide transfected cells and SDPR-SEQ ID NO: 43 peptide transfected cells, both treated with chymotrypsin at various concentrations. The results of exposure to protease indicated that a peptide of SEQ ID NO: 43 was partially resistant to cleavage by proteases, such as trypsin and chymotrypsin.

FIG. 20 illustrates high performance liquid chromatograph (HPLC) traces of peptides after pepsin digestion. The solid trace shows a reaction of peptide and pepsin that was quenched at alkaline pH and run under non-reducing conditions. The dashed trace shows a reaction of peptide and pepsin that was quenched at alkaline pH and run under reducing conditions. The assay was carried out by reconstituting 5 mg of porcine pepsin (Sigma Aldrich P7012) in 1 mL of cold H₂O. 4 μl of this stock solution was added to each reaction, such that each reaction condition contained 50 U of pepsin. Because pepsin can require a low pH environment for enzyme activity, a stock solution of Simulated Gastric Fluid (SGF) (pH 1.05; 2% (w/v) sodium chloride in 0.7% (v/v) hydrochloric acid) was made. Two reaction conditions were prepared: one with peptide that was reduced with 10 mM DTT after digestion (reducing conditions) and one with peptide that was non-reduced after digestion (non-reducing conditions). In a 96-well Corning V plate, 3 μl of stock concentration of 0.5 mM peptide was added to 78 μl of SGF, 4 μl of fresh 5 mg/mL pepsin was then added to each peptide reaction, and then each sample was thoroughly mixed. The samples were incubated for 30 minutes at 37.5° C. in a shaker. After digestion, 15 μl of 0.5 M Tris base (pH 10.6) was added to each sample to inhibit digestion. 10 mM DTT was either added to samples as the reducing condition or not added as the non-reducing condition. HPLC analysis was performed on an Aglient 1260 Infinity system. Of the original 100 μl sample, 90 μl was loaded onto a Poroshell 120 B Column with a 5% to 45% ACN gradient in 10 mins. The total HPLC run time was 16.25 minutes. Proteolysis was determined by comparing the experimental reduced trace (dashed) in FIG. 20 to the control reduced trace in FIG. 18 (dashed). As seen in the figure, of the 46 peptides tested, 29 were resistant to pepsin digestion, such as peptides of SEQ ID NO: 29 and SEQ ID NO: 45.

FIG. 21 illustrates high performance liquid chromatograph (HPLC) traces of peptides after trypsin digestion. The solid trace shows a reaction of peptide and trypsin that was quenched at neutral pH with excess trypsin inhibitor and run under non-reducing conditions. The dashed trace shows a reaction of peptide and trypsin that was quenched at neutral pH with excess trypsin inhibitor and run under reducing conditions. The assay was carried out by reconstituting 1 mg porcine trypsin (Sigma Aldrich 6567) in 1 mL of 1 mM HCl. Trypsin was provided at a concentration of 10,000 u/mL and thus, 5 μl of stock solution was equivalent to using 50 U of trypsin in each reaction. Soybean trypsin inhibitor (SA T9128) was prepared at 1 mg/mL of in H₂O. Two peptide reactions were prepared: one with peptide that was reduced with 10 mM DTT after digestion (reducing conditions) and one with peptide that was non-reduced after digestion (non-reducing conditions). The reactions contained 3 μl of stock concentration of 0.5 mM peptide in 87 μl of 1×PBS in a 96-well Corning V plate. 5 μl of 1 mg/mL trypsin was added and the samples were incubated for 30 minutes at 37.5° C. in a shaker. After digestion, 5 μl of 1 mg/mL inhibitor was added to each reaction to inhibit trypsin digestion. 10 mM DTT was either added to samples as the reducing condition or not added as the non-reducing condition. HPLC analysis was done on an Aglient 1260 Infinity system. Of the original 100 μl sample, 90 μl was loaded onto a Poroshell 120 B Column with a 5% to 45% ACN gradient in 10 mins. The total run time was 16.25 minutes. Proteolysis was determined by comparison of the experimental reduced trace (dashed) in FIG. 21 to the control reduced trace in FIG. 18 (dashed). As seen in the figure, of the 46 peptides tested, only peptides of SEQ ID NO: 27 and SEQ ID NO: 57 were fully resistant to trypsin digestion.

### Example 16

### Determination of Peptide Stability at High Temperatures Using Circular Dichroism

This example shows peptides of this disclosure are stable in extreme heat. Protein secondary structures were assessed using Circular Dichrosim (CD). CD spectra were measured with a Jasco J-720W spectropolarimeter using a 1.0 mm path length cell. Protein samples in 10 mM phosphate buffer (pH=7.4) were at 25-30 μM protein concentration. Samples were analyzed at wavelength ranges of 260-190 nM. Data were expressed in terms of relative ellipticity [θ]; (mdeg). To determine thermal stability of proteins, samples were subjected to incremental increase in temperature at a ramp of 2° C./min from 20° C. to 95° C. Stability and protein unfolding were monitored at 220 and 215 for α-helix and β-sheet secondary structures, respectively. Data are expressed in terms of relative ellipticity [θ], reported in mdeg. For SEQ ID NO: 43 and its point mutant variants, no changes to the α-helix-dominated structure were observed upon heating up to 95° C. FIG. 17A illustrates that the CD spectra of SEQ ID NO: 43 demonstrated that the structure was dominated by α-helical elements, and that this secondary structure signature was identical before (Pre) and after (Post) incubation at 95° C. The inset shows relative ellipticity at 220 nm during heating from 20° C. to 95° C. FIG. 17B illustrates that the circular dichroism spectra of SEQ ID NO: 44 demonstrated the structure is dominated by α-helical elements, and that this secondary structure signature was identical before (Pre) and after (Post) incubation at 95° C. This inset also shows relative ellipticity at 220 nm during heating from 20° C. to 95° C. FIG. 17C illustrates that the circular dichroism spectra of SEQ ID NO: 45 demonstrated the structure was dominated by α-helical elements, and that this secondary structure signature was similar before (Pre) and after (Post) incubation at 95° C. Again, the inset shows relative ellipticity at 220 nm during heating from 20° C. to 95° C. Additionally, protein melting temperature (Tm) determination was performed by monitoring protein unfolding using SYPRO Orange dye (Molecular Probes). In brief, 0.1 mg/mL protein sample in 20 μL total volume PBS buffer were mixed with 24, of 10× SYPRO Orange dye. Dye intercalation into the hydrophobic protein core following protein unfolding was assayed using the C1000 Touch Thermal Cycler with CFX96 Deep Well Real-Time System (BioRad). Samples were heated from 20° C. to 95° C. with stepwise increments of 0.5° C. per minute and a 5 sec hold step for every point, followed by fluorescence reading. Tm were calculated by analyzing the derivatives of Relative florescence Units (RFU). FIG. 17D illustrates this SYPRO Orange melting assay of Peptides. Human siderocalin (HuScn) demonstrated an expected melting temperature of 79° C., as interpreted by the peak of its RFU vs temperature slope. Conversely, no melting temperature could be determined for the three peptides tested (SEQ ID NO: 43, SEQ ID NO: 44, and SEQ ID NO: 45). Thus, the SYPRO Orange thermal shift assay showed no evidence of protein unfolding, and therefore, SEQ ID NO: 43 and its point variants were shown to be stable at high temperatures.

### Example 17

### Determination of Peptide Structure and Stability in Various pH Buffers Using Circular Dichroism

This example shows determination of peptide stability in neutral, denaturing, and acidic pH conditions using circular dichroism. Protein secondary structures were assessed using Circular Dichrosim (CD).

CD spectra were measured with a Jasco J-720W spectropolarimeter using a 1.0 mm path length cell. Lyophilized peptide samples were resuspended in ultra pure water. CD runs were carried out by diluting peptides in 20 mM phosphate buffer saline (pH 7.4), 20 mM phosphate buffer saline (pH 7.4) supplemented with 1% sodium dodecyl sulfate (SDS), or phosphate buffer saline at pH 4. Peptides were diluted to 15-25 μM in concentration. Data are shown in terms of total molar ellipticity [θ]; (deg cm² dmol⁻¹).

FIG. 22 illustrates CD results of various peptides of the disclosure, indicating elements of the secondary structure of these peptides. For example, CD shows that peptides of the disclosure can vary in types of folds, yet many of these different can be stable.

Peptides were analyzed in PBS by circular dichroism, and these results show secondary structure features. Furthermore, the far UV CD spectra of a peptide of SEQ ID NO: 10, SEQ ID NO: 3, SEQ ID NO: 29, SEQ ID NO: 27, SEQ ID NO: 24, SEQ ID NO: 55, SEQ ID NO: 30, SEQ ID NO: 5, SEQ ID NO: 6, SEQ ID NO: 8, SEQ ID NO: 12, SEQ ID NO: 35, SEQ ID NO: 36, SEQ ID NO: 37, SEQ ID NO: 18, SEQ ID NO: 54, SEQ ID NO: 56, or SEQ ID NO: 57 was used to classify its topology as a hitchin.

FIG. 23 illustrates circular dichroism analysis of a peptide of SEQ ID NO: 27 after incubation in phosphate buffer saline at pH 7.2, phosphate buffer saline at pH 7.2 with 1% sodium dodecyl sulfate (SDS), or phosphate buffer saline at pH 4. Results from CD showed that a peptide of SEQ ID NO: 27 retained most of its secondary structure at all the tested conditions, indicating stability of the peptide in these conditions.

FIG. 24 illustrates circular dichroism analysis of a peptide of SEQ ID NO: 37 after incubation in phosphate buffer saline at pH 7.2, phosphate buffer saline at pH 7.2 with 1% sodium dodecyl sulfate (SDS), or phosphate buffer saline at pH 4. Results from CD showed that a peptide of SEQ ID NO: 37 retained its secondary structure in the pH 7.2 and pH 4 conditions, but lost secondary structure with the addition of 1% SDS (a detergent). Thus, since the peptide of SEQ ID NO: 37 was not able to maintain its tertiary structure in SDS as compared to the peptide of SEQ ID NO: 27 in FIG. 23, the peptide of SEQ ID NO: 37 may not be as stable.

Furthermore, these results illustrate that some peptides, such as SEQ ID NO: 27, are robust in folding and resistant to stresses, such as denaturation by sodium dodecyl sulfate (SDS).

### Example 18

### Correlation of Peptides Exhibiting Proper Cell Surface Folding, Soluble Protein Folding, and Trypsin Resistance

This example shows a correlation between cell surface folding, soluble protein folding, and trypsin resistance of peptides. Peptides with superior stability were determined by evaluating their folded structure at the cell surface and as soluble proteins. Folded structure further contributed to enhanced trypsin resistance. Nearly 10,000 peptides were identified as cysteine rich, as comprising 6, 8, or 10 cysteines within a 50 amino acid length, and as knottins, defensins, or a peptide of other types. These peptides were classified and screened for superior folding stability.

FIG. 26 illustrates identification of stable peptides identified using a surface display peptide folding assay. Peptides were displayed on the surface of 293F cells. FIG. 26A illustrates the taxonomic diversity of the full library that was screened to identify stable peptides. Only classes with greater than 300 library members are specifically named in this pie chart. FIG. 26B illustrates a dot plot, which on the x-axis shows the protein content displayed at the surface of cells transduced with a surface display GFP FasL(SDGF) vector comprising a peptide that were untreated and on the y-axis shows the protein content displayed at the surface of cells transduced with a surface display GFP FasL(SDGF) vector comprising a peptide that are treated with trypsin as a percentage of untreated surface protein content. The dot plot represents a total of about 4,300 peptides that passed read abundance thresholds out of the approximately 10,000 peptides that were initially cloned into surface display GFP FasL (SDGF) vectors. The diagonal line bisecting samples defines a cutoff between “high protein content and/or trypsin resistant” peptides and “low protein content and/or trypsin sensitive” peptides, which served to classify peptides as well-folded (high content/trypsin resistant) or poorly folded (low content/trypsin sensitive). Peptides were further expressed as secreted proteins and were classified by HPLC as 1-2 peaks (circle), 3+ peaks (square), or 0 peaks (diamonds). The number of peaks observed in the chromatogram correlate with the folding of the peptide in which peptides with 1-2 peaks were well-folded, peptides with 3+ peakes were poorly folded, and peptides with 0 peaks means the peptide was not seen and therefor it was inferred to be poorly folded. Pluses indicate peptides for which no HPLC chromatograms data was generated. Large X's are peptides that are shown in the HPLC chromatograms of FIG. 26D and the table of FIG. 26E. Well-folded peptides appear in the top right quadrant and poorly-folded peptides appear in the bottom left quadrant. The data from both the high-throughput surface display assay and HPLC analysis of soluble peptides was compared against each other to generate a concordance score. For example, a surface-displayed peptide in the top-right quadrant of the surface display peptide folding assay plot (see FIG. 26B) (indicating it was well-folded) and produced 1-2 peaks as a soluble peptide by HPLC analysis (see FIG. 26D) (also indicating it was well folded) exhibits concordant behavior (both results indicated the peptide was well-folded). Similarly a surface-displayed peptide in the bottom-left quadrant of the surface display peptide folding assay (indicating it was poorly folded) and produced 3+ or 0 peaks by HPLC analysis (also indicating it was poorly folded) also exhibits concordant behavior (both results indicated the peptide was poorly folded). Statistical significance of assessing peptide folding by a concordant score was calculated by shuffling the HPLC classifications and repeating concordance scoring over 1 million times. The real concordance scores of peptides were higher than the shuffled score at each repetition, yielding a P<1×10⁻⁶ and indicating the surface display peptide folding assay can identify a peptide that can be folded properly when it is a soluble peptide.

FIG. 26C illustrates a bar graph showing the breakdown of the tested secreted proteins (from FIG. 26B) classified by HPLC (0 Peaks; 3+ Peaks; or 1-2 Peaks). Peptides were categorized by peptide category: All Peptides; Peptides with high protein content/trypsin resistant (High Content/Trypsin Resistant); and Peptides with low protein content/trypsin sensitive (Low Content/Trypsin Sensitive). The correlation between surface folding and HPLC classification (combining 3+ and 0 peaks into one group) was highly significant.

FIG. 26D illustrates HPLC traces of various peptides of this disclosure under native (thin line) or reducing (thick line) conditions. The peptide sequence for each “Plot” is shown in FIG. 26E. FIG. 26E shows a table of each peptide that was tested by HPLC and for which HPLC chromatograms are shown in FIG. 26D. The table shows Plot number, the number of HPLC peaks that were observed, protein content, trypsin resistance, SEQ ID NO, and sequence. High protein content (in arbitrary units, calculated from the high throughput sequencing and flow cytometry data) indicates that a significant amount of peptide continued to be present at the cell surface, even after exposure to trypsin. Higher trypsin resistance (protein content in the trypsin treated peptides as a percent of the content seen in untreated peptides) indicates peptide sequences, which showed superior resistance to cleavage by the enzyme.

Trypsin resistance values were obtained from a high throughput sequencing assay, in which cells transduced with a surface display GFP FasL(SDGF) vector comprising a peptide were treated with trypsin or left untreated and sorted by flow cytometry into one of four tubes based on peptide surface abundance (each peptide comprised His tags tagged on the peptide's C-terminus, which were detected by a fluorescently labeled antibody specific for His tag). The amount of fluorescence in the four samples (relative fluorescence units (RFUs)) per treatment were recorded and the sorted samples were deep sequenced. The distribution of a given peptide in the sequence data from the four sorted samples was combined to estimate the fluorescence of cells expressing the peptide. Fluorescence correlates to surface abundance of the peptide (protein content) in the untreated and treated conditions. Thus, a high protein content indicated good expression of the peptide and if the protein content was similar between untreated and treated groups, it was also deemed to by trypsin resistant.

### Example 19

### Treatment of Inflammatory Bowel Disease

This example shows treatment of inflammatory bowel disease with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has inflammatory bowel disease. In the case of the peptide-active agent conjugate, the active agent is a steroid or an immunomodulating agent, such as prednisone, budesonide, azathiprine, or methotrexate. The peptide itself can be modified to have anti-inflammatory or immunomodulatory activities, such as by acting on ion channels or inhibiting proteases (e.g., serine proteases, ubiquitin proteasome system inhibitors) that are involved in the pathology of inflammatory bowel disease. Alternatively, the peptide itself can be modified to have TNF inhibitor activity.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the inflammatory bowel disease is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 20

### Treatment of a Crohn's Disease

This example shows treatment of Crohn's Disease with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has Crohn's disease. In the case of the peptide-active agent conjugate, the active agent is a steroid or an immunomodulating agent, such as prednisone, budesonide, sulfasalazine, or methotrexate. The peptide itself can be modified to have anti-inflammatory or immunomodulatory activities, such as by acting to modulate TRPC6 ion channels or by acting to inhibit protease activity or TNF activity.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the Crohn's disease is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 21

### Treatment of Colon Cancer

This example shows treatment of colon cancer with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has colon cancer. In the case of the peptide-active agent conjugate, the active agent is any anti-cancer drug, such as fluorouracil, gemcitabine, or mafosphamide (a cyclophosphamide pro drug). The peptide itself can be modified to have anti-cancer activity.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the colon cancer is treated.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 22

### Treatment of Enteric Pathogen

This example shows treatment of an enteric pathogen with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has an enteric pathogen, such as a bacterium, a virus, a parasite, or another organism that infects the gastrointestinal tract. In the case of the peptide-active agent conjugate, the active agent is any antibiotic, such as carbapenems, penicillins, quinolines, fluoroquinolones, aminoglycosides, amoxicillin, or tetracyline, any antimicrobial, any antiparasitic, or any antiviral agent. The peptide itself is modified to have antimicrobial activity, such as modulating proton pump inhibitors, against any enteric pathogen, such as a bacterium (e.g., Helicobactor pylori, Escherichia coli, or Campylobacter), a virus, a parasite, or another organism that infects the intestines.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the disease caused by the enteric pathogen is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 23

### Treatment of Cancers Expressing Guanylyl Cyclase C

This example shows treatment of cancers expressing guanylyl cyclase C (GCC), such as colorectal carcinomas and upper gastrointestinal tract adenocarcinomas, with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has a cancer expressing GCC. The GCC of the cancer is bound by the peptide or by the peptide of the peptide-active agent conjugate. Alternatively, the peptide itself is modified to bind to GCC. In the case of the peptide-active agent conjugate, the active agent is a chemotherapeutic agent, such as irinotecan, capecitabine, oxaliplatin, fluorouracil, leucovorin, or regorafenib.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the cancer expressing GCC is treated.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 24

### Agonism or Antagonism of Ion Channels to Treat Irritable Bowel Syndrome

This example shows treatment of irritable bowel by agonizing or antagonizing ion channels in the gastrointestinal tract with any peptide (SEQ ID NO: 1-SEQ ID NO: 166) or peptide-active agent conjugate of this disclosure. A peptide of interest is recombinantly expressed or chemically synthesized either alone or as a fusion or conjugate with an active agent. The peptide or the peptide-active agent conjugate is orally administered to a subject in need thereof. The subject in need thereof is a human or a non-human animal. The subject in need thereof has irritable bowel syndrome. The voltage gated sodium (NaV) ion channels, calcium (CaV) ion channels, potassium (KV, KCa) ion channels, chloride (Cl−) ion channels, nonselective ion channels (transient receptor potentials (TRPs)), chloride channel type-2 (ClC-2)3 ion channels, or cystic fibrosis transmembrane conductance regulator (CFTR) chloride channels in the gastrointestinal tract are agonized or antagonzed by the peptide or peptide-active agent conjugate ((Beyder, A., Therap Adv Gastroenterol., 5(1): 5-21 (2012); Jun, J. Y., J Neurogastroenterol Motil., 19(3): 277-8 (2013) Alternatively, the peptide itself is modified to bind to these ion channels.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the irritable bowel is relieved.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 25

### Engineered Resistant Peptides

This example describes engineering of a peptide of this disclosure to home to tumors and/or have antimicribial properties. A peptide of SEQ ID NO: 27, SEQ NO: 57, SEQ ID NO: 24, SEQ ID NO: 6, SEQ ID NO: 10, SEQ ID NO: 12, or SEQ ID NO: 31 is selected for it resistant properties as disclosed herein for use as a scaffold for engineering tumor homing and/or antimicrobial properties into the peptide. The peptide is then engineered to have a homing property, such as homing to a tumor, and/or an antimicrobial property. Additionally, the peptide is engineered so as to maintain its resistant properties, such temperature stability, protease resistance, and/or reduction resistance. The engineering of the peptide is accomplished by rational design, computational-guided design, or random mutagenesis that replaces native amino acids with those selected by computational software or researchers to increase binding to a tumor and/or increase antimicrobial properties, while maintaining its resistant properties. Iterative rounds of evolution using the above and related techniques are used to engineer peptides that have both resistant properties and tumor homing and/or antimicrobial properties. The engineered peptide is then tested for binding affinity to tumors and in vivo biodistribution in a tumor bearing mouse using quantitative whole body autoradiography or liquid scintillation, and/or for antimicrobial properties using bacterial growth arrest assays (e.g., culturing the peptide with a microorganism and analyzing for microorganism death or arrested growth), and resistant properties as described herein. The engineered peptide is used either as a therapeutic itself or is used with an active agent in the form of an engineered peptide-active agent conjugate. The engineered peptide or the engineered peptide-active agent conjugate is orally administered to a subject in need thereof. The subject in need thereof is a human or a non-human animal.

Enhanced stability, resistance to denaturation, reduction, or cleavage by enzymes, and increased tumor homing and/or increased antimicrobial properties are exhibited by the engineered peptide or engineered peptide-active agent conjugate after oral administration. Consequently, the engineered peptide or engineered peptide-active agent conjugate is stable long enough to deliver the active agent or to act on the target to exhibit a therapeutic effect, rather than being degraded quickly and thus having no effect, as well as able to target a tumor and/or exhibit antimicrobial properties. A therapeutic effect is exhibited by the engineered peptide or engineered peptide-active agent conjugate and the disease is treated.

### Example 26

### Resistant Property Grafting into Peptides

This example describes the grafting of resistant properties of peptides of this disclosure into a peptide. A selected knottin (e.g., selected from a library of over 200,000 identified native knottins) is used as a scaffold for a peptide-based therapeutic of the present invention. The peptide is grafted to have at least one enhanced resistant property, such as temperature stability, protease resistance, and/or reduction resistance. The graft is based on the conserved amino acids of SEQ ID NO: 27, SEQ ID NO: 57, SEQ ID NO: 24, SEQ ID NO: 6, SEQ ID NO: 10, and SEQ ID NO: 12 that confer increased resistant properties and the placement of the graft into the peptide is accomplished by computational design that replaces native amino acids with those selected by computational software or researchers so that the peptide will have the resistant properties. The resulting grafted peptides are then tested for the resistant properties as described herein. The grafted peptides with increased resistant properties are then used in as a therapy or in any therapeutic application of the present disclosure.

Alternatively, if a peptide is being cleaved by a protease, the peptide can be mutated to prevent the cleavage. For example, if a peptide is being cleaved by trypsin at a particular location, that particular location on the peptide is removed or is mutated to a lysine or arginine to prevent the trypsin cleavage.

### Example 27

### Multiply Resistant Peptide Variants

This example shows knotted peptides are optimized to be more stress-resistant (e.g., reduction resistance, protease resistance, low pH resistance, and/or elevated temperature resistance) by enhancing or modifying amino acids of the knotted peptides to conform to the modifications or mutations of the highly stress-resistant peptides disclosed herein to generate additional stable peptide scaffolds.

The basis for converting a less resistant peptide to a more resistant peptide based on the “Subtype A” structure is shown in FIG. 27D and FIG. 27E. For example, substituting a Thr amino acid at amino acid position 6 to a Ile amino acid in SEQ ID NO: 56 to make a variant peptide of SEQ ID NO: 56 comprising the amino acid sequence of GSGVPIDVKCRGSPQCIQPCKDAGMRFGKCMNGKCHCTPK (SEQ ID NO: 74) or further substituting an Ile amino acid at amino acid position 17 to a Leu amino acid to make a variant peptide of SEQ ID NO: 56 comprising the amino acid sequence of GSGVPIDVKCRGSPQCLQPCKDAGMRFGKCMNGKCHCTPK (SEQ ID NO: 75).

Thus, when a peptide (e.g., SEQ ID NO: 56) that is similar to the more resistant peptides of FIG. 27D is mutated to match the more resistant peptides, the resistant properties of the resulting variant peptides (e.g., SEQ ID NO: 74 and SEQ ID NO: 75) are enhanced. Furthermore, three dimensional modeling and crystal structure data indicated that an Ile amino acid substitution at amino acid position 6 of a peptide that is similar to the more resistant peptides of FIG. 27D is located at one end of the peptide, which can act as a gateway to sterically restrict access to the cystine to protect it from degradation, and thus further indicates that its resistant properties are increased when changing the Thr amino acid to an Ile amino acid. Additionally, a Leu amino acid at amino acid position 27 is conserved in all peptides of FIG. 27D and FIG. 27E and is adjacent to a cystine. Thus, the resistant properties of a peptide that is similar to the peptides of FIG. 27D is increased when it comprises a mutation to this Leu amino acid. (Note that the amino acid position number is based on the sequence alignments in FIG. 27.)

The basis for converting a less-resistant peptide to a more resistant peptide based on the “Subtype B” structure is shown in FIG. 27F and FIG. 27G. For example, substituting a Ile amino acid at amino acid position 6 to a Thr amino acid, an Ala amino acid at amino acid position 23 to an Arg amino acid, and an Ile amino acid at amino acid position 24 to a Phe amino acid in SEQ ID NO: 37 to make a variant peptide of SEQ ID NO: 37 comprising the amino acid sequence of GSVFTNVKCRGSPECLPKCKERFGKSAGKCMNGKCKCYP (SEQ ID NO: 76). (Note that the amino acid position number is based on the sequence alignments in FIG. 27.)

Thus, when a peptide (e.g., SEQ ID NO: 37) that is similar to the more resistant peptides of FIG. 27G is mutated to match the more resistant peptides, the resistant properties of the resulting variant peptides (e.g., SEQ ID NO: 76) are enhanced. Furthermore, three dimensional modeling and crystal structure data indicated that a Thr amino acid substitution at amino acid position 6, an Arg amino acid amino acid substitution at amino acid position 23, and a Phe amino acid substution at amino acid position 24 of a peptide that is similar to the more resistant peptides of FIG. 27G is located at one end of the peptide, which can act as a gateway to sterically restrict access to the cystine to protect it from degradation, and thus further indicates that its resistant properties are increased with these three amino acid substitutions.

### Example 28

### Stability of Peptides for Manufacture, Storage, and Distribution

This example shows the stability of any peptide (SEQ ID NO: 1-SEQ ID NO: 166) of this disclosure when manufactured, stored, and/or distributed without freezing or refrigeration. A peptide of interest is recombinantly expressed or chemically synthesized. The peptide is stored and/or distributed without freezing or without refrigeration. The peptide is exposed to 20° C., 25° C., 30° C., 40° C., 45° C., 50° C., 70° C., or 100° C. during storage and/or distribution. The peptide is stored for 1 day, 1 week, 1 month, 3 months, 6 months, 1 year, 2 years, 3 years, or 4 years before distribution and/or use. In some cases, the peptide exposed to 60-75% relative humidity or higher. After storage and/or distribution, the peptide is minimally degraded, meaning at least 70% of the peptide remains intact. Additionally, the peptide maintains at least 90% or 95% purity or at least 90% or 95% potency when assayed. The peptide is administered to a subject in need thereof. The subject in need thereof is a human or a non-human animal.

After manufacture, storage, and/or distribution, enhanced stability and resistance to denaturation, reduction, or cleavage by enzymes is exhibited by the peptide or peptide-active agent conjugate after administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the targeted disease is relieved. Therefore, the peptide or peptide-active agent conjugate is stored at room temperature or higher temperatures while maintaining product purity and potency, which removes the requirement for cold chain handling before administration.

The peptide can be any one of a peptide of SEQ ID NO: 27, SEQ ID NO: 24, SEQ ID NO: 6, SEQ ID NO: 10, SEQ ID NO: 31, or SEQ ID NO: 57.

### Example 29

### Solved Crystal Structures of Peptides

This example illustrates the solved crystal structures of peptide homologs including peptides of SEQ ID NO: 3, SEQ ID NO: 27, SEQ ID NO: 22, SEQ ID NO: 34, SEQ ID NO: 10, SEQ ID NO: 12, SEQ ID NO: 13, SEQ ID NO: 36, SEQ ID NO: 37, SEQ ID NO: 20, SEQ ID NO: 51, and SEQ ID NO: 47. For each peptide, the peptide was resuspended at a target concentration of 80 mg/mL. Crystallization screening was performed at room temperature by vapor diffusion, with 1:1 protein solution:reservoir solution sitting drops, set up using the Nextal JCSG+, PEGs, and (NH₄)₂SO₄ factorial suites (Qiagen) and sub-microliter robotics (TTP Labtech mosquito). Diffraction data were collected from single crystals using a Rigaku MicroMax-007 HF home source or beamline 5.0.1 at the Advanced Light Source (Lawrence Berkley National Laboratory, Berkeley, Calif.). Initial phases were determined either by molecular replacement (MR), using PHASER (McCoy, A. J., J Appl Crystallogr., 40(Pt 4): 658-674 (2007)) in the CCP4 program suite (Winn, M. D., Acta Crystallogr D Biol Crystallogr., 67(Pt 4): 235-42 (2011)) using homologous structures from the RCSB PDB (Berman, H. M., Nucleic Acids Res., 28(1): 235-42 (2000)) as search models, or sulfur single-wavelength anomalous diffraction (sSAD) (Liu, Q., Acta Crystallogr D Biol Crystallogr., 69(Pt 7): 1314-32 (2013)), using CuKalpha radiation to maximize the anomalous signal, and determining sulfur substructures with SHELX (Sheldrick, G. M., Acta Crystallogr D Biol Crystallogr., 66 (Pt 4): 479-85 (2010)). For sSAD phasing, Bijvoet pair measurement was optimized by collecting data through 5° wedges with alternating phi rotations of 180°, in 1° oscillations. Data were reduced and scaled with HKL2000 (Otwinowski, Z., Methods Enzymol., 276: 307-326 (1997)). Iterative cycles of model building and refinement were performed with COOT (Emsely, P., Acta Crystallogr D Biol Crystallogr., 60(Pt 12 Pt 1): 2126-32 (2004)) and REFMAC (Murshudov, G. N., Acta Crystallogr D Biol Crystallogr., 53(Pt 3): 240-55 (1997)). Structure validation was performed with MolProbity (Davis, I. W., Nucleic Acids Res., 35(Web Server issue): W375-83 (2007)).

Similarly, the crystal structure of any one of peptides of SEQ ID NO: 1-SEQ ID NO: 2, SEQ ID NO: 4-SEQ ID NO: 9, SEQ ID NO: 11, SEQ ID NO: 14-SEQ ID NO: 19; SEQ ID NO: 21, SEQ ID NO: 23-SEQ ID NO: 26; SEQ ID NO: 28-SEQ ID NO: 33, SEQ ID NO: 35, SEQ ID NO: 38-SEQ ID NO: 46, SEQ ID NO: 48-SEQ ID NO: 50, and SEQ ID NO: 52-SEQ ID NO: 166 are solved using the above methods.

### Example 30

### Peptide-Active Agent Conjugate

This example shows synthesis of a peptide-active agent conjugate using a peptide of SEQ ID NO: 31. A peptide of SEQ ID NO: 31 is recombinantly expressed or chemically synthesized either alone or as a fusion or conjugate with an active agent. The SEQ ID NO: 31 peptide or the SEQ ID NO: 31 peptide of the peptide active agent conjugate is engineered to modulate ion channels, have antimicrobial properties, and/or kill tumor cells. The SEQ ID NO: 31 peptide or peptide active agent conjugate is administered orally.

Enhanced stability and resistance to denaturation, reduction, or cleavage by enzymes is exhibited by the peptide or peptide-active agent conjugate after oral administration. Consequently, the peptide or peptide-active agent conjugate is stable long enough to deliver the active agent or to act on the target to exhibit a therapeutic effect, rather than being degraded quickly and thus having no effect. A therapeutic effect is exhibited by the peptide or peptide-active agent conjugate and the disease is relieved.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-372. (canceled)

373. A method comprising administering a compound comprising a peptide to a subject, wherein at least 70% of the peptide remains intact after exposure to pepsin at a temperature of at least 20° C. for at least 5 minutes and the peptide comprises at least 6 cysteine residues.

374. The method of claim 373, wherein the peptide remains intact when exposed to pepsin at a concentration of 47 U/ml and a temperature of 23° C. for 5 minutes as measured by high performance liquid chromatography.

375. The method of claim 373, wherein the peptide has at least one of the following characteristics:
(a) at least 70% of the peptide remains intact after exposure to pepsin at a temperature of at least 23° C. for at least 5 minutes;
(b) at least 70% of the peptide remains intact after exposure to dithiothreitol (DTT) at a concentration of 5 mM and a temperature of at least 20° C. for at least 5 minutes, as measured by high performance liquid chromatography;
(c) at least 70% of the peptide remains intact after exposure to reduced glutathione (GSH) at a concentration of 5 mM and a temperature of at least 20° C. for at least 5 minutes, as measured by high performance liquid chromatography;
(d) at least 70% of the peptide remains intact after exposure to trypsin at a concentration of 0.5 U/ml and a temperature of at least 20° C. for at least 5 minutes, as measured by high performance liquid chromatography;
(e) at least 70% of the peptide remains intact after exposure to simulated gastric fluid (SGF; pH 1.05; 2% (w/v) sodium chloride in 0.7% (v/v) hydrochloric acid) and a temperature of at least 20° C. for at least 5 minutes, as measured by high performance liquid chromatography;
(f) at least 70% of the peptide remains intact after exposure to a pH of 5 and a temperature of at least 20° C. for at least 5 minutes, as measured by high performance liquid chromatography;
(g) at least 70% of the peptide remains intact after passage through the mouth, stomach, small intestine, or the large intestine, as measured by tandem high performance liquid chromatography and liquid scintillation counting;
(h) at least 70% of the peptide remains intact after exposure to the combination of simulated gastric fluid (SGF; pH 1.05; 2% (w/v) sodium chloride in 0.7% (v/v) hydrochloric acid) with 0.5 U/ml pepsin, 100 mM Tris, and 10 mM DTT and a temperature of at least 20° C. for at least 5 minutes, as measured by high performance liquid chromatography;
(i) at least 70% of the peptide remains intact after exposure to at least 70° C. for at least 5 minutes, as measured by high performance liquid chromatography; or
(j) at least 70% of the peptide remains intact after exposure to at least 100° C. for at least 5 minutes, as measured by high performance liquid chromatography.

376. The method of claim 373, wherein the peptide comprises three or more disulfide bridges formed between cysteine residues, wherein one of the disulfide bridges passes through a loop formed by two other disulfide bridges.

377. The method of claim 373, wherein the peptide comprises a structural proline amino acid residue.

378. The method of claim 373, wherein the peptide comprises at least 3 positively charged amino acid residues.

379. The method of claim 373, wherein the peptide comprises a sequence motif of leucine-X₁-X₂-leucine-phenylalanine (LX₁X₂LF).

380. The method of claim 373, wherein the peptide comprises:
a) at least 90% sequence identity to SEQ ID NO: 114; or
b) at least 90% sequence identity to any one of SEQ ID NO: 110, SEQ ID NO: 140, SEQ ID NO: 89, SEQ ID NO: 85; SEQ ID: 107, SEQ ID NO: 93, SEQ ID NO: 95, SEQ ID NO: 160, SEQ ID NO: 161, SEQ ID NO: 126, SEQ ID NO: 127, SEQ ID NO: 128, SEQ ID NO: 162, SEQ ID NO: 163, SEQ ID NO: 94, SEQ ID NO: 102, SEQ ID NO: 103, SEQ ID NO: 100, SEQ ID NO: 92, SEQ ID NO: 104, SEQ ID NO: 105, SEQ ID NO: 87, SEQ ID NO: 90, SEQ ID NO: 91, SEQ ID NO: 96, SEQ ID NO: 98, SEQ ID NO: 99, SEQ ID NO: 85, SEQ ID NO: 101, SEQ ID NO: 115, SEQ ID NO: 108, SEQ ID NO: 119, SEQ ID NO: 120, SEQ ID NO: 121, SEQ ID NO: 131, SEQ ID NO: 132, SEQ ID NO: 112, SEQ ID NO: 134, SEQ ID NO: 135, SEQ ID NO: 136, SEQ ID NO: 137, SEQ ID NO: 138, SEQ ID NO: 139, SEQ ID NO: 106, or SEQ ID NO: 88.

381. The method of claim 373, wherein the peptide achieves an average t½ of 0.1 hours-168 hours in a subject after administering the peptide to the subject.

382. The method of claim 373, wherein the compound is administered via oral administration, inhalation, intranasal administration, topical administration, intravenous administration, subcutaneous administration, intra-articular administration, intramuscular administration, intraperitoneal administration, intra-synovial administration, vaginal administration, rectal administration, pulmonary administration, ocular administration, buccal administration, sublingual administration, intrathecal administration, or any combination thereof, to a subject.

383. The method of claim 373, wherein the peptide is linked to an active agent.

384. The method of claim 383, wherein the active agent is: a peptide, an oligopeptide, a polypeptide, a polynucleotide, a polyribonucleotide, a DNA, a cDNA, a ssDNA, a RNA, a dsRNA, a micro RNA, an oligonucleotide, an antibody, an antibody fragment, an aptamer, a cytokine, an enzyme, a growth factor, a chemokine, a neurotransmitter, a chemical agent, a fluorophore, a metal, a metal chelate, an X-ray contrast agent, a PET agent, a radioisotope, a photosensitizer, a radiosensitizer, a radionuclide chelator, a therapeutic small molecule, a steroid, a corticosteroid, an anti-inflammatory agent, an immune modulator, a protease inhibitor, an amino sugar, a chemotherapeutic agent, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, a glucocorticoid, an anti-cytokine agent, a pain-reducing agent, a dendrimer, a fatty acid, an Fc region, siderocalin, or any combination thereof.

385. The method of claim 384, wherein the steroid is triamcinolone, triamcinolone hexacetonide, budesonide, or dexamethasone.

386. The method of claim 373, wherein administering the compound treats the subject.

387. The method of claim 373, wherein the subject has a gastrointestinal infection or chronic gastrointestinal disease.

388. The method of claim 387, wherein the gastrointestinal infection is a bacterial infection, prokaryotic infection, yeast infection, or fungal infection.

389. The method of claim 387, wherein the chronic gastrointestinal disease is irritable bowel syndrome, inflammatory bowel disease, Crohn's disease, gastroesophageal reflux disease, ulcerative colitis, or constipation.

390. The method of claim 373, wherein the subject has a cancer, and wherein the cancer is colorectal cancer, stomach cancer, or esophageal cancer.

391. The method of claim 373, wherein the subject has an inflammation, a cancer, a degradation, a growth disturbance, genetic, a tear, an infection, an injury, a rheumatic condition, an immune system disorder, a kidney disease, lung disease, a condition of aging, a degenerative brain condition, a degenerative body condition, a childhood condition, a hepatic disease, a pulmonary disease, a pancreatic condition, or a gastrointestinal condition.

392. The method of claim 391, wherein the kidney disease is acute kidney injury or chronic kidney disease.

393. The method of claim 373, wherein the peptide homes to cartilage, kidneys, proximal tubules of the kidneys, or tumors.

394. The method of claim 373, wherein the peptide enters a cell, and wherein the peptide is active intracellularly.

395. The method of claim 373, wherein the peptide is formulated in a pharmaceutical composition.

396. The method of claim 395, wherein the pharmaceutical composition further includes a permeation enhancer and the permeation enhancer increases oral absorption.

397. The method of claim 396, wherein the permeation enhancer is SNAC, 5-CNAC, sodium caprylate, an aromatic alcohol, EDTA, a sodium alkyl sulfate, or a citrate.

398. The method of claim 383, wherein the peptide linked to the active agent brings or enters a cell and wherein the active agent is delivered to the cell or is active intracellularly.

399. The method of claim 383, wherein the peptide linked to the active agent is formulated in a pharmaceutical formulation.

400. The method of claim 399, wherein the pharmaceutical composition further includes a permeation enhancer and the permeation enhancer increases oral absorption.

401. The method of claim 400, wherein the permeation enhancer is SNAC, 5-CNAC, sodium caprylate, an aromatic alcohol, EDTA, a sodium alkyl sulfate, or a citrate.

402. A compound comprising a peptide for use in a treatment of a gastrointestinal disorder, wherein at least 70% of the peptide remains intact after exposure to pepsin at a temperature of at least 20° C. for at least 5 minutes and the peptide comprises at least 6 cysteine residues.

403. The peptide of claim 402 having at least one of the following characteristics:
(a) at least 70% of the peptide remains intact after exposure to pepsin at a temperature of at least 23° C. for at least 5 minutes;
(b) at least 70% of the peptide remains intact after exposure to dithiothreitol (DTT) at a concentration of 5 mM and a temperature of at least 20° C. for at least 5 minutes, as measured by high performance liquid chromatography;
(c) at least 70% of the peptide remains intact after exposure to reduced glutathione (GSH) at a concentration of 5 mM and a temperature of at least 20° C. for at least 5 minutes, as measured by high performance liquid chromatography;
(d) at least 70% of the peptide remains intact after exposure to trypsin at a concentration of 0.5 U/ml and a temperature of at least 20° C. for at least 5 minutes, as measured by high performance liquid chromatography;
(e) at least 70% of the peptide remains intact after exposure to simulated gastric fluid (SGF; pH 1.05; 2% (w/v) sodium chloride in 0.7% (v/v) hydrochloric acid) and a temperature of at least 20° C. for at least 5 minutes, as measured by high performance liquid chromatography;
(f) at least 70% of the peptide remains intact after exposure to a pH of 5 and a temperature of at least 20° C. for at least 5 minutes, as measured by high performance liquid chromatography;
(g) at least 70% of the peptide remains intact after passage through the mouth, stomach, small intestine, or the large intestine, as measured by tandem high performance liquid chromatography and liquid scintillation counting;
(h) at least 70% of the peptide remains intact after exposure to the combination of simulated gastric fluid (SGF; pH 1.05; 2% (w/v) sodium chloride in 0.7% (v/v) hydrochloric acid) with 0.5 U/ml pepsin, 100 mM Tris, and 10 mM DTT and a temperature of at least 20° C. for at least 5 minutes, as measured by high performance liquid chromatography;
(i) at least 70% of the peptide remains intact after exposure to at least 70° C. for at least 5 minutes, as measured by high performance liquid chromatography; or
(j) at least 70% of the peptide remains intact after exposure to at least 100° C. for at least 5 minutes, as measured by high performance liquid chromatography.

404. A compound comprising a peptide and a permeation enhancer, wherein at least 70% of the peptide remains intact after exposure to pepsin at a temperature of at least 20° C. for at least 5 minutes and the peptide comprises at least 6 cysteine residues.

405. The peptide of claim 404 having at least one of the following characteristics:
(a) at least 70% of the peptide remains intact after exposure to pepsin at a temperature of at least 23° C. for at least 5 minutes;
(b) at least 70% of the peptide remains intact after exposure to dithiothreitol (DTT) at a concentration of 5 mM and a temperature of at least 20° C. for at least 5 minutes, as measured by high performance liquid chromatography;
(c) at least 70% of the peptide remains intact after exposure to reduced glutathione (GSH) at a concentration of 5 mM and a temperature of at least 20° C. for at least 5 minutes, as measured by high performance liquid chromatography;
(d) at least 70% of the peptide remains intact after exposure to trypsin at a concentration of 0.5 U/ml and a temperature of at least 20° C. for at least 5 minutes, as measured by high performance liquid chromatography;
(e) at least 70% of the peptide remains intact after exposure to simulated gastric fluid (SGF; pH 1.05; 2% (w/v) sodium chloride in 0.7% (v/v) hydrochloric acid) and a temperature of at least 20° C. for at least 5 minutes, as measured by high performance liquid chromatography;
(f) at least 70% of the peptide remains intact after exposure to a pH of 5 and a temperature of at least 20° C. for at least 5 minutes, as measured by high performance liquid chromatography;
(g) at least 70% of the peptide remains intact after passage through the mouth, stomach, small intestine, or the large intestine, as measured by tandem high performance liquid chromatography and liquid scintillation counting;
(h) at least 70% of the peptide remains intact after exposure to the combination of simulated gastric fluid (SGF; pH 1.05; 2% (w/v) sodium chloride in 0.7% (v/v) hydrochloric acid) with 0.5 U/ml pepsin, 100 mM Tris, and 10 mM DTT and a temperature of at least 20° C. for at least 5 minutes, as measured by high performance liquid chromatography;
(i) at least 70% of the peptide remains intact after exposure to at least 70° C. for at least 5 minutes, as measured by high performance liquid chromatography; or
(j) at least 70% of the peptide remains intact after exposure to at least 100° C. for at least 5 minutes, as measured by high performance liquid chromatography.

406. The peptide of claim 404, wherein the permeation enhancer is SNAC, 5-CNAC, sodium caprylate, an aromatic alcohol, EDTA, a sodium alkyl sulfate, or a citrate.


SEQUENCE LISTING (182 sequences)

SEQ ID NO: 1 - protein, 34 aa, Artificial Sequence
  Description of Artificial Sequence Synthetic polypeptide
GSDCLPHLRRCRADNDCCGRRCRRRGTNAERRCR

SEQ ID NO: 2 - protein, 39 aa, Artificial Sequence
  Description of Artificial Sequence Synthetic polypeptide
GSQFTNVSCTTSRECWSVCQRLHNTSRGRCMNRRCRCYS

SEQ ID NO: 3 - protein, 39 aa, Artificial Sequence
  Description of Artificial Sequence Synthetic polypeptide
GSISIGIKCSPSIDLCEGQCRIRKYFTGYCSGDTCHCSG

SEQ ID NO: 4 - protein, 35 aa, Artificial Sequence
  Description of Artificial Sequence Synthetic polypeptide
GSGDCLPHLKRCKENNDCCSKKCKRRGANPEKRCR

SEQ ID NO: 5 - protein, 37 aa, Artificial Sequence
  Description of Artificial Sequence Synthetic polypeptide
GSNFKVEGACSKPCRKYCIDKGARNGKCINGRCHCYY

SEQ ID NO: 6 - protein, 40 aa, Artificial Sequence
  Description of Artificial Sequence Synthetic polypeptide
GSQKILSNRCNNSSECIPHCIRIFGTRAAKCINRKCYCYP

SEQ ID NO: 7 - protein, 44 aa, Artificial Sequence
  Description of Artificial Sequence Synthetic polypeptide
GSDRDSCIDKSRCSKYGYYQECQDCCKKAGHNGGTCMFFKCKCA

SEQ ID NO: 8 - protein, 34 aa, Artificial Sequence
  Description of Artificial Sequence Synthetic polypeptide
GSAVCNLKRCQLSCRSLGLLGKCIGDKCECVKHG

SEQ ID NO: 9 - protein, 39 aa, Artificial Sequence
  Description of Artificial Sequence Synthetic polypeptide
GSQFCGTNGKPCVNGQCCGALRCVVTYHYADGVCLKMNP

SEQ ID NO: 10 - protein, 38 aa, Artificial Sequence
  Description of Artificial Sequence Synthetic polypeptide
GSRPTDIKCSASYQCFPVCKSRFGKTNGRCVNGLCDCF

SEQ ID NO: 11 - protein, 37 aa, Artificial Sequence
  Description of Artificial Sequence Synthetic polypeptide
GSNCAGYMRECKEKLCCSGYVCSSRWKWCVLPAPWRR

SEQ ID NO: 12 - protein, 39 aa, Artificial Sequence
  Description of Artificial Sequence Synthetic polypeptide
GSQFTDVKCTGSKQCWPVCKQMFGKPNGKCMNGKCRCYS

SEQ ID NO: 13 - protein, 40 aa, Artificial Sequence
  Description of Artificial Sequence Synthetic polypeptide
GSAEIIRCSGTRECYAPCQKLTGCLNAKCMNKACKCYGCV

SEQ ID NO: 14 - protein, 38 aa, Artificial Sequence
  Description of Artificial Sequence Synthetic polypeptide
GSSDYCSNDFCFFSCRRDRCARGDCENGKCVCKNCHLN

SEQ ID NO: 15 - protein, 41 aa, Artificial Sequence
  Description of Artificial Sequence Synthetic polypeptide
GSCIGEGVPCDENDPRCCFGLVCLKPTLHGIWYKSYYCYKK

SEQ ID NO: 16 - protein, 33 aa, Artificial Sequence
  Description of Artificial Sequence Synthetic polypeptide
GSSCAKPGEMCMRIKCCDGQCGCNRGTGRCFCK

SEQ ID NO: 17 - protein, 34 aa, Artificial Sequence
  Description of Artificial Sequence Synthetic polypeptide
GSACQFWSCNSSCISRGYRQGYCWGIQYKYCQCQ

SEQ ID NO: 18 - protein, 40 aa, Artificial Sequence
  Description of Artificial Sequence Synthetic polypeptide
GSQVSTNKKCSNTSQCYKTCEKVVGVAAGKCMNGKCICYP

SEQ ID NO: 19 - protein, 56 aa, Artificial Sequence
  Description of Artificial Sequence Synthetic polypeptide
GSQDKCKKVYENYPVSKCQLANQCNYDCKLDKHARSGECFYDEKRNLQCICDYCEY

SEQ ID NO: 20 - protein, 24 aa, Artificial Sequence
  Description of Artificial Sequence Synthetic peptide
GSGHACYRNCWREGNDEETCKERC

SEQ ID NO: 21 - protein, 44 aa, Artificial Sequence
  Description of Artificial Sequence Synthetic polypeptide
GSRCQLQGFNCVVRSYGLPTIPCCRGLTCRSYFPGSTYGRCQRY

SEQ ID NO: 22 - protein, 38 aa, Artificial Sequence
  Description of Artificial Sequence Synthetic polypeptide
GSMCMPCFTTRPDMAQQCRACCKGRGKCFGPQCLCGYD

SEQ ID NO: 23 - protein, 39 aa, Artificial Sequence
  Description of Artificial Sequence Synthetic polypeptide
GSEVIRCSGSKQCYGPCKQQTGCTNSKCMNKVCKCYGCG

SEQ ID NO: 24 - protein, 39 aa, Artificial Sequence
  Description of Artificial Sequence Synthetic polypeptide
GSVRIPVSCKHSGQCLKPCKDAGMRFGKCMNGKCDCTPK

SEQ ID NO: 25 - protein, 37 aa, Artificial Sequence
  Description of Artificial Sequence Synthetic polypeptide
GSACLAEYQKCEGSTVPCCPGLSCSAGRFRKTKLCTK

SEQ ID NO: 26 - protein, 30 aa, Artificial Sequence
  Description of Artificial Sequence Synthetic polypeptide
GSVGCEECPMHCKGKNANPTCDDGVCNCNV

SEQ ID NO: 27 - protein, 39 aa, Artificial Sequence
  Description of Artificial Sequence Synthetic polypeptide
GSGVPINVKCRGSRDCLDPCKKAGMRFGKCINSKCHCTP

SEQ ID NO: 28 - protein, 38 aa, Artificial Sequence
  Description of Artificial Sequence Synthetic polypeptide
GSRCPPCFTTNPNMEADCRKCCGGRGYCASYQCICPGG

SEQ ID NO: 29 - protein, 37 aa, Artificial Sequence
  Description of Artificial Sequence Synthetic polypeptide
GSSEKDCIKHLQRCRENKDCCSKKCSRRGTNPEKRCR

SEQ ID NO: 30 - protein, 40 aa, Artificial Sequence
  Description of Artificial Sequence Synthetic polypeptide
GSQVQTNVKCQGGSCASVCRREIGVAAGKCINGKCVCYRN

SEQ ID NO: 31 - protein, 39 aa, Artificial Sequence
  Description of Artificial Sequence Synthetic polypeptide
GSGVPINVRCRGSRDCLDPCRRAGMRFGRCINSRCHCTP

SEQ ID NO: 32 - protein, 36 aa, Artificial Sequence
  Description of Artificial Sequence Synthetic polypeptide
GSSCAKPRENCNRMNILCCRGECVCPTFGDCFCYGD

SEQ ID NO: 33 - protein, 39 aa, Artificial Sequence
  Description of Artificial Sequence Synthetic polypeptide
GSDCVRFWGKCSQTSDCCPHLACKSKWPRNICVWDGSVG

SEQ ID NO: 34 - protein, 35 aa, Artificial Sequence
  Description of Artificial Sequence Synthetic polypeptide
GSGCFGYKCDYYKGCCSGYVCSPTWKWCVRPGPGR

SEQ ID NO: 35 - protein, 40 aa, Artificial Sequence
  Description of Artificial Sequence Synthetic polypeptide
GSQIDTNVKCSGSSKCVKICIDRYNTRGAKCINGRCTCYP

SEQ ID NO: 36 - protein, 37 aa, Artificial Sequence
  Description of Artificial Sequence Synthetic polypeptide
GSVVIGQRCYRSPDCYSACKKLVGKATGKCTNGRCDC

SEQ ID NO: 37 - protein, 39 aa, Artificial Sequence
  Description of Artificial Sequence Synthetic polypeptide
GSVFINVKCRGSPECLPKCKEAIGKSAGKCMNGKCKCYP

SEQ ID NO: 38 - protein, 38 aa, Artificial Sequence
  Description of Artificial Sequence Synthetic polypeptide
GSECLEIFKACNPSNDQCCKSSKLVCSRKTRWCKYQIG

SEQ ID NO: 39 - protein, 42 aa, Artificial Sequence
  Description of Artificial Sequence Synthetic polypeptide
GSPDEYIERAKECCKKGDIQCCLRYFEESGDPNVMLICLFCP

SEQ ID NO: 40 - protein, 42 aa, Artificial Sequence
  Description of Artificial Sequence Synthetic polypeptide
GSPDEYIERAKECCKKGDIQCCLRYFEESGDPNVMLICAFCP

SEQ ID NO: 41 - protein, 42 aa, Artificial Sequence
  Description of Artificial Sequence Synthetic polypeptide
GSPDEYIERAKECCKKGDIQCCLRYFEESGDPNVMLICLACP

SEQ ID NO: 42 - protein, 39 aa, Artificial Sequence
  Description of Artificial Sequence Synthetic polypeptide
GSFGLYDNQCATSDACSAICKYWTGSGQGKCQNNQCRCY

SEQ ID NO: 43 - protein, 42 aa, Artificial Sequence
  Description of Artificial Sequence Synthetic polypeptide
GSPDEYIERAKECCKKQDIQCCLRIFDESKDPNVMLICLFCW

SEQ ID NO: 44 - protein, 42 aa, Artificial Sequence
  Description of Artificial Sequence Synthetic polypeptide
GSPDEYIERAKECCKKQDIQCCLRIFDESGDPNVMLICLFCW

SEQ ID NO: 45 - protein, 42 aa, Artificial Sequence
  Description of Artificial Sequence Synthetic polypeptide
GSPDEYIERAKECCKKQDIQCCLRIFDESKDPNVMLICLFCP

SEQ ID NO: 46 - protein, 46 aa, Artificial Sequence
  Description of Artificial Sequence Synthetic polypeptide
GSDCKYKFENWGACDGGTGTKVRQGTLKKARYNAQCQETIRVTKPC

SEQ ID NO: 47 - protein, 59 aa, Artificial Sequence
  Description of Artificial Sequence Synthetic polypeptide
GSAQEPVKGPVSTKPGSCPIILIRCAMLNPPNRCLKDTDCPGIKKCCEGSCGMACFVPQ

SEQ ID NO: 48 - protein, 43 aa, Artificial Sequence
  Description of Artificial Sequence Synthetic polypeptide
GSGIGDPVTCLKSGAICHPVFCPRRYKQIGTCGLPGTKCCKKP

SEQ ID NO: 49 - protein, 40 aa, Artificial Sequence
  Description of Artificial Sequence Synthetic polypeptide
GSSFGLCRLRRGFCARGRCRFPSIPIGRCSRFVQCCRRVW

SEQ ID NO: 50 - protein, 38 aa, Artificial Sequence
  Description of Artificial Sequence Synthetic polypeptide
GSLFCKGGSCHFGGCPSHLIKVGSCFGFRSCCKWPWNA

SEQ ID NO: 51 - protein, 38 aa, Artificial Sequence
  Description of Artificial Sequence Synthetic polypeptide
GSMCMPCFTTDHQMARKCDDCCGGKGRGKCYGPQCLCR

SEQ ID NO: 52 - protein, 34 aa, Artificial Sequence
  Description of Artificial Sequence Synthetic polypeptide
GSEGDCPISEAIKCVEKCKEKVEVCEPGVCKCSG

SEQ ID NO: 53 - protein, 38 aa, Artificial Sequence
  Description of Artificial Sequence Synthetic polypeptide
GSLCLPCFTTHHRLADQCDICCGGDGRGKCYGPQCLCR

SEQ ID NO: 54 - protein, 31 aa, Artificial Sequence
  Description of Artificial Sequence Synthetic polypeptide
GSVSCEDCPEHCATKDQRAKCDNDRCVCEPK

SEQ ID NO: 55 - protein, 34 aa, Artificial Sequence
  Description of Artificial Sequence Synthetic polypeptide
GSGIVCKVCKIICGMQGKKVNICKAPIKCKCKKG

SEQ ID NO: 56 - protein, 40 aa, Artificial Sequence
  Description of Artificial Sequence Synthetic polypeptide
GSGVPTDVKCRGSPQCIQPCKDAGMRFGKCMNGKCHCTPK

SEQ ID NO: 57 - protein, 40 aa, Artificial Sequence
  Description of Artificial Sequence Synthetic polypeptide
GSGVIINVKCKISRQCLEPCKKAGMRFGKCMNGKCHCTPK

SEQ ID NO: 58 - protein, 32 aa, Artificial Sequence
  Description of Artificial Sequence Synthetic polypeptide
GSAPPCKRDVDCSFECPKGGFCNDRLGTCDCF

SEQ ID NO: 59 - protein, 39 aa, Artificial Sequence
  Description of Artificial Sequence Synthetic polypeptide
GSRQPCSYYDGVCRDKSDVNCKYIAFTYCENPNQRCCYY

SEQ ID NO: 60 - protein, 39 aa, Artificial Sequence
  Description of Artificial Sequence Synthetic polypeptide
GSERECVGENGHCRSWYNDCCDGYYCSCMQPPNCICRNN

SEQ ID NO: 61 - protein, 37 aa, Artificial Sequence
  Description of Artificial Sequence Synthetic polypeptide
GSKEICERPNGSCRDFCLETEIHVGRCLNSQPCCLPL

SEQ ID NO: 62 - protein, 42 aa, Artificial Sequence
  Description of Artificial Sequence Synthetic polypeptide
GSYTDCTESGQNLCLCEGSNVCGKGNKCILGSQGKDNQCVTG

SEQ ID NO: 63 - protein, 41 aa, Artificial Sequence
  Description of Artificial Sequence Synthetic polypeptide
GSPMTCEQAMASCEHTMCGYCQGPLYMTCIGITTDPECGLP

SEQ ID NO: 64 - protein, 43 aa, Artificial Sequence
  Description of Artificial Sequence Synthetic polypeptide
GSKDYCPGICNVAAVPDCDTLCISLGYSGGYCRAGRICCCNPK

SEQ ID NO: 65 - protein, 42 aa, Artificial Sequence
  Description of Artificial Sequence Synthetic polypeptide
GSYASCEAAEADCIHDDCFSEDTYTDVCQESCQYMYDNCMDD

SEQ ID NO: 66 - protein, 36 aa, Artificial Sequence
  Description of Artificial Sequence Synthetic polypeptide
GSGICACRRRFCLNFEQFSGYCRVNGARYVRCCSRR

SEQ ID NO: 67 - protein, 35 aa, Artificial Sequence
  Description of Artificial Sequence Synthetic polypeptide
GSDLVCYCRKRGCKRREHMNGTCRRGHLMYTLCCR

SEQ ID NO: 68 - protein, 50 aa, Artificial Sequence
  Description of Artificial Sequence Synthetic polypeptide
GSERGCGLLMDACDGKSTFCCSGYNCSPTWKWCVLDCPNLFLLPPTKTLC

SEQ ID NO: 69 - protein, 45 aa, Artificial Sequence
  Description of Artificial Sequence Synthetic polypeptide
GSSVPCVSTRGSCKPPAPACCHPCASCQCRFFRSACSCRVLNVNC

SEQ ID NO: 70 - protein, 37 aa, Artificial Sequence
  Description of Artificial Sequence Synthetic polypeptide
GSRGQCWSYSNCRAVCRDEGYVSGHCNYFGGACWCAS

SEQ ID NO: 71 - protein, 34 aa, Artificial Sequence
  Description of Artificial Sequence Synthetic polypeptide
GSLHRCRIYGTACADCCLARDPYCAWDGNSCSRF

SEQ ID NO: 72 - protein, 40 aa, Artificial Sequence
  Description of Artificial Sequence Synthetic polypeptide
GSSDLCSTRQSRFRDYHCRCYSAWEGACCQTLRPSRCQKR

SEQ ID NO: 73 - protein, 38 aa, Artificial Sequence
  Description of Artificial Sequence Synthetic polypeptide
GSVDGCQETCTKMIRCQFFTYSLFPEDCRGEKCKCSLR

SEQ ID NO: 74 - protein, 40 aa, Artificial Sequence
  Description of Artificial Sequence Synthetic polypeptide
GSGVPIDVKCRGSPQCIQPCKDAGMRFGKCMNGKCHCTPK

SEQ ID NO: 75 - protein, 40 aa, Artificial Sequence
  Description of Artificial Sequence Synthetic polypeptide
GSGVPIDVKCRGSPQCLQPCKDAGMRFGKCMNGKCHCTPK

SEQ ID NO: 76 - protein, 39 aa, Artificial Sequence
  Description of Artificial Sequence Synthetic polypeptide
GSVFTNVKCRGSPECLPKCKERFGKSAGKCMNGKCKCYP

SEQ ID NO: 77 - protein, 39 aa, Artificial Sequence
  Description of Artificial Sequence Synthetic polypeptide
GSVRIPVSCRHSGQCLRPCRDAGMRFGRCMNGRCDCTPR

SEQ ID NO: 78 - protein, 38 aa, Artificial Sequence
  Description of Artificial Sequence Synthetic polypeptide
GSRPTDIRCSASYQCFPVCRSRFGRTNGRCVNGLCDCF

SEQ ID NO: 79 - protein, 37 aa, Artificial Sequence
  Description of Artificial Sequence Synthetic polypeptide
GSSERDCIRHLQRCRENRDCCSRRCSRRGTNPERRCR

SEQ ID NO: 80 - protein, 35 aa, Artificial Sequence
  Description of Artificial Sequence Synthetic polypeptide
GSGDCLPHLRRCRENNDCCSRRCRRRGANPERRCR

SEQ ID NO: 81 - protein, 36 aa, Artificial Sequence
  Description of Artificial Sequence Synthetic polypeptide
GSMCIPCFTTNPNMAAKCNACCGSRRGSCRGPQCIC

SEQ ID NO: 82 - protein, 37 aa, Artificial Sequence
  Description of Artificial Sequence Synthetic polypeptide
GSGCLEFWWKCNPNDDKCCRPKLKCSKLFKLCNFSFG

SEQ ID NO: 83 - protein, 38 aa, Artificial Sequence
  Description of Artificial Sequence Synthetic polypeptide
GSECRYWLGTCSKTGDCCSHLSCSPKHGWCVWDWTFRK

SEQ ID NO: 84 - protein, 32 aa, Artificial Sequence
  Description of Artificial Sequence Synthetic polypeptide
DCLPHLRRCRADNDCCGRRCRRRGTNAERRCR

SEQ ID NO: 85 - protein, 37 aa, Artificial Sequence
  Description of Artificial Sequence Synthetic polypeptide
QFTNVSCTTSRECWSVCQRLHNTSRGRCMNRRCRCYS

SEQ ID NO: 86 - protein, 37 aa, Artificial Sequence
  Description of Artificial Sequence Synthetic polypeptide
ISIGIKCSPSIDLCEGQCRIRKYFTGYCSGDTCHCSG

SEQ ID NO: 87 - protein, 33 aa, Artificial Sequence
  Description of Artificial Sequence Synthetic polypeptide
GDCLPHLKRCKENNDCCSKKCKRRGANPEKRCR

SEQ ID NO: 88 - protein, 35 aa, Artificial Sequence
  Description of Artificial Sequence Synthetic polypeptide
NFKVEGACSKPCRKYCIDKGARNGKCINGRCHCYY

SEQ ID NO: 89 - protein, 38 aa, Artificial Sequence
  Description of Artificial Sequence Synthetic polypeptide
QKILSNRCNNSSECIPHCIRIFGTRAAKCINRKCYCYP

SEQ ID NO: 90 - protein, 42 aa, Artificial Sequence
  Description of Artificial Sequence Synthetic polypeptide
DRDSCIDKSRCSKYGYYQECQDCCKKAGHNGGTCMFFKCKCA

SEQ ID NO: 91 - protein, 32 aa, Artificial Sequence
  Description of Artificial Sequence Synthetic polypeptide
AVCNLKRCQLSCRSLGLLGKCIGDKCECVKHG

SEQ ID NO: 92 - protein, 37 aa, Artificial Sequence
  Description of Artificial Sequence Synthetic polypeptide
QFCGTNGKPCVNGQCCGALRCVVTYHYADGVCLKMNP

SEQ ID NO: 93 - protein, 36 aa, Artificial Sequence
  Description of Artificial Sequence Synthetic polypeptide
RPTDIKCSASYQCFPVCKSRFGKTNGRCVNGLCDCF

SEQ ID NO: 94 - protein, 35 aa, Artificial Sequence
  Description of Artificial Sequence Synthetic polypeptide
NCAGYMRECKEKLCCSGYVCSSRWKWCVLPAPWRR

SEQ ID NO: 95 - protein, 37 aa, Artificial Sequence
  Description of Artificial Sequence Synthetic polypeptide
QFTDVKCTGSKQCWPVCKQMFGKPNGKCMNGKCRCYS

SEQ ID NO: 96 - protein, 38 aa, Artificial Sequence
  Description of Artificial Sequence Synthetic polypeptide
AEIIRCSGTRECYAPCQKLTGCLNAKCMNKACKCYGCV

SEQ ID NO: 97 - protein, 36 aa, Artificial Sequence
  Description of Artificial Sequence Synthetic polypeptide
SDYCSNDFCFFSCRRDRCARGDCENGKCVCKNCHLN

SEQ ID NO: 98 - protein, 39 aa, Artificial Sequence
  Description of Artificial Sequence Synthetic polypeptide
CIGEGVPCDENDPRCCFGLVCLKPTLHGIWYKSYYCYKK

SEQ ID NO: 99 - protein, 31 aa, Artificial Sequence
  Description of Artificial Sequence Synthetic polypeptide
SCAKPGEMCMRIKCCDGQCGCNRGTGRCFCK

SEQ ID NO: 100 - protein, 32 aa, Artificial Sequence
  Description of Artificial Sequence Synthetic polypeptide
ACQFWSCNSSCISRGYRQGYCWGIQYKYCQCQ

SEQ ID NO: 101 - protein, 38 aa, Artificial Sequence
  Description of Artificial Sequence Synthetic polypeptide
QVSTNKKCSNTSQCYKTCEKVVGVAAGKCMNGKCICYP

SEQ ID NO: 102 - protein, 54 aa, Artificial Sequence
  Description of Artificial Sequence Synthetic polypeptide
QDKCKKVYENYPVSKCQLANQCNYDCKLDKHARSGECFYDEKRNLQCICDYCEY

SEQ ID NO: 103 - protein, 22 aa, Artificial Sequence
  Description of Artificial Sequence Synthetic peptide
GHACYRNCWREGNDEETCKERC

SEQ ID NO: 104 - protein, 42 aa, Artificial Sequence
  Description of Artificial Sequence Synthetic polypeptide
RCQLQGFNCVVRSYGLPTIPCCRGLTCRSYFPGSTYGRCQRY

SEQ ID NO: 105 - protein, 36 aa, Artificial Sequence
  Description of Artificial Sequence Synthetic polypeptide
MCMPCFTTRPDMAQQCRACCKGRGKCFGPQCLCGYD

SEQ ID NO: 106 - protein, 37 aa, Artificial Sequence
  Description of Artificial Sequence Synthetic polypeptide
EVIRCSGSKQCYGPCKQQTGCTNSKCMNKVCKCYGCG

SEQ ID NO: 107 - protein, 37 aa, Artificial Sequence
  Description of Artificial Sequence Synthetic polypeptide
VRIPVSCKHSGQCLKPCKDAGMRFGKCMNGKCDCTPK

SEQ ID NO: 108 - protein, 35 aa, Artificial Sequence
  Description of Artificial Sequence Synthetic polypeptide
ACLAEYQKCEGSTVPCCPGLSCSAGRFRKTKLCTK

SEQ ID NO: 109 - protein, 28 aa, Artificial Sequence
  Description of Artificial Sequence Synthetic peptide
VGCEECPMHCKGKNANPTCDDGVCNCNV

SEQ ID NO: 110 - protein, 37 aa, Artificial Sequence
  Description of Artificial Sequence Synthetic polypeptide
GVPINVKCRGSRDCLDPCKKAGMRFGKCINSKCHCTP

SEQ ID NO: 111 - protein, 36 aa, Artificial Sequence
  Description of Artificial Sequence Synthetic polypeptide
RCPPCFTTNPNMEADCRKCCGGRGYCASYQCICPGG

SEQ ID NO: 112 - protein, 35 aa, Artificial Sequence
  Description of Artificial Sequence Synthetic polypeptide
SEKDCIKHLQRCRENKDCCSKKCSRRGTNPEKRCR

SEQ ID NO: 113 - protein, 38 aa, Artificial Sequence
  Description of Artificial Sequence Synthetic polypeptide
QVQTNVKCQGGSCASVCRREIGVAAGKCINGKCVCYRN

SEQ ID NO: 114 - protein, 37 aa, Artificial Sequence
  Description of Artificial Sequence Synthetic polypeptide
GVPINVRCRGSRDCLDPCRRAGMRFGRCINSRCHCTP

SEQ ID NO: 115 - protein, 34 aa, Artificial Sequence
  Description of Artificial Sequence Synthetic polypeptide
SCAKPRENCNRMNILCCRGECVCPTFGDCFCYGD

SEQ ID NO: 116 - protein, 37 aa, Artificial Sequence
  Description of Artificial Sequence Synthetic polypeptide
DCVRFWGKCSQTSDCCPHLACKSKWPRNICVWDGSVG

SEQ ID NO: 117 - protein, 33 aa, Artificial Sequence
  Description of Artificial Sequence Synthetic polypeptide
GCFGYKCDYYKGCCSGYVCSPTWKWCVRPGPGR

SEQ ID NO: 118 - protein, 38 aa, Artificial Sequence
  Description of Artificial Sequence Synthetic polypeptide
QIDTNVKCSGSSKCVKICIDRYNTRGAKCINGRCTCYP

SEQ ID NO: 119 - protein, 35 aa, Artificial Sequence
  Description of Artificial Sequence Synthetic polypeptide
VVIGQRCYRSPDCYSACKKLVGKATGKCTNGRCDC

SEQ ID NO: 120 - protein, 37 aa, Artificial Sequence
  Description of Artificial Sequence Synthetic polypeptide
VFINVKCRGSPECLPKCKEAIGKSAGKCMNGKCKCYP

SEQ ID NO: 121 - protein, 36 aa, Artificial Sequence
  Description of Artificial Sequence Synthetic polypeptide
ECLEIFKACNPSNDQCCKSSKLVCSRKTRWCKYQIG

SEQ ID NO: 122 - protein, 40 aa, Artificial Sequence
  Description of Artificial Sequence Synthetic polypeptide
PDEYIERAKECCKKGDIQCCLRYFEESGDPNVMLICLFCP

SEQ ID NO: 123 - protein, 40 aa, Artificial Sequence
  Description of Artificial Sequence Synthetic polypeptide
PDEYIERAKECCKKGDIQCCLRYFEESGDPNVMLICAFCP

SEQ ID NO: 124 - protein, 40 aa, Artificial Sequence
  Description of Artificial Sequence Synthetic polypeptide
PDEYIERAKECCKKGDIQCCLRYFEESGDPNVMLICLACP

SEQ ID NO: 125 - protein, 37 aa, Artificial Sequence
  Description of Artificial Sequence Synthetic polypeptide
FGLYDNQCATSDACSAICKYWTGSGQGKCQNNQCRCY

SEQ ID NO: 126 - protein, 40 aa, Artificial Sequence
  Description of Artificial Sequence Synthetic polypeptide
PDEYIERAKECCKKQDIQCCLRIFDESKDPNVMLICLFCW

SEQ ID NO: 127 - protein, 40 aa, Artificial Sequence
  Description of Artificial Sequence Synthetic polypeptide
PDEYIERAKECCKKQDIQCCLRIFDESGDPNVMLICLFCW

SEQ ID NO: 128 - protein, 40 aa, Artificial Sequence
  Description of Artificial Sequence Synthetic polypeptide
PDEYIERAKECCKKQDIQCCLRIFDESKDPNVMLICLFCP

SEQ ID NO: 129 - protein, 44 aa, Artificial Sequence
  Description of Artificial Sequence Synthetic polypeptide
DCKYKFENWGACDGGTGTKVRQGTLKKARYNAQCQETIRVTKPC

SEQ ID NO: 130 - protein, 57 aa, Artificial Sequence
  Description of Artificial Sequence Synthetic polypeptide
AQEPVKGPVSTKPGSCPIILIRCAMLNPPNRCLKDTDCPGIKKCCEGSCGMACFVPQ

SEQ ID NO: 131 - protein, 41 aa, Artificial Sequence
  Description of Artificial Sequence Synthetic polypeptide
GIGDPVTCLKSGAICHPVFCPRRYKQIGTCGLPGTKCCKKP

SEQ ID NO: 132 - protein, 38 aa, Artificial Sequence
  Description of Artificial Sequence Synthetic polypeptide
SFGLCRLRRGFCARGRCRFPSIPIGRCSRFVQCCRRVW

SEQ ID NO: 133 - protein, 36 aa, Artificial Sequence
  Description of Artificial Sequence Synthetic polypeptide
LFCKGGSCHFGGCPSHLIKVGSCFGFRSCCKWPWNA

SEQ ID NO: 134 - protein, 36 aa, Artificial Sequence
  Description of Artificial Sequence Synthetic polypeptide
MCMPCFTTDHQMARKCDDCCGGKGRGKCYGPQCLCR

SEQ ID NO: 135 - protein, 32 aa, Artificial Sequence
  Description of Artificial Sequence Synthetic polypeptide
EGDCPISEAIKCVEKCKEKVEVCEPGVCKCSG

SEQ ID NO: 136 - protein, 36 aa, Artificial Sequence
  Description of Artificial Sequence Synthetic polypeptide
LCLPCFTTHHRLADQCDICCGGDGRGKCYGPQCLCR

SEQ ID NO: 137 - protein, 29 aa, Artificial Sequence
  Description of Artificial Sequence Synthetic peptide
VSCEDCPEHCATKDQRAKCDNDRCVCEPK

SEQ ID NO: 138 - protein, 32 aa, Artificial Sequence
  Description of Artificial Sequence Synthetic polypeptide
GIVCKVCKIICGMQGKKVNICKAPIKCKCKKG

SEQ ID NO: 139 - protein, 38 aa, Artificial Sequence
  Description of Artificial Sequence Synthetic polypeptide
GVPTDVKCRGSPQCIQPCKDAGMRFGKCMNGKCHCTPK

SEQ ID NO: 140 - protein, 38 aa, Artificial Sequence
  Description of Artificial Sequence Synthetic polypeptide
GVIINVKCKISRQCLEPCKKAGMRFGKCMNGKCHCTPK

SEQ ID NO: 141 - protein, 30 aa, Artificial Sequence
  Description of Artificial Sequence Synthetic polypeptide
APPCKRDVDCSFECPKGGFCNDRLGTCDCF

SEQ ID NO: 142 - protein, 37 aa, Artificial Sequence
  Description of Artificial Sequence Synthetic polypeptide
RQPCSYYDGVCRDKSDVNCKYIAFTYCENPNQRCCYY

SEQ ID NO: 143 - protein, 37 aa, Artificial Sequence
  Description of Artificial Sequence Synthetic polypeptide
ERECVGENGHCRSWYNDCCDGYYCSCMQPPNCICRNN

SEQ ID NO: 144 - protein, 35 aa, Artificial Sequence
  Description of Artificial Sequence Synthetic polypeptide
KEICERPNGSCRDFCLETEIHVGRCLNSQPCCLPL

SEQ ID NO: 145 - protein, 40 aa, Artificial Sequence
  Description of Artificial Sequence Synthetic polypeptide
YTDCTESGQNLCLCEGSNVCGKGNKCILGSQGKDNQCVTG

SEQ ID NO: 146 - protein, 39 aa, Artificial Sequence
  Description of Artificial Sequence Synthetic polypeptide
PMTCEQAMASCEHTMCGYCQGPLYMTCIGITTDPECGLP

SEQ ID NO: 147 - protein, 41 aa, Artificial Sequence
  Description of Artificial Sequence Synthetic polypeptide
KDYCPGICNVAAVPDCDTLCISLGYSGGYCRAGRICCCNPK

SEQ ID NO: 148 - protein, 40 aa, Artificial Sequence
  Description of Artificial Sequence Synthetic polypeptide
YASCEAAEADCIHDDCFSEDTYTDVCQESCQYMYDNCMDD

SEQ ID NO: 149 - protein, 34 aa, Artificial Sequence
  Description of Artificial Sequence Synthetic polypeptide
GICACRRRFCLNFEQFSGYCRVNGARYVRCCSRR

SEQ ID NO: 150 - protein, 33 aa, Artificial Sequence
  Description of Artificial Sequence Synthetic polypeptide
DLVCYCRKRGCKRREHMNGTCRRGHLMYTLCCR

SEQ ID NO: 151 - protein, 48 aa, Artificial Sequence
  Description of Artificial Sequence Synthetic polypeptide
ERGCGLLMDACDGKSTFCCSGYNCSPTWKWCVLDCPNLFLLPPTKTLC

SEQ ID NO: 152 - protein, 43 aa, Artificial Sequence
  Description of Artificial Sequence Synthetic polypeptide
SVPCVSTRGSCKPPAPACCHPCASCQCRFFRSACSCRVLNVNC

SEQ ID NO: 153 - protein, 35 aa, Artificial Sequence
  Description of Artificial Sequence Synthetic polypeptide
RGQCWSYSNCRAVCRDEGYVSGHCNYFGGACWCAS

SEQ ID NO: 154 - protein, 32 aa, Artificial Sequence
  Description of Artificial Sequence Synthetic polypeptide
LHRCRIYGTACADCCLARDPYCAWDGNSCSRF

SEQ ID NO: 155 - protein, 38 aa, Artificial Sequence
  Description of Artificial Sequence Synthetic polypeptide
SDLCSTRQSRFRDYHCRCYSAWEGACCQTLRPSRCQKR

SEQ ID NO: 156 - protein, 36 aa, Artificial Sequence
  Description of Artificial Sequence Synthetic polypeptide
VDGCQETCTKMIRCQFFTYSLFPEDCRGEKCKCSLR

SEQ ID NO: 157 - protein, 38 aa, Artificial Sequence
  Description of Artificial Sequence Synthetic polypeptide
GVPIDVKCRGSPQCIQPCKDAGMRFGKCMNGKCHCTPK

SEQ ID NO: 158 - protein, 38 aa, Artificial Sequence
  Description of Artificial Sequence Synthetic polypeptide
GVPIDVKCRGSPQCLQPCKDAGMRFGKCMNGKCHCTPK

SEQ ID NO: 159 - protein, 37 aa, Artificial Sequence
  Description of Artificial Sequence Synthetic polypeptide
VFTNVKCRGSPECLPKCKERFGKSAGKCMNGKCKCYP

SEQ ID NO: 160 - protein, 37 aa, Artificial Sequence
  Description of Artificial Sequence Synthetic polypeptide
VRIPVSCRHSGQCLRPCRDAGMRFGRCMNGRCDCTPR

SEQ ID NO: 161 - protein, 36 aa, Artificial Sequence
  Description of Artificial Sequence Synthetic polypeptide
RPTDIRCSASYQCFPVCRSRFGRTNGRCVNGLCDCF

SEQ ID NO: 162 - protein, 35 aa, Artificial Sequence
  Description of Artificial Sequence Synthetic polypeptide
SERDCIRHLQRCRENRDCCSRRCSRRGTNPERRCR

SEQ ID NO: 163 - protein, 33 aa, Artificial Sequence
  Description of Artificial Sequence Synthetic polypeptide
GDCLPHLRRCRENNDCCSRRCRRRGANPERRCR

SEQ ID NO: 164 - protein, 34 aa, Artificial Sequence
  Description of Artificial Sequence Synthetic polypeptide
MCIPCFTTNPNMAAKCNACCGSRRGSCRGPQCIC

SEQ ID NO: 165 - protein, 35 aa, Artificial Sequence
  Description of Artificial Sequence Synthetic polypeptide
GCLEFWWKCNPNDDKCCRPKLKCSKLFKLCNFSFG

SEQ ID NO: 166 - protein, 36 aa, Artificial Sequence
  Description of Artificial Sequence Synthetic polypeptide
ECRYWLGTCSKTGDCCSHLSCSPKHGWCVWDWTFRK

SEQ ID NO: 167 - protein, 41 aa, Artificial Sequence
  Description of Artificial Sequence Synthetic polypeptide
  MOD_RES (3)..(3): Gly, Gln, Val or absent
  MOD_RES (4)..(4): Val, Arg, Lys or absent
  MOD_RES (5)..(5): Pro, Ile, Phe or absent
  MOD_RES (6)..(6): Ile, Thr or Leu
  MOD_RES (7)..(7): Asn, Asp, Pro or Ser
  MOD_RES (8)..(8): Val, Ile or Asn
  MOD_RES (9)..(9): Lys, Arg or Ser
  MOD_RES (11)..(11): Arg, Lys, Asn, Ser or Thr
  MOD_RES (12)..(12): Gly, Ile, His, Asn or Ala
  MOD_RES (14)..(14): Arg, Lys, Gly, Ser or Tyr
  MOD_RES (15)..(15): Asp, Gln or Glu
  MOD_RES (17)..(17): Leu, Ile, Phe or Trp
  MOD_RES (18)..(18): Pro, Asp, Glu, Arg or Lys
  MOD_RES (19)..(19): Pro, Val or His
  MOD_RES (21)..(21): Arg, Lys or Ile
  MOD_RES (22)..(22): Lys, Arg, Asp, Ser or Gln
  MOD_RES (23)..(23): Ala, Ile, Arg, Lys or Met
  MISC_FEATURE (24)..(24): May or may not be present
  MOD_RES (26)..(26): Met, Lys, Arg or Thr
  MOD_RES (27)..(27): Arg, Lys, Thr or Pro
  MOD_RES (28)..(28): Phe, Asn or Ala
  MOD_RES (29)..(29): Gly or Ala
  MOD_RES (30)..(30): Lys or Arg
  MOD_RES (32)..(32): Ile, Met or Val
  MOD_RES (34)..(34): Ser, Gly, Arg or Lys
  MOD_RES (35)..(35): Lys, Arg or Leu
  MOD_RES (37)..(37): His, Asp, Tyr, Arg or Lys
  MOD_RES (39)..(39): Thr, Tyr or Phe
  MOD_RES (40)..(40): Pro, Ser or absent
  MOD_RES (41)..(41): Lys, Arg or absent
GSXXXXXXXCXXSXXCXXXCXXXFGXXXXXCXNXXCXCXXX

SEQ ID NO: 168 - protein, 40 aa, Artificial Sequence
  Description of Artificial Sequence Synthetic polypeptide
  MOD_RES (3)..(3): Gly, Gln, Val or absent
  MOD_RES (4)..(4): Val, Arg, Lys or absent
  MOD_RES (5)..(5): Pro, Ile, Phe or absent
  MOD_RES (7)..(7): Asn, Asp, Pro or Ser
  MOD_RES (9)..(9): Lys or Arg
  MOD_RES (11)..(11): Arg, Lys, Asn, Ser or Thr
  MOD_RES (12)..(12): Gly, Ile, His, Asn or Ala
  MOD_RES (14)..(14): Arg, Lys, Gly, Ser or Tyr
  MOD_RES (18)..(18): Pro, Asp, Glu, Lys or Arg
  MOD_RES (21)..(21): Lys or Arg
  MOD_RES (22)..(22): Lys, Arg, Asp, Ser or Gln
  MOD_RES (26)..(26): Lys or Arg
  MOD_RES (29)..(29): Lys or Arg
  MOD_RES (31)..(31): Ile, Met or Val
  MOD_RES (34)..(34): Lys or Arg
  MOD_RES (36)..(36): His, Asp, Tyr, Lys or Arg
  MOD_RES (40)..(40): Lys, Arg or absent
GSXXXIXVXCXXSXQCLXPCXXAGMXFGXCXNGXCXCTPX

SEQ ID NO: 169 - protein, 40 aa, Artificial Sequence
  Description of Artificial Sequence Synthetic polypeptide
  MOD_RES (5)..(5): Pro or Ile
  MOD_RES (9)..(9): Lys or Arg
  MOD_RES (11)..(11): Lys or Arg
  MOD_RES (12)..(12): Gly or Ile
  MOD_RES (14)..(14): Lys or Arg
  MOD_RES (15)..(15): Asp or Gln
  MOD_RES (18)..(18): Asp or Glu
  MOD_RES (21)..(21): Lys or Arg
  MOD_RES (22)..(22): Lys or Arg
  MOD_RES (26)..(26): Lys or Arg
  MOD_RES (29)..(29): Lys or Arg
  MOD_RES (31)..(31): Ile or Met
  MOD_RES (33)..(33): Ser or Gly
  MOD_RES (34)..(34): Lys or Arg
  MOD_RES (40)..(40): Lys, Arg or absent
GSGVXINVXCXXSXXCLXPCXXAGMXFGXCXNXXCHCTPX

SEQ ID NO: 170 - protein, 40 aa, Artificial Sequence
  Description of Artificial Sequence Synthetic polypeptide
  MOD_RES (3)..(3): Gly or Val
  MOD_RES (4)..(4): Val, Arg or Lys
  MOD_RES (5)..(5): Pro, Ile or absent
  MOD_RES (7)..(7): Asn or Pro
  MOD_RES (9)..(9): Lys, Ser or Arg
  MOD_RES (11)..(11): Lys or Arg
  MOD_RES (12)..(12): Gly, Ile or His
  MOD_RES (14)..(14): Arg, Gly or Lys
  MOD_RES (15)..(15): Gln or Asp
  MOD_RES (18)..(18): Asp, Glu, Lys or Arg
  MOD_RES (21)..(21): Lys or Arg
  MOD_RES (22)..(22): Lys, Asp or Arg
  MOD_RES (29)..(29): Lys or Arg
  MOD_RES (31)..(31): Met or Ile
  MOD_RES (33)..(33): Gly or Ser
  MOD_RES (34)..(34): Lys or Arg
  MOD_RES (36)..(36): His or Asp
  MOD_RES (40)..(40): Lys or Arg
GSXXXIXVXCXXSXXCLXPCXXAGMRFGXCXNXXCXCTPX

SEQ ID NO: 171 - protein, 40 aa, Artificial Sequence
  Description of Artificial Sequence Synthetic polypeptide
  MISC_FEATURE (3)..(3): May or may not be present
  MOD_RES (4)..(4): Lys, Arg or absent
  MOD_RES (5)..(5): Ile, Pro or Phe
  MOD_RES (6)..(6): Thr or Leu
  MOD_RES (7)..(7): Asp or Ser
  MOD_RES (8)..(8): Asn, Ile or Val
  MOD_RES (9)..(9): Lys or Arg
  MOD_RES (11)..(11): Asn, Ser or Thr
  MOD_RES (12)..(12): Asn, Ala or Gly
  MOD_RES (14)..(14): Ser, Tyr, Lys or Arg
  MOD_RES (15)..(15): Gln or Glu
  MOD_RES (17)..(17): Ile, Phe or Trp
  MOD_RES (19)..(19): Val or His
  MOD_RES (21)..(21): Lys, Ile or Arg
  MOD_RES (22)..(22): Arg, Ser, Gln or Lys
  MOD_RES (23)..(23): Ile, Arg, Met or Lys
  MOD_RES (26)..(26): Lys, Thr or Arg
  MOD_RES (27)..(27): Arg, Thr, Pro or Lys
  MOD_RES (28)..(28): Asn or Ala
  MOD_RES (29)..(29): Gly or Ala
  MOD_RES (30)..(30): Lys or Arg
  MOD_RES (32)..(32): Ile, Val or Met
  MOD_RES (34)..(34): Gly, Arg or Lys
  MOD_RES (35)..(35): Lys, Leu or Arg
  MOD_RES (37)..(37): Tyr, Asp, Arg or Lys
  MOD_RES (39)..(39): Tyr or Phe
  MOD_RES (40)..(40): Pro, Ser or absent
GSQXXXXXXCXXSXXCXPXCXXXFGXXXXXCXNXXCXCXX

SEQ ID NO: 172 - protein, 39 aa, Artificial Sequence
  Description of Artificial Sequence Synthetic polypeptide
  MOD_RES (1)..(1): Gly, Gln, Val or absent
  MOD_RES (2)..(2): Val, Arg, Lys or absent
  MOD_RES (3)..(3): Pro, Ile, Phe or absent
  MOD_RES (4)..(4): Ile, Thr or Leu
  MOD_RES (5)..(5): Asn, Asp, Pro or Ser
  MOD_RES (6)..(6): Val, Ile or Asn
  MOD_RES (7)..(7): Lys, Arg or Ser
  MOD_RES (9)..(9): Arg, Lys, Asn, Ser or Thr
  MOD_RES (10)..(10): Gly, Ile, His, Asn or Ala
  MOD_RES (12)..(12): Arg, Lys, Gly, Ser or Tyr
  MOD_RES (13)..(13): Asp, Gln or Glu
  MOD_RES (15)..(15): Leu, Ile, Phe or Trp
  MOD_RES (16)..(16): Pro, Asp, Glu, Arg or Lys
  MOD_RES (17)..(17): Pro, Val or His
  MOD_RES (19)..(19): Arg, Lys or Ile
  MOD_RES (20)..(20): Lys, Arg, Asp, Ser or Gln
  MOD_RES (21)..(21): Ala, Ile, Arg, Lys or Met
  MISC_FEATURE (22)..(22): May or may not be present
  MOD_RES (24)..(24): Met, Lys, Arg or Thr
  MOD_RES (25)..(25): Arg, Lys, Thr or Pro
  MOD_RES (26)..(26): Phe, Asn or Ala
  MOD_RES (27)..(27): Gly or Ala
  MOD_RES (28)..(28): Lys or Arg
  MOD_RES (30)..(30): Ile, Met or Val
  MOD_RES (32)..(32): Ser, Gly, Arg or Lys
  MOD_RES (33)..(33): Lys, Arg or Leu
  MOD_RES (35)..(35): His, Asp, Tyr, Arg or Lys
  MOD_RES (37)..(37): Thr, Tyr of Phe
  MOD_RES (38)..(38): Pro, Ser or absent
  MOD_RES (39)..(39): Lys, Arg or absent
XXXXXXXCXXSXXCXXXCXXXFGXXXXXCXNXXCXCXXX

SEQ ID NO: 173 - protein, 38 aa, Artificial Sequence
  Description of Artificial Sequence Synthetic polypeptide
  MOD_RES (1)..(1): Gly, Gln, Val or absent
  MOD_RES (2)..(2): Val, Arg, Lys or absent
  MOD_RES (3)..(3): Pro, Ile, Phe or absent
  MOD_RES (5)..(5): Asn, Asp, Pro or Ser
  MOD_RES (7)..(7): Lys or Arg
  MOD_RES (9)..(9): Arg, Lys, Asn, Ser or Thr
  MOD_RES (10)..(10): Gly, Ile, His, Asn or Ala
  MOD_RES (12)..(12): Arg, Lys, Gly, Ser or Tyr
  MOD_RES (16)..(16): Pro, Asp, Glu, Lys or Arg
  MOD_RES (19)..(19): Lys or Arg
  MOD_RES (20)..(20): Lys, Arg, Asp, Ser or Gln
  MOD_RES (24)..(24): Lys or Arg
  MOD_RES (27)..(27): Lys or Arg
  MOD_RES (29)..(29): Ile, Met or Val
  MOD_RES (32)..(32): Lys or Arg
  MOD_RES (34)..(34): His, Asp, Tyr, Lys or Arg
  MOD_RES (38)..(38): Lys, Arg or absent
XXXIXVXCXXSXQCLXPCXXAGMXFGXCXNGXCXCTPX

SEQ ID NO: 174 - protein, 38 aa, Artificial Sequence
  Description of Artificial Sequence Synthetic polypeptide
  MOD_RES (3)..(3): Pro or Ile
  MOD_RES (7)..(7): Lys or Arg
  MOD_RES (9)..(9): Lys or Arg
  MOD_RES (10)..(10): Gly or Ile
  MOD_RES (12)..(12): Lys or Arg
  MOD_RES (13)..(13): Asp or Gln
  MOD_RES (16)..(16): Asp or Glu
  MOD_RES (19)..(19): Lys or Arg
  MOD_RES (20)..(20): Lys or Arg
  MOD_RES (24)..(24): Lys or Arg
  MOD_RES (27)..(27): Lys or Arg
  MOD_RES (29)..(29): Ile or Met
  MOD_RES (31)..(31): Ser or Gly
  MOD_RES (32)..(32): Lys or Arg
  MOD_RES (38)..(38): Lys, Arg or absent
GVXINVXCXXSXXCLXPCXXAGMXFGXCXNXXCHCTPX

SEQ ID NO: 175 - protein, 38 aa, Artificial Sequence
  Description of Artificial Sequence Synthetic polypeptide
  MOD_RES (1)..(1): Gly or Val
  MOD_RES (2)..(2): Val, Arg or Lys
  MOD_RES (3)..(3): Pro, Ile or absent
  MOD_RES (5)..(5): Asn or Pro
  MOD_RES (7)..(7): Lys, Ser or Arg
  MOD_RES (9)..(9): Lys or Arg
  MOD_RES (10)..(10): Gly, Ile or His
  MOD_RES (12)..(12): Arg, Gly or Lys
  MOD_RES (13)..(13): Gln or Asp
  MOD_RES (16)..(16): Asp, Glu, Lys or Arg
  MOD_RES (19)..(19): Lys or Arg
  MOD_RES (20)..(20): Lys, Asp or Arg
  MOD_RES (27)..(27): Lys or Arg
  MOD_RES (29)..(29): Met or Ile
  MOD_RES (31)..(31): Gly or Ser
  MOD_RES (32)..(32): Lys or Arg
  MOD_RES (34)..(34): His or Asp
  MOD_RES (38)..(38): Lys or Arg
XXXIXVXCXXSXXCLXPCXXAGMRFGXCXNXXCXCTPX

SEQ ID NO: 176 - protein, 38 aa, Artificial Sequence
  Description of Artificial Sequence Synthetic polypeptide
  MISC_FEATURE (1)..(1): May or may not be present
  MOD_RES (2)..(2): Lys, Arg or absent
  MOD_RES (3)..(3): Ile, Pro or Phe
  MOD_RES (4)..(4): Thr or Leu
  MOD_RES (5)..(5): Asp or Ser
  MOD_RES (6)..(6): Asn, Ile or Val
  MOD_RES (7)..(7): Lys or Arg
  MOD_RES (9)..(9): Asn, Ser or Thr
  MOD_RES (10)..(10): Asn, Ala or Gly
  MOD_RES (12)..(12): Ser, Tyr, Lys or Arg
  MOD_RES (13)..(13): Gln or Glu
  MOD_RES (15)..(15): Ile, Phe or Trp
  MOD_RES (17)..(17): Val or His
  MOD_RES (19)..(19): Lys, Ile or Arg
  MOD_RES (20)..(20): Arg, Ser, Gln or Lys
  MOD_RES (21)..(21): Ile, Arg, Met or Lys
  MOD_RES (24)..(24): Lys, Thr or Arg
  MOD_RES (25)..(25): Arg, Thr, Pro or Lys
  MOD_RES (26)..(26): Asn or Ala
  MOD_RES (27)..(27): Gly or Ala
  MOD_RES (28)..(28): Lys or Arg
  MOD_RES (30)..(30): Ile, Val or Met
  MOD_RES (32)..(32): Gly, Arg or Lys
  MOD_RES (33)..(33): Lys, Leu or Arg
  MOD_RES (35)..(35): Tyr, Asp, Arg or Lys
  MOD_RES (37)..(37): Tyr or Phe
  MOD_RES (38)..(38): Pro, Ser or absent
QXXXXXXCXXSXXCXPXCXXXFGXXXXXCXNXXCXCXX

SEQ ID NO: 177 - protein, 39 aa, Artificial Sequence
  Description of Artificial Sequence Synthetic polypeptide
GSGVPINVRSRGSRDSLDPSRRAGMRFGRSINSRSHSTP

SEQ ID NO: 178 - protein, 5 aa, Artificial Sequence
  Description of Artificial Sequence Synthetic peptide
  MOD_RES (2)..(3): Any amino acid
LXXLF

SEQ ID NO: 179 - protein, 81 aa, Artificial Sequence
  Description of Artificial Sequence Synthetic polypeptide
  MOD_RES (2)..(16): Any amino acid
  MISC_FEATURE (2)..(16): This region may encompass 0-15 residues
  MOD_RES (18)..(32): Any amino acid
  MISC_FEATURE (18)..(32): This region may encompass 0-15 residues
  MOD_RES (34)..(48): Any amino acid
  MISC_FEATURE (34)..(48): This region may encompass 0-15 residues
  MOD_RES (50)..(64): Any amino acid
  MISC_FEATURE (50)..(64): This region may encompass 0-15 residues
  MOD_RES (66)..(80): Any amino acid
  MISC_FEATURE (66)..(80): This region may encompass 0-15 residues
CXXXXXXXXXXXXXXXCXXXXXXXXXXXXXXXCXXXXXXXXXXXXXXXCXXXXXXXXXXXXXXXCXXXXXXXXXXXXXXXC

SEQ ID NO: 180 - protein, 6 aa, Artificial Sequence
  Description of Artificial Sequence Synthetic 6xHhis tag
HHHHHH

SEQ ID NO: 181 - protein, 4 aa, Artificial Sequence
  Description of Artificial Sequence Synthetic polypeptide
GAGA

SEQ ID NO: 182 - protein, 28 aa, Artificial Sequence
  Description of Artificial Sequence Synthetic peptide
  MISC_FEATURE (1)..(28): This sequence may encompass 1-7 ′Gly Gly Gly Ser′ repeating units
GGGSGGGSGGGSGGGSGGGSGGGSGGGS